(19) United States
(12) Patent Application Publication
(10) Pub. No.: US 2014/0219992 A1
(43) Pub. Date: Aug. 7, 2014

(54) Metal Sensitive Mutants of Matrix Metalloproteases and uses thereof

(71) Applicants: Rudolph D. Paladini, Encinitas, CA (US); Ge Wei, San Diego, CA (US); H. Michael Shepard, San Diego, CA (US)

(72) Inventors: Rudolph D. Paladini, Encinitas, CA (US); Ge Wei, San Diego, CA (US); H. Michael Shepard, San Diego, CA (US)

(21) Appl. No.: 13/999,061

(22) Filed: Jan. 7, 2014

Related U.S. Application Data
(60) Provisional application No. 61/848,646, filed on Jan. 7, 2013.

(51) Int. Cl. A61K 38/48; A61K 33/06

(52) U.S. Cl. CPC A61K 38/4886; A61K 33/06
USPC 424/94.67

(57) ABSTRACT

Provided herein are methods of using modified matrix metalloprotease (MMP) enzymes that exhibit regulated activity in the presence of calcium. The methods include conditionally controlling the activity of the MMPs through the use of calcium to treat fibrotic diseases or conditions involving a component of the extracellular matrix (ECM).

### RELATED APPLICATIONS

Benefit of priority is claimed to U.S. Provisional Application Ser. No. 61/848,646, filed Jan. 7, 2013, to Rudolph D. Paladini, Ge Wei, and H. Michael Shepard, entitled “METAL SENSITIVE MUTANTS OF MATRIX METALLOPROTEASES AND USES THEREOF.”

This application is related to International PCT Application No. (Attorney Dkt. No. 33320.003098.WO01/3098PC), filed the same day herewith, to Rudolph D. Paladini, Ge Wei, and H. Michael Shepard, entitled “METAL SENSITIVE MUTANTS OF MATRIX METALLOPROTEASES AND USES THEREOF,” which also claims priority to U.S. Provisional Application Ser. No. 61/848,646.

This application also is related to U.S. application Ser. No. 12/660,894, filed Mar. 5, 2010, to Louis Bookbinder, Gregory I. Frost, Gilbert Keller, Gerhard Johann Frey, Hwai Wen Chang and Jay Milton Short, entitled “TEMPERATURE SENSITIVE MUTANTS OF MATRIX METALLOPROTEASES AND USES THEREOF,” which claims priority to U.S. Provisional Application Ser. No. 61/209,366, filed Mar. 6, 2009.

The subject matter of each of the above-noted applications is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy #1 and Copy #2), the contents of which are incorporated by reference in their entirety. The computer-readable file on each of the aforementioned compact discs, created on Jan. 7, 2014, is identical, 712 kilobytes in size, and titled 3098SEQ.001.txt.

### FIELD OF THE INVENTION

Provided herein are methods of using modified matrix metalloprotease (MMP) enzymes that exhibit regulated activity in the presence of calcium. The methods include conditionally controlling the activity of the MMPs through the use of calcium to treat fibrotic diseases or conditions involving a component of the extracellular matrix (ECM).

### BACKGROUND

The extracellular matrix (ECM) provides structural support for cells and tissues. Defects or changes in the extracellular matrix as a result of excessive deposition or accumulation of ECM components, such as collagen, can lead to fibrotic disease or conditions. Among these are collagen-mediated diseases or conditions characterized by the presence of abundant collagen fibers. Often the only approved treatment for such diseases or conditions is surgery, which can be highly invasive. These include, for example, needle aponeurotomy for the treatment of Dupuytren's syndrome or liposuction for cellulite, which are highly invasive. Other treatments, such as the use of bacterial collagenase, is associated with side effects due to prolonged degradation of collagen. Hence, there is a need for alternative treatments of fibrotic diseases and conditions. Accordingly, it is among the objects herein to provide alternative methods for the treatment of fibrotic diseases and conditions.

### SUMMARY

Provided are methods of treating a fibrotic disease or condition by administering, to a locus of a subject to be treated, a modified matrix metalloprotease (MMP) or a catalytically active fragment thereof to degrade a component of the extracellular matrix to effect treatment of the disease or condition, wherein the modified MMP contains a modification in an unmodified MMP polypeptide or catalytically active fragment thereof that is an amino acid insertion, deletion or replacement, and the modification confers, to the modified MMP or catalytically active fragment thereof, reduced activity in the presence of physiological levels of extracellular calcium compared to its activity in the presence of a calcium concentration that is greater than the physiological level, whereby the activity of the modified MMP decreases upon exposure to physiological conditions. The methods provided further include administering, at or near the same locus, a composition containing calcium in a concentration that is greater than physiological levels of extracellular calcium, whereby the modified MMP is conditionally active after administration so that the component of the extracellular matrix is degraded for a limited time to thereby treat the disease or condition.

In any of the methods herein, the modified MMP and calcium composition can be administered separately or together in the same composition. The calcium composition can be administered prior to, intermittently with, subsequently to, or simultaneously with administration of the modified MMP or catalytically active fragment. In examples of methods where the modified MMP and calcium composition are administered together, the administered composition contains the modified MMP and can contain calcium in a concentration that is greater than physiological levels of extracellular calcium. In any of the examples of the methods provided herein, the modified MMP or catalytically active fragment is an active enzyme that cleaves a component of the ECM.

In any of the methods herein, the modified MMP or catalytically active fragment used in the methods provided herein exhibits reduced activity in the presence of physiological levels of extracellular calcium compared to its activity in the presence of a calcium concentration that is greater than the physiological level. Physiological levels include calcium concentrations of about 1 mM to 1.3 mM calcium. Thus, exemplary calcium concentrations that are greater than physiological levels include, but are not limited to, 1.5 mM to 100 mM, 2 mM to 50 mM, 2 mM to 25 mM, 5 mM to 50 mM, 5 mM to 25 mM, 5 mM to 15 mM, and 8 mM to 12 mM calcium. Exemplary calcium concentrations that are greater than physiological levels also include calcium concentrations that are at least or about at least or 1.5 mM, including at least or about at least 2.0 mM, 2.5 mM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5, mM, 9.0 mM, 9.5 mM, 10.0 mM, 11 mM, 12 mM, 13 mM, 14 mM, 15 mM, 16 mM, 17 mM, 18 mM, 19 mM and 20 mM. Thus, a composition containing calcium in a concentration that is greater than physiological levels of extracellular calcium includes, but is not limited to, a composition containing 1.5 mM to 100 mM, 2 mM to 50 mM, 2 mM to 25 mM, 5 mM to 50 mM, 5 mM to 25 mM, 5 mM to 15 mM, or 8 mM to 12 mM calcium. Also, a composition containing calcium in a concentration that is greater than physiological levels of extracellular calcium can contain at least or about at least or 1.5 mM, including at least or about at least 2.0 mM, 2.5 mM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5, mM, 9.0 mM, 9.5 mM, 10.0 mM, 11 mM, 12 mM, 13 mM, 14 mM, 15 mM, 16 mM, 17 mM, 18 mM, 19 mM and 20 mM. In particular, a composition that contains at least or about at least 10 mM calcium are provided in the methods herein.

Also provided are pharmaceutical compositions for temporal or conditional cleavage of a component of the ECM for use in treating a fibrotic disease or condition, wherein the pharmaceutical composition contains a modified matrix metalloprotease (MMP) or a catalytically active fragment thereof and a concentration of calcium that is greater than the physiological concentration. In such examples, the modified MMP contains a modification in an unmodified MMP polypeptide or catalytically active fragment thereof that is an amino acid insertion, deletion or replacement; the modified MMP degrades a component of the extracellular matrix to thereby effect treatment of the disease or condition; and the modification confers to the modified MMP or catalytically active fragment thereof reduced activity in the presence of physiological levels of extracellular calcium compared to its activity in the presence of a calcium concentration that is greater than the physiological level, whereby the activity of the modified MMP decreases upon exposure to physiological conditions.

Also provided are uses of a pharmaceutical composition for formulation of a medicament for temporal or conditional cleavage of a component of the ECM for treatment of a fibrotic disease or condition, where the composition contains a modified matrix metalloprotease (MMP) or a catalytically active fragment thereof and a concentration of calcium that is greater than the physiological concentration. In such examples, the modified MMP contains a modification in an unmodified MMP polypeptide or catalytically active fragment thereof that is an amino acid insertion, deletion or replacement; the modified MMP degrades a component of the extracellular matrix to thereby effect treatment of the disease or condition; and the modification confers to the modified MMP or catalytically active fragment thereof reduced activity in the presence of physiological levels of extracellular calcium compared to its activity in the presence of a calcium concentration that is greater than the physiological level, whereby the activity of the modified MMP decreases upon exposure to physiological conditions.

In any of the pharmaceutical compositions or uses herein, the modified MMP or catalytically active fragment contained in the provided pharmaceutical compositions for use in treating a fibrotic disease or condition, or used to formulate a medicament for treatment of a fibrotic disease or condition, exhibits reduced activity in the presence of physiological levels of extracellular calcium compared to its activity in the presence of a calcium concentration that is greater than the physiological level. Such physiological levels of calcium include calcium concentrations of about 1 mM to 1.3 mM calcium. Thus, exemplary calcium concentrations that are greater than physiological levels include, but are not limited to, 1.5 mM to 100 mM, 2 mM to 50 mM, 2 mM to 25 mM, 5 mM to 50 mM, 5 mM to 25 mM, 5 mM to 15 mM, and 8 mM to 12 mM calcium. Hence, the pharmaceutical compositions containing calcium concentrations that are greater than physiological levels include calcium concentrations that are at least or about at least or 1.5 mM, including at least or about at least 2.0 mM, 2.5 mM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5, mM, 9.0 mM, 9.5 mM, 10.0 mM, 11 mM, 12 mM, 13 mM, 14 mM, 15 mM, 16 mM, 17 mM, 18 mM, 19 mM and 20 mM. Thus, the provided pharmaceutical compositions, or uses of a pharmaceutical composition to form a medicament, containing calcium in a concentration that is greater than physiological levels of extracellular calcium include, but are not limited to, those containing 1.5 mM to 100 mM, 2 mM to 50 mM, 2 mM to 25 mM, 5 mM to 50 mM, 5 mM to 25 mM, 5 mM to 15 mM, or 8 mM to 12 mM calcium. Also, the provided pharmaceutical compositions or uses contain calcium in a concentration that is greater than physiological levels of extracellular calcium that is at least or about at least or 1.5 mM, including at least or about at least 2.0 mM, 2.5 mM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5, mM, 9.0 mM, 9.5 mM, 10.0 mM, 11 mM, 12 mM, 13 mM, 14 mM, 15 mM, 16 mM, 17 mM, 18 mM, 19 mM and 20 mM. In particular, a pharmaceutical composition, or use of a pharmaceutical composition to form a medicament, as provided herein, can contain at least or about at least 10 mM calcium.

In any of the methods, pharmaceutical compositions or uses herein, the modified MMP or catalytically active fragment administered in the provided methods can exhibit at least 5%, such as at least 10%, 15%, 20%, 25%, 30%, 40%, 50%, 60%, 70%, 80%, 90%, 95%, 100%, 110%, 120%, 130%, 140%, 150%, 160%, 170%, 180%, 190%, 200%, 250%, 300%, 350%, 400%, 450%, 500% or more, of the activity of the unmodified MMP at calcium concentrations greater than physiological levels of calcium. In any of the methods provided, the modified MMP or catalytically active fragment thereof can exhibit reduced activity at physiological levels of extracellular calcium compared to the activity of the corresponding unmodified MMP, which does not contain the modification(s). In methods wherein the modified MMP or catalytically active fragment exhibits reduced activity compared to the unmodified MMP at physiological levels of calcium, the modified MMP or catalytically active fragment can exhibit less than 100% of the activity of the corresponding unmodified MMP not containing the modification(s). Included are methods of administering a modified MMP that exhibits less than 95%, 90%, 85%, 80%, 75%, 70%, 65%, 60%, 55%, 50%, 45%, 40%, 35%, 30%, 25%, 20%, 15%, 10%, 5% or less of the activity of the corresponding unmodified MMP not containing the modification(s).

In any of the methods, pharmaceutical compositions or uses herein, modifications of the MMP or catalytically active fragment thereof can include amino acid replacements. Any of the provided methods include administering a modified MMP or catalytically active fragment that contains a modification at or near an amino acid that is a metal-binding site. Included are methods of treating a fibrotic disease or condition by administering a modified MMP or catalytically active fragment that contains a modification at or near a zinc- or calcium-binding site. The administered modified MMP or catalytically active fragment can contain a modification at an amino acid corresponding to a position selected from among 102, 103, 104, 105, 106, 107, 108, 136, 137, 138, 139, 140, 141, 142, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 196, 197, 198, 199, 201, 202, 203, 204, 205, 206, 207, 208, 209, 210, 211, 212, 263, 264, 265, 266, 267, 268, 269, 307, 308, 309, 310, 311, 312, 313, 356, 357, 358, 359, 360, 361, 362, 405, 406, 407, 408, 409, 410 and 411 with reference to amino acid positions set forth in SEQ ID NO:2, wherein corresponding amino acid positions are identified by alignment of the MMP polypeptide with the polypeptide set forth in SEQ ID NO:2.

In any of the methods, pharmaceutical compositions or uses herein, exemplary modifications of the MMP or catalytically active fragment thereof administered in the provided methods include an amino acid replacement selected from among replacement with: R at a position corresponding to position 102; K at a position corresponding to position 102; V at a position corresponding to position 102; M at a position corresponding to position 102; P at a position corresponding to position 102; N at a position corresponding to position 102; G at a position corresponding to position 102; L at a position corresponding to position 102; D at a position corresponding to position 102; S at a position corresponding to position 102; F at a position corresponding to position 102; A at a position corresponding to position 102; E at a position corresponding to position 102; Q at a position corresponding to position 102; C at a position corresponding to position 102; N at position corresponding to position 103; E at a position corresponding to position 104; T at a position corresponding to position 104; R at a position corresponding to position 104; D at a position corresponding to position 104; Q at a position corresponding to position 104; V at a position corresponding to position 104; Y at a position corresponding to position 104; H at a position corresponding to position 104; L at a position corresponding to position 104; A at a position corresponding to position 104; M at a position corresponding to position 104; A at a position corresponding to position 105; C at a position corresponding to position 105; F at a position corresponding to position 105; G at a position corresponding to position 105; I at a position corresponding to position 105; L at a position corresponding to position 105; M at a position corresponding to position 105; N at a position corresponding to position 105; P at a position corresponding to position 105; R at a position corresponding to position 105; S at a position corresponding to position 105; T at a position corresponding to position 105; V at a position corresponding to position 105; W at a position corresponding to position 105; E at a position corresponding to position 105; M at a position corresponding to position 106; A at a position corresponding to position 106; Y at a position corresponding to position 106; V at a position corresponding to position 106; I at a position corresponding to position 106; L at a position corresponding to position 107; T at a position corresponding to position 107; S at a position corresponding to position 107; R at a position corresponding to position 107; M at a position corresponding to position 107; V at a position corresponding to position 107; D at a position corresponding to position 107; A at a position corresponding to position 107; K at a position corresponding to position 107; G at a position corresponding to position 107; P at a position corresponding to position 108; G at a position corresponding to position 108; E at a position corresponding to position 108; A at a position corresponding to position 108; Y at a position corresponding to position 108; K at a position corresponding to position 108; C at a position corresponding to position 108; S at a position corresponding to position 108; S at a position corresponding to position 108; F at a position corresponding to position 108; I at a position corresponding to position 108; L at a position corresponding to position 108; N at a position corresponding to position 108; D at a position corresponding to position 136; M at a position corresponding to position 136; N at a position corresponding to position 136; A at a position corresponding to position 136; L at a position corresponding to position 136; P at a position corresponding to position 136; T at a position corresponding to position 136; R at a position corresponding to position 136; S at a position corresponding to position 136; H at a position corresponding to position 136; E at a position corresponding to position 136; A at a position corresponding to position 137; R at a position corresponding to position 137; G at a position corresponding to position 137; K at a position corresponding to position 137; H at a position corresponding to position 137; P at a position corresponding to position 137; S at a position corresponding to position 137; L at a position corresponding to position 137; W at a position corresponding to position 137; F at a position corresponding to position 137; T at a position corresponding to position 137; Y at a position corresponding to position 137; E at a position corresponding to position 137; G at a position corresponding to position 138; R at a position corresponding to position 139; V at a position corresponding to position 139; M at a position corresponding to position 139; C at a position corresponding to position 139; P at a position corresponding to position 139; P at a position corresponding to position 139; S at a position corresponding to position 139; L at a position corresponding to position 139; I at a position corresponding to position 139; H at a position corresponding to position 139; A at a position corresponding to position 139; G at a position corresponding to position 139; F at a position corresponding to position 139; N at a position corresponding to position 139; W at a position corresponding to position 139; Y at a position corresponding to position 139; E at a position corresponding to position 139; E at a position corresponding to position 141; I at a position corresponding to position 141; R at a position corresponding to position 141; S at a position corresponding to position 141; L at a position corresponding to position 141; A at a position corresponding to position 141; D at a position corresponding to position 141; W at a position corresponding to position 141; H at a position corresponding to position 141; N at a position corresponding to position 141; L at a position corresponding to position 142; M at a position corresponding to position 142; V at a position corresponding to position 142; T at a position corresponding to position 146; N at a position corresponding to position 146; Q at a position corresponding to position 146; K at a position corresponding to position 146; S at a position corresponding to position 146; D at a position corresponding to position 146; A at a position corresponding to position 146; Y at a position corresponding to position 146; V at a position corresponding to position 146; R at a position corresponding to position 147; F at a position corresponding to position 147; H at a position corresponding to position 147; W at a position corresponding to position 147; T at a position corresponding to position 147; C at a position corresponding to position 147; S at a position corresponding to position 147; V at a position corresponding to position 147; Q at a position corresponding to position 147; M at a position corresponding to position 147; R at a position corresponding to position 148; R at a position corresponding to position 148; I at a position corresponding to position 148; T at a position corresponding to position 148; G at a position corresponding to position 148; G at a position corresponding to position 148; V at a position corresponding to position 148; A at a position corresponding to position 148; A at a position corresponding to position 148; W at a position corresponding to position 148; P at a position corresponding to position 148; S at a position corresponding to position 148; N at a position corresponding to position 148; S at a position corresponding to position 150; E at a position corresponding to position 150; G at a position corresponding to position 150; M at a position corresponding to position 150; M at a position corresponding to position 150; T at a position corresponding to position 150; W at a position corresponding to position 150; A at a position corresponding to position 150; N at a position corresponding to position 150; K at a position corresponding to position 150; L at a position corresponding to position 150; L at a position corresponding to position 150; V at a position corresponding to position 150; D at a position corresponding to position 150; H at a position corresponding to position 150; G at a position corresponding to position 152; C at a position corresponding to position 152; F at a position corresponding to position 152; L at a position corresponding to position 152; L at a position corresponding to position 152; L at a position corresponding to position 152; P at a position corresponding to position 152; R at a position corresponding to position 152; H at a position corresponding to position 152; T at a position corresponding to position 152; Y at a position corresponding to position 152; K at a position corresponding to position 152; D at a position corresponding to position 152; W at a position corresponding to position 152; I at a position corresponding to position 152; A at a position corresponding to position 152; S at a position corresponding to position 152; R at a position corresponding to position 152; G at a position corresponding to position 153; H at a position corresponding to position 153; V at a position corresponding to position 153; T at a position corresponding to position 153; P at a position corresponding to position 153; F at a position corresponding to position 153; D at a position corresponding to position 153; Q at a position corresponding to position 153; Y at a position corresponding to position 153; L at a position corresponding to position 154; C at a position corresponding to position 154; S at a position corresponding to position 154; I at a position corresponding to position 154; M at a position corresponding to position 155; H at a position corresponding to position 156; L at a position corresponding to position 156; E at a position corresponding to position 156; A at a position corresponding to position 156; W at a position corresponding to position 156; C at a position corresponding to position 156; P at a position corresponding to position 156; P at a position corresponding to position 156; V at a position corresponding to position 156; V at a position corresponding to position 156; K at a position corresponding to position 156; S at a position corresponding to position 156; G at a position corresponding to position 156; T at a position corresponding to position 156; Y at a position corresponding to position 156; R at a position corresponding to position 156; M at a position corresponding to position 156; K at a position corresponding to position 157; D at a position corresponding to position 157; F at a position corresponding to position 157; R at a position corresponding to position 157; H at a position corresponding to position 157; L at a position corresponding to position 157; N at a position corresponding to position 157; N at a position corresponding to position 157; Y at a position corresponding to position 157; S at a position corresponding to position 157; T at a position corresponding to position 157; A at a position corresponding to position 157; A at a position corresponding to position 157; Q at a position corresponding to position 157; P at a position corresponding to position 157; P at a position corresponding to position 157; V at a position corresponding to position 157; V at a position corresponding to position 157; M at a position corresponding to position 157; S at a position corresponding to position 158; Y at a position corresponding to position 158; R at a position corresponding to position 158; L at a position corresponding to position 158; V at a position corresponding to position 158; V at a position corresponding to position 158; C at a position corresponding to position 158; A at a position corresponding to position 158; W at a position corresponding to position 158; I at a position corresponding to position 158; F at a position corresponding to position 158; Q at a position corresponding to position 158; T at a position corresponding to position 158; G at a position corresponding to position 158; K at a position corresponding to position 158; N at a position corresponding to position 158; D at a position corresponding to position 158; R at a position corresponding to position 159; S at a position corresponding to position 159; Q at a position corresponding to position 159; P at a position corresponding to position 159; V at a position corresponding to position 159; K at a position corresponding to position 159; A at a position corresponding to position 159; Y at a position corresponding to position 159; E at a position corresponding to position 159; T at a position corresponding to position 159; M at a position corresponding to position 159; I at a position corresponding to position 159; W at a position corresponding to position 159; W at a position corresponding to position 159; L at a position corresponding to position 159; C at a position corresponding to position 159; A at a position corresponding to position 160; H at a position corresponding to position 160; N at a position corresponding to position 160; W at a position corresponding to position 160; R at a position corresponding to position 160; M at a position corresponding to position 160; Q at a position corresponding to position 160; V at a position corresponding to position 160; S at a position corresponding to position 160; E at a position corresponding to position 160; L at a position corresponding to position 160; T at a position corresponding to position 160; S at a position corresponding to position 161; C at a position corresponding to position 161; L at a position corresponding to position 161; R at a position corresponding to position 161; R at a position corresponding to position 161; G at a position corresponding to position G; W at a position corresponding to position 161; Y at a position corresponding to position 161; E at a position corresponding to position 161; P at a position corresponding to position 161; T at a position corresponding to position 161; H at a position corresponding to position 161; I at a position corresponding to position 161; V at a position corresponding to position 161; F at a position corresponding to position 161; Q at a position corresponding to position 161; S at a position corresponding to position 164; W at a position corresponding to position 166; D at a position corresponding to position 167; R at a position corresponding to position 167; A at a position corresponding to position 167; S at a position corresponding to position 167; S at a position corresponding to position 167; F at a position corresponding to position 167; Y at a position corresponding to position 167; P at a position corresponding to position 167; T at a position corresponding to position 167; V at a position corresponding to position 167; L at a position corresponding to position 167; M at a position corresponding to position 167; N at a position corresponding to position 167; G at a position corresponding to position 167; K at a position corresponding to position 167; E at a position corresponding to position 167; R at a position corresponding to position 168; L at a position corresponding to position 170; R at a position corresponding to position 170; R at a position corresponding to position 170; I at a position corresponding to position 170; T at a position corresponding to position 170; Q at a position corresponding to position 170; G at a position corresponding to position 170; S at a position corresponding to position 170; H at a position corresponding to position 170; M at a position corresponding to position 170; K at a position corresponding to position 170; S at a position corresponding to position 171; M at a position corresponding to position 171; N at a position corresponding to position 171; P at a position corresponding to position 171; R at a position corresponding to position 171; Y at a position corresponding to position 171; A at a position corresponding to position 171; Q at a position corresponding to position 171; H at a position corresponding to position 171; L at a position corresponding to position 171; W at a position corresponding to position 171; C at a position corresponding to position 171; K at a position corresponding to position 171; E at a position corresponding to position 171; D at a position corresponding to position 171; Y at a position corresponding to position 172; T at a position corresponding to position 172; P at a position corresponding to position 172; A at a position corresponding to position 172; L at a position corresponding to position 172; Q at a position corresponding to position 172; E at a position corresponding to position 172; M at a position corresponding to position 172; D at a position corresponding to position 172; V at a position corresponding to position 172; R at a position corresponding to position 172; W at a position corresponding to position 172; N at a position corresponding to position 172; C at a position corresponding to position 173; L at a position corresponding to position 173; K at a position corresponding to position 173; W at a position corresponding to position 173; W at a position corresponding to position 173; S at a position corresponding to position 173; A at a position corresponding to position 173; R at a position corresponding to position 173; N at a position corresponding to position 173; T at a position corresponding to position 173; D at a position corresponding to position 173; V at a position corresponding to position 173; F at a position corresponding to position 173; M at a position corresponding to position 173; Y at a position corresponding to position 173; P at a position corresponding to position 173; I at a position corresponding to position 175; T at a position corresponding to position 175; N at a position corresponding to position 175; V at a position corresponding to position 175; S at a position corresponding to position 175; R at a position corresponding to position 175; G at a position corresponding to position 175; A at a position corresponding to position 175; F at a position corresponding to position 175; C at a position corresponding to position 175; Q at a position corresponding to position 175; Y at a position corresponding to position 175; L at a position corresponding to position 175; H at a position corresponding to position 175; P at a position corresponding to position 175; E at a position corresponding to position 175; F at a position corresponding to position 176; Q at a position corresponding to position 176; V at a position corresponding to position 176; T at a position corresponding to position 176; C at a position corresponding to position 176; L at a position corresponding to position 176; P at a position corresponding to position 179; L at a position corresponding to position 179; E at a position corresponding to position 179; G at a position corresponding to position 179; G at a position corresponding to position 179; S at a position corresponding to position 179; A at a position corresponding to position 179; K at a position corresponding to position 179; T at a position corresponding to position 179; I at a position corresponding to position 179; R at a position corresponding to position 179; N at a position corresponding to position 179; W at a position corresponding to position 179; Q at a position corresponding to position 179; V at a position corresponding to position 179; C at a position corresponding to position 179; M at a position corresponding to position 180; P at a position corresponding to position 180; K at a position corresponding to position 180; Y at a position corresponding to position 180; Q at a position corresponding to position 180; R at a position corresponding to position 180; A at a position corresponding to position 180; T at a position corresponding to position 180; I at a position corresponding to position 180; F at a position corresponding to position 180; C at a position corresponding to position 180; G at a position corresponding to position 180; S at a position corresponding to position 180; N at a position corresponding to position 180; D at a position corresponding to position 180; S at a position corresponding to position 181; Q at a position corresponding to position 181; A at a position corresponding to position 181; T at a position corresponding to position 181; E at a position corresponding to position 181; C at a position corresponding to position 182; P at a position corresponding to position 182; P at a position corresponding to position 182; S at a position corresponding to position 182; T at a position corresponding to position 182; R at a position corresponding to position 182; D at a position corresponding to position 182; A at a position corresponding to position 182; F at a position corresponding to position 182; L at a position corresponding to position 182; I at a position corresponding to position 182; Y at a position corresponding to position 182; Q at a position corresponding to position 182; W at a position corresponding to position 182; M at a position corresponding to position 182; G at a position corresponding to position 182; K at a position corresponding to position 183; W at a position corresponding to position 183; W at a position corresponding to position 183; E at a position corresponding to position 183; A at a position corresponding to position 183; T at a position corresponding to position 183; N at a position corresponding to position 183; H at a position corresponding to position 183; V at a position corresponding to position 183; C at a position corresponding to position 183; M at a position corresponding to position 183; G at a position corresponding to position 183; S at a position corresponding to position 183; S at a position corresponding to position 185; C at a position corresponding to position 197; V at a position corresponding to position 201; M at a position corresponding to position 201; E at a position corresponding to position 203; A at a position corresponding to position 204; M at a position corresponding to position 205; I at a position corresponding to position 205; A at a position corresponding to position 207; M at a position corresponding to position 207; D at a position corresponding to position 208; V at a position corresponding to position 208; P at a position corresponding to position 208; G at a position corresponding to position 208; A at a position corresponding to position 208; K at a position corresponding to position 208; N at a position corresponding to position 208; F at a position corresponding to position 208; Q at a position corresponding to position 208; W at a position corresponding to position 208; T at a position corresponding to position 208; E at a position corresponding to position 208; C at a position corresponding to position 208; R at a position corresponding to position 208; L at a position corresponding to position 208; T at a position corresponding to position 210; P at a position corresponding to position 211; R at a position corresponding to position 211; K at a position corresponding to position 211; G at a position corresponding to position 211; M at a position corresponding to position 211; M at a position corresponding to position 211; N at a position corresponding to position 211; N at a position corresponding to position 211; V at a position corresponding to position 211; Q at a position corresponding to position 211; S at a position corresponding to position 211; A at a position corresponding to position 211; E at a position corresponding to position 212; T at a position corresponding to position 212; N at a position corresponding to position 212; S at a position corresponding to position 212; P at a position corresponding to position 212; Q at a position corresponding to position 212; F at a position corresponding to position 212; H at a position corresponding to position 212; and Y at a position corresponding to position 212, with reference to amino acid positions set forth in SEQ ID NO:2, wherein corresponding amino acid positions are identified by alignment of the MMP polypeptide with the polypeptide set forth in SEQ ID NO:2.

In any of the methods, pharmaceutical compositions or uses provided herein, the modified MMP or catalytically active fragment thereof is modified at an amino acid that is a calcium binding site. For example, in any of the methods herein, the method includes administering a modified MMP, whereby (i) the modified MMP contains an amino acid replacement in an unmodified MMP polypeptide or catalytically active fragment thereof at an amino acid residue that is a calcium-binding site; (ii) administration of the composition effects degradation of a component of the extracellular matrix to effect treatment of the disease or condition; (iii) the amino acid replacement confers to the modified MMP or catalytically active fragment thereof reduced activity in the presence of physiological levels of extracellular calcium compared to its activity in the presence of a calcium concentration that is greater than the physiological level, whereby its activity decreases upon exposure to physiological conditions such that the modified MMP is conditionally active after administration so that the component of the extracellular matrix is degraded for a limited time.

In any of the methods, pharmaceutical compositions or uses herein, the modified MMP or catalytically active fragment can contain a modification at a calcium-binding site that is at a position corresponding to the amino acid at position 105, 139, 156, 157, 159, 161, 171, 173, 175, 179, 180, 182, 266, 310, 359 or 408, with reference to the amino acid positions set forth in SEQ ID NO:2, wherein corresponding amino acid positions are identified by alignment of the MMP polypeptide with the polypeptide set forth in SEQ ID NO:2. Such modifications, with reference to MMP-1, include, but are not limited to, D105, D139, D156, G157, G159, N161, G171, G173, D175, D179, E180, E182, D266, E310, D359, and D408. In any example of the provided methods, the modification(s) can include an amino acid replacement at a position corresponding to the amino acid at position 105, 156, 179, 180, or 182, with reference to the amino acid positions set forth in SEQ ID NO:2.

Among the modified MMPs or catalytically active fragments thereof used in any of the provided methods, pharmaceutical compositions or uses herein, are modified MMPs in which the unmodified MMP contains an acidic amino acid at the modified amino acid position and the acidic amino acid is replaced by an amino acid residue that is a non-acidic amino acid residue. The acidic amino acid can be, for example, aspartic acid (D) or glutamic acid (E). The modified MMPs or catalytically active fragments include replacement of the acidic amino acid by: a neutral amino acid that is cysteine (C), asparagine (N), glutamine (Q), threonine (T), tyrosine (Y), serine (S) or glycine (G); a hydrophobic amino acid that is phenylalanine (F), methionine (M), tryptophan (W), isoleucine (I), valine (V), leucine (L), alanine (A) and proline (P); or a basic amino acid that is histidine (H), lysine (K) or arginine (R). Thus, exemplary amino acid replacements of the modified MMP active fragment, used in the provided method, include a replacement of A at a position corresponding to position 105; I at a position corresponding to position 105; N at a position corresponding to position 105; L at a position corresponding to position 105; G at a position corresponding to position 105; R at a position corresponding to position 156; H at a position corresponding to position 156; K at a position corresponding to position 156; T at a position corresponding to position 156; N at a position corresponding to position 179; T at a position corresponding to position 180; F at a position corresponding to position 180; and T at a position corresponding to position 182, with reference to the amino acid positions set forth in SEQ ID NO:2.

For example, in any of the methods, pharmaceutical compositions or uses herein, among these are modified MMPs or catalytic fragments that contain an amino acid replacement that is a replacement with T at a position corresponding to position 156, a replacement with N at a position corresponding to position 179, or a replacement with T at a position corresponding to position 156 and a replacement with N at a position corresponding to position 179.

In examples of any of the methods, pharmaceutical compositions or uses herein, also among these are modified MMPs or catalytic fragments that contain an amino acid replacement at an amino acid position corresponding to position 159 with reference to amino acid positions set forth in SEQ ID NO:2, wherein the amino acid replacement is replacement by a hydrophobic amino acid residue and corresponding amino acid positions are identified by alignment of the MMP polypeptide with the polypeptide set forth in SEQ ID NO:2. Such amino replacements include replacement by a hydrophobic amino acid such as phenylalanine (F), methionine (M), tryptophan (W), isoleucine (I), valine (V), leucine (L), alanine (A) or proline (P); or replacement by a neutral amino acid such as cysteine (C), asparagine (N), glutamine (Q), threonine (T), tyrosine (Y), serine (S) or glycine (G). An example of such a modification includes an amino acid replacement of V at a position corresponding to position 159, with reference to SEQ ID NO:2. The modified MMP polypeptide or catalytic fragment having a replacement of the amino acid of V at a position corresponding to position 159 also can contain an amino acid replacement of K at the amino acid position corresponding to position 208.

In any of the methods, pharmaceutical compositions or uses herein, also among the modified MMPs or catalytically active fragments thereof used in the provided methods that contain a replacement of an acidic amino acid at the modified amino acid position, is a modified MMP or catalytically active fragment thereof that contains an amino acid modification that is a replacement of the amino acid at a position corresponding to position 227 with glutamic acid (E), with reference to the amino acid positions set forth in SEQ ID NO:2. Included among these modified MMPs are those which contain a hydrophobic amino acid at the replaced position, such as valine (V). Thus, the replacement V227E, with reference to amino acid positions set forth in SEQ ID NO:2, is included among the modified MMPs and catalytic fragments which can be used in the methods provided herein.

In particular examples of any of the methods, pharmaceutical compositions or uses herein, the modified MMP polypeptide or fragment used in the provided methods can additionally include an amino acid replacement that is any replacement corresponding to the replacements set forth in Table 7, with reference to the amino acid position numbering set forth in SEQ ID NO:2.

In any of the provided methods, pharmaceutical compositions or uses herein, the methods can employ a modified MMP or catalytically active fragment with contains the sequence of amino acids set forth in any of SEQ ID NOS:162-167, or a sequence of amino acids that exhibits at least 70% sequence identity to any of SEQ ID NOS:162-167 and contains the amino acid replacement(s). Such a modified MMP or catalytically active fragment can be a sequence of amino acids that is at least 75%, 80%, 85%, 86%, 87%, 88%, 89%, 90%, 91%, 92%, 93%, 94%, 95%, 96%, 97%, 98%, 99% or more, sequence identity to any of SEQ ID NOS:162-167.

In any of the methods, pharmaceutical compositions or uses herein, the modified MMP or catalytically active fragment used in the methods provided herein exhibits reduced activity, such as less than 90%, 85%, 80%, 75%, 70%, 65%, 60%, 55%, 50%, 45%, 40%, 35%, 30%, 25%, 20%, 15%, 10%, 5%, 2% or less, matrix metalloprotease activity in the presence of physiological levels of extracellular calcium as compared to its activity in the presence of a calcium concentration that is greater than the physiological level. The modified MMP or catalytically active fragment can exhibit greater than 0.5-fold, 1-fold, 1.5-fold, 2-fold, 2.5-fold, 3-fold, 4-fold, 5-fold, 6-fold, 7-fold, 8-fold, 9-fold, 10-fold, 11-fold, 12-fold, 13-fold, 14-fold, 15-fold or 20-fold decreased matrix metalloprotease activity in the presence of physiological levels of extracellular calcium compared to its activity in the presence of a calcium concentration that is greater than the physiological level. In particular, the modified MMP or catalytically active fragment, used in the provided methods, exhibits at least 2-fold decreased matrix metalloprotease activity in the presence of physiological levels of extracellular calcium compared to its activity in the presence of a calcium concentration that is greater than the physiological level.

In any of the provided methods, pharmaceutical compositions or uses herein, the MMP effects temporal or conditional cleavage of a component of the ECM (e.g., collagen) for treatment of a fibrotic disease or condition by degrading a component of the extracellular matrix. The fibrotic disease or condition can be a collagen-mediated disease or condition, where the modified MMP or catalytically active fragment exhibits collagen cleavage activity to cleave the collagen component of the extracellular matrix. Such a component of the extracellular matrix can be selected from among collagen type I, collagen type II, collagen type III, collagen type IV, collagen type VI, collagen type VII, collagen type VIII, collagen type IX, collagen type X, collagen type XI and collagen type XIV. In particular, collagen type I, collagen type III, or collagen type I and collagen type III can be degraded in the provided methods to effect treatment of a collagen-mediated disease or condition.

In any of the methods, pharmaceutical compositions or uses herein, the modified MMP or catalytically active fragment that confers collagen cleavage activity can be a modified MMP that contains a modification in an unmodified MMP polypeptide or catalytically active fragment thereof selected from metalloprotease-1 (MMP-1), matrix metalloprotease-2 (MMP-2), matrix metalloprotease-3 (MMP-3), matrix-metalloprotease-7 (MMP-7), matrix metalloprotease-8 (MMP-8), matrix metalloprotease-9 (MMP-9), matrix metalloprotease-10 (MMP-10), matrix metalloprotease-11 (MMP-11), matrix-metalloprotease-12 (MMP-12), matrix metalloprotease 13 (MMP-13), matrix metalloprotease-14 (MMP-14), matrix metalloprotease-16 (MMP-16), matrix metalloprotease-18 (MMP-18), matrix metalloprotease-19, matrix metalloprotease-25 (MMP-25), and matrix metalloprotease-26 (MMP-26), or a catalytically active fragment thereof. Wherein the component of the extracellular matrix to be degraded is collagen type I, the unmodified MMP or catalytically active fragment can be selected from among, for example, a MMP-1, MMP-2, MMP-7, MMP-8, MMP-12, MMP-13, MMP-14 and MMP-18, or catalytically active fragment thereof. Wherein the component of the extracellular matrix to be degraded is collagen type III, the unmodified MMP or catalytically active fragment can be selected from among, for example, a MMP-1, MMP-2, MMP-3, MMP-8, MMP-10, MMP-13, MMP-14 and MMP-16, or catalytically active fragment thereof. Wherein collagen type I and collagen type III are the components of the extracellular matrix to be degraded, the unmodified MMP or catalytically active fragment can be selected from among, for example, a MMP-1, MMP-2, MMP-8, MMP-13 and MMP-14, or catalytically active fragment thereof.

In any of the methods, pharmaceutical compositions or uses herein, the modified MMP or catalytically active fragment can be a modified MMP that contains a modification in an unmodified MMP polypeptide that contains the sequence of amino acids set forth in any of SEQ ID NOS:5, 132, 133, 134, 135, 137, 138, 140, 143 or 145 or a catalytically active fragment thereof, or a sequence of amino acids that exhibits at least 85% sequence identity to any of SEQ ID NOS:5, 132, 133, 134, 135, 137, 138, 140, 143 or 145 or a catalytically active fragment thereof. This includes an unmodified MMP or catalytically active fragment which contains a sequence of amino acids that exhibits at least 86%, 87%, 88%, 89%, 90%, 91%, 92%, 93%, 94%, 95%, 96%, 97%, 98%, 99% or more sequence identity to any of SEQ ID NOS:5, 132, 133, 134, 135, 137, 138, 140, 143 or 145 or a catalytically active fragment thereof.

In particular, in any of the methods, pharmaceutical compositions or uses herein, the unmodified MMP polypeptide or catalytically active fragment is a MMP-1 polypeptide that contains the sequence of amino acids set forth in SEQ ID NO:5, or is a catalytically active fragment thereof, or a sequence of amino acids that exhibits at least 85% sequence identity to SEQ ID NO:5 or a catalytically active fragment thereof. This includes an unmodified polypeptide that contains a sequence of amino acids that exhibits at least 86%, 87%, 88%, 89%, 90%, 91%, 92%, 93%, 94%, 95%, 96%, 97%, 98%, 99% or more, sequence identity to SEQ ID NO:5 or a catalytically active fragment thereof. In any of the methods, pharmaceutical compositions or uses herein, the catalytically active fragment can be a fragment that contains the catalytic domain or a catalytically active portion of the catalytic domain.

In any of the methods, pharmaceutical compositions or uses herein, the modified MMP or catalytically active fragment can exhibit at least 85% sequence identity to the unmodified MMP, and hence, can contain up to 1, 2, 3, 4, 5, 6, 7, 8, 9, 10, 11, 12, 13, 14, 15, 16, 17, 18, 19, 20 or more modifications compared to the unmodified MMP. An example of such a modified polypeptide is a modified MMP or catalytically active fragment that contains only one amino acid replacement to confer reduced activity in the presence of physiological levels of extracellular calcium compared to its activity in the presence of a calcium concentration that is greater than the physiological level.

In any of the above methods, pharmaceutical compositions or uses herein, the modified MMP or catalytically active fragment can be a mature enzyme or a catalytically active fragment that contains only the catalytically active domain or a catalytically active portion of the catalytic domain. The modified MMP or catalytically active fragment can lack all or a portion of a proline-rich linker and/or a hemopexin domain. The modified MMP or catalytically active fragment also can be a zymogen that is processed to a mature enzyme immediately before administration. The zymogen can be processed, for example, by a processing agent. Exemplary processing agents include plasmin, plasma kallikrein, trypsin-1, trypsin-2, neutrophil elastase, cathepsin G, tryptase, chymase, proteinase-3, furin, urinary plasminogen activator (u-PA), an active MMP, 4-aminophenylmercuric acetate (APMA), HgCl₂, N-ethylmaleimide, sodium dodecyl sulfate (SDS), a chaotropic agent, oxidized glutathione, reactive oxygen, Au(I) slat, acidic pH and heat. The processing agent can optionally be purified away from the modified MMP prior to administration.

In any of the methods, pharmaceutical compositions or uses herein, the modified MMP or catalytically active fragment administered in any of the provided methods also can exhibit temperature sensitivity. For example, the modified MMP or catalytically active fragment can exhibit a ratio of at least 1.2 of metalloprotease activity at 25° C. as compared to at 37° C. This includes a modified MMP or catalytically active fragment that exhibits a ratio of metalloprotease activity at 25° C. as compared to at 37° C. of at least 1.3, 1.4, 1.5, 1.6, 1.7, 1.8, 1.9, 2.0, 3.5, 4.0, 5.0, 6.0, 7.0, 8.0. 9.0, 10.0, 11.0, 12.0, 13.0, 14.0, 15.0, 20.0, 30.0, 40.0 or more.

In any of the methods herein, the composition(s) used in the provided methods can be administered to the extracellular matrix (ECM) of the subject. The modified MMP or catalytically active fragment can be administered at physiological temperature or below physiological temperature, for example, at or below 25° C. Thus, the composition containing the modified MMP that is to be administered in the provided methods can have a temperature that is at or below 25° C. The modified MMP or catalytically active fragment can optionally be mixed with a composition that has a temperature lower than the physiological temperature of the body immediately before administration. The ECM of the subject (i.e., locus of administration) also optionally can be cooled to below the physiological temperature of the body, and can be maintained below the physiological temperature of the body for a predetermined time.

In examples of any of the methods herein, the activity of the modified MMP or catalytically active fragment can be reduced in the provided methods by administering a calcium-chelating agent. Such calcium-chelating agents include, but are not limited to, ethylenediaminetetraacetic acid (EDTA), ethylene glycol tetraacetic acid (EGTA), 1,2-bis(o-aminophenoxy)ethane-N,N,N′,N′-tetraacetic acid (BAPTA) or derivatives thereof. The calcium-chelating agent can be administered at a concentration of 1 μM to 100 mM, 5 μM to 50 mM or 10 μM to 20 mM prior to, intermittently with, subsequently to, or simultaneously with administration of the composition containing the modified MMP or catalytically active fragment.

In any of the provided methods herein, the methods can effect treatment of a fibrotic disease or condition by administering the modified MMP or catalytically active fragment at a therapeutically effective amount that can be selected from among a range of from or from about 10 μg to 100 mg, 50 μg to 75 mg, 100 μg to 50 mg, 250 μg to 25 mg, 500 μg to 10 mg, 1 mg to 5 mg and 2 mg to 4 mg. The therapeutically effective amount of the modified MMP or catalytically active fragment can be administered by any method, including subcutaneous, intramuscular, intralesional, intradermal, topical, transdermal, intravenous, oral, and rectal administration. In particular, the provided methods permit subcutaneous administration. Hence, in any of the examples of pharmaceutical compositions or uses herein, the composition can be formulated to contain an amount of MMP that is selected from among a range of from or from about 10 μg to 100 mg, 50 μg to 75 mg, 100 μg to 50 mg, 250 μg to 25 mg, 500 μg to 10 mg, 1 mg to 5 mg and 2 mg to 4 mg.

In particular, in any of the methods, pharmaceutical compositions or uses herein, the fibrotic disease or condition is a collagen-mediated disease or condition. Such a collagen-mediated disease or condition can be associated with irregular formation of collagen fibers and the fibrotic disease or condition can be treated by methods of administering a modified MMP to sever the fibers. These irregularly-formed collagen fibers can be fibrous septae, fibrous scars or fibrous plaques which can be formed by type I collagen and/or type III collagen; and degrading the collagen type I and/or collagen type III, as set forth in the provided methods, effects severance of the fibers. Examples of such collagen-mediated diseases or conditions, including those that are associated with irregular formation of collagen fibers include, for example, cellulite; Dupuytren's disease; Peyronie's disease; Ledderhose fibrosis; stiff joints, such as frozen shoulder; existing scars, such as surgical adhesions, keloids, hypertrophic scars and depressed scars; scleroderma, lymphedema, and collagenous colitis. A fibrotic disease or condition also can include herniated protruding discs.

### BRIEF DESCRIPTION OF THE FIGURES

FIG. 1 sets forth the amino acid sequence of MMP-1 and indicates the residues of identified regions and domains, such as the propeptide, the catalytic domain, linker region, Hemopexin-like domains 1-4, and the cysteine switch. The sites of zinc ion binding are indicated by open triangles and Z1 (catalytic zinc) and Z2 (structural zinc). The calcium-binding residues are indicated by closed triangles and C1, C2, C3, or C4. The active site glutamate is indicated by an open circle.

FIGS. 2A-2C depict exemplary alignments of the human MMP-1 zymogen amino acid sequence with the amino acid sequences of the zymogen forms of other human MMP polypeptides. The alignments are of Pro-MMP-1 (SEQ ID NO:2) and Pro-MMP-8 (SEQ ID NO:17; FIG. 2A); Pro-MMP-13 (SEQ ID NO:20; FIG. 2B); and Pro-MMP-18 (SEQ ID NO:23; FIG. 2C). The catalytic domains are underlined, and calcium- and zinc-binding residues within the catalytic domain are indicated by triangles. Closed triangles indicate calcium-binding residues, open triangles indicate zinc-binding residues, and the active site glutamate is indicated by an open circle. Exemplary corresponding residues are highlighted. The extent of conservation between the aligned sequences is indicated below the alignment as follows: a: “*” indicates the residues are identical, a “:” indicates a conserved substitution with respect to the MMP-1 sequence, and a “.” indicates a semi-conservative substitution with respect to the MMP-1 sequence.

FIGS. 3A-3C depict exemplary alignments of the human MMP-1 zymogen amino acid sequence with species variants of MMP-1 zymogen polypeptides. The alignments are of human Pro-MMP-1 (SEQ ID NO:2) and bovine Pro-MMP-1 (SEQ ID NO:11; FIG. 3A); pig Pro-MMP-1 (SEQ ID NO:9; FIG. 3B); and mouse Pro-MMP-1a (SEQ ID NO:14; FIG. 3C). The catalytic domains are underlined, and calcium- and zinc-binding residues within the catalytic domain are indicated by triangles. Closed triangles indicate calcium-binding residues, open triangles indicate zinc-binding residues, and the active site glutamate is indicated by an open circle. Exemplary corresponding residues are highlighted. The extent of conservation between the aligned sequences is indicated below the alignment as follows: a “*” indicates the residues are identical, a “:” indicates a conserved substitution with respect to the MMP-1 sequence, and a “.” indicates a semi-conservative substitution with respect to the MMP-1 sequence.

FIG. 4 depicts exemplary alignments of the human MMP-1 zymogen amino acid sequence with the mature active form of MMP-1 lacking the prodomain. The alignments are of human Pro-MMP-1 (SEQ ID NO:2) and mature MMP-1 (SEQ ID NO:5). The catalytic domains are underlined, and calcium- and zinc-binding residues within the catalytic domain are indicated by triangles. Closed triangles indicate calcium-binding residues, open triangles indicate zinc-binding residues, and the active site glutamate is indicated by an open circle. Exemplary corresponding residues are highlighted. The extent of conservation between the aligned sequences is indicated below the alignment as follows: a “*” indicates the residues are identical, a “:” indicates a conserved substitution with respect to the MMP-1 sequence, and a “.” indicates a semi-conservative substitution with respect to the MMP-1 sequence.

### DETAILED DESCRIPTION

### Outline

A. Definitions

B. Matrix Metalloproteinases and Calcium-Dependent Conditional Activity
1. Matrix metalloprotease activity in the extracellular matrix
2. Regulation of MMP activity
   a. Metal binding
   b. Endogenous inhibitors
3. Role of collagen in ECM diseases and conditions
4. Conditional regulation of MMP activity by calcium-dependence

C. Matrix Metalloproteinases (MMPs): MMP-1 and Other MMP Collagenases
1. Matrix metalloproteinase-1 (MMP-1)
   a. Catalytic domain
      i. Zn²⁺ ions
      ii. Ca²⁺ ions
   b. Linker region and hemopexin-like domain
   c. MMP-1 substrates
2. Other collagenases

D. Calcium-Sensitive Modified Matrix Metalloprotease Polypeptides (csMMP)
1. Modifications at or near a metal binding site
   Amino acids involved in calcium coordination
2. Modification of a residue corresponding to position 227
3. Combination mutants
4. Additional modifications

E. Methods of Producing Nucleic Acids Encoding csMMPs and Polypeptides Thereof
1. Vectors and cells
2. Expression
   a. Prokaryotic cells
   b. Yeast cells
   c. Insect cells
   d. Mammalian cells
   e. Plants
3. Purification techniques
4. Methods of activation

F. Pharmaceutical Compositions, Dosages and Formulations
1. Compositions for conditional activity
2. Formulations
   a. Injectables, solutions and emulsions
      Lyophilized powders
   b. Topical administration
   c. Compositions for other routes of administration
3. Dosages and administration

G. Methods of Assessing csMMP Activity
1. Methods of assessing enzymatic activity
2. Methods of assessing degradation of ECM component
   a. In vitro assays
   b. In vivo assays
   c. Non-human animal models

H. Methods of Conditional Activation for Treating Diseases or Defects of the ECM
1. Selecting modified MMP
2. Methods of conditional activation
   a. Calcium
   b. Temperature
3 Exemplary fibrotic diseases and conditions (e.g., collagen-mediated diseases and conditions)
   a. Cellulite
   b. Dupuytren's disease
   c. Peyronie's disease
   d. Ledderhose fibrosis
   e. Stiff joints
   f. Existing Scars
      i. Surgical adhesions
      ii. Keloids
      iii. Hypertrophic scars
   g. Scleroderma
   h. Lymphedema
   i. Collagenous colitis
2. Spinal pathologies

I. Combination Therapies
1. Anesthesia and vasoconstrictors
2. Dispersion agent
   Hyaluronidases
J. Examples

### A. DEFINITIONS

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, the extracellular matrix (ECM) refers to the extracellular space within tissues that is formed as a complex meshwork structure that surrounds and provides structural support to cells of specialized tissues and organs. The ECM is made up of structural proteins such as collagen and elastin, specialized proteins such as fibronectin, and proteoglycans. The exact biochemical composition varies from tissue to tissue. In the skin, for example, it is the dermal layer that contains the ECM. Reference to the “interstitium,” or “interstitial space” is used interchangeably herein to refer to the ECM.

As used herein, a component of the ECM refers to any material produced by cells of connective tissue and secreted into the interstitium. For purposes herein, reference to ECM components refers to proteins and glycoproteins, and not to other cellular components or other components of the ECM. Exemplary ECM components include, but are not limited to, matrix proteins such as collagen, fibronectin, elastin and proteoglycans.

As used herein, a matrix degrading enzyme refers to any enzyme that degrades one or more components of the ECM. Matrix-degrading enzymes include proteases, which are enzymes that catalyze the hydrolysis of covalent peptide bonds. Matrix-degrading enzymes include any known to one of skill in the art. Exemplary matrix-degrading enzymes include matrix metalloproteases, allelic or species variants, or other variants thereof.

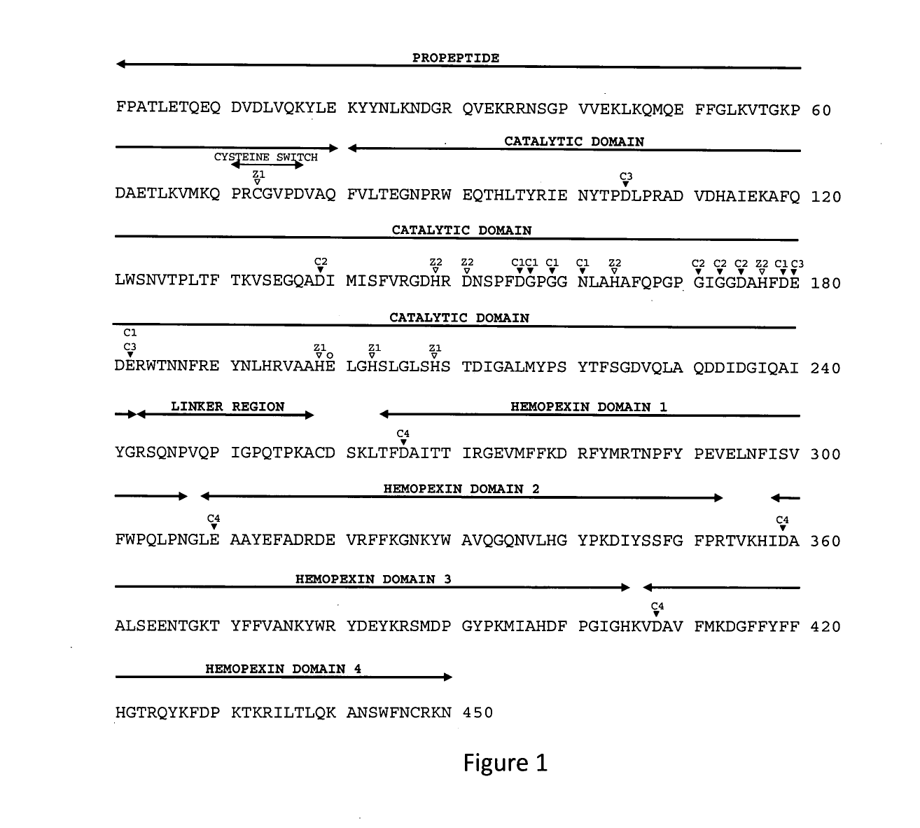

As used herein, a matrix metalloprotease (MMP) refers to a type of matrix-degrading enzyme that is a zinc- and calcium-dependent endopeptidase that contains an active site Zn²⁺ required for activity. MMPs include enzymes that degrade components of the ECM, including, but not limited to, collagen, fibronectin, elastin and proteoglycans. MMPs generally contain a propeptide, a catalytic domain, a proline linker and a hemopexin (also called hemopexin-like C-terminal) domain. Some MMPs contain additional domains. Exemplary MMPs are set forth in Table 3. Reference to a MMP includes all forms, for example, the precursor form (containing the signal sequence), the proenzyme form (containing the propeptide, but no signal sequence), the processed active form (lacking the signal and propeptide), and forms thereof lacking one or more domains. For example, reference to a MMP refers to MMPs containing only the catalytically active domain. Domains of an exemplary MMP (MMP-1) are identified in FIG. 1. MMPs also include allelic or species variants, or other variants thereof.

As used herein, a modified matrix-degrading enzyme or a modified MMP (also interchangeably referred to as a variant or mutant) refers to an enzyme that has one or more modifications in the primary amino acid sequence as compared to a wild-type enzyme. The one or more mutations can be one or more amino acids replacements (substitutions), insertions, deletions, and any combination thereof. A modified enzyme includes those with 1, 2, 3, 4, 5, 6, 7, 8, 9, 10, 11, 12, 13, 14, 15, 16, 17, 18, 19, 20 or more modified positions. The modifications can provide altered properties of the enzyme. Exemplary of modifications include those described herein that confer increased calcium-dependence for activity of the enzyme, i.e., are calcium-sensitive. Other modifications can include those that confer altered substrate specificity, stability and/or sensitivity to inhibitors, such as TIMPs (tissue inhibitors of metalloproteases), or confer sensitivity to other conditions, such as temperature and/or pH. A modified enzyme typically has 60%, 70%, 80%, 85%, 90%, 91%, 92%, 93%, 94%, 95%, 96%, 97%, 98%, 99%, or more, sequence identity to a corresponding sequence of amino acids of a wild-type enzyme not including the modification(s). Typically, a modified enzyme retains an activity or sufficient activity (e.g., degradation of an ECM component) of a wild-type enzyme. It is understood that modifications conferring temperature sensitivity retain an activity or sufficient activity at the requisite temperature compared to a wild-type enzyme at the physiologic temperature.

As used herein, an unmodified matrix-metalloprotease (MMP) refers to a starting polypeptide that is selected for modification as provided herein. The starting polypeptide can be a naturally-occurring, wild-type form of a polypeptide. In addition, the starting polypeptide can be altered or mutated, such that it differs from a native wild-type isoform, but is nonetheless referred to herein as a starting unmodified polypeptide relative to the subsequently modified polypeptides produced herein. Thus, existing proteins known in the art that have been modified to have a desired increase or decrease in a particular activity or property compared to an unmodified reference protein can be selected and used as the starting unmodified polypeptide. For example, a protein that has been modified from its native form by one or more single amino acid changes, and possesses either an increase or decrease in a desired property, such as a change in an amino acid residue or residues to alter glycosylation, can be selected for modification, and hence referred to herein as unmodified, for further modification. An unmodified MMP polypeptide includes human and non-human MMPs, and also includes various forms thereof, including the precursor, zymogen, mature or catalytically active fragment thereof. Exemplary unmodified hyaluronan-degrading enzymes are any set forth in SEQ ID NOS:17, 20, 23, 26, 29, 32, 35, 38, 41, 44, 47, 50, 53, 56, 59, 62, 65, 68, 71, 74, 77, 80, 83, or mature or catalytically active forms thereof, or variants of any of such forms, such as those that exhibit at least 60%, 70%, 75%, 80%, 85%, 86%, 87%, 88%, 89%, 90%, 91%, 92%, 93%, 94%, 95%, 96%, 97%, 98%, 99%, or more, sequence identity to any of SEQ ID NOS:17, 20, 23, 26, 29, 32, 35, 38, 41, 44, 47, 50, 53, 56, 59, 62, 65, 68, 71, 74, 77, 80, or 83. It is understood that an unmodified MMP generally is one that does not contain the modification(s), such as amino acid replacement(s), of a modified MMP. The unmodified form can be a mature form or a form that can be processed for administration as a mature form (e.g., a zymogen form). Exemplary of an unmodified MMP is a mature MMP, such as any MMP polypeptide set forth in any of SEQ ID NOS:5, 132, 133, 134, 135, 137, 138, 140, 143 or 145 or a catalytically active fragment thereof, or any form or variant thereof that has a sequence of amino acids that exhibits at least 70%, 75%, 80%, 85%, 86%, 87%, 88%, 89%, 90%, 91%, 92%, 93%, 94%, 95%, 96%, 97%, 98%, 99%, or more, sequence identity to any of SEQ ID NOS:5, 132, 133, 134, 135, 137, 138, 140, 143 or 145, or a catalytically active fragment thereof.

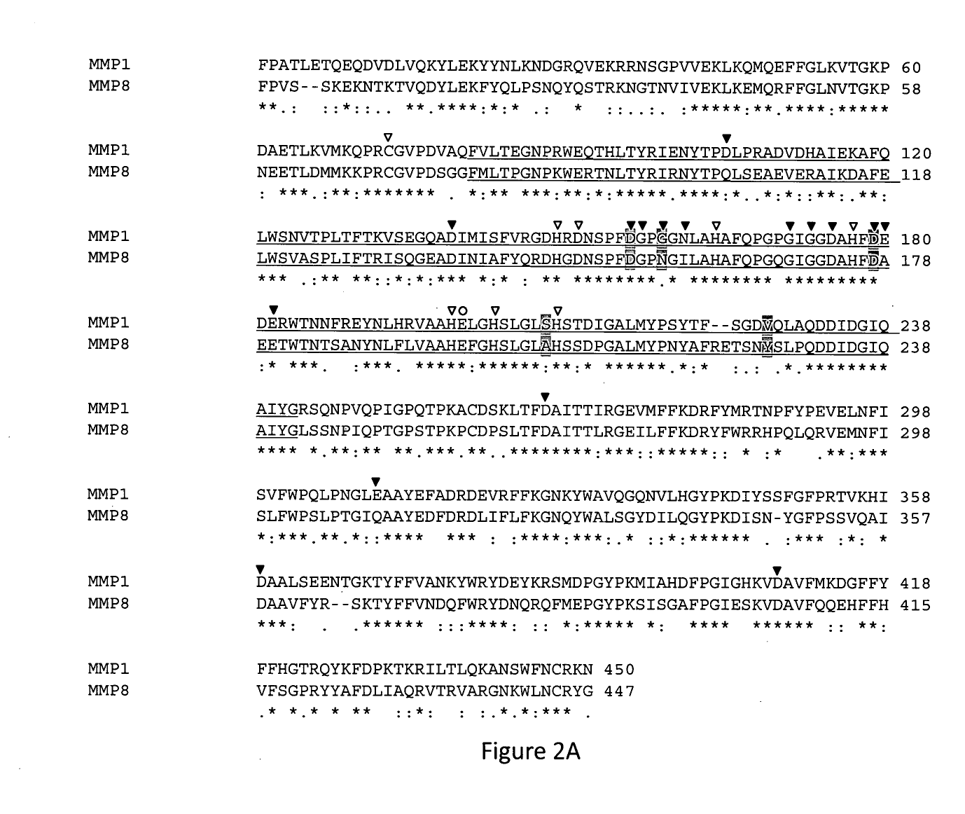

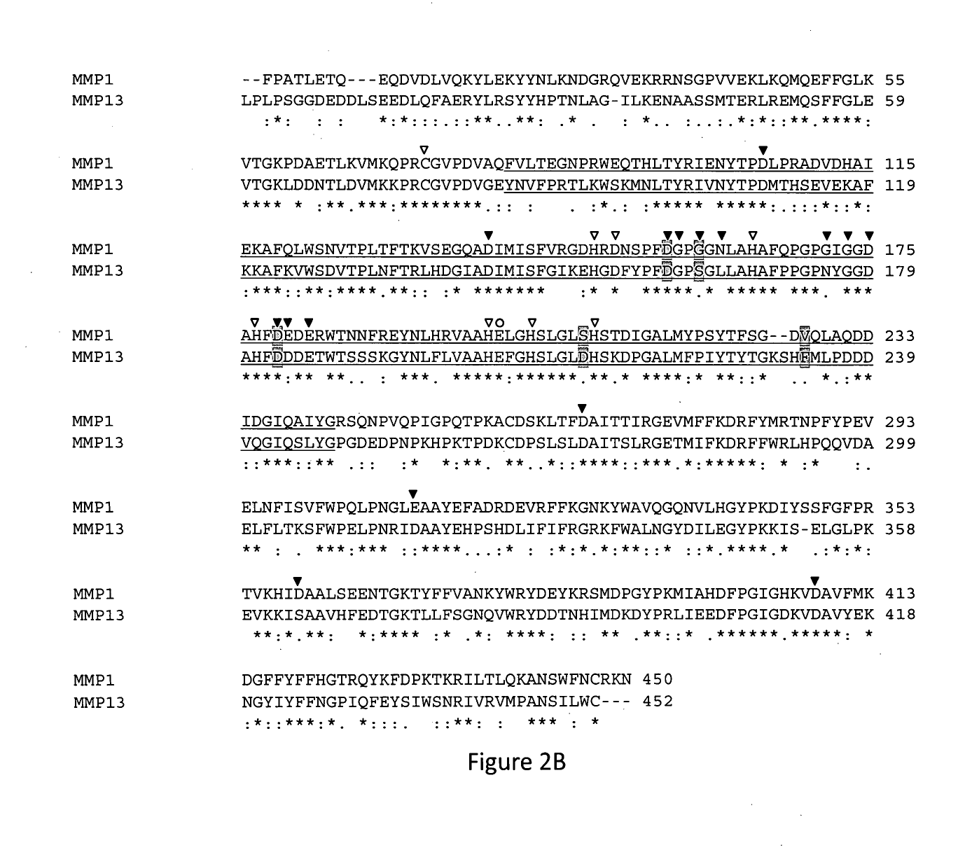

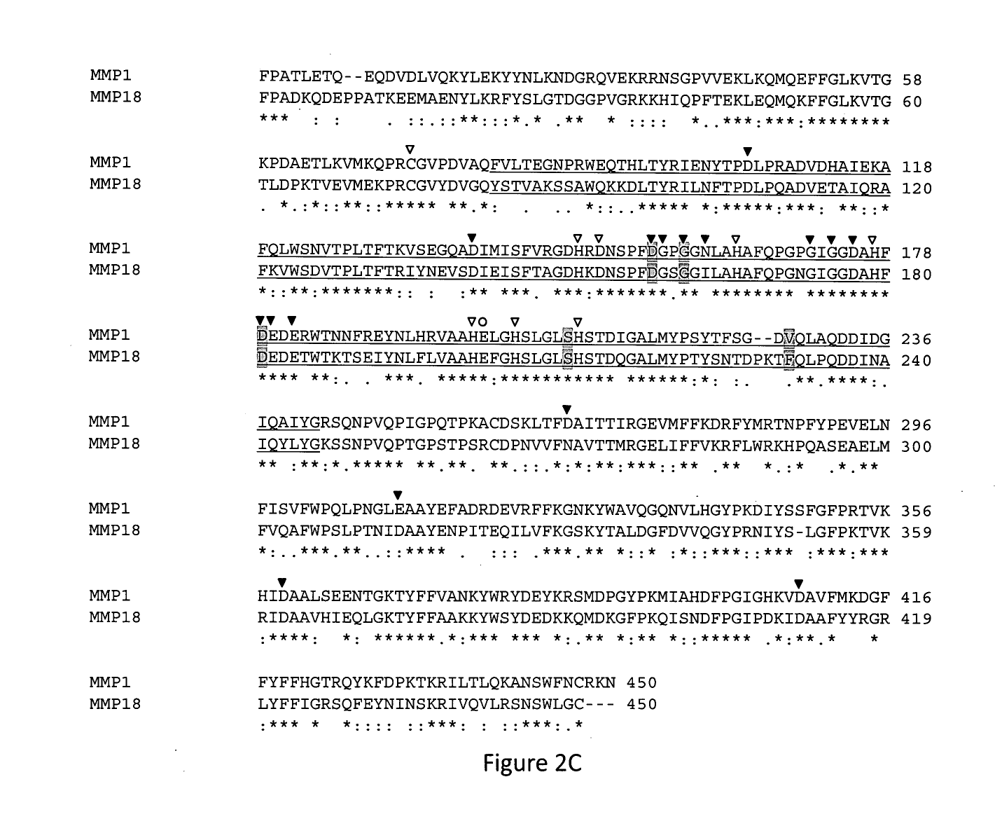

As used herein, “at a position corresponding to” or recitation that nucleotides or amino acid positions “correspond to” nucleotides or amino acid positions in a disclosed sequence, such as set forth in the Sequence Listing, refers to nucleotides or amino acid positions identified upon alignment with the disclosed sequence to maximize identity using a standard alignment algorithm, such as the GAP algorithm. For purposes herein, exemplary positions for modification are with reference to positions set forth in SEQ ID NO:2, and alignment of a MMP to identify a position corresponding to the noted position, is to the amino acid sequence set forth in any of SEQ ID NO:2 (see, e.g., FIGS. 2A-2C, 3A-3C, and 4). By aligning the sequences, one skilled in the art can identify corresponding residues, for example, using conserved and identical amino acid residues as guides. In general, to identify corresponding positions, the sequences of amino acids are aligned so that the highest order match is obtained (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carrillo et al. (1988) SIAM J Applied Math 48:1073). FIGS. 2A-2C illustrate exemplary alignments and identification of exemplary corresponding residues for replacement.

As used herein, a calcium-sensitive (cs) mutant or mutation or variant or modification with reference to a matrix metalloprotease (e.g., csMMP) refers to a polypeptide that is modified so that it exhibits higher dependency on the presence of calcium (Ca²⁺) for enzymatic activity, and thus exhibits increased calcium sensitivity, compared to the unmodified or reference MMP that does not contain the modification(s). A calcium-sensitive mutant exhibits reduced activity in the presence of physiological calcium concentrations (e.g., 1-1.3 mM Ca²⁺) compared to in the presence of calcium concentrations that are greater than physiological levels (e.g., 10 mM Ca²⁺). For purposes herein, a csMMP is generally a MMP that exhibits reduced activity or is substantially inactive in the physiologic environment of the ECM unless exposed to high concentrations of calcium greater than physiological levels. For example, the concentration of calcium at which a csMMP is active is a Ca²⁺ concentration of about or greater than about 2 mM to 100 mM Ca²⁺. Calcium-sensitive mutants used in the methods provided herein exhibit enzymatic activity at physiological concentrations of calcium that is or is about 1%, 2%, 3%, 4%, 5%, 6%, 7%, 8%, 9%, 10%, 20%, 30%, 40%, 50%, 60%, 70%, 80% or 90% up to less then 100% of the activity in the presence of calcium at greater than physiological concentrations. The calcium-sensitive mutants used in the methods provided herein also exhibit 1%, 2%, 3%, 4%, 5%, 6%, 7%, 8%, 9%, 10%, 20%, 30%, 40%, 50%, 60%, 70%, 80% or 90%, up to less then 100%, of the activity at physiological concentrations of calcium as compared to the enzyme that is not modified (e.g., wild-type enzyme) at physiological concentrations of calcium, for example in the interstitial space (e.g., 1-1.3 mM Ca²⁺).

As used herein, the ratio of enzymatic activity at high concentrations of calcium greater than physiological levels compared to physiological calcium concentrations refers to the relation of enzymatic activity at the high concentrations (e.g., 10 mM Ca²⁺) and physiological concentrations (e.g., 1-1.3 mM Ca²⁺) of calcium. It is expressed by the quotient of the division of the activity at high calcium concentrations by the activity at physiological calcium concentrations. It is understood that in determining enzymatic activity and the ratio of enzymatic activity, the enzymatic activity at the high and physiological calcium concentrations is measured under the same assay conditions, except for the difference in calcium concentration.

As used herein, physiological calcium concentration or physiological calcium levels refers to calcium concentrations maintained in the body, in particular, in the interstitial or extracellular space, such as in the extracellular matrix. Such concentrations are approximately 1-1.3 mM Ca²⁺, for example, at or about 1, 1.1, 1.2, or 1.3 mM Ca²⁺. It is understood that the normal range of calcium concentration varies depending on factors such as the particular organ, circulating hormone (e.g., parathyroid hormone and calcitonin) levels in the subject, subject vitamin D levels and other factors. For example, several medical conditions can alter calcium concentrations. For example, hyperparathyroidism, malignancy, vitamin D metabolic disorders, disorders related to high bone-turnover rates, and renal failure can result in elevated blood calcium levels (hypercalcemia) which can result in elevated interstitial calcium concentrations. In contrast, hypocalcemia can result from parathyroid hormone deficiency (e.g., hypoparathyroidism), vitamin D deficiency, high magnesium levels (hypermagnesemia) or low magnesium levels (hypomagnesemia), or chronic renal failure. For purposes herein, the physiological calcium concentration is the concentration of calcium that exists for a non-fasting subject that is not experiencing hypercalcemia or hypocalcemia.

For purposes herein, “high calcium concentration,” or “high concentrations of calcium” refers to concentrations of calcium that are greater than physiological levels of calcium at the site of treatment. For example, the physiological calcium concentration in the interstitial space is approximately 1 to 1.3 mM Ca²⁺. Thus, high calcium concentrations include calcium concentrations that are greater than 1 to 1.3 mM Ca²⁺, for example at or greater than 2 mM, 3 mM, 4 mM, 5 mM, 6 mM, 7 mM, 8 mM, 9 mM, 10 mM, 15 mM, 20 mM, 25 mM, 50 mM, 100 mM or more than 100 mM Ca²⁺. Typically, a high calcium concentration is one that contains sufficient calcium to enable csMMP activity.

As used herein, reference to an enzyme that is conditionally active refers to an enzyme whose activity is regulated by an exogenous factor such that it is active for a limited time or for a limited duration because the activity of the enzyme dissipates and/or is neutralized over time as the exogenous factor diffuses or becomes unavailable. Generally, the exogenous factor is a factor or agent that is not present at the site of administration. In particular, the exogenous factor or agent is one that is not present in the physiological environment of the ECM of a subject. Thus, by virtue of the absence of the exogenous factor required for activity, the activity of the enzyme is reduced and can be rendered sufficiently inactive to achieve a therapeutic effect.

As used herein, “temporal or conditional cleavage of a component of the ECM,” with reference to a pharmaceutical composition, means that the pharmaceutical composition is calcium-sensitive and exhibits conditional activity that is regulated by calcium concentration.

As used herein, “conditional activity” or “regulated activity,” with reference to the presence of calcium, means that the activity of the enzyme is regulated by calcium, such that it is active for a limited time or for a limited duration as the calcium diffuses away or becomes unavailable. With reference to modified MMPs provided herein, conditional activity is conferred by the requirement for high concentrations greater than physiological concentrations of calcium for activity. Accordingly, the high concentration of calcium greatef than physiological concentrations of calcium is not present in the physiological environment of the ECM of a subject, where the concentrations of calcium are physiological levels (e.g., 1-1.3 mM). By virtue of the fact that sufficient calcium concentrations are not present at the site of administration of a csMMP enzyme, additional Ca²⁺ must be added exogenously. Over time, the Ca²⁺ will dissipate, such that sufficient levels of Ca²⁺ are no longer present to maintain the activity of the enzyme. Hence, the csMMP enzyme will be active for a limited or predetermined time upon administration, and thus is a conditionally active enzyme that can be temporally controlled by calcium availability.

As used herein, “limited time” or “limited duration” with the reference to activity of an enzyme means that the activity of the enzyme is restricted in duration or time, for example, because the activity lessens or is reduced over a time period. The specific extent of time until the restricted activity is a function of the particular conditions regulating activity of the enzyme. For example, for purposes herein, the activity of an enzyme is restricted by exposure to physiological concentrations of calcium.

As used herein, “predetermined time” means a limited time that is known before and can be controlled. The dissipation and/or neutralization of an activation condition required for an enzyme's activity can be titrated or otherwise empirically determined so that the time required for an active enzyme to become inactive is known. For purposes herein, for example, an enzyme can be active for a predetermined time that is or is about up to 1 minute, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, or 4 hours, or more. The predetermined time can be controlled by the subject or the treating physician, for example, by administering a formulation containing sufficiently high concentrations of calcium to render the enzyme active for the pre-determined amount of time. Further, it is understood that reversible enzymes used in the methods provided herein can be re-activated by exposure to high calcium concentrations (e.g., 10 mM Ca²⁺), and thereby can be active for an additional predetermined time.

As used herein, reversible refers to a modified enzyme whose activity is dependent on calcium, and whose activity is capable of being recovered or partially recovered upon exposure to deprivation of calcium, followed by re-exposure to a high calcium concentration (e.g., 10 mM Ca²⁺). Hence, the activity of a reversible calcium-sensitive enzyme, once it is deprived of calcium and then re-exposed to activating calcium concentrations, is the same or substantially retained as compared to the activity of the enzyme exposed only to high calcium concentrations (e.g., 10 mM Ca²⁺) and is greater then the activity of the enzyme exposed only to physiological concentrations of calcium. For example, upon return to conditions of high calcium from low (e.g., physiological) levels of calcium, reversible enzymes exhibit at or about 120%, 125%, 130%, 140%, 150%, 160%, 170%, 180%, 200% or more of the activity of the enzyme exposed only to the physiological calcium concentrations and retain the activity of the enzyme exposed only to high calcium concentrations (e.g., 10 mM Ca²⁺).

As used herein, irreversible or nonreversible refers to a modified enzyme whose enzymatic activity is dependent on high calcium concentrations (e.g., 10 mM Ca²⁺), but whose activity is not capable of being recovered following exposure to physiological calcium concentrations and re-exposure to high calcium concentrations (e.g., 10 mM Ca²⁺). Hence, the activity of an irreversible enzyme once it is deprived of sufficient calcium is less than the activity of the enzyme exposed only to high calcium concentrations (e.g., 10 mM Ca²⁺) and also is less than or the same or substantially the same as the activity of the enzyme only in the presence of physiological calcium concentrations (e.g., 1-1.3 mM Ca²⁺). For example, upon return to high calcium concentrations, irreversible enzymes exhibit at or about 20%, 30%, 40%, 50%, 60%, 70%, 80%, 90%, 100%, 105%, 110%, 115%, or 120% of the activity at physiological calcium concentrations and less than 100% of the activity of the enzyme exposed only to high calcium concentrations (e.g., 10 mM Ca²⁺).

As used herein, a domain refers to a portion (a sequence of three or more, generally 5 or 7 or more amino acids) of a polypeptide that is structurally and/or functionally distinguishable or definable. For example, a domain includes those portions that can form an independently folded structure within a protein made up of one or more structural motifs (e.g., combinations of alpha helices and/or beta strands connected by loop regions) and/or that is recognized by virtue of a functional activity, such as kinase activity. A protein can have one, or more than one, distinct domains. For example, a domain can be identified, defined or distinguished by homology of the sequence therein to related family members, such as homology and motifs that define an extracellular domain. In another example, a domain can be distinguished by its function, such as by enzymatic activity, e.g., kinase activity, or an ability to interact with a biomolecule, such as DNA binding, ligand binding, and dimerization. A domain independently can exhibit a function or activity such that the domain independently, or fused to another molecule, can perform an activity, such as, for example, proteolytic activity or ligand binding. A domain can be a linear sequence of amino acids or a non-linear sequence of amino acids from the polypeptide. Many polypeptides contain a plurality of domains. For example, the domain structure of MMPs is set forth in FIG. 1. Those of skill in the art are familiar with domains and can identify them by virtue of structural and/or functional homology with other such domains.

As used herein, a catalytic domain refers to any part of a polypeptide that exhibits a catalytic or enzymatic function. Such domains or regions typically interact with a substrate to result in catalysis thereof. For MMPs, the catalytic domain contains a zinc-binding motif, which contains the Zn²⁺ ion bound by three histidine residues and is represented by the conserved sequence HExGHxxGxxH (SEQ ID NO:85).

As used herein, a proline rich linker (also called the hinge region) refers to a flexible hinge or linker region that has no determinable function. Such a region is typically is found between domains or regions and contributes to the flexibility of a polypeptide.

As used herein, a hemopexin-binding domain or hemopexin-like C-terminal domain refers to the C-terminal region of MMP. It is a four-bladed β-propeller structure which is involved in protein-protein interactions. For example, the hemopexin-binding domain of MMPs interacts with various substrates and also interacts with inhibitors, for example, tissue inhibitors of metalloproteases (TIMPs).

As used herein, “consisting essentially of” or recitation that a polypeptide consists essentially of a particular domain, for example, the catalytic domain, means that the only MMP portion of the polypeptide is the domain or a catalytically active portion thereof. The polypeptide optionally can include additional non-MMP-derived sequences of amino acids, typically at least 3, 4, 5, 6 or more, such as by insertion into another polypeptide or linkage thereto.

As used herein, a “zymogen” refers to an enzyme that is an inactive precursor of and requires some change, such as chemical modification or proteolysis of the polypeptide, to become active. Some zymogens also require the addition of co-factors such as, but not limited to, pH, ionic strength, metal ions, reducing agents, or temperature for activation. Zymogens include the proenzyme form of enzymes. Hence, zymogens generally are inactive and can be converted to a mature polypeptide by chemical modification or catalytic or autocatalytic cleavage of the proregion from the zymogen in the presence or absence of additional cofactors.

As used herein, a prosegment or proregion or propeptide refers to a region or a segment that is cleaved to produce a mature protein. A propeptide is a sequence of amino acids positioned at the amino terminus of a mature polypeptide and can be as little as a few amino acids or can be a multidomain structure. This can include segments that function to suppress the enzymatic activity by masking the catalytic machinery. Propeptides also can act to maintain the stability of an enzyme.

As used herein, a “processing agent” refers to an agent that activates a MMP by facilitating removal of the propeptide or proregion from the zymogen or inactive form of the enzyme. A processing agent includes chemical agents, proteases and other agents such as acidic pH or heat. Exemplary processing agents include, but are not limited to, trypsin, furin, or 4-aminophenylmercuric acetate (APMA). Other exemplary processing agents are listed in Table 8.

As used herein, an active enzyme refers to an enzyme that exhibits enzymatic activity. For purposes herein, active enzymes are those that cleave any one or more components of the ECM, such as collagen. Active enzymes include those that are processed from the zymogen form into the mature form.

As used herein, a “catalytically active fragment” refers to a polypeptide fragment that contains the catalytically active domain of the enzyme sufficient to exhibit activity. Hence, a catalytically active fragment is the portion that, under appropriate conditions (e.g., the presence of sufficient calcium concentrations), can exhibit catalytic activity. For example, a catalytically active fragment of a csMMP-1 (containing at least one mutation that confers a phenotype of increased calcium dependency) exhibits activity when it is provided at the requisite calcium concentration (e.g., 10 mM), but exhibits substantially reduced or no activity at physiological calcium concentrations (e.g., 1-1.3 mM). Typically, a catalytically active fragment is a contiguous sequence of amino acids of a MMP polypeptide that contains the catalytic domain and also the requisite portion of the molecule to recognize the substrate required for enzymatic activity. For example, the hemopexin domain of MMPs can be involved in binding and cleavage of substrates, and hence at least a portion of the hemopexin domain can be required for a fragment of a MMP to exhibit catalytic activity against a substrate. Typically, a catalytically active fragment of a MMP contains at least 100, 200, 300, 400, 500, or more, amino acid residues.

As used herein, reference to the “mature” form or “processed mature” form of an enzyme refers to enzymes that do not include the prosegment or proregion of the enzyme. It can be produced from the zymogen or proenzyme by activation cleavage in which a prosegment or proregion of the proenzyme is processed to produce the mature form. Hence, a processed mature enzyme lacks the sequence of amino acids that correspond to the prosegment or proregion. It is understood that reference to a processed mature form of an enzyme includes synthetic sequences, and thus does not necessarily require that the enzyme actually is processed to remove the prosegment or proregion. It is understood that any MMP enzyme that lacks the prosegment or proregion sequence is a mature enzyme. For example, SEQ ID NO:5 is the mature sequence of MMP-1. The processed mature form of an enzyme can exhibit activity, and is thus an active enzyme, under appropriate conditions. For example, under physiological conditions, the mature form of MMP-1 is an active enzyme. In contrast, csMMP-1 variants used in the methods provided herein exhibit substantially reduced or no activity at physiological calcium concentrations (e.g., 1-1.3 mM Ca²⁺ in the interstitial space), but are enzymatically active when exposed to increased Ca²⁺ concentrations (e.g., concentrations greater than 2 mM Ca²⁺, such as 10 mM Ca²⁺). Thus, the csMMP-1 is conditionally active.

As used herein, a “therapeutically effective amount” or a “therapeutically effective dose” refers to an agent, compound, material, or composition containing a compound that is at least sufficient to produce a therapeutic effect.

As used herein, sub-epidermal administration refers to any administration that results in delivery of the enzyme under the outer-most layer of the skin. Sub-epidermal administration does not include topical application onto the outer layer of the skin. Examples of sub-epidermal administration includes, but are not limited to, subcutaneous, intramuscular, intralesional and intradermal routes of administration.

As used herein, substrate refers to a molecule that is cleaved by an enzyme. Minimally, a target substrate includes a peptide containing the cleavage sequence recognized by the protease, and therefore can be two, three, four, five, six or more residues in length. A substrate also includes a full-length protein, allelic variant, isoform or any portion thereof that is cleaved by an enzyme. Additionally, a substrate includes a peptide or protein containing an additional moiety that does not affect cleavage of the substrate by the enzyme. For example, a substrate can include a four-amino acid peptide, or a full-length protein chemically linked to a fluorogenic moiety.

As used herein, collagen refers to a group of naturally occurring proteins that are made up of three polypeptide strands that are twisted together into a triple-helix structure. Each triple-helix associates to form collagen fibrils. Collagens are the most abundant protein in mammals, and have functions in tissue assembly or maintenance. Collagens are the main component of connective tissues. In particular, collagen is found in fibrous tissues, such as tendon, ligament and skin, and also is abundant in cornea, cartilage, bone, blood vessels, the gut and intervertebral disc. In particular, in the skin, collagen occurs in the extracellular matrix as elongated fibrils (generally made up of type I and type III collagens). Collagen is produced by fibroblasts.

As used herein, type I collagen refers to the type of collagen that is primarily found in bone, skin and tendons.

As used herein, type III collagen refers to the type of collagen that is the main component of reticular fibers, typically found in the skin, lung and aorta.

As used herein, cleavage refers to the breaking of peptide bonds or other bonds by an enzyme that results in one or more degradation products.

As used herein, “degrading,” “cleaving” or “proteolysis,” used interchangeably herein, with reference to a component of the ECM, refers to the ability of a matrix metalloprotease to catalyze the hydrolysis of covalent peptidic bonds in a matrix protein substrate, such as a collagen. This results in one or more degradation products that are fragments of the protein substrate. For example, certain collagenase MMPs (e.g., MMP-1) can degrade triple-helical fibrillar collagens into ¾ and ¼ fragments.

As used herein, activity refers to a functional activity or activities of a polypeptide or portion thereof associated with a full-length (complete) protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, enzymatic activity or catalytic activity or cleavage activity refers to the activity of a protease as assessed in proteolytic assays (e.g., in vitro proteolytic assays) that detect proteolysis of a selected substrate.

As used herein, “matrix metalloprotease activity” refers to the enzymatic activity of a protease as assessed in proteolytic assays (e.g., in vitro proteolytic assays) that detect proteolysis or cleavage of a matrix protein, and in particular a matrix protein of the extracellular matrix.

As used herein, “collagenase activity” refers to the enzymatic activity of a protease as assessed in proteolytic assays (e.g., in vitro proteolytic assay) that detect proteolysis or cleavage of a collagen.

As used herein, catalytic efficiency or k_{cat}/Kₘ is a measure of the efficiency with which a protease cleaves a substrate and is measured under steady state conditions as is well known to those skilled in the art. Kₘ is the Michaelis-Menton constant ([S] at one-half Vₘₐₓ) and k_{cat} is the Vₘₐₓ/[E_{T}], where E_{T} is the final enzyme concentration. The parameters k_{cat}, Kₘ and k_{cat}/Kₘ can be calculated by graphing the inverse of the substrate concentration versus the inverse of the velocity of substrate cleavage, and fitting to the Lineweaver-Burk equation (1/velocity=(Kₘ/Vₘₐₓ)(1/[S])+1/Vₘₐₓ; where Vₘₐₓ [E_{T}]k_{cat}). Any method to determine the rate of increase of cleavage over time in the presence of various concentrations of substrate can be used to calculate the specificity constant. For example, a substrate is linked to a fluorogenic moiety, which is released upon cleavage by a protease. By determining the rate of cleavage at different enzyme concentrations, k_{cat} can be determined for a particular protease.

As used herein, an inactive enzyme refers to an enzyme that exhibits substantially no activity (i.e., catalytic activity or cleavage activity), such as less than 10% of the maximum activity of the enzyme. The enzyme can be inactive by virtue of its conformation, the absence of sufficient calcium required for its activity, or the presence of an inhibitor or any other condition or factor or form that renders the enzyme substantially inactive.

As used herein, “retains an activity” refers to the activity exhibited by a csMMP polypeptide at a particular concentration of calcium compared to at another calcium concentration or to another polypeptide. For example, it is the activity a csMMP polypeptide exhibits as compared to an unmodified MMP polypeptide of the same form and under the same conditions. It also can be the activity a csMMP polypeptide exhibits as compared to the csMMP polypeptide at different calcium concentrations. Generally, a csMMP polypeptide that retains an activity exhibits increased or decreased activity compared to an unmodified polypeptide under the same conditions or compared to the unmodified polypeptide under different conditions. For example, the csMMP polypeptide can retain 40%, 50%, 60%, 70%, 80%, 90%, 100%, 110%, 120%, 130%, 140%, 150%, 160%, 170%, 180%, 190%, 200%, 300%, 400%, 500% or more of the enzymatic activity of its unmodified MMP counterpart.

As used herein, a human protein is one encoded by a nucleic acid molecule, such as DNA, present in the genome of a human, including all allelic variants and conservative variations thereof. A variant or modification of a protein is a human protein if the modification is based on the wild-type or prominent sequence of a human protein.

As used herein, hyaluronidase refers to an enzyme that degrades hyaluronic acid. Hyaluronidases include bacterial hyaluronidases (EC 4.2.99.1), hyaluronidases from leeches, other parasites, and crustaceans (EC 3.2.1.36), and mammalian-type hyaluronidases (EC 3.2.1.35). Hyaluronidases also include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include any set forth in any of SEQ ID NOS:106-129. Exemplary human hyaluronidases include HYAL1 (SEQ ID NO:93), HYAL2 (SEQ ID NO:94), HYAL3 (SEQ ID NO:95), HYAL4 (SEQ ID NO:96), and PH20 (SEQ ID NO:97). Also included amongst hyaluronidases are soluble human PH20 and soluble rHuPH20.

Reference to hyaluronidases includes precursor hyaluronidase polypeptides and mature hyaluronidase polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 65%, 70%, 75%, 80%, 85%, 90%, 95%, 96%, 97%, 98%, 99% or more sequence identity to the precursor polypeptide set forth in SEQ ID NO:97 or the mature form thereof. Hyaluronidases also include those that contain chemical or posttranslational modifications and those that do not contain chemical or posttranslational modifications. Such modifications include, but are not limited to, PEGylation, albumination, glycosylation, farnesylation, carboxylation, hydroxylation, phosphorylation, and other polypeptide modifications known in the art.

As used herein, soluble human PH20 or sHuPH20 includes mature polypeptides lacking all or a portion of the glycosylphosphatidylinositol (GPI) attachment site at the C-terminus, such that upon expression the polypeptides are soluble. Exemplary sHuPH20 polypeptides include mature polypeptides having an amino acid sequence set forth in any one of SEQ ID NOS:100-105, or a sequence of amino acids that exhibit at least 75%, 80%, 85%, 90%, 91%, 92%, 93%, 94%, 95%, 96%, 97%, 98%, 99%, or more, sequence identity to any of SEQ ID NOS:100-105. The precursor polypeptides for such exemplary sHuPH20 polypeptides include an amino acid signal sequence. Exemplary of a precursor is set forth in SEQ ID NO:97, which contains a 35 amino acid signal sequence at amino acid positions 1-35.

As used herein, soluble rHuPH20 refers to a soluble form of human PH20 that is recombinantly expressed in Chinese Hamster Ovary (CHO) cells. Soluble rHuPH20 is encoded by nucleic acid that includes the signal sequence and is set forth in SEQ ID NO:99. Also included are DNA molecules that are allelic variants thereof and other soluble variants. The nucleic acid encoding soluble rHuPH20 is expressed in CHO cells which secrete the mature polypeptide. As produced in the culture medium, there is heterogeneity at the C-terminus so that the product includes a mixture of species of SEQ ID NOS:100-105. Corresponding allelic variants and other variants also are included. Other variants can have 60%, 70%, 80%, 85%, 90%, 91%, 92%, 93%, 94%, 95%, 96%, 97%, 98%, 99% or more sequence identity with any of SEQ ID NOS:100-105, as long as they retain a hyaluronidase activity and are soluble.

As used herein, hyaluronidase activity refers to any activity exhibited by a hyaluronidase polypeptide. Such activities can be tested in vitro and/or in vivo and include, but are not limited to, enzymatic activity, such as to effect cleavage of hyaluronic acid, ability to act as a dispersing or spreading agent, and antigenicity.

As used herein, the residues of naturally occurring α-amino acids are the residues of those 20 α-amino acids found in nature which are incorporated into protein by the specific recognition of the charged tRNA molecule with its cognate mRNA codon in humans.

As used herein, nucleic acids include DNA, RNA and analogs thereof, including peptide nucleic acids (PNA) and mixtures thereof. Nucleic acids can be single or double-stranded. When referring to probes or primers, which are optionally labeled, such as with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally, a probe or primer contains at least 14, 16 or 30 contiguous nucleotides of sequence complementary to or identical to a gene of interest. Probes and primers can be 10, 20, 30, 50, 100 or more nucleic acids long.

As used herein, a peptide refers to a polypeptide that is from 2 to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the a-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-3559 (1969), and adopted in 37 C.F.R. §§1.821-1.822, abbreviations for amino acid residues are shown in Table 1:

TABLE 1
Table of Correspondence

SYMBOL

| 1-Letter | 3-Letter | Amino Acid |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic Acid |
| Z | Glx | Glu and/or Gln |
| W | Trp | Tryptophan |
| R | Arg | Arginine |
| D | Asp | Aspartic Acid |
| N | Asn | Asparagine |
| B | Asx | Asn and/or Asp |
| C | Cys | Cysteine |
| X | Xaa | Unknown or Other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence (Table 1), and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues, to an amino-terminal group such as NH₂ or to a carboxyl-terminal group such as COOH.

As used herein, “naturally occurring amino acids” refers to the 20 L-amino acids that occur in polypeptides.

As used herein, “non-natural amino acid” refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally-occurring amino acids and include, but are not limited to, the D-stereoisomers of amino acids. Exemplary non-natural amino acids are described herein and are known to those of skill in the art.

As used herein, suitable conservative substitutions of amino acids are known to those of skill in the art and can be made generally without altering the biological activity of the resulting molecule. Those of skill in the art recognize that, in general, single amino acid substitutions in non-essential regions of a polypeptide do not substantially alter biological activity (see e.g., Watson et al. Molecular Biology of the Gene, 4th Edition, 1987, The Benjamin/Cummings Pub. co., p. 224). Such substitutions can be made in accordance with those set forth in Table 2 as follows:

TABLE 2
Conservative and semi-conservative amino acid substitutions

| | | Exemplary |
|---|---|---|
| Original | Exemplary conservative | semi-conservative |
| residue | substitution | substitution |
| Ala (A) | Ser; Thr | Cys; Gly; Pro; Val |
| Arg (R) | Gln; His; Lys | Asp; Glu |
| Asn (N) | Asp; Gln; His; Lys; Glu | Arg; Gly; Ser; Thr |
| Asp (D) | Asn; Gln; Glu | Gly; His; Lys; Ser |
| Cys (C) | | Ala; Ser |
| Gln (Q) | Arg; Asn; Asp; Glu; His; Lys | Ser |
| Glu (E) | Asp; Asn; Gln; Lys | Arg; His; Ser |
| Gly (G) | | Ala; Asp; Asn; Ser |
| His (H) | Arg; Asn; Gln; Lys; Tyr | Asp; Glu; Phe |
| Ile (I) | Leu; Val; Met; Phe | |
| Leu (L) | Ile; Met; Phe; Val | |
| Lys (K) | Arg; Asn; Gln; Glu; His | Asp; Ser; Thr |
| Met (M) | Ile; Leu; Phe; Val | |
| Phe (F) | Ile; Met; Leu; Trp; Tyr | His; Val |
| Pro (P) | | Ala; Ser; Thr |
| Ser (S) | Ala; Thr | Asp; Asn; Cys; Gln; Glu; Gly; |
| | | Lys; Pro |
| Thr (T) | Ala; Ser | Asn; Lys; Pro; Val |
| Trp (W) | Phe; Tyr | |
| Tyr (Y) | His; Phe; Trp | |
| Val (V) | Ile; Leu; Met | Ala; Phe; Thr |

Other substitutions also are permissible and can be determined empirically or in accord with known conservative substitutions.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus, all nucleotides within a double-stranded polynucleotide molecule cannot be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative or semi-conservative differences and/or frequent substitutions in amino acids (or nucleotides). Conservative differences are those that preserve the physicochemical properties of the residues involved. Semi-conservative differences are those that preserve the steric conformation (i.e., size and/or shape) of the residues involved. Alignments can be global (alignment of the compared sequences over the entire length of the sequences and including all residues) or local (the alignment of a portion of the sequences that includes only the most similar region or regions).

As used herein, “sequence identity” refers to the number of identical or similar amino acids or nucleotide bases in a comparison between a test and a reference polypeptide or polynucleotide. Sequence identity can be determined by sequence alignment of nucleic acid or protein sequences to identify regions of similarity or identity. For purposes herein, sequence identity is generally determined by alignment to identify identical residues. Alignment can be local or global. Reference herein to sequence identity is generally a global alignment where the full-length of each sequence is compared. Matches, mismatches and gaps can be identified between compared sequences. Gaps are null amino acids or nucleotides inserted between the residues of aligned sequences so that identical or similar characters are aligned. Generally, there can be internal and terminal gaps. Sequence identity can be determined by taking into account gaps as the number of identical residues/length of the shortest sequence×100. When using gap penalties, sequence identity can be determined with no penalty for end gaps (e.g., terminal gaps are not penalized). Alternatively, sequence identity can be determined without taking into account gaps as the number of identical positions/length of the total aligned sequence×100.

As used herein, a “global alignment” is an alignment that aligns two sequences from beginning to end, aligning each letter in each sequence only once. An alignment is produced, regardless of whether or not there is similarity or identity between the sequences. For example, 50% sequence identity based on “global alignment” means that in an alignment of the full sequence of two compared sequences each of 100 nucleotides in length, 50% of the residues are the same. It is understood that global alignment also can be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman et al. (1970) J Mol. Biol. 48:443). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a “local alignment” is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. (1981) 2:482). For example, 50% sequence identity based on “local alignment” means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include: (1) a unary comparison matrix (containing a value of 1 for identities and 0 for non-identities) and the weighted comparison matrix of Gribskov et al. (1986) Nucl. Acids Res. 14: 6745, as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Whether any two nucleic acid molecules have nucleotide sequences or any two polypeptides have amino acid sequences that are at least 80%, 85%, 90%, 95%, 96%, 97%, 98% or 99% “identical,” or other similar variations reciting a percent identity, can be determined using known computer algorithms based on local or global alignment (see e.g., wikipedia.org/wiki/Sequence_alignment_software, providing links to dozens of known and publicly available alignment databases and programs). Generally, for purposes herein, sequence identity is determined using computer algorithms based on global alignment, such as the Needleman-Wunsch Global Sequence Alignment tool available from NCBI/BLAST (blast.ncbi.nlm.nih.gov/Blast.cgi?CMD=Web&Page_TYPE=BlastHome); LAlign (William Pearson implementing the Huang and Miller algorithm (Adv. Appl. Math. (1991) 12:337-357)); and the program from Xiaoqui Huang available at deepc2.psi.iastate.edu/aat/align/align.html. Local alignment also can be used when the sequences being compared are substantially the same length.

Therefore, as used herein, the term “identity” represents a comparison or alignment between a test and a reference polypeptide or polynucleotide. In one non-limiting example, “at least 90% identical to” refers to percent identities from 90 to 100% relative to the reference polypeptide or polynucleotide. Identity at a level of 90% or more is indicative of the fact that, assuming for exemplification purposes a test and reference polypeptide or polynucleotide length of 100 amino acids or nucleotides are compared, no more than 10% (i.e., 10 out of 100) of amino acids or nucleotides in the test polypeptide or polynucleotide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences also can be due to deletions or truncations of amino acid residues. Differences are defined as nucleic acid or amino acid substitutions, insertions or deletions. Depending on the length of the compared sequences, at the level of homologies or identities above about 85-90%, the result can be independent of the program and gap parameters set; such high levels of identity can be assessed readily, often without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to two or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)—PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g., by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g., an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically, the two molecules are selected such that they will hybridize under conditions of high stringency.

As used herein, “substantially identical to a product” means sufficiently similar so that the property of interest is sufficiently unchanged so that the substantially identical product can be used in place of the product.

As used herein, it also is understood that the terms “substantially identical” or “similar” vary with the context as understood by those skilled in the relevant art.

As used herein, an allelic variant or allelic variation references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequences. The term “allelic variant” also is used herein to denote a protein encoded by an allelic variant of a gene. Typically, the reference form of the gene encodes a wild-type form and/or predominant form of a polypeptide from a population or single reference member of a species. Typically, allelic variants, which include variants between and among species typically have at least 80%, 90% or greater amino acid identity with a wild-type and/or predominant form from the same species; the degree of identity depends upon the gene and whether comparison is interspecies or intraspecies. Generally, intraspecies allelic variants have at least about 80%, 85%, 90% or 95% identity or greater with a wild-type and/or predominant form, including 96%, 97%, 98%, 99% or greater identity with a wild-type and/or predominant form of a polypeptide. Reference to an allelic variant herein generally refers to variations in proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include substitutions, deletions, and insertions of nucleotides. An allele of a gene also can be a form of a gene containing a mutation.

As used herein, species variants refer to variants in polypeptides among different species, including different mammalian species, such as mouse and human.

As used herein, a splice variant refers to a variant produced by differential processing of a primary transcript of genomic DNA that results in more than one type of mRNA.

As used herein, modification is in reference to modification of a sequence of amino acids of a polypeptide or a sequence of nucleotides in a nucleic acid molecule and includes deletions, insertions, and replacements of amino acids and nucleotides, respectively. Methods of modifying a polypeptide are routine to those of skill in the art, such as by using recombinant DNA methodologies.

As used herein, the term promoter means a portion of a gene containing DNA sequences that provide for the binding of RNA polymerase and initiation of transcription. Promoter sequences are commonly, but not always, found in the 5′ non-coding region of genes.

As used herein, isolated or purified polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

The term substantially free of cellular material includes preparations of proteins in which the protein is separated from cellular components of the cells from which it is isolated or recombinantly-produced. In one embodiment, the term substantially free of cellular material includes preparations of enzyme proteins having less that about 30% (by dry weight) of non-enzyme proteins (also referred to herein as a contaminating protein), generally less than about 20% of non-enzyme proteins or 10% of non-enzyme proteins or less that about 5% of non-enzyme proteins. When the enzyme protein is recombinantly produced, it also is substantially free of culture medium, i.e., culture medium represents less than about or at 20%, 10% or 5% of the volume of the enzyme protein preparation.

As used herein, the term substantially free of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight) 20%, 10%, 5% or less of chemical precursors or non-enzyme chemicals or components.

As used herein, synthetic, with reference to, for example, a synthetic nucleic acid molecule or a synthetic gene or a synthetic peptide, refers to a nucleic acid molecule or polypeptide molecule that is produced by recombinant methods and/or by chemical synthesis methods.

As used herein, production by recombinant means by using recombinant DNA methods means the use of the well known methods of molecular biology for expressing proteins encoded by cloned DNA.

As used herein, vector (or plasmid) refers to discrete elements that are used to introduce a heterologous nucleic acid into cells for either expression or replication thereof. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal, or those which integrate into the host cell genome.

As used herein, vector also includes “virus vectors” or “viral vectors.” Viral vectors are engineered viruses that are operatively linked to exogenous genes to transfer (as vehicles or shuttles) the exogenous genes into cells.

As used herein, operably or operatively linked, when referring to DNA segments, means that the segments are arranged so that they function in concert for their intended purposes, e.g., transcription initiates in the promoter and proceeds through the coding segment to the terminator.

As used herein, the term assessing is intended to include quantitative and qualitative determination in the sense of obtaining an absolute value for the activity of a protease, or a domain thereof, present in the sample, and also of obtaining an index, ratio, percentage, visual, or other value indicative of the level of the activity. Assessment can be direct or indirect and the chemical species actually detected need not of course be the proteolysis product itself but can for example be a derivative thereof or some further substance. For example, detection of a cleavage product of a substrate, such as by SDS-PAGE and protein staining with Coomassie blue.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures. Biological activities can be observed in in vitro systems designed to test or use such activities. Thus, for purposes herein, a biological activity of a protease is its catalytic activity in which a polypeptide is hydrolyzed.

As used herein, “equivalent,” when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide. When equivalent refers to a property, the property does not need to be present to the same extent (e.g., two peptides can exhibit different rates of the same type of enzymatic activity), but the activities are usually substantially the same.

As used herein, “modulate” and “modulation” or “alter” refer to a change of an activity of a molecule, such as a protein. Exemplary activities include, but are not limited to, biological activities, such as signal transduction. Modulation can include an increase in the activity (i.e., up-regulation or agonist activity) a decrease in activity (i.e., down-regulation or inhibition) or any other alteration in an activity (such as a change in periodicity, frequency, duration, kinetics or other parameter). Modulation can be context dependent and typically modulation is compared to a designated state, for example, the wild-type protein, the protein in a constitutive state, or the protein as expressed in a designated cell type or condition.

As used herein, a composition refers to any mixture. It can be a solution, suspension, liquid, powder, paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination that optionally includes other elements, such as additional reagents and instructions for use of the combination or elements thereof.

As used herein, “locus” with reference to administration refers to the particular site or place that administration occurs. For example, a locus can refer to a site of injection or infusion of an agent.

As used herein, “disease or disorder” refers to a pathological condition in an organism resulting from a cause or condition including, but not limited to, infections, acquired conditions, and genetic conditions, and characterized by identifiable symptoms. Diseases and disorders of interest herein are those involving components of the ECM, for example, diseases or disorders involving collagen.

As used herein, a fibrotic disease or condition refers to a disease or condition associated with the irregular formation of fibrous tissue that is generated due to the production, accumulation or overproduction of a component of the ECM, in particular a collagen. For example, the pathogenesis of fibrotic diseases and conditions can be due to the irregular formation of collagen fibers, such as the formation of fibrous septae, fibrous scars, fibrous plaques, fibrous nodes or nodules or fibrous cords. Exemplary of fibrotic diseases and conditions include, but are not limited to, localized scleroderma, Peyronie's Disease, Dupuytren's contracture, hypertrophic scarring, keloids, cellulite, frozen shoulders, existing scars, lymphedema, lipoma and other diseases and conditions described herein or known in the art.

As used herein, an ECM-mediated disease or condition is one where any one or more ECM components is involved in the pathology or etiology. For purposes herein, an ECM-mediated disease or condition includes those that are caused by an increased deposition or accumulation of one or more ECM components, such as collagen. Such conditions include, but are not limited to, cellulite, Duputyren's syndrome, Peyronie's disease, frozen shoulders, existing scars such as keloids, scleroderma and lymphedema.

As used herein, collagen-mediated disease or condition is a fibrotic disease or condition that is caused by the production, overproduction or accumulation of collagen fibers. In particular, the collagen-mediated disease or condition is an ECM-mediated disease or condition involving type I or type III collagen.

As used herein, “treating” a subject with a disease or condition means that the subject's symptoms are partially or totally alleviated, or remain static following treatment. Hence, treatment encompasses prophylaxis, therapy and/or cure. Prophylaxis refers to prevention of a potential disease and/or a prevention of worsening of symptoms or progression of a disease.

As used herein, a pharmaceutically effective agent includes any therapeutic agent or bioactive agent, including, but not limited to, for example, anesthetics, vasoconstrictors, dispersing agents, conventional therapeutic drugs, including small molecule drugs, and therapeutic proteins.

As used herein, treatment means any manner in which the symptoms of a condition, disorder or disease or other indication thereof is/are ameliorated or otherwise beneficially altered.

As used herein, therapeutic effect means an effect resulting from treatment of a subject that alters, typically improves or ameliorates, the symptoms of a disease or condition, or that cures a disease or condition. A therapeutically effective amount refers to the amount of a composition, molecule or compound which results in a therapeutic effect following administration to a subject. A therapeutically effective amount effects treatment.

As used herein, the term “subject” refers to an animal, including a mammal, such as a human being.

As used herein, a patient refers to a human subject.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, prevention or prophylaxis refers to methods in which the risk of developing disease or condition is reduced.

As used herein, an effective amount is the quantity of a therapeutic agent necessary for preventing, curing, ameliorating, arresting or partially arresting a symptom of a disease or disorder.

As used herein, unit dose form refers to physically discrete units suitable for human and animal subjects and packaged individually as is known in the art.

As used herein, a single dosage formulation refers to a formulation for direct administration.

As used herein, an “article of manufacture” is a product that is made and sold. As used throughout this application, the term is intended to encompass modified MMPs contained in articles of packaging.

As used herein, fluid refers to any composition that can flow. Fluids thus encompass compositions that are in the form of semi-solids, pastes, solutions, aqueous mixtures, gels, lotions, creams and other such compositions.

As used herein, a cellular extract or lysate refers to a preparation or fraction which is made from a lysed or disrupted cell.

As used herein, animal includes any animal, such as, but not limited to, primates, including humans, gorillas and monkeys; rodents, such as mice and rats; fowl, such as chickens; ruminants, such as goats, cows, deer, sheep; ovine, such as pigs, and other animals. Non-human animals exclude humans as the contemplated animal. The enzymes provided herein are from any source, animal, plant, prokaryotic and fungal. Most enzymes are of animal origin, including mammalian origin.

As used herein, a control refers to a sample that is substantially identical to the test sample, except that it is not treated with a test parameter, or, if it is a plasma sample, it can be from a normal volunteer not affected with the condition of interest. A control also can be an internal control.

As used herein, the singular forms “a,” “an” and “the” include plural referents unless the context clearly dictates otherwise. Thus, for example, reference to a compound, comprising “an extracellular domain” includes compounds with one or a plurality of extracellular domains.

As used herein, ranges and amounts can be expressed as “about” a particular value or range. “About” also includes the exact amount. Hence “about 5 bases” means “about 5 bases” and also “5 bases.”

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, the abbreviations for any protective groups, amino acids and other compounds are, unless indicated otherwise, in accord with their common usage, recognized abbreviations, or the IUPAC-IUB Commission on Biochemical Nomenclature (see e.g., Biochem. (1972) 11:1726).

### B. MATRIX METALLOPROTEINASES AND CALCIUM-DEPENDENT CONDITIONAL ACTIVITY

Provided herein are methods and uses for conditionally controlling the activity of modified matrix metalloproteinases (MMPs), such as matrix metalloproteinase-1 (MMP-1), by virtue of their dependence on calcium for activity. As described herein, the calcium-sensitive MMPs (csMMPs) used in the methods and uses herein are modified MMP polypeptides that exhibit substantially reduced activity at physiological levels of extracellular calcium (e.g., from about 1 mM to 1.3 mM) compared to the activity of the corresponding unmodified MMP not containing the modification(s). The csMMPs exhibit greater activity at higher concentrations of calcium than the same csMMP at physiologic calcium concentrations. Thus, in the methods herein, the csMMPs can be delivered to a subject in the presence of concentrations of calcium that are greater than the physiologic levels of calcium (e.g., greater than 2 mM), wherein the csMMPs are active. As the local concentration of calcium equilibrates to physiologic levels, the activity of the MMP is reduced, thereby achieving conditional or temporal activity of the enzyme.

Since csMMPs are MMPs that degrade proteins that are part of the extracellular matrix, such as collagen, the methods provided herein can be used to conditionally degrade one or more components of the extracellular matrix (ECM) by regulating calcium concentration in the local environment of a csMMP. For example, csMMP compositions formulated in the presence of concentrations of calcium greater than physiological levels of extracellular calcium, such as greater than 1 mM, and generally greater than 2 mM calcium (e.g., 2 mM to 100 mM, such as at least 5 mM or 10 mM), can be delivered to a subject to conditionally degrade one or more components of the extracellular matrix (ECM). In particular, provided herein are methods to treat ECM-mediated diseases or conditions, and in particular fibrotic diseases or conditions, in which a component of the ECM is involved in the etiology or progression of the disease or condition, such as cellulite, Dupuytren's syndrome and Peyronie's disease. For example, the methods provided herein are used to treat fibrotic diseases or conditions that are collagen-mediated diseases or conditions, for example, cellulite, by calcium-dependent temporal regulation of a csMMP.

1. Matrix Metalloproteinase Activity in the Extracellular Matrix

Most MMPs are involved in degradation of the extracellular matrix (ECM). For example, many of these enzymes can cleave components of the basement membrane and extracellular matrix, such as collagen (e.g., collagen I) and other substrates (see e.g., Table 3). MMPs are involved in tissue remodeling, for example, in processes such as wound healing, pregnancy and angiogenesis. In addition, MMPs also can process a number of cell-surface cytokines, receptors and other soluble proteins. The proteolytic activity of MMPs acts as an effector mechanism of tissue remodeling in physiologic and pathologic conditions, and as a modulator of inflammation. The excess synthesis and production of MMPs leads to accelerated degradation of the ECM which is associated with a variety of diseases and conditions such as, for example, bone homeostasis, arthritis, cancer, multiple sclerosis and rheumatoid arthritis. In the context of neuroinflammatory diseases, MMPs have been implicated in processes such as (a) blood-brain barrier (BBB) and blood-nerve barrier opening, (b) invasion of neural tissue by blood-derived immune cells, (c) shedding of cytokines and cytokine receptors, and (d) direct cellular damage in diseases of the peripheral and central nervous system (Leppert et al. (2001) Brain Res. Rev. 36(2-3):249-257; Borkakoti et al. (1998) Prog. Biophys. Mol. Biol. 70(1):73-94). The enzymes are specifically regulated by endogenous inhibitors called tissue inhibitors of matrix metalloproteinases (TIMPs).

Table 3 sets forth exemplary MMPs, and also sets forth exemplary target substrates for each enzyme. Reference to such substrates is for reference and exemplification, and are not intended to represent an exhaustive list of all target substrates. One of skill in the art knows or can empirically determine ECM target substrates for a desired enzyme using routine assays, such as any described herein. The Table also sets forth the sequence identifiers (SEQ ID NOS) for the nucleotide sequence and encoded amino acid sequence of the precursor polypeptide for each of the exemplary proteases are listed in the Table. The sequence identifiers (SEQ ID NOS) for the amino acid sequence of the preproprotein and the zymogen-activated processed mature form of the protein (lacking the propeptide) also are listed in the Table. The location of domains also is indicated. Those of skill in the art are familiar with such domains and can identify them by virtue of structural and/or functional homology with other such domains. It is understood that polypeptides and the description of domains thereof are theoretically derived based on homology analysis and alignments with similar polypeptides. Thus, the exact locus can vary, and is not necessarily the same for each polypeptide. Variations of MMPs also exist among allelic and species variants and other variants known in the art, and such variants also are contemplated for modification as csMMPs as described herein below.

TABLE 3
Matrix Metalloproteinases

| | | | SEQ ID NO | | | |
|---|---|---|---|---|---|---|
| | | | | | Zymogen | Mature |
| | | GenBank | Precursor | | (pro form) | (processed |
| Protease | Substrate | No. | nt | aa | aa | form) |

Collagenases:

| | | | | | | |
|---|---|---|---|---|---|---|
| MMP-1 (interstitial | collagen I, II, III, VII, | P03956, | 4 | 1 | 2 | 5 |
| collagenase-1) | VIII, X, XI, gelatin, | NM_002421 | | (ss aa 1-19; | | |
| | proteoglycan, | | | pp aa 20-99) | | |
| | fibronectin, | | | | | |
| | glycoprotein | | | | | |
| MMP-8 (neutrophil | collagen I, II, III, | P22894 | 15 | 16 | 17 | 132 |
| collagenase; | aggrecan | NM_002424 | | (ss aa 1-20; | | |
| collagenase-2) | | | | pp aa 21-100) | | |
| MMP-13 | collagen I, II, III, IV, | P45452 | 18 | 19 | 20 | 133 |
| (collagenase -3) | VI, IX, X, XIV, | NM_002427 | | (ss aa 1-19; | | |
| | gelatin, proteoglycan, | | | pp aa 20-103) | | |
| | fibronectin, | | | | | |
| | glycoprotein | | | | | |
| MMP-18 | collagen I | Xenopus | 21 | 22 | 23 | 134 |
| (collagenase-4) | | laevis | | (ss aa 1-17; | | |
| | | O13065 | | pp aa 18-99) | | |

Gelatinases:

| | | | | | | |
|---|---|---|---|---|---|---|
| MMP-2 | gelatins, collagen I, | P08253 | 24 | 25 | 26 | 135 |
| (gelatinase A) | II, III, IV, V, VII, X, | NM_004530 | | (ss aa 1-29; | | |
| | XI, elastin, | | | pp 30-109) | | |
| | fibronectin, laminin, | | | | | |
| | proteoglycan, | | | | | |
| | glycoprotein | | | | | |
| MMP-9 | gelatin, collagen IV, | P14780 | 27 | 28 | 29 | 136 |
| (gelatinase B) | V, VI, XIV, elastin, | NM_004994 | | (ss aa 1-19; | | |
| | laminin, | | | pp aa 20-93) | | |
| | proteoglycan, | | | | | |
| | glycoprotein | | | | | |

Stromelysins:

| | | | | | | |
|---|---|---|---|---|---|---|
| MMP-3 | fibronectin, elastin, | P08254 | 30 | 31 | 32 | 137 |
| (stromelysin-1) | laminin, | NM_002422 | | (ss aa 1-17; | | |
| | gelatin, proteoglycan, | | | pp aa 18-99) | | |
| | glycoprotein, | | | | | |
| | collagen III, IV, V, | | | | | |
| | VII, IX, X, XI | | | | | |
| MMP-10 | collagen III, IV, V, | P09238 | 33 | 34 | 35 | 138 |
| (stromelysin-2) | elastin, gelatin, | NM_002425 | | (ss aa 1-17; | | |
| | fibronectin, aggrecan | | | pp aa 18-98) | | |
| MMP-11 | Gelatin, fibronectin, | P24347 | 36 | 37 | 38 | 139 |
| (stromelysin-3) | laminin, | X57766 | | (ss aa 1-31; | | |
| | collagen IV | | | pp aa 32-97) | | |

Matrilysins:

| | | | | | | |
|---|---|---|---|---|---|---|
| MMP-7 | fibronectin, laminin, | P09237 | 39 | 40 | 41 | 140 |
| (matrilysin) | elastin, gelatin, | NM_002423 | | (ss aa 1-17; | | |
| | collagen I, IV, | | | pp aa 18-94) | | |
| | proteoglycan, | | | | | |
| | glycoprotein | | | | | |
| MMP-26 | collagen IV, | Q9NRE1 | 42 | 43 | 44 | 141 |
| (matrilysin-2) | fibronectin, gelatin, | NM_021801 | | (ss aa 1-17; | | |
| | proteoglycan | | | pp aa 18-89) | | |

Metalloelastase:

| | | | | | | |
|---|---|---|---|---|---|---|
| MMP-12 | elastin, fibronectin, | P39900 | 45 | 46 | 47 | 142 |
| (metalloelastase) | laminin, collagen I, | NM_002426 | | (ss aa 1-16; | | |
| | IV, V, gelatin, | | | pp aa 17-105) | | |
| | proteoglycan, | | | | | |
| | glycoprotein | | | | | |

Membrane-type MMPs:

| | | | | | | |
|---|---|---|---|---|---|---|
| MMP-14 | Collagen I, II, III, | P50281 | 48 | 49 | 50 | 143 |
| (MT1-MMP) | gelatin, aggrecan, | NM_004995 | | (ss aa 1-20; | | |
| Transmembrane | fibronectin, laminin, | | | pp aa 21-111) | | |
| | proteoglycan, glycoprotein | | | | | |
| MMP-15 | aggrecan, fibronectin, | P51511 | 51 | 52 | 53 | 144 |
| (MT2-MMP) | laminin, glycoprotein | NM_002428 | | (ss aa 1-41; | | |
| Transmembrane | | | | pp aa 42-131) | | |
| MMP-16 | Collagen III, | P51512 | 54 | 55 | 56 | 145 |
| (MT3-MMP) | fibronectin, laminin, | NM_005941 | | (ss aa 1-31; | | |
| Transmembrane | gelatin, proteoglycan | | | pp aa 32-119) | | |
| MMP-17 | gelatin | Q9ULZ9 | 57 | 58 | 59 | 146 |
| (MT4-MMP) | | AB021225 | | (ss aa 1-38; | | |
| GPI anchor | | | | pp aa 39-128) | | |
| MMP-24 | fibronectin, gelatin, | Q9Y5R2 | 60 | 61 | 62 | 147 |
| (MT5-MMP) | proteoglycan | NM_006690 | | (ss aa 1-52; | | |
| Transmembrane | | | | pp aa 53-155) | | |
| MMP-25 | collagen IV, gelatin, | Q9NPA2 | 63 | 64 | 65 | 148 |
| (MT6-MMP) | fibronectin, | NM_022468 | | (ss aa 1-21; | | |
| GPI anchor | proteoglycan | | | pp aa 22-107) | | |

Enamelysin:

| | | | | | | |
|---|---|---|---|---|---|---|
| MMP-20 | aggrecan | O60882 | 66 | 67 | 68 | 149 |
| (enamelysin) | | Y12779 | | (ss aa 1-22; | | |
| | | | | pp aa 23-107) | | |

Other:

| | | | | | | |
|---|---|---|---|---|---|---|
| MMP-19 | collagen IV, gelatin, | Q99542 | 69 | 70 | 71 | 150 |
| | laminin, aggrecan, | NM_002429 | | (ss aa 1-18; | | |
| | fibronectin, | | | pp aa 19-97) | | |
| | glycoprotein | | | | | |
| MMP-21 | gelatin | Q8N119 | 72 | 73 | 74 | 151 |
| | | NM_147191 | | (ss aa 1-24; | | |
| | | | | pp aa 25-144) | | |
| MMP-23 | gelatin | O75900 | 75 | 76 | 76 | 152 |
| CA-MMP | | AJ005256 | | (pp aa 1-78) | | |
| MMP-27 | gelatin | Q9H306 | 78 | 79 | 80 | 153 |
| CMMP | | NM_022122 | | (ss aa 1-17; | | |
| | | | | pp aa 18-98) | | |
| MMP-28 | | Q9H239 | 81 | 82 | 83 | 154 |
| (epilysin) | | NM_024302 | | (ss aa 1-22; | | |
| | | | | pp aa 23-122) | | |

2. Regulation of MMP Activity

Under normal physiological conditions, MMP activity can be regulated at various stages: during transcription, proteolytic processing (i.e., activation) of proenzyme forms (e.g., proMMP-1 (discussed below)), as well as by inhibition of enzyme activity by a specific or broad range of chemical inhibitors, or by endogenous inhibitors. In addition, the availability of metal ions, such as zinc and calcium, also can regulate MMP catalytic activity, for example by altering the tertiary structure and stability of the enzyme.

a. Metal Binding

The presence of metal ions is required for MMP catalytic activity. The catalytic domain of MMPs (further discussed below) binds two zinc ions and one to three calcium ions. One of the zinc ions is required for catalytic activity. The importance of zinc ions to MMP catalysis has been examined by metal replacement. For example, transition metals (e.g., CO²⁺, Mn²⁺, Cd²⁺ and Ni²⁺) can be used as substitutes for the catalytic zinc, resulting in retained, albeit reduced, protease activity, but altered substrate specificity (Cha et al. (1998) J. Biol. Inorg. Chem. 3:353-359).

MMP enzymes can contain multiple calcium binding sites in its catalytic domain, including high-affinity and low-affinity calcium binding sites. The dissociation constants for high-affinity binding regions are in the nanomolar range, whereas low-affinity dissociation constants are in the micromolar range. For example, MMP-26 requires the presence of at least 120 μM calcium for enzyme activity (Lee et al. (2007) Biochem. J. 403:31-42). Ca²⁺ binding of MMP-1 is cooperative, with a Hill coefficient of 2.9 and 50% saturation at 400 μM Ca²⁺ (Zhang et al. (1997) J. Biol. Chem. 272:1444-1447). The dependence of catalytic activity on Ca²⁺ concentration also is cooperative, with a Hill coefficient of 1.7-2.0 and a midpoint Ca²⁺ concentration of 0.2 μM (Zhang et al. (1997) J. Biol. Chem. 272:1444-1447).

The calcium dependence of the enzymatic activity is a result of the dependence of MMP tertiary structure on the presence of calcium. For example, calcium ions can modulate MMP catalytic activity through conformational changes in the active site (Lee et al. (2007) Biochem. J. 403:31-42; Zhang et al. (1997) J. Biol. Chem. 272:1444-1447). Insufficient calcium concentrations lead to interdomain conformational changes which lead to increased protein flexibility and decreased thermostability of MMPs (Ohvayashi et al. (2012) Appl. Environ. Microbiol. 78(16):5839-5844). In addition, MMP stability is regulated by Ca²⁺ binding, as the Ca²⁺-stabilized active conformation of MMPs is more resistant to denaturants and less susceptible to proteolysis (Lowry et al. (1992) Proteins Struc. Funct. Genet. 12:42-48; Housley et al. (1993) J. Biol. Chem. 268:4481-4487).

b. Endogenous Inhibitors

Endogenous inhibitors also regulate MMP activity. For example, exemplary of endogenous inhibitors are tissue inhibitors of metalloproteinases (TIMPs) (Nagase and Woessner (1999) J. Biol. Chem. 274(31):21491-21494; Vu and Werb (2000) Genes Dev. 14(17):2123-2133; Baker et al. (2002) J. Cell Science 115:3719-3727). Four TIMPs have been identified in vertebrates: TIMP-1, TIMP-2, TIMP-3, and TIMP-4. TIMPs (21 to 29 kDa) are secreted proteins that inhibit MMPs by binding the active site and chelating the active-site zinc (Visse and Nagase (2003) Circ. Res. 92:827-839). The expression of TIMPs is regulated during development and tissue remodeling.

In addition to TIMPs, proteins such as macroglobulin, thrombospondin-1 and thrombospondin-2 can inhibit MMP activity by forming complexes with MMPs and facilitating their removal from the extracellular environment (Baker et al. (2002) J. Cell Science 115:3719-3727). For example, α2-macroglobin, a 772 kDa protein, contains a cleavable ‘bait’ region that, once cleaved by a MMP (e.g., MMP-1), causes a conformational change that entraps the proteinase, which becomes covalently anchored by transacylation. The MMP-α2-macroglobin complex can then bind the low density lipoprotein receptor related protein (LDL-RP), which results in receptor-mediated endocytosis and degradation (Baker et al. (2002) J. Cell Sci. 115:3719-3727; Barrett (1981) Methods Enzymol. 80:77-754; Feldman et al. (1985) Biochem. 82:5700-5704).

3. Role of Collagen in ECM Diseases and Conditions

Among the primary substrates of many MMPs is collagen, and in particular collagen I. Collagen is the main source of structural support for multicellular animals. The mechanical strength of collagen depends on a highly regulated mechanism of intermolecular crosslinking. Collagen is a major structural constituent of mammalian organisms and makes up a large portion of the total protein content of the skin and other parts of the animal body. However, collagen has been associated with the etiology or progression of various diseases or conditions. For example, numerous diseases and conditions are associated with excess collagen deposition due, for example, to erratic accumulation of fibrous tissue rich in collagen, or other causes. Certain diseases and conditions result from defects or changes in the architecture of the extracellular matrix due to aberrant expression or production of collagen. For example, in some inflammatory conditions, such as those that occur upon wound healing, cytokines are secreted, which stimulate fibroblasts to secrete collagen. The collagen can then accumulate as they are locally deposited, and the cross-linking of collagen molecules can result in a wide range of fibrotic conditions.

Collagen-mediated diseases or conditions (also referred to as fibrotic tissue disorders) are known to one of skill in the art (see e.g., published U.S. Application No. 2007/0224183; U.S. Pat. Nos. 6,353,028; 6,060,474; 6,566,331; 6,294,350). Excess collagen deposition is a feature in several chronic inflammatory diseases and in other diseases and conditions, including, but not limited to, fibrotic diseases or conditions resulting in scar formation, Dupuytren's syndrome, Peyronie's disease, Ledderhose fibrosis, adhesive capsulitis (e.g., frozen shoulder), stiff joints, scleroderma, localized scleroderma (e.g., sclerodactyly), lymphedema, interstitial cystitis (IC), telangiectasia, Barrett's metaplasia, pneumatosis cystoides intestinalis, collagenous colitis, ventricular hypertrophy (e.g., left ventricular hypertrophy (LVH), atherosclerosis, arterial stenosis, and scars, such as scars resulting from among surgical adhesions or keloids, hypertrophic scars and depressed scars. Excessive collagen deposition can produce unwanted binding and distortion of normal tissue architecture, leading to disfiguring conditions of the skin, such as wrinkling, cellulite formation and neoplastic fibrosis.

Several of the collagen-mediated diseases or conditions above are characterized by the presence of abundant fibrous septae of collagen, which are constructed from cross-linked collagen filaments (also called cords or cables), or collagen plaques. For example, cellulite is a prominent condition that is characterized by alterations in the connective tissue matrix resulting in an abnormal fibrous septae network of collagen in addition to adipogenicity (Rawlings et al. (2006) Int. J. Cos. Science 28:175-190).

Mechanical stability of collagen septae is established by intermolecular cross-linking between collagen strands to form fibrils. The tensile strength of the collagen fiber is related to the collagen crosslink type, concentration of crosslinks and the type of collagen. The stiffness (or toughness) of the tissue containing the collagen fiber(s) also is a function of the type of collagen in the fiber and crosslinking. Under healthy conditions, collagen septae serve to stiffen soft cellular tissue and also to provide structure by physically compartmentalizing the tissue, for example, to establish planes of ingress for small blood vessels. Changes in the distribution, quantity and relative proportions of collagens in these tissues can lead to formation of additional collagen septae, enlargement of present collagen septae, and/or formation of collagen plaques. These abnormal structures can lead to altered cell phenotypes, architectural distortion of the tissue with altered blood flow, impaired nutrient diffusion, and altered cell signaling (Wells, R. G., “Function and metabolism of collagen and other extracellular matrix proteins,” in The Textbook of Hepatology: from Basic Science to Clinical Practice. 3rd ed.; Rodés J, editor; Oxford: Blackwell Publishing; 2007).

4. Conditional Regulation of MMP Activity by Calcium-Dependence

Diseases and conditions of the ECM that are characterized by aberrant expression or overproduction of one or more collagens or other ECM component, resulting in their accumulation and unwanted deposition, can be treated by enzymes that degrade collagen or other ECM component. For example, certain bacteria, such as Clostridium, produce collagenases. Bacterial collagenase (e.g., from clostridium histolyticum), is an enzyme active at neutral pH that degrades collagen, and has been clinically tested for multiple indications, including Dupuytren's contraction, Peyronie's Disease, frozen shoulder, cellulite, lipoma, and others. For example, bacterial collagenases have been used to treat collagen-mediated conditions such as cellulite (see e.g., US 2007/0224184); Dupuytren's syndrome (see e.g., U.S. Pat. Nos. RE39,941; 5,589,171; 6,086,872; 7,811,560); Peyronie's disease (see e.g., U.S. Pat. No. 6,022,539), and to promote wound healing (see e.g., U.S. Publication Nos. 2003/0170225, 2006/0222639 and 2008/0145357 and U.S. Pat. Nos. 6,074,664 and 7,641,900). It is approved for the treatment of Dupuytren's contracture in the United States (marketed as XIAFLEX®), Europe (marketed as XIAPEX®) and Canada. In particular, bacterial collagenase marketed as XIAFLEX® contains purified collagenase isolated and purified from the fermentation of Clostridium histolyticum bacteria and is made of two microbial collagenases: collagenase AUX-I and collagenase AUX-II.

Collagenase is capable of irreversibly cleaving collagens, for example, collagens of type I, II and III. Tissue destruction caused by these collagenases also has been linked to the pathogenesis of human diseases with which the collagenase-producing bacteria are associated (Harrington (1996) Infect Immun. 64(6):1885-1891). Accordingly, it is not unexpected that the prolonged and unregulated activity of collagenase can result in excessive collagen degradation, which can lead to side-effects due to undesirable tissue destruction, including prolonged or unwanted degradation of ligaments, scar tissue and tendon. Bacterial collagenases also differ from vertebrate collagenases in that they exhibit broader substrate specificity. Bacterial collagenase can degrade most collagen types, with multiple cleavages in triple helical regions. Unlike vertebrate collagenases, bacterial collagenases can degrade both native and denatured forms of collagen, and several also have the ability to effectively degrade type IV collagen which is known to contain regions of non-helical conformation (Mookhtiar and Van Wart (1992) Matrix Suppl. 1:116-126). Limited activity against type IV collagen, found in basement cell membranes, can account for bleeding side effects of underlying endothelium. Hence, administration of collagenase (e.g., bacterial collagenase) risks side effects associated with prolonged activity and limits the dosages that can be administered. For example, prolonged activation of administered collagenase can result in unwanted side effects, such as swelling, pain, bruising, pruritus, loss of tissue tensile properties, and substantial bleeding at sites of injection (Bedair et al. (2007) J. Appl. Physiol. 102:2338-2345; Hurst et al. (2009) New England Journal of Medicine 361:968-979; and U.S. Patent Publication No. 2010/0003237).

Because MMPs are inherently calcium-dependent endopeptidases, the activity of MMPs requires a minimum concentration of calcium for activity. It is found herein that MMPs can be modified to increase the minimum concentration of calcium required for activity. For example, modified MMP polypeptides are provided herein that exhibit an increase in sensitivity to calcium concentrations, for example, so that the MMP requires increased levels of calcium for catalytic activity. Increasing the calcium dependency of MMPs can achieve temporary enzyme activation, thereby regulating the duration of csMMP enzymatic action on extracellular matrix (ECM) components, and overcome the limitations and side effects of uncontrolled MMP activity.

In particular, since the physiological level of calcium in the ECM is less than 2 mM, it is found herein that select modified MMP polypeptides that exhibit calcium sensitive activity as a function of calcium concentration can be utilized to achieve temporal regulation of the MMP in physiologic environments. As described in the Examples and elsewhere herein, modified MMPs are identified that exhibit reduced activity at physiologic calcium levels (e.g., less than 2 mM), while exhibiting greater activity at higher concentrations of calcium (e.g., greater than 2 mM, such as at least 5 mM or at least 10 mM or greater). In contrast, unmodified MMPs, such as unmodified or wild-type MMP-1, exhibit substantially similar activity across a broad range of calcium concentrations, including substantial activity at physiologic levels of calcium. Thus, unlike wild-type MMP polypeptides, the activity of csMMP polypeptides can be temporally regulated by controlling local concentrations of calcium. This can avoid uncontrolled or prolonged degradation of collagen and other ECM components that can be associated with adverse side effects of treatment.

By virtue of the temporal activation of such enzymes upon in vivo administration, the treatment of such diseases and conditions is regulated to limit the enzymatic degradation of the matrix components. For example, by limiting the duration of action of matrix degradation, unwanted side effects associated with uncontrolled protein degradation are minimized. This is an advantage of the present method of treating an ECM-mediated disease or condition over existing collagenase treatments, because methods of treating diseases and/or conditions of the ECM using calcium sensitive MMPs can reduce deleterious side effects associated with unwanted prolonged activation of enzymes by regulating csMMP activity with local calcium levels.

For example, the methods of using the modified csMMP polypeptides provided herein that exhibit calcium sensitivity and are conditionally active, can be used to treat ECM-mediated diseases and disorders. For example, such csMMP polypeptides are active at calcium concentrations that are higher than the normal extracellular calcium concentrations in the ECM. Thus, when administered to the ECM in the presence of high calcium, the enzymes exhibit activity. In one example, before administration, a csMMP can be reconstituted in a buffer containing concentrations of calcium that are higher than normal extracellular physiological levels. The csMMP exhibits activity when exposed to high calcium concentrations (e.g., 10 mM Ca²⁺). As the calcium steadily diffuses, decreasing the calcium concentration local to the csMMPs, and approaching or reaching physiologic levels of extracellular calcium, the activity of the csMMP is reduced. Thus, the csMMP exhibits conditional activity, conditioned upon maintenance of an increased calcium concentration. For example, the activity of the csMMP can be controlled for a predetermined time by maintaining calcium concentrations in the ECM that are higher than the normal physiological calcium concentration.

The following sections describe in further detail the methods and uses provided herein of temporal or conditional activity of a MMP polypeptide by regulating local calcium. For example, described below are exemplary modified MMP polypeptides that are csMMPs and compositions thereof for use in the methods. Also described are exemplary collagen-mediated diseases and conditions that can be treated by any of the csMMP polypeptides provided herein. The description is exemplified based on csMMP-1 polypeptides, but as described herein, can be adapted by one of skill in the art based on the description herein for use of other csMMP polypeptides in the methods for treating any collagen-mediated disease or condition in which a substrate of the MMP is involved in the etiology or progression of disease.

### C. MATRIX METALLOPROTEINASES (MMPs): MMP-1 AND OTHER MMP COLLAGENASES

Matrix metalloproteinases (MMPs) are a family of zinc-dependent and calcium-dependent endopeptidases. There are over 25 MMPs known and they are grouped into different families depending on function, substrate specificity and/or sequence similarity. The families of MMPs include collagenases, gelatinases, stromelysins, matrilysins, metaloelastases, membrane-type MMPs, and enamelysins (see e.g., Table 4).

MMPs share similar structure made up of common domains. The basic structure of MMPs is made up of the following homologous domains: 1) a signal peptide which directs MMPs to the secretory or plasma membrane insertion pathway, which is removed in the endoplasmic reticulum to yield latent proenzymes (with the exception of MMP-23 which lacks the pro-domain and is secreted in its active form); 2) a pro-domain that confers latency to the enzymes by occupying the active site zinc, making the catalytic enzyme inaccessible to substrates; 3) a catalytic domain, containing a Zn²⁺ ion which is required for proteolytic activity; 4) one or more hemopexin (Hpx) domains which mediate interactions with substrates (and inhibitors, such as tissue inhibitors of metalloproteinases (TIMPs)) and confer specificity of the enzyme; and 5) a proline-rich, flexible hinge region of approximately 75 amino acids, which has no known structure and links the catalytic and the hemopexin domains. The domain organization of MMPs is summarized in Table 4 below.

MMP-7, -26, and -23 are exceptions to this basic domain structure (Table 4). MMP-7 and MMP-26 lack the hemopexin domain, yet display specificity in substrate degradation. MMP-23, also called cysteine array MMP, lacks a functional pro-domain and the hemopexin domain; instead, it has a cysteine-rich domain followed by an immunoglobulin-like domain and a type II transmembrane domain in the N-terminal part of the polypeptide.

Additional structural domains are characteristic of certain MMP subgroups. For example, the membrane-type MMPs contain an additional transmembrane domain and a small cytoplasmic domain (MMP-14, MMP-15, MMP-16, and MMP-24) or a glycosylphosphatidyl inositol linkage (MMP-17 and MMP-25), which anchors these proteins to the cell surface. MMP-2 and MMP-9 contain up to three tandem repeats of fibronectin type II modules that confer gelatin-binding properties to these enzymes.

TABLE 4
Domain structure of MMPs

| | signal | type II | pro- | | furin | | | | | type I | | | Cys | IgG- |
|---|---|---|---|---|---|---|---|---|---|---|---|---|---|---|
| | pep- | trans-mb | pep- | V | clvg | Cat | Fn | | Hpx | trans-mb | GPI | Cp | array | like |
| Class: MMP | tide | domain | tide | insert | site | domain | repeats | Hinge | domain | domain | anchor | domain | region | domain |
| Matrilysins | + | − | + | − | − | + | − | − | − | − | − | − | | − |
| MMP-7, -26 | | | | | | | | | | | | | | |
| Collagenases: | + | − | + | − | − | + | − | + | + | − | − | − | | − |
| MMP-1, -8, | | | | | | | | | | | | | | |
| -13, -18 | | | | | | | | | | | | | | |
| Stromelysins: | | | | | | | | | | | | | | |
| MMP-3, -10 | | | | | | | | | | | | | | |
| Other MMPs: | | | | | | | | | | | | | | |
| MMP-12, -19, | | | | | | | | | | | | | | |
| -20, -27 | | | | | | | | | | | | | | |
| Gelatinases: | + | − | + | − | − | + | + | + | + | − | − | − | | − |
| MMP-2, -9 | | | | | | | | | | | | | | |
| Other MMP: | + | − | + | + | + | + | − | + | + | − | − | − | | − |
| MMP-21 | | | | | | | | | | | | | | |
| Other MMPs: | + | − | + | − | + | + | − | + | + | − | − | − | | − |
| MMP-11, -28 | | | | | | | | | | | | | | |
| Membrane | + | − | + | − | + | + | − | + | + | + | − | + | | − |
| Type MMPs: | | | | | | | | | | | | | | |
| MMP-14, -15, | | | | | | | | | | | | | | |
| 16, -24 | | | | | | | | | | | | | | |
| Membrane | + | − | + | − | + | + | − | + | + | − | + | − | | − |
| Type MMPs: | | | | | | | | | | | | | | |
| MMP-17, -25 | | | | | | | | | | | | | | |
| Other MMP: | + | + | − | − | + | + | − | − | − | − | − | − | + | + |
| MMP-23 | | | | | | | | | | | | | | |

Abbreviations:
V = vitronectin
Cat = catalytic
Fn = fibronectin
Hpx = hemopexin
Cp = cytoplasmic

MMPs (with the exception of MMP-23) are synthesized and secreted as latent zymogens. Zymogen activation prevents unwanted protein degradation that could occur if proteases were always present in active form. The propeptide of zymogen forms of MMPs ranges in size from about 80-100 residues in length and serves to stabilize the zymogen form and also to prevent catalytic activity of the MMP enzyme.

The propeptide of MMP zymogens forms a globular structure containing a three helix fold that is stabilized by hydrophobic interactions and hydrogen bonds (Morgunova et al. (1999) Science. 284:1667-1670). The highly conserved motif, PRCxxPD (SEQ ID NO:84), is located in the C-terminal portion of the propeptide and contains a cysteine residue, which confers latency to the proenzyme. The sulfhydryl group of the cysteine residue coordinates with the catalytic Zn²⁺ ion, bound in the active site of the enzyme (discussed further below), sterically blocking the active site of the protease and preventing access of substrates to the catalytic center (Van Wart et al. (1990) Proc. Natl. Acad. Sci U.S.A. 87:5578-5582; Nagase and Woessner (1999) 1 Biol. Chem. 274:21491-21494; Birkedal-Hansen (1995) Curr. Opin. Cell Biol. 7:728-735). The loop between the second and third helices of the propeptide assists in stabilizing the zymogen form by shielding the catalytic cleft with its hydrophobic side-chains, thereby preventing access of solvent molecules that could disrupt the activity-inhibiting cysteine-Zn²⁺ interaction.

The loop between the first and second helices, the residues of which vary between MMPs, provide a “bait region” that is cleaved by activating proteases (e.g., trypsin or other MMPs). Proteases also can cleave loops, between the second and third helices. Upon cleavage of one or both of these loop regions by activating proteases, the propeptide structure is disrupted, and the shielding of the catalytic cleft is withdrawn, allowing water to enter and hydrolyze the cysteine-Zn²⁺ coordination, leading to activation via a “cysteine-switch” mechanism (Van Wart and Birkedal-Hansen (1990) Proc. Natl. Acad. Sci. U.S.A. 87:5578-5582). Final processing, to completely remove the propeptide, typically involves autoproteolytic cleavage by the target MMP. Activation of MMPs also can be entirely autocatalytic, for example, following interruption of the cysteine to zinc coordination effected by exposure of the enzyme to sulfhydryl-reacting compounds (exemplary reactive compounds are set forth in Section E.4).

As discussed above, activation of MMP zymogens requires processing, which generally involves removal of the propeptide (e.g., positions 1-80 of SEQ ID NO:2) and/or conformational changes of the enzyme to generate a processed mature form. Processing of the enzyme by removal of the propeptide is required for activity of MMPs. For normal MMPs (e.g., wild-type) that are not conditionally active as are those used in the methods provided herein, the processed mature form is an active enzyme. Thus, it is understood that wild-type MMPs in their processed mature form are enzymatically active, and thus for these enzymes this is the active form. csMMPs provided herein, however, additionally require the presence of sufficient calcium to be fully active.

1. Matrix Metalloproteinase-1 (MMP-1)

MMP-1, also called interstitial collagenase, is a member of the MMP family. Like the other MMPs, the structure of MMP-1 is maintained by binding several Zn²⁺ and Ca²⁺ ions and requires a Zn²⁺ ion bound in the active site for catalytic activity. MMP-1 is expressed by several types of cells, including fibroblasts, and participates in the degradation of ECM components following secretion into the interstitial space. Degradation of the ECM is an important step in tissue remodeling, for example, in processes such as embryogenesis, tissue repair and remodeling, angiogenesis, organ morphogenesis and wound healing.

MMP-1 is encoded by a nucleic acid molecule set forth in SEQ ID NO:4, resulting in a preproenzyme (SEQ ID NO:1), which is cotranslationally processed to remove a signal peptide to generate an inactive precursor, known as a zymogen or proenzyme (proMMP-1; SEQ ID NO:2). The secreted MMP-1 zymogen is a multidomain enzyme that contains a prodomain of 80 amino acids (corresponding to amino acid residues 1-80 of SEQ ID NO:2), a catalytic domain of 162 amino acids (corresponding to amino acid residues 81-242 of the sequence of amino acids set forth in SEQ ID NO:2), a 16-residue linker (corresponding to amino acid residues 243-258 of the sequence of amino acids set forth in SEQ ID NO:2), and a hemopexin (Hpx) domain of 189 amino acid residues (corresponding to amino acid residues 259-450 of the sequence of amino acids set forth in SEQ ID NO:2) (Jozic et al. (2005) J. Biol. Chem. 280(10):9578-9785). Upon processing, as described above, the propeptide is removed, resulting in a processed mature form having a sequence of amino acids set forth in SEQ ID NO:5.

As noted above, MMP-1 cleaves collagen type I and collagen type III, which are the most abundant proteins of the skin. These collagen types are associated with many of the conditions of the ECM as described herein in Section B.2. In contrast, collagen type IV is a major component of the basal lamina of blood vessels. Hence, targeting of type IV collagen, for example, can lead to leaky blood vessels, which can be a side effect of treatments that are meant to target the extracellular matrix as described herein if the treatment does not selectively target collagens of type I or III. For example, bacterial collagenase, which has been used as treatment for cellulite, can induce hemorrhages through cleavage of type IV collagen in addition to type I and/or type III collagen (see e.g., Vargaftig et al. (2005) Inflammation Res. 6:627-635). Thus, an advantage of the use of MMP-1, and in particular csMMP-1 that can be conditionally and temporally controlled, is that it does not cleave type IV collagen when used as a therapeutic agent to treat conditions of the ECM.

a. Catalytic Domain

The catalytic domain of MMP-1 is similar in structure to other MMPs and has a shallow active site cleft that separates a smaller “lower subdomain” and a larger “upper subdomain.” The catalytic domain encompasses a characteristic five-stranded, highly twisted beta-sheet, flanked by three surface loops on its convex side and two alpha-helices on its concave side, followed by a third alpha helix following the specificity loop (Maskos, K. (2005) Biochimie. 87:240-263). Integral to the structure of MMP-1 is the binding of two Zn²⁺ ions and three Ca²⁺ ions. Zn²⁺- and Ca²⁺-binding residues within the catalytic domain are highly conserved among MMP family members (Maskos, K. (2005) Biochimie. 87:240-263).

i. Zn²⁺ Ions

Like other matrix metalloproteinases (MMPs), MMP-1 contains a Zn²⁺ ion at the active center of the enzyme that is required for catalytic activity. This “catalytic zinc” is bound to the conserved MMP zinc binding motif, HExGHxxGxxH (SEQ ID NO:85; corresponding to amino acid residues 199-209 of SEQ ID NO:2), in the active site, which is followed by a methionine turn which also is conserved among MMPs (Bode et al. (1993) FEBS Lett. 331:134-140). The imidazole side-chains of the histidine residues within the zinc binding motif at the active site of MMP-1 are ligands to the Zn²⁺. During catalysis, the catalytic Zn²⁺ promotes nucleophilic attack on the carbonyl carbon by the oxygen atom of a water molecule at the active site. An active-site base (a glutamate residue in carboxypeptidases) facilitates this reaction by extracting a proton from the attacking water molecule. Thus, the glutamate (E) residue (amino acid 200 of SEQ ID NO:2) activates a zinc-bound H₂O molecule, thereby providing the nucleophile that cleaves peptide bonds. Mutation of any one of the histidines, within the zinc binding motif, ablates MMP-1 catalytic activity.

In addition to the catalytic zinc MMP-1 possesses a second “structural zinc” ion. Three histidine residues, corresponding to amino acid residues 149, 164, and 177 of SEQ ID NO:2, and a glutamate residue, corresponding to amino acid residue 151 of SEQ ID NO:2, form a tetrahedral coordination sphere to bind the second zinc, which confers stability to the tertiary structure of MMP-1.

ii. Ca²⁺ Ions

In addition to interactions with zinc ions, the structure of the catalytic domain of MMP-1 also is maintained by the binding of three Ca²⁺ ions. One Ca²⁺ molecule also binds to the Hpx-like domain. While calcium does not directly participate in proteolysis, the binding of calcium molecules plays an important role in the stabilization of the tertiary structure of the enzyme, thereby regulating catalytic activity (Seltzer et al. (1976) Arch. Biochem. Biophys. 173:355-361; Housley et al. (1993) J. Biol. Chem. 268:4481-4487). In the absence of calcium, MMP-1, for example, is catalytically inactive and adopts a partially unfolded state with native secondary structure but altered tertiary structure (Zhang et al. (1997) J. Biol. Chem. 272:1444-1447).

The Ca²⁺-binding sites are characterized as being highly conserved Glu- and Asp-rich regions. On either side of the structural zinc binding motif, MMP-1 contains two calcium binding sites. Asp156, Gly157, Gly159, N161, Asp179 and Glu182, of SEQ ID NO:2, are involved in binding the first calcium ion which is probably the most tightly bound calcium ion. This calcium packs the S-shaped loop, between the third and fourth strands of the twisted beta sheet, against the side-chain carboxylate groups of the fifth strand of the beta sheet.

The second calcium ion is located between the loop between the fourth and fifth strands, and the third strand of the twisted beta sheet. This calcium ion is coordinated in an octahedral manner by the carbonyl groups of residues Asp139, Gly171 and Gly173 and the carboxylate oxygen of Asp175, and a water molecule.

The loop following the fifth strand of the twisted beta sheet encircles a third calcium ion, which is held by the carbonyl groups of Glu180 and Glu182 together with the carboxylate oxygen of Asp105 and Glu180 and two water molecules in a pentagonal bipyramid.

MMP-1 binding of these three calcium ions is important for the maintenance of the tertiary, but not secondary, structure of the enzyme (Zhang et al. (1997) J. Biol. Chem. 272:144-1447). The tertiary structure of MMP-1 is important, in particular active MMP-1, for catalytic activity, thermal stability, and resistance to denaturants and proteolysis (Housley et al. (1993) J. Biol. Chem. 268: 4481-4487; Lee et al. (2007) Biochem. J. 403:31-42; Lowry et al. (1992) Proteins Struc. Funct. Genet. 12:42-48; Ohvayashi et al. (2012) Appl. Environ. Microbiol. 78(16):5839-5844; Zhang et al. (1997) J. Biol. Chem. 272:144-1447).

b. Linker Region and Hemopexin-Like Domain

The linker, or hinge region, of MMP-1 is a flexible, proline-rich segment of 16 amino acid residues that links the catalytic domain with the hemopexin (Hpx)-like domain, corresponding to amino acid residues 243-258 of the sequence of amino acids set forth in SEQ ID NO:2. This region is important for the stability of MMP-1 and is involved in the degradation of complex substrates, such as fibrillar collagen, which requires concerted action of the catalytic and hemopexin domains (Chung et al. (2004) EMBO J. 23:3020-3030; Overall, C. (2002) Mol. Biotechnol. 22:51-86). The hinge region also may contribute to collagen binding and unwinding activities of MMP-1 (Tam et al. (2004) J. Biol. Chem. 279:43336-43344).

MMP-1 also contains a hemopexin (Hpx)-like C-terminal domain, containing 189 amino acid residues (corresponding to amino acid residues 259-450 of the sequence of amino acids set forth in SEQ ID NO:2), that functions in protein-protein interactions and is important for substrate recognition and interactions with inhibitors, in particular tissue inhibitors of metalloproteinases (TIMPs). The Hpx-like domain is important for the processing of collagen (Murphy et al. (1992) J. Biol. Chem. 267:9612-9618). Other protein-protein interactions mediated by the hemopexin domain are important for enzyme activation, localization, internalization, and degradation (Nagase (1997) Biol. Chem. 378:151-160).

c. MMP-1 Substrates

As an endopeptidase, MMP-1 is capable of cleaving several ECM-related substrates, including structural and non-structural components of the ECM and components of the basement membrane, as set forth in Table 3 (Hagemann et al. (2012) World J. Clin. Oncol. 3(5):67-79). In particular, MMP-1 cleaves interstitial fibrillar collagen. Collagens are the major structural proteins of connective tissues such as skin, tendon, ligament, bone, cartilage, blood vessels and basement membranes. Collagen is composed of a triple helix, which generally consists of two identical chains (α1) and an additional chain that differs slightly in its chemical composition (α2). Interstitial collagens I, II and III are the most abundant collagens, and they provide the scaffolding of the tissue and guide cells to migrate, proliferate and differentiate. Collagen degradation is integral to several biological processes such as embryogenesis, tissue repair and remodeling, angiogenesis, organ morphogenesis and wound healing. MMP-1 binds collagen molecules, locally unwinds the triple helical structure, and hydrolyzes collagen peptide bonds, e.g., at the Gly775-Ile776 or Gly775-Leu776 peptide bonds of the α1 and α2 chains of collagen I, resulting in a three-quarter length N-terminal fragment and a one-quarter length C-terminal fragment. The collagen fragments are unstable at body temperature and undergo denaturation into gelatin, rendering them susceptible to further digestion by other non-specific tissue proteinases.

Degradation of collagen fibrils is not the only result of MMP-1 cleavage of collagen, as the collagen fragments have been linked to other biological functions, such as controlling cellular behavior during tissue remodeling. For example, collagen cleavage products have been implicated in activation and recruitment of osteoclasts during bone remodeling (Zhao et al. (1999) J. Clin. Invest. 103:517-524), epithelial cell migration during wound healing (Pilcher et al. (1997) J. Cell Biol. 137:1445-1457) and apoptosis of amniotic fibroblasts before the onset of labor (Lei et al. (1996) J. Clin. Invest. 98:1971-1978). However, aberrant collagen cleavage is associated with progression of diseases such as arthritis, cancer, atherosclerosis, aneurysm and fibrosis (Woessner (1998) in Matrix Metalloproteinases, Parks W. C. and Mecham, R. P. (eds), pp 1-14; Brinckerhoff and Matrisian, (2002) Nat. Rev. Mol. Cell. Biol. 3:207-214).

MMP-1 collagenolytic activity is essentially mediated through the (183)RWTNNFREY(191) motif (SEQ ID NO:86) of the catalytic domain in concert with the C-terminal hemopexin domain (Chung et al. (2000) J. Biol. Chem. 275(38):29610-29617). Active MMP-1 hydrolyzes type I collagen into N-terminal and C-terminal fragments. However, it hydrolyzes type III collagen 10-fold faster than type I collagen (Wilhelm et al. (1984) Coll. Relat. Res. 4(2):129-152). MMP-1 also cleaves α-chains of native type II collagen (Fields et al. (1987) J. Biol. Chem. 262:6221-6226), type VII collagen (Seltzer et al. (1989) J. Biol. Chem. 264:3822-3826), type X collagen (Schmid et al. (1986) J. Biol. Chem. 261:4184-4189), type VIII (Gadher et al. (1989) Matrix 9:109-115), α2-macroglubulin (Sottrup-Jensen and Birkedal-Hansen (1989) J. Biol. Chem. 264:393-401), gelatin and casein (Fields et al. (1990) Biochem. 29:6670-6677), α1-proteinase inhibitor and α1-antichymotrypsin (Desrochers et al. (1991) J. Clin. Invest. 87:2258-2265), tenascin (Imai et al. (1994) FEBS Lett. 352:216-218) and IL-1β (Ito et al. (1996) J. Biol. Chem. 271:14657-14660).

2. Other Collagenases

Collagenase activity also can be conferred by MMPs other than MMP-1. While several MMPs have the capability of degrading collagen, MMP-8, MMP-13 and MMP-18, in particular, are categorized as collagenases based on their preference for collagen as a substrate. Bacterial collagenase also degrades collagen. As there are multiple forms of collagen, collagenases vary with respect to their collagen-substrate specificities. Collagenases also differ in expression patterns.

MMP-8, also called Neutrophil Collagenase, is naturally expressed in exclusively inflammatory conditions. While MMP-1 degrades type III collagen more efficiently than type I or type II collagen, MMP-8 preferentially degrades type I collagen over type III or type II collagen. MMP-8 is highly expressed in the postpartum uterus, and it is thought to be involved in the postpartum involution of the uterus. MMP-8 also is the predominant collagenase expressed in ulcers and healing wounds. A sequence alignment of MMP-1 and MMP-8 is set forth in FIG. 2A.

MMP-13, also called Collagenase-3, is expressed in the skeleton during embryonic development. MMP-13 preferentially cleaves type II collagen, and is thus involved in the turnover of connective tissue. A sequence alignment of MMP-1 and MMP-13 is set forth in FIG. 2B.

MMP-18, also called Collagenase-4, is found in Xenopus laevis and degrades type I collagen. MMP-18 is expressed in metamorphosing tadpoles and its expression is localized to the tail during tail resorption. MMP-18 also may play a role in hind limb morphogenesis and digestive tract remodeling. A sequence alignment of MMP-1 and MMP-18 is set forth in FIG. 2C.

### D. CALCIUM-SENSITIVE MODIFIED MATRIX METALLOPROTEASE POLYPEPTIDES (csMMP)

Provided herein are methods that utilize modified MMP polypeptides or catalytically active fragments thereof, such as modified MMP-1 polypeptides or catalytically active fragments thereof, that exhibit calcium-sensitive activity in the presence of high calcium concentrations greater than physiological levels, and decreased activity in the presence of physiological levels of calcium. Such modified MMP polypeptides or catalytically active fragments thereof are also referred to herein as calcium-sensitive MMP (csMMP) polypeptides. Hence, the csMMPs can degrade one or more components of the ECM, in particular a collagen (e.g., type I or type III) in a calcium-dependent manner, whereby the modified MMP polypeptide is conditionally active in the presence of calcium concentrations greater than present in a physiological environment and activity decreases upon exposure to physiological conditions.

Accordingly, the activity of such modified MMPs can be modulated by the concentration of calcium, rendering them therapeutic molecules whose activity can be controlled in vivo for therapeutic applications such as fibrotic diseases. By virtue of the ability to degrade a component of the ECM, and in particular a collagen, the csMMPs are used in methods herein to treat fibrotic diseases or other conditions that are associated with accumulated or unwanted presence of a component of the ECM (e.g., a collagen). The csMMP, however, are active for only a limited time upon administration to a subject, such as administration to the ECM of a subject, thereby avoiding prolonged degradation of components of the extracellular matrix, which is a problem with other collagenase treatments. Thus, the activation of the csMMPs, for example upon administration to the body, can be temporally and conditionally controlled by virtue of changes in calcium concentrations in the local environment of the csMMPs. For example, the activation of the enzyme is temporally controlled by administering the csMMPs in the presence of high calcium, and as the calcium diffuses through the tissue (or is absorbed or taken up by nearby cells) in vivo, the extracellular calcium concentration returns to physiological levels (approximately 1-1.3 mM Ca²⁺), which results in reduced activity or inactivation of the csMMP.

In particular, the csMMPs provided herein are active at high concentrations of calcium greater than physiological levels, such as greater than 1.5 mM, such as from or from about 1.5 mM to 100 mM, 2 mM to 50 mM, 2 mM to 25 mM, 5 mM to 50 mM, 5 mM to 25 mM, 5 mM to 15 mM or 8 mM to 12 mM, for example at least or about 1.5 mM, 2.0 mM, 2.5 mM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5, mM, 9.0 mM, 9.5 mM, 10.0 mM, 11 mM, 12 mM, 13 mM, 14 mM, 15 mM, 16 mM, 17 mM, 18 mM, 19 mM or 20 mM, and generally at least or about 10 mM. Compared to the activity at the high calcium concentrations greater than physiological levels, the csMMPs exhibit reduced activity at physiological levels of calcium of from or from about 1-1.3 mM Ca²⁺. Thus, the csMMPs used in the methods herein are less active or inactive in the presence of physiological levels of calcium than at higher calcium concentrations. For example, the csMMPs exhibits less than 90%, 85%, 80%, 75%, 70%, 65%, 60%, 55%, 50%, 45%, 40%, 35%, 30%, 25%, 20%, 15%, 10%, 5%, 2% or less matrix metalloprotease activity in the presence of physiological levels of extracellular calcium compared to its activity in the presence of calcium greater than the physiological level. The activity of the csMMP in the presence of physiological levels of calcium is generally at least 0.5-fold, 1-fold, 1.5-fold, 2-fold, 2.5-fold, 3-fold, 4-fold, 5-fold, 6-fold, 7-fold, 8-fold, 9-fold, 10-fold, 11-fold, 12-fold, 13-fold, 14-fold, 15-fold, or 20-fold less than the activity in the presence of levels of calcium greater than the physiological level. For example, the csMMPs used in the methods provided herein, have a ratio of activity at high calcium concentrations greater than the physiological level (e.g., 10 mM Ca²⁺) compared to physiological levels of calcium (e.g., 1-1.3 mM Ca²⁺) that is or is about 1.1, 1.2, 1.3, 1.4, 1.5, 1.6, 1.7, 1.8, 1.9, 2.0, 2.5, 3.0, 3.5, 4.0, 4.5, 5, 5.5, 6, 6.5, 7, 7.5, 8, 8.5, 9, 9.5, 10, 15, 20, 30, 40, 50 or more.

The modified MMP polypeptides used in the methods provided herein are generated by introducing modifications to a starting, unmodified reference MMP polypeptide (e.g., MMP-1). The unmodified MMP polypeptide is one that cleaves a component of the ECM, such as any of the MMP polypeptides set forth in Table 3. In particular, the unmodified MMP polypeptide is one that degrades or cleaves a collagen, such as a matrix metalloprotease-1 (MMP-1), matrix metalloprotease-2 (MMP-2), matrix metalloprotease-3 (MMP-3), matrix-metalloprotease-7 (MMP-7), matrix metalloprotease-8 (MMP-8), matrix metalloprotease-9 (MMP-9), matrix metalloprotease-10 (MMP-10), matrix metalloprotease-11 (MMP-11), matrix-metalloprotease-12 (MMP-12), matrix metalloprotease-13 (MMP-13), matrix metalloprotease-14 (MMP-14), matrix metalloprotease-16 (MMP-16), matrix metalloprotease-18 (MMP-18), matrix metalloprotease-19 (MMP-19), matrix metalloprotease-25 (MMP-25), and matrix metalloprotease-26 (MMP-26), or a catalytically active fragment thereof. For example, the collagen can be a collagen type I, collagen type II, collagen type III, collagen type IV, collagen type VI, collagen type VII, collagen type VIII, collagen type IX, collagen type X, collagen type XI and collagen type XIV. In one example, the unmodified MMP polypeptide is one that degrades or cleaves a collagen type I, such as a MMP-1, MMP-2, MMP-7, MMP-8, MMP-12, MMP-13, MMP-14 and MMP-18, or catalytically active fragment thereof. In another example, the unmodified MMP polypeptide is one that degrades or cleaves a collagen type III, such as a MMP-1, MMP-2, MMP-3, MMP-8, MMP-10, MMP-13, MMP-14 and MMP-16. In a further example, the unmodified MMP polypeptide is one that degrades or cleaves collagen type I and collagen type III, such as a MMP-1, MMP-2, MMP-8, MMP-13 and MMP-14. In a particular example, the unmodified MMP polypeptide is a MMP-1 polypeptide.

The modification can be an amino acid replacement or substitution, addition, or deletion of amino acids, or any combination thereof. For example, modified MMP-1 polypeptides used in the methods provided herein include those with 1, 2, 3, 4, 5, 6, 7, 8, 9, 10, 11, 12, 13, 14, 15, 16, 17, 18, 19, 20 or more modified positions. Also used in the methods provided herein are modified MMP-1 polypeptides with two or more modifications compared to a starting reference MMP-1 polypeptide. Modified MMP-1 polypeptides include those with 2, 3, 4, 5, 6, 7, 8, 9, 10, 11, 12, 13, 14, 15, 16, 17, 18, 19, 20 or more modified positions. In particular, modified csMMP polypeptides, used in the methods provided herein, contain 1, 2, 3, 4, 5, 6, 7, 8, 9 or 10 amino acid modifications. In some examples, modified MMP-1 polypeptides contain only a single modification. In other examples, modified MMP-1 polypeptides contain two, three, four, five or six modifications. In additional examples, any modification(s) provided herein can be combined with any other modification known to one of skill in the art so long as the resulting modified MMP-1 polypeptide retains increased calcium dependency for enzymatic activity when it is in its processed mature form.

The modifications provided herein can be made by standard recombinant DNA techniques such as are routine to one of skill in the art. Any method known in the art to effect mutation of any one or more amino acids in a target protein can be employed. Methods include standard site-directed mutagenesis (using e.g., a kit, such as QuikChange, available from Stratagene) of encoding nucleic acid molecules, or solid phase polypeptide synthesis methods.

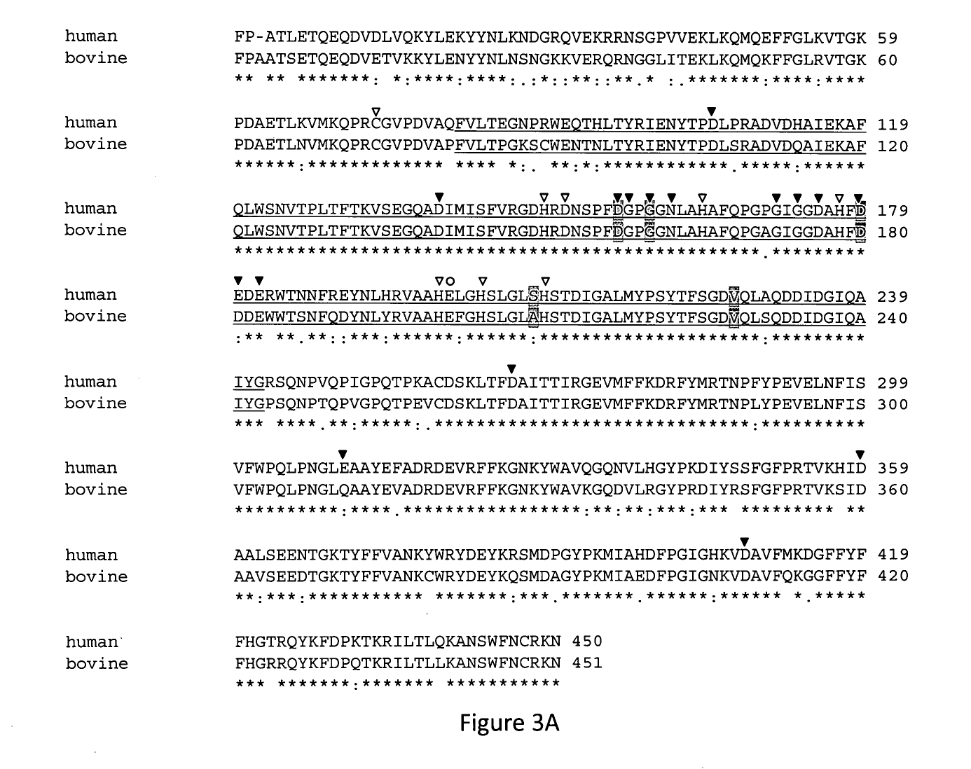

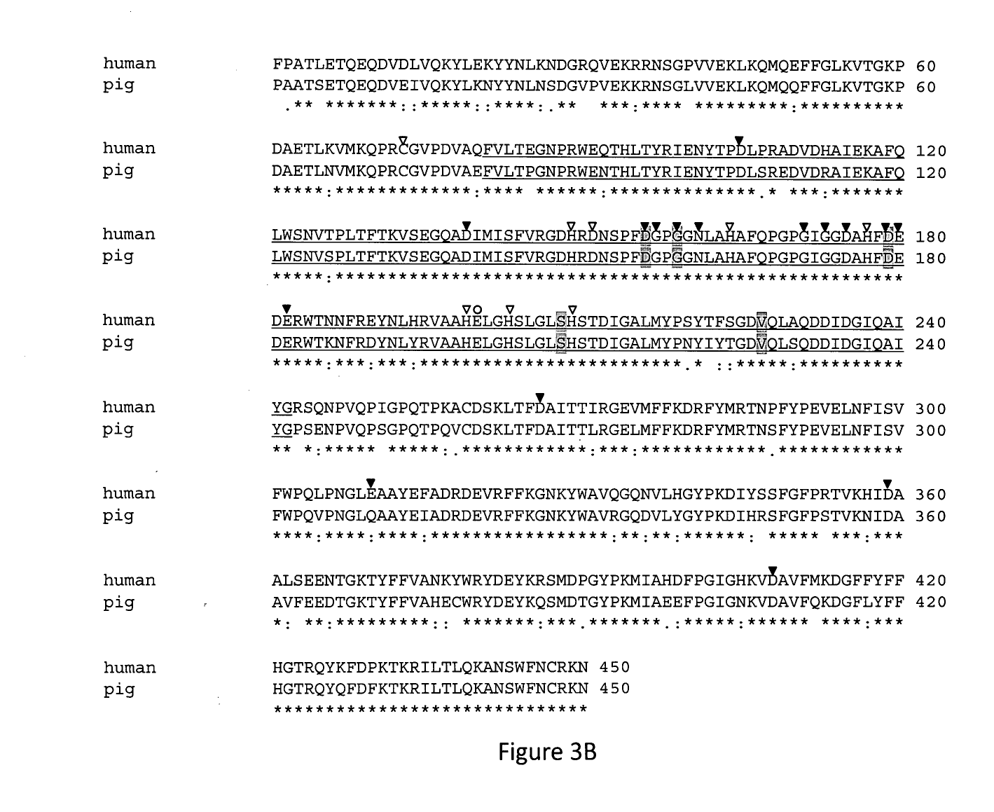

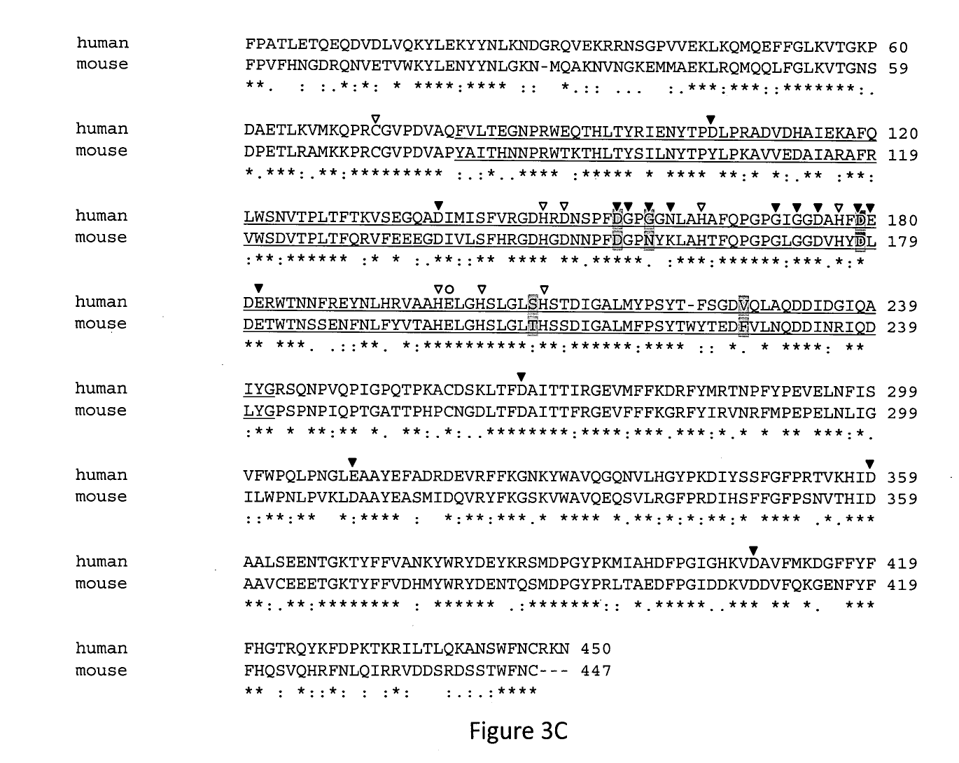

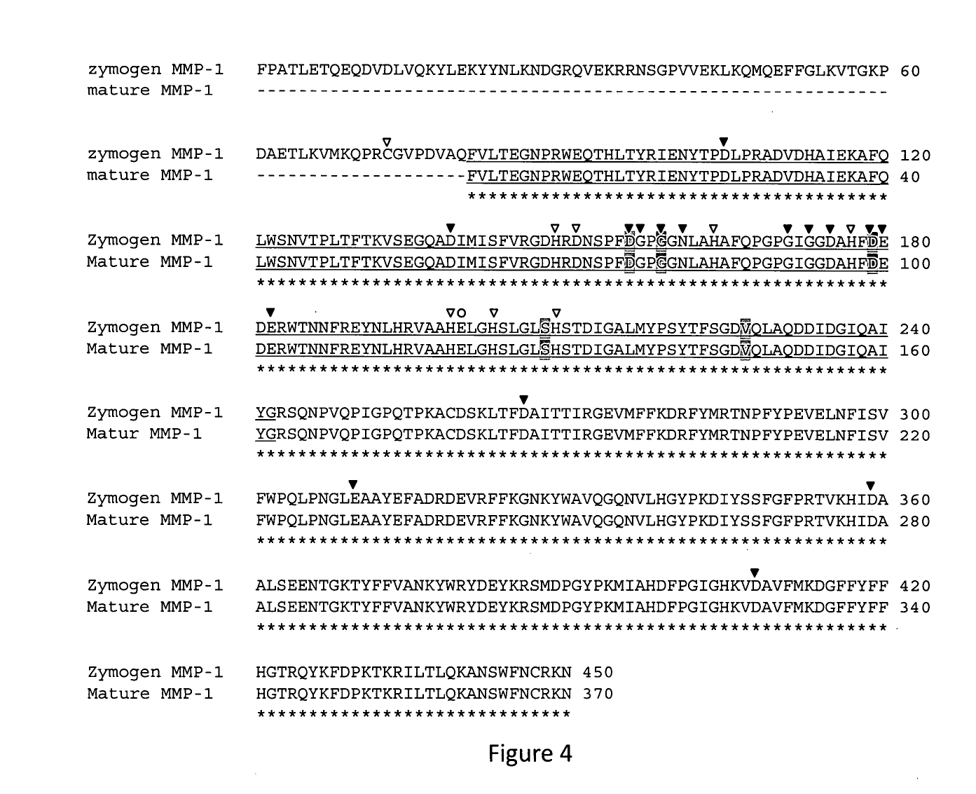

For purposes herein, reference to positions and amino acids for modification, including amino acid replacement or replacements, described herein, are with reference to the human MMP-1 polypeptide set forth in SEQ ID NO:2. Corresponding positions in another MMP polypeptide, or another form of a MMP polypeptide (e.g., mature form) can be identified by alignment of the MMP polypeptide with the reference MMP-1 polypeptide set forth in SEQ ID NO:2. For example, FIGS. 2 and 3 depict alignments of exemplary MMP polypeptides with SEQ ID NO:2, and identification of exemplary corresponding positions. FIG. 4 depicts alignment of the zymogen form set forth in SEQ ID NO:2 and the mature form thereof, lacking the prodomain, set forth in SEQ ID NO:5, and identification of exemplary corresponding positions. For purposes of modification (e.g., amino acid replacement), the corresponding amino acid residue can be any amino acid residue, and need not be identical to the residue set forth in SEQ ID NO:2. The corresponding amino acid residue identified by alignment with residues in SEQ ID NO:2 can be an amino acid residue that is identical to SEQ ID NO:2, or is a conservative or semi-conservative amino acid residue thereto (see e.g., FIGS. 2A-2C and 3A-3C). It also is understood that the exemplary replacements provided herein can be made at the corresponding residue in any MMP polypeptide, so long as the replacement results in a MMP polypeptide that exhibits dependency on increased calcium concentrations for activity. Based on this description and the description elsewhere herein, it is within the level of one of skill in the art to generate a modified MMP polypeptide containing any one or more of the described mutations and test the modified polypeptide for increased calcium sensitivity (i.e., increased calcium dependency for activity) as described herein.

For example, any of the modifications described herein with reference to SEQ ID NO:2 can be made in another MMP polypeptide by identifying the corresponding amino acid residue in another MMP polypeptide, such as any set forth in SEQ ID NOS:17, 20, 23, 26, 29, 32, 35, 38, 41, 44, 47, 50, 53, 56, 59, 62, 65, 68, 71, 74, 76, 80, 83, or mature or catalytically active form thereof or variants of any of such forms, such as those that exhibit at least 60%, 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:17, 20, 23, 26, 29, 32, 35, 38, 41, 44, 47, 50, 53, 56, 59, 62, 65, 68, 71, 74, 76, 80, and 83. For example, the modifications can be made in a mature or active form of any of the above polypeptides, such as any MMP polypeptide set forth in any of SEQ ID NOS:5, 132, 133, 134, 135, 137, 138, 140, 143 or 145 or a catalytically active fragment thereof, or any form or variant thereof that has a sequence of amino acids that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:5, 132, 133, 134, 135, 137, 138, 140, 143 or 145 or a catalytically active fragment thereof, so long as the modified MMP exhibits increased activity at levels of calcium greater than physiological levels than at physiological concentrations of calcium.

Modifications in a MMP polypeptide, for example a MMP-1 polypeptide, can be made to any form of a MMP polypeptide, including inactive (e.g., zymogen) or processed mature forms (active form), catalytically active fragments, allelic and species variants, splice variants, variants known in the art, or hybrid or chimeric MMP-1 polypeptides. Such forms of MMP are set forth in Table 3 above. For example, modifications can be made in a mature or active form of a MMP polypeptide, such as any MMP polypeptide set forth in any of SEQ ID NOS:5, 132, 133, 134, 135, 137, 138, 140, 143 or 145 or a catalytically active fragment thereof, or any form or variant thereof that has a sequence of amino acids that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:5, 132, 133, 134, 135, 137, 138, 140, 143 or 145 or a catalytically active fragment thereof, so long as the modified MMP exhibits increased activity at levels of calcium greater than physiological levels than at physiological concentrations of calcium.

With reference to MMP-1, modifications can be made in a precursor MMP-1 polypeptide set forth in SEQ ID NO:1, an inactive pro-enzyme MMP-1 containing the propeptide (zymogen form) set forth in SEQ ID NO:2, a mature MMP-1 polypeptide lacking the propeptide set forth in SEQ ID NO:5, or any variant (e.g., species, allelic or modified variant) or active fragment thereof that has 40%, 50%, 60%, 70%, 80%, 90%, 91%, 92%, 93%, 94%, 95%, 96%, 97%, 98%, or 99% sequence identity to any of the MMP-1 polypeptides set forth in SEQ ID NOS:1, 2 or 5, so long as the modified MMP polypeptide retains increased calcium dependency for enzymatic activity. Allelic variants and other variants of MMP-1 polypeptides include, but are not limited to, any MMP-1 polypeptide containing any one or more amino acid variants set forth in SEQ ID NOS:6-8 and 87. Exemplary species variants for modification herein include, but are not limited to, pig, rabbit, bovine, horse, rat, and mouse, for example, those set forth in any of SEQ ID NOS:9-14. Modifications also can be in a MMP-1 polypeptide lacking one or more domains, as long as the MMP-1 polypeptide retains increased calcium dependency for enzymatic activity. For example, modifications can be in a MMP-1 polypeptide that includes only the catalytic domain (corresponding to amino acids 81-242 of the proenzyme MMP-1 polypeptide set forth in SEQ ID NO:2). Modifications also can be made in a MMP-1 polypeptide lacking all or a portion of the proline rich linker (corresponding to amino acids 243-258 of the proenzyme MMP-1 polypeptide set forth in SEQ ID NO:2) and/or lacking all or a portion of the hemopexin binding domain (corresponding to amino acids 259-450 of the proenzyme MMP-1 polypeptide set forth in SEQ ID NO:2).

It is understood that while modifications can be made with reference to any form of a MMP polypeptide, for purposes of the methods herein, the modified MMP is administered in an active form that exhibits matrix metalloprotease activity to cleave a component of the ECM. Such form generally is a mature enzyme form lacking the prodomain, such as a processed form of a zymogen, or can be a catalytically active fragment thereof. For example, activity of MMP polypeptides is typically exhibited in its processed mature form following cleavage of the propeptide and/or intermolecular and intramolecular processing of the enzyme to remove the propeptide (see e.g., Visse et al. (2003) Cir. Res. 92:827-839). Hence, any modified polypeptide provided herein that is a zymogen proenzyme can be activated by a processing agent to generate a processed mature MMP-1 polypeptide. As noted elsewhere herein, csMMPs provided herein also require calcium concentrations above or greater than physiological levels (e.g., 1-1.3 mM) to be fully active. The modified MMP or catalytically active fragment thereof exhibits at least 5%, 10%, 15%, 20%, 25%, 30%, 40%, 50%, 60%, 70%, 80%, 90%, 95%, 100%, 110%, 120%, 130%, 140%, 150%, 160%, 170%, 180%, 190%, 200%, 250%, 300%, 350%, 400%, 450%, or 500% or more of the activity of the unmodified MMP not containing the modification(s) (e.g., amino acid replacement(s)) when present at concentrations of calcium greater than physiological levels of calcium.

Modified MMP-1 polypeptides provided herein can be assayed for enzymatic activity under various conditions (e.g., at high and low levels of calcium) to identify those that confer increased calcium dependency for enzymatic activity. For example, MMP polypeptides that are normally active at physiological calcium concentrations (e.g., 1 mM Ca²⁺) and at high calcium concentrations (e.g., 10 mM Ca²⁺) are modified and enzymes are selected that are substantially active at calcium concentrations higher than physiological extracellular calcium concentrations (e.g., more than 1 mM Ca²⁺; such as at least or about at least 2 mM, 3 mM, 4 mM, 5 mM, 6 mM, 7 mM, 8 mM, 9 mM, 10 mM, 15 mM, 20 mM, 25 mM, 50 mM, 100 mM or greater than 100 mM), but that are less active or inactive at physiological calcium concentrations. Such modified enzymes can be used as conditionally active matrix-degrading enzymes where the condition for activity is high calcium (e.g., more than 1 mM Ca²⁺).

Exemplary modifications in a MMP polypeptide, for example a MMP-1 polypeptide, with reference to positions corresponding to positions in SEQ ID NO:2 are provided below. In the subsections below, non-limiting examples of modified MMP polypeptides that exhibit calcium-sensitive activity in the presence of high calcium concentrations greater than physiological levels and decreased activity in the presence of physiological levels of calcium, and encoding nucleic acid molecules, are described.

1. Modifications at or Near a Metal Binding Site

Among the modified csMMP polypeptides provided herein for use in the methods herein are csMMP polypeptides containing one or more amino acid modifications in a starting, unmodified MMP, at amino acids at or near a metal binding site. Typically, the modification is an amino acid replacement. In particular, the modification, such as amino acid replacement or replacements, can be at or near (i.e., within 3 residues of) any one or more positions corresponding to any of the residues directly involved with zinc or calcium coordination. As described above, MMP proteins contain an active site zinc that is coordinated by imidazole side-chains of histidine residues at positions corresponding to positions 199, 203, and 209 with reference to positions set forth in SEQ ID NO:2. A second zinc ion is coordinated by histidine residues corresponding to positions 149, 164 and 177 with reference to SEQ ID NO:2. Binding of zinc to MMP serves as a stabilizing and/or structural function of the molecule. In addition to zinc, three calcium molecules bind to the catalytic domain and one to the hemopexin domain, which are coordinated by amino acids enriched in acidic side chains at positions corresponding to positions 156, 157, 159, 161, 179 and 182 (first calcium, C1); 139, 171, 173 and 175 (second calcium, C2); 105, 180 and 182 (third calcium, C3); and 266, 310, 359 and 408 (fourth calcium, C4), each with reference to positions set forth in SEQ ID NO:2. The calcium molecules have also been shown to play an important role in domain stabilization and in the regulation of catalytic activity. The amino acids important for the binding of the zinc and calcium molecules, and in particular those within the catalytic domain, are highly conserved among MMP family members (Maskos et al. (2005) Biochimie 87:249-263).

For wild-type or unmodified MMP polypeptides, Ca²⁺ ions are known to be required for the activity of MMPs (Nagai et al. (1966) Biochem. 5:3123-3130); Jeffrey et al. (1970) Biochem. 9:268-273). Sufficient activity, however, is achieved at physiological concentrations of calcium of 1 mM with higher concentrations of calcium not further increasing activity. While Ca²⁺ ions do not directly participate in proteolysis, it is integral in the stabilization of the tertiary structure of the enzyme. For example, circular dichroism analysis of human MMP-1 revealed that in the absence of calcium, the protein is in a catalytically inactive and partially unfolded state with a native secondary structure, but altered tertiary structure. Over time, in the absence of calcium, the enzymatic loss is irreversible, and in the case of MMP-3, results in autocatalysis.

It is found herein that modification at or near residues associated with metal binding, and in particular residues involved in calcium coordination, effect destabilization and degradation of the protein in the presence of physiological levels of calcium (e.g., about 1 mM Ca²⁺), but not in the presence of higher concentrations of calcium greater than the physiological level. This effect is especially evident at physiological temperatures (e.g., about or 37° C.). This negative effect on protein stability correlates to a reduction or abolishment of activity to digest substrate, such as collagen (e.g., collagen type I), under conditions that exist in the physiological environment (e.g., 1 mM Ca²⁺, 37° C.). Hence, the results show that the activity of such modified MMP polypeptides can be modulated under physiological conditions in vivo by calcium concentration, where activity is achieved at concentrations of calcium greater than physiological levels, but is reduced at levels of calcium present in the physiological environment (e.g., about 1 mM). This is in contrast to wild-type or unmodified MMPs that are normally active at physiological concentrations of calcium, and whose activity is not further modulated by higher concentrations of calcium.

In such examples of the above modified MMP polypeptides having a modification at or near a metal binding site, the amino acid replacement or replacements can be at any one or more positions corresponding to any of the following positions: 102, 103, 104, 105, 106, 107, 108, 136, 137, 138, 139, 140, 141, 142, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 196, 197, 198, 199, 201, 202, 203, 204, 205, 206, 207, 208, 209, 210, 211, 212, 263, 264, 265, 266, 267, 268, 269, 307, 308, 309, 310, 311, 312, 313, 356, 357, 358, 359, 360, 361, 362, 405, 406, 407, 408, 409, 410, 411 with reference to positions set forth in SEQ ID NO:2. For example, the amino acid positions can be replacements at one or more positions corresponding to tyrosine (Y) 102, T103, P104, D105, L106, P107, R108, G136, Q137, A138, D139, I140, M141, I142, R146, G147, D148, H149, R150, D151, N152, S153, P154, F155, D156, G157, P158, G159, G160, N161, L162, A163, H164, A165, F166, Q167, P168, G169, P170, G171, I172, G173, G174, D175, A176, H177, F178, D179, E180, D181, E182, R183, W184, T185, V196, A197, A198, H199, L201, G202, H203, S204, L205, G206, L207, S208, H209, S210, T211, D212, L263, T264, F265, D266, A267, I268, T269, N307, G308, L309, E310, A311, A312, Y313, K356, H357, I358, D359, A360, A361, L362, H405, K406, V407, D408, A409, V410, F411, with reference to amino acid positions set forth in SEQ ID NO:2.

In any of the above provided modified MMP polypeptides that have an amino acid replacement at a position that is at or near a metal binding site, the amino acid replacement can be to any other amino acid so long as the resulting modified MMP polypeptide exhibits calcium sensitivity at concentrations of calcium greater than the physiological concentration, and reduced activity at physiological concentrations of calcium. For example, amino acid replacements include replacement of amino acids to an acidic (D or E); basic (H, K or R); neutral (C, N, Q, T, Y, S, G) or hydrophobic (F, M, W, I, V, L A, P) amino acid residue. For example, amino acid replacements at the noted positions include replacement by amino acid residues E, H, R, C, Q, T, S, G, M, W, I, V, L, A, P, N, F, D, Y or K.

For example, exemplary of such modified MMPs provided herein, such as a modified MMP-1 polypeptide, are those that contain at least one amino acid replacement from among replacement with: R at a position corresponding to position 102; K at a position corresponding to position 102; V at a position corresponding to position 102; M at a position corresponding to position 102; P at a position corresponding to position 102; N at a position corresponding to position 102; G at a position corresponding to position 102; L at a position corresponding to position 102; D at a position corresponding to position 102; S at a position corresponding to position 102; F at a position corresponding to position 102; A at a position corresponding to position 102; E at a position corresponding to position 102; Q at a position corresponding to position 102; C at a position corresponding to position 102; N at position corresponding to position 103; E at a position corresponding to position 104; T at a position corresponding to position 104; R at a position corresponding to position 104; D at a position corresponding to position 104; Q at a position corresponding to position 104; V at a position corresponding to position 104; Y at a position corresponding to position 104; H at a position corresponding to position 104; L at a position corresponding to position 104; A at a position corresponding to position 104; M at a position corresponding to position 104; A at a position corresponding to position 105; C at a position corresponding to position 105; F at a position corresponding to position 105; G at a position corresponding to position 105; I at a position corresponding to position 105; L at a position corresponding to position 105; M at a position corresponding to position 105; N at a position corresponding to position 105; P at a position corresponding to position 105; R at a position corresponding to position 105; S at a position corresponding to position 105; T at a position corresponding to position 105; V at a position corresponding to position 105; W at a position corresponding to position 105; E at a position corresponding to position 105; M at a position corresponding to position 106; A at a position corresponding to position 106; Y at a position corresponding to position 106; V at a position corresponding to position 106; I at a position corresponding to position 106; L at a position corresponding to position 107; T at a position corresponding to position 107; S at a position corresponding to position 107; R at a position corresponding to position 107; M at a position corresponding to position 107; V at a position corresponding to position 107; D at a position corresponding to position 107; A at a position corresponding to position 107; K at a position corresponding to position 107; G at a position corresponding to position 107; P at a position corresponding to position 108; G at a position corresponding to position 108; E at a position corresponding to position 108; A at a position corresponding to position 108; Y at a position corresponding to position 108; K at a position corresponding to position 108; C at a position corresponding to position 108; S at a position corresponding to position 108; S at a position corresponding to position 108; F at a position corresponding to position 108; I at a position corresponding to position 108; L at a position corresponding to position 108; N at a position corresponding to position 108; D at a position corresponding to position 136; M at a position corresponding to position 136; N at a position corresponding to position 136; A at a position corresponding to position 136; L at a position corresponding to position 136; P at a position corresponding to position 136; T at a position corresponding to position 136; R at a position corresponding to position 136; S at a position corresponding to position 136; H at a position corresponding to position 136; E at a position corresponding to position 136; A at a position corresponding to position 137; R at a position corresponding to position 137; G at a position corresponding to position 137; K at a position corresponding to position 137; H at a position corresponding to position 137; P at a position corresponding to position 137; S at a position corresponding to position 137; L at a position corresponding to position 137; W at a position corresponding to position 137; F at a position corresponding to position 137; T at a position corresponding to position 137; Y at a position corresponding to position 137; E at a position corresponding to position 137; G at a position corresponding to position 138; R at a position corresponding to position 139; V at a position corresponding to position 139; M at a position corresponding to position 139; C at a position corresponding to position 139; P at a position corresponding to position 139; P at a position corresponding to position 139; S at a position corresponding to position 139; L at a position corresponding to position 139; I at a position corresponding to position 139; H at a position corresponding to position 139; A at a position corresponding to position 139; G at a position corresponding to position 139; F at a position corresponding to position 139; N at a position corresponding to position 139; W at a position corresponding to position 139; Y at a position corresponding to position 139; E at a position corresponding to position 139; E at a position corresponding to position 141; I at a position corresponding to position 141; R at a position corresponding to position 141; S at a position corresponding to position 141; L at a position corresponding to position 141; A at a position corresponding to position 141; D at a position corresponding to position 141; W at a position corresponding to position 141; H at a position corresponding to position 141; N at a position corresponding to position 141; L at a position corresponding to position 142; M at a position corresponding to position 142; V at a position corresponding to position 142; T at a position corresponding to position 146; N at a position corresponding to position 146; Q at a position corresponding to position 146; K at a position corresponding to position 146; S at a position corresponding to position 146; D at a position corresponding to position 146; A at a position corresponding to position 146; Y at a position corresponding to position 146; V at a position corresponding to position 146; R at a position corresponding to position 147; F at a position corresponding to position 147; H at a position corresponding to position 147; W at a position corresponding to position 147; T at a position corresponding to position 147; C at a position corresponding to position 147; S at a position corresponding to position 147; V at a position corresponding to position 147; Q at a position corresponding to position 147; M at a position corresponding to position 147; R at a position corresponding to position 148; R at a position corresponding to position 148; I at a position corresponding to position 148; T at a position corresponding to position 148; G at a position corresponding to position 148; G at a position corresponding to position 148; V at a position corresponding to position 148; A at a position corresponding to position 148; A at a position corresponding to position 148; W at a position corresponding to position 148; P at a position corresponding to position 148; S at a position corresponding to position 148; N at a position corresponding to position 148; S at a position corresponding to position 150; E at a position corresponding to position 150; G at a position corresponding to position 150; M at a position corresponding to position 150; M at a position corresponding to position 150; T at a position corresponding to position 150; W at a position corresponding to position 150; A at a position corresponding to position 150; N at a position corresponding to position 150; K at a position corresponding to position 150; L at a position corresponding to position 150; L at a position corresponding to position 150; V at a position corresponding to position 150; D at a position corresponding to position 150; H at a position corresponding to position 150; G at a position corresponding to position 152; C at a position corresponding to position 152; F at a position corresponding to position 152; L at a position corresponding to position 152; L at a position corresponding to position 152; L at a position corresponding to position 152; P at a position corresponding to position 152; R at a position corresponding to position 152; H at a position corresponding to position 152; T at a position corresponding to position 152; Y at a position corresponding to position 152; K at a position corresponding to position 152; D at a position corresponding to position 152; W at a position corresponding to position 152; I at a position corresponding to position 152; A at a position corresponding to position 152; S at a position corresponding to position 152; R at a position corresponding to position 152; G at a position corresponding to position 153; H at a position corresponding to position 153; V at a position corresponding to position 153; T at a position corresponding to position 153; P at a position corresponding to position 153; F at a position corresponding to position 153; D at a position corresponding to position 153; Q at a position corresponding to position 153; Y at a position corresponding to position 153; L at a position corresponding to position 154; C at a position corresponding to position 154; S at a position corresponding to position 154; I at a position corresponding to position 154; M at a position corresponding to position 155; H at a position corresponding to position 156; L at a position corresponding to position 156; E at a position corresponding to position 156; A at a position corresponding to position 156; W at a position corresponding to position 156; C at a position corresponding to position 156; P at a position corresponding to position 156; P at a position corresponding to position 156; V at a position corresponding to position 156; V at a position corresponding to position 156; K at a position corresponding to position 156; S at a position corresponding to position 156; G at a position corresponding to position 156; T at a position corresponding to position 156; Y at a position corresponding to position 156; R at a position corresponding to position 156; M at a position corresponding to position 156; K at a position corresponding to position 157; D at a position corresponding to position 157; F at a position corresponding to position 157; R at a position corresponding to position 157; H at a position corresponding to position 157; L at a position corresponding to position 157; N at a position corresponding to position 157; N at a position corresponding to position 157; Y at a position corresponding to position 157; S at a position corresponding to position 157; T at a position corresponding to position 157; A at a position corresponding to position 157; A at a position corresponding to position 157; Q at a position corresponding to position 157; P at a position corresponding to position 157; P at a position corresponding to position 157; V at a position corresponding to position 157; V at a position corresponding to position 157; M at a position corresponding to position 157; S at a position corresponding to position 158; Y at a position corresponding to position 158; R at a position corresponding to position 158; L at a position corresponding to position 158; V at a position corresponding to position 158; V at a position corresponding to position 158; C at a position corresponding to position 158; A at a position corresponding to position 158; W at a position corresponding to position 158; I at a position corresponding to position 158; F at a position corresponding to position 158; Q at a position corresponding to position 158; T at a position corresponding to position 158; G at a position corresponding to position 158; K at a position corresponding to position 158; N at a position corresponding to position 158; D at a position corresponding to position 158; R at a position corresponding to position 159; S at a position corresponding to position 159; Q at a position corresponding to position 159; P at a position corresponding to position 159; V at a position corresponding to position 159; K at a position corresponding to position 159; A at a position corresponding to position 159; Y at a position corresponding to position 159; E at a position corresponding to position 159; T at a position corresponding to position 159; M at a position corresponding to position 159; I at a position corresponding to position 159; W at a position corresponding to position 159; W at a position corresponding to position 159; L at a position corresponding to position 159; C at a position corresponding to position 159; A at a position corresponding to position 160; H at a position corresponding to position 160; N at a position corresponding to position 160; W at a position corresponding to position 160; R at a position corresponding to position 160; M at a position corresponding to position 160; Q at a position corresponding to position 160; V at a position corresponding to position 160; S at a position corresponding to position 160; E at a position corresponding to position 160; L at a position corresponding to position 160; T at a position corresponding to position 160; S at a position corresponding to position 161; C at a position corresponding to position 161; L at a position corresponding to position 161; R at a position corresponding to position 161; R at a position corresponding to position 161; G at a position corresponding to position G; W at a position corresponding to position 161; Y at a position corresponding to position 161; E at a position corresponding to position 161; P at a position corresponding to position 161; T at a position corresponding to position 161; H at a position corresponding to position 161; I at a position corresponding to position 161; V at a position corresponding to position 161; F at a position corresponding to position 161; Q at a position corresponding to position 161; S at a position corresponding to position 164; W at a position corresponding to position 166; D at a position corresponding to position 167; R at a position corresponding to position 167; A at a position corresponding to position 167; S at a position corresponding to position 167; S at a position corresponding to position 167; F at a position corresponding to position 167; Y at a position corresponding to position 167; P at a position corresponding to position 167; T at a position corresponding to position 167; V at a position corresponding to position 167; L at a position corresponding to position 167; M at a position corresponding to position 167; N at a position corresponding to position 167; G at a position corresponding to position 167; K at a position corresponding to position 167; E at a position corresponding to position 167; R at a position corresponding to position 168; L at a position corresponding to position 170; R at a position corresponding to position 170; R at a position corresponding to position 170; I at a position corresponding to position 170; T at a position corresponding to position 170; Q at a position corresponding to position 170; G at a position corresponding to position 170; S at a position corresponding to position 170; H at a position corresponding to position 170; M at a position corresponding to position 170; K at a position corresponding to position 170; S at a position corresponding to position 171; M at a position corresponding to position 171; N at a position corresponding to position 171; P at a position corresponding to position 171; R at a position corresponding to position 171; Y at a position corresponding to position 171; A at a position corresponding to position 171; Q at a position corresponding to position 171; H at a position corresponding to position 171; L at a position corresponding to position 171; W at a position corresponding to position 171; C at a position corresponding to position 171; K at a position corresponding to position 171; E at a position corresponding to position 171; D at a position corresponding to position 171; Y at a position corresponding to position 172; T at a position corresponding to position 172; P at a position corresponding to position 172; A at a position corresponding to position 172; L at a position corresponding to position 172; Q at a position corresponding to position 172; E at a position corresponding to position 172; M at a position corresponding to position 172; D at a position corresponding to position 172; V at a position corresponding to position 172; R at a position corresponding to position 172; W at a position corresponding to position 172; N at a position corresponding to position 172; C at a position corresponding to position 173; L at a position corresponding to position 173; K at a position corresponding to position 173; W at a position corresponding to position 173; W at a position corresponding to position 173; S at a position corresponding to position 173; A at a position corresponding to position 173; R at a position corresponding to position 173; N at a position corresponding to position 173; T at a position corresponding to position 173; D at a position corresponding to position 173; V at a position corresponding to position 173; F at a position corresponding to position 173; M at a position corresponding to position 173; Y at a position corresponding to position 173; P at a position corresponding to position 173; I at a position corresponding to position 175; T at a position corresponding to position 175; N at a position corresponding to position 175; V at a position corresponding to position 175; S at a position corresponding to position 175; R at a position corresponding to position 175; G at a position corresponding to position 175; A at a position corresponding to position 175; F at a position corresponding to position 175; C at a position corresponding to position 175; Q at a position corresponding to position 175; Y at a position corresponding to position 175; L at a position corresponding to position 175; H at a position corresponding to position 175; P at a position corresponding to position 175; E at a position corresponding to position 175; F at a position corresponding to position 176; Q at a position corresponding to position 176; V at a position corresponding to position 176; T at a position corresponding to position 176; C at a position corresponding to position 176; L at a position corresponding to position 176; P at a position corresponding to position 179; L at a position corresponding to position 179; E at a position corresponding to position 179; G at a position corresponding to position 179; G at a position corresponding to position 179; S at a position corresponding to position 179; A at a position corresponding to position 179; K at a position corresponding to position 179; T at a position corresponding to position 179; I at a position corresponding to position 179; R at a position corresponding to position 179; N at a position corresponding to position 179; W at a position corresponding to position 179; Q at a position corresponding to position 179; V at a position corresponding to position 179; C at a position corresponding to position 179; M at a position corresponding to position 180; P at a position corresponding to position 180; K at a position corresponding to position 180; Y at a position corresponding to position 180; Q at a position corresponding to position 180; R at a position corresponding to position 180; A at a position corresponding to position 180; T at a position corresponding to positionl 80; I at a position corresponding to position 180; F at a position corresponding to position 180; C at a position corresponding to position 180; G at a position corresponding to position 180; S at a position corresponding to position 180; N at a position corresponding to position 180; D at a position corresponding to position 180; S at a position corresponding to position 181; Q at a position corresponding to position 181; A at a position corresponding to position 181; T at a position corresponding to position 181; E at a position corresponding to position 181; C at a position corresponding to position 182; P at a position corresponding to position 182; P at a position corresponding to position 182; S at a position corresponding to position 182; T at a position corresponding to position 182; R at a position corresponding to position 182; D at a position corresponding to position 182; A at a position corresponding to position 182; F at a position corresponding to position 182; L at a position corresponding to position 182; I at a position corresponding to position 182; Y at a position corresponding to position 182; Q at a position corresponding to position 182; W at a position corresponding to position 182; M at a position corresponding to position 182; G at a position corresponding to position 182; K at a position corresponding to position 183; W at a position corresponding to position 183; W at a position corresponding to position 183; E at a position corresponding to position 183; A at a position corresponding to position 183; T at a position corresponding to position 183; N at a position corresponding to position 183; H at a position corresponding to position 183; V at a position corresponding to position 183; C at a position corresponding to position 183; M at a position corresponding to position 183; G at a position corresponding to position 183; S at a position corresponding to position 183; S at a position corresponding to position 185; C at a position corresponding to position 197; V at a position corresponding to position 201; M at a position corresponding to position 201; E at a position corresponding to position 203; A at a position corresponding to position 204; M at a position corresponding to position 205; I at a position corresponding to position 205; A at a position corresponding to position 207; M at a position corresponding to position 207; D at a position corresponding to position 208; V at a position corresponding to position 208; P at a position corresponding to position 208; G at a position corresponding to position 208; A at a position corresponding to position 208; K at a position corresponding to position 208; N at a position corresponding to position 208; F at a position corresponding to position 208; Q at a position corresponding to position 208; W at a position corresponding to position 208; T at a position corresponding to position 208; E at a position corresponding to position 208; C at a position corresponding to position 208; R at a position corresponding to position 208; L at a position corresponding to position 208; T at a position corresponding to position 210; P at a position corresponding to position 211; R at a position corresponding to position 211; K at a position corresponding to position 211; G at a position corresponding to position 211; M at a position corresponding to position 211; M at a position corresponding to position 211; N at a position corresponding to position 211; N at a position corresponding to position 211; V at a position corresponding to position 211; Q at a position corresponding to position 211; S at a position corresponding to position 211; A at a position corresponding to position 211; E at a position corresponding to position 212; T at a position corresponding to position 212; N at a position corresponding to position 212; S at a position corresponding to position 212; P at a position corresponding to position 212; Q at a position corresponding to position 212; F at a position corresponding to position 212; H at a position corresponding to position 212; and Y at a position corresponding to position 212, with reference to amino acid positions set forth in SEQ ID NO:2.

As described above, it is understood that the replacements can be made in a corresponding position in another MMP polypeptide as determined by alignment therewith with the sequence set forth in SEQ ID NO:2 (see e.g., FIGS. 2A-2C and 3A-3C), whereby the corresponding position is the aligned position. For example, any of the above modifications (e.g., amino acid replacements) can be made at a corresponding position of a MMP polypeptide, such as any set forth in SEQ ID NOS:17, 20, 23, 26, 29, 32, 35, 38, 41, 44, 47, 50, 53, 56, 59, 62, 65, 68, 71, 74, 76, 80, 83, or mature or catalytically active form thereof or variants of any of such forms, such as those that exhibit at least 60%, 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:17, 20, 23, 26, 29, 32, 35, 38, 41, 44, 47, 50, 53, 56, 59, 62, 65, 68, 71, 74, 76, 80, 83. For example, the modifications (e.g., amino acid replacements) can be made in a mature or catalytically active form of any of the above polypeptides, such as any MMP polypeptide set forth in any of SEQ ID NOS:5, 132, 133, 134, 135, 137, 138, 140, 143 or 145 or a catalytically active fragment thereof, or any form or variant thereof that has a sequence of amino acids that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:5, 132, 133, 134, 135, 137, 138, 140, 143 or 145 or a catalytically active fragment thereof, so long as the modified MMP exhibits increased activity at a calcium concentration greater than physiological levels compared to activity at physiological concentrations of calcium. As described above, it is within the level of the skilled artisan to determine corresponding positions by alignment of a MMP polypeptide with reference to MMP-1 zymogen form set forth in SEQ ID NO:2. For example, Table 5 sets forth the amino acid positions at or near a metal binding site in a mature MMP-1 polypeptide lacking the propeptide that correspond to the above positions with reference to MMP-1 set forth in SEQ ID NO:2 (see also FIG. 4).

In particular, modifications herein, such as any of the above amino acid replacements, are made in a MMP-1 polypeptide, such as in a precursor MMP-1 polypeptide set forth in SEQ ID NO:1, an inactive pro-enzyme MMP-1 containing the propeptide (zymogen form) set forth in SEQ ID NO:2, a mature MMP-1 polypeptide lacking the propeptide set forth in SEQ ID NO:5, or any variant (e.g., species, allelic or modified variant) or active fragment thereof that has 40%, 50%, 60%, 70%, 80%, 90%, 91%, 92%, 93%, 94%, 95%, 96%, 97%, 98%, or 99% sequence identity to any of the MMP-1 polypeptides set forth in SEQ ID NOS:1, 2 or 5, so long as the modified MMP polypeptide retains increased calcium dependency for enzymatic activity. In particular examples herein, modifications, such as amino acid replacements, for use in the methods herein are at positions at or near a metal binding site in a mature MMP-1 polypeptide set forth in SEQ ID NO:5, or a catalytically active fragment thereof or polypeptide that exhibits at least 40%, 50%, 60%, 70%, 80%, 90%, 91%, 92%, 93%, 94%, 95%, 96%, 97%, 98%, or 99% sequence identity to SEQ ID NO:5, so long as the modified MMP polypeptide retains increased calcium dependency for enzymatic activity.

TABLE 5
Residues At Or Near a Metal-Binding Site

| Corresponding | Position | Position | |
|---|---|---|---|
| hMMP-1 | (zymogen numbering; | (mature numbering; | Metal |
| Residue | SEQ ID NO: 2) | SEQ ID NO: 5) | Binding |
| Y | 102 | 22 | near Ca²⁺ |
| T | 103 | 23 | near Ca²⁺ |
| P | 104 | 24 | near Ca²⁺ |
| D | 105 | 25 | Ca²⁺ |
| L | 106 | 26 | near Ca²⁺ |
| P | 107 | 27 | near Ca²⁺ |
| R | 108 | 28 | near Ca²⁺ |
| G | 136 | 56 | near Ca²⁺ |
| Q | 137 | 57 | near Ca²⁺ |
| A | 138 | 58 | near Ca²⁺ |
| D | 139 | 59 | Ca²⁺ |
| I | 140 | 60 | near Ca²⁺ |
| M | 141 | 61 | near Ca²⁺ |
| I | 142 | 62 | near Ca²⁺ |
| R | 146 | 66 | near Zn²⁺ |
| G | 147 | 67 | near Zn²⁺ |
| D | 148 | 68 | near Zn²⁺ |
| H | 149 | 69 | Zn²⁺ |
| R | 150 | 70 | near Zn²⁺ |
| D | 151 | 71 | Zn²⁺ |
| N | 152 | 72 | near Zn²⁺ |
| S | 153 | 73 | near Ca²⁺ |
| P | 154 | 74 | near Ca²⁺ |
| F | 155 | 75 | near Ca²⁺ |
| D | 156 | 76 | Ca²⁺ |
| G | 157 | 77 | Ca²⁺ |
| P | 158 | 78 | near Ca²⁺ |
| G | 159 | 79 | Ca²⁺ |
| G | 160 | 80 | near Ca²⁺ |
| N | 161 | 81 | Ca²⁺ |
| L | 162 | 82 | near Ca²⁺/ |
| | | | Zn²⁺ |
| A | 163 | 83 | near Ca²⁺/ |
| | | | Zn²⁺ |
| H | 164 | 84 | Zn²⁺ |
| A | 165 | 85 | near Zn²⁺ |
| F | 166 | 86 | near Zn²⁺ |
| Q | 167 | 87 | near Zn²⁺ |
| P | 168 | 88 | near Ca²⁺ |
| G | 169 | 89 | near Ca²⁺ |
| P | 170 | 90 | near Ca²⁺ |
| G | 171 | 91 | Ca²⁺ |
| I | 172 | 92 | near Ca²⁺ |
| G | 173 | 93 | Ca²⁺ |
| G | 174 | 94 | near Ca²⁺ |
| D | 175 | 95 | Ca²⁺ |
| A | 176 | 96 | near Ca²⁺/ |
| | | | Zn²⁺ |
| H | 177 | 97 | Zn²⁺ |
| F | 178 | 98 | near Ca²⁺/ |
| | | | Zn²⁺ |
| D | 179 | 99 | Ca²⁺ |
| E | 180 | 100 | Ca²⁺ |
| D | 181 | 101 | near Ca²⁺ |
| E | 182 | 102 | Ca²⁺ |
| R | 183 | 103 | near Ca²⁺ |
| W | 184 | 104 | near Ca²⁺ |
| T | 185 | 105 | near Ca²⁺ |
| V | 196 | 116 | near Zn²⁺ |
| A | 197 | 117 | near Zn²⁺ |
| A | 198 | 118 | near Zn²⁺ |
| H | 199 | 119 | Zn²⁺ |
| E | 200 | 120 | near Zn²⁺- |
| | | | active site |
| L | 201 | 121 | near Zn²⁺ |
| G | 202 | 122 | near Zn ²⁺ |
| H | 203 | 123 | Zn²⁺ |
| S | 204 | 124 | near Zn²⁺ |
| L | 205 | 125 | near Zn²⁺ |
| G | 206 | 126 | near Zn²⁺ |
| L | 207 | 127 | near Zn²⁺ |
| S | 208 | 128 | near Zn²⁺ |
| H | 209 | 129 | Zn²⁺ |
| S | 210 | 130 | near Zn²⁺ |
| T | 211 | 131 | near Zn²⁺ |
| D | 212 | 132 | near Zn²⁺ |
| L | 263 | 183 | near Ca²⁺ |
| T | 264 | 184 | near Ca²⁺ |
| F | 265 | 185 | near Ca²⁺ |
| D | 266 | 186 | Ca²⁺ |
| A | 267 | 187 | near Ca²⁺ |
| I | 268 | 188 | near Ca²⁺ |
| T | 269 | 189 | near Ca²⁺ |
| N | 307 | 227 | near Ca²⁺ |
| G | 308 | 228 | near Ca²⁺ |
| L | 309 | 229 | near Ca²⁺ |
| E | 310 | 230 | Ca²⁺ |
| A | 311 | 231 | near Ca²⁺ |
| A | 312 | 232 | near Ca²⁺ |
| Y | 313 | 233 | near Ca²⁺ |
| K | 356 | 276 | near Ca²⁺ |
| H | 357 | 277 | near Ca²⁺ |
| I | 358 | 278 | near Ca²⁺ |
| D | 359 | 279 | Ca²⁺ |
| A | 360 | 280 | near Ca²⁺ |
| A | 361 | 281 | near Ca²⁺ |
| L | 362 | 282 | near Ca²⁺ |
| H | 405 | 325 | near Ca²⁺ |
| K | 406 | 326 | near Ca²⁺ |
| V | 407 | 327 | near Ca²⁺ |
| D | 408 | 328 | Ca²⁺ |
| A | 409 | 329 | near Ca²⁺ |
| V | 410 | 330 | near Ca²⁺ |
| F | 411 | 331 | near Ca²⁺ |

Amino Acids Involved in Calcium Coordination

In particular examples herein, modified MMPs provided herein, such as a modified MMP-1 polypeptides, are those that contain at least one amino acid replacement at or near (i.e., within 3 residues of) any one or more positions corresponding to any of the residues directly involved with calcium coordination. In such examples, the amino acid replacement or replacements can be at any one or more positions corresponding to any of the following positions: 102, 103, 104, 105, 106, 107, 108, 136, 137, 138, 139, 140, 141, 142, 153, 154, 155, 156, 157, 158, 159, 160, 161, 162, 163, 164, 168, 169, 170, 171, 172, 173, 174, 175, 176, 177, 178, 179, 180, 181, 182, 183, 184, 185, 263, 264, 265, 266, 267, 268, 269, 307, 308, 309, 310, 311, 312, 313, 356, 357, 358, 359, 360, 361, 362, 405, 406, 407, 408, 409, 410, 411 with reference to positions set forth in SEQ ID NO:2. For example, the amino acid replacements can be replacements at one or more positions corresponding to position tyrosine (Y) 102, T103, P104, D105, L106, P107, R108, G136, Q137, A138, D139, I140, M141, I142, 5153, P154, F155, D156, G157, P158, G159, G160, N161, L162, A163, H164, P168, G169, P170, G171, I172, G173, G174, D175, A176, H177, F178, D179, E180, D181, E182, R183, W184, T185, L263, T264, F265, D266, A267, I268, T269, N307, G308, L309, E310, A311, A312, Y313, K356, H357, I358, D359, A360, A361, L362, H405, K406, V407, D408, A409, V410, F411, with reference to amino acid positions set forth in SEQ ID NO:2. With reference to SEQ ID NO:5, the amino acid replacements can be replacements at one or more positions tyrosine (Y) 22, T23, P24, D25, L26, P27, R28, G56, Q57, A58, D59, I60, M61, I62, S73, P74, F75, D76, G77, P78, G79, G80, N81, L82, A83, H84, P88, G89, P90, G91, I92, G93, G94, D95, A96, H97, F98, D99, E100, D101, E102, R103, W104, T105, L183, T184, F185, D186, A187, I188, T189, N227, G228, L229, E230, A231, A232, Y233, K276, H277, I278, D279, A280, A281, L282, H325, K326, V327, D328, A329, V330, F331, with reference to amino acid positions set forth in SEQ ID NO:5. The modified MMP polypeptide, such as a modified MMP-1 polypeptide, can contain an amino acid replacement at the indicated position, such as any amino acid replacement set forth in Table 5, and in particular any amino acid replacement as set forth herein above at the corresponding position with reference to the positions set forth in SEQ ID NO:2.

In some examples, the amino acid replacement or replacements include at least one amino acid replacement at a position directly involved with calcium coordination. In such examples, the amino acid replacement or replacements include at least one amino acid replacement or replacements at any one or more positions corresponding to any of the following positions: 105, 139, 156, 157, 159, 161, 171, 173, 175, 179, 180, 182, 266, 310, 359, 408, with reference to positions set forth in SEQ ID NO:2. For example, the amino acid positions can be replacements at one or more positions corresponding to replacement of (D) at position 105 (D105), D139, D156, G157, G159, N161, G171, G173, D175, D179, E180, E182, D266, E310, D359 or D408, with reference to amino acid positions set forth in SEQ ID NO:2. With reference to SEQ ID NO:5, the amino acid replacements can be replacements at one or more positions D25, D59, D76, G77, G79, N81, G91, G93, D95, D99, E100, E102, D186, E230, D279 or D328, with reference to amino acid positions set forth in SEQ ID NO:5. The modified MMP polypeptide, such as a modified MMP-1 polypeptide, can contain an amino acid replacement at the indicated position, such as any amino acid replacement set forth in Table 5, and in particular any amino acid replacement as set forth herein above at the corresponding position with reference to the positions set forth in SEQ ID NO:2.

In particular examples herein, the unmodified MMP has an acidic amino acid (e.g., aspartic acid or glutamic acid) at the modified positions, such as for example, an amino acid position corresponding to any of positions 105, 139, 156, 175, 179, 180, 182, 266, 310, 359 or 408 with reference to positions set forth in SEQ ID NO:2, and in particular at an amino acid position corresponding to any of positions 105, 156, 179, 180 or 182. In such examples, the amino acid replacement is replacement by a non-acidic amino acid residue at the modified position. In one example, the modified MMP, such as a modified MMP-1 polypeptide, contains replacement of an acidic amino acid (e.g., aspartic acid or glutamic acid) at a position corresponding to any of positions 105, 139, 156, 175, 179, 180, 182, 266, 310, 359 or 408, and generally corresponding to any of positions 105, 156, 179, 180 or 182, with a neutral amino acid residue that is a cysteine (C), asparagine (N), glutamine (Q), threonine (T), tyrosine (Y), serine (S) or glycine (G). In particular examples, the replacement is with a threonine or asparagine. In another example, the modified MMP, such as a modified MMP-1 polypeptide, contains replacement of an acidic amino acid (e.g., aspartic acid or glutamic acid) at a position corresponding to any of positions 105, 139, 156, 175, 179, 180, 182, 266, 310, 359 or 408, and generally at a position corresponding to any of positions 105, 156, 179, 180 or 182, with a hydrophobic amino acid that is a phenylalanine (F), methionine (M), tryptophan (W), isoleucine (I), valine (V), leucine (L), alanine (A) or proline (P). In a further example, the modified MMP, such as a modified MMP-1 polypeptide, contains replacement of an acidic amino acid (e.g., aspartic acid or glutamic acid) at a position corresponding to any of positions 105, 139, 156, 175, 179, 180, 182, 266, 310, 359 or 408, and generally at a position corresponding to any of positions 105, 156, 179, 180 or 182, with a basic amino acid that is a histidine (H), lysine (K) or arginine (R).

In another example herein, the unmodified MMP has a neutral amino acid (e.g., a cysteine, asparagine, glutamine, threonine, tyrosine, serine or glycine) at the modified position, such as for example, an amino acid position corresponding to any of positions 157, 159, 161, 171, 173, with reference to positions set forth in SEQ ID NO:2, and in particular at an amino acid position corresponding to position 159. In such examples, the amino acid replacement is replacement by a hydrophobic amino acid residue at the modified position. In one example, the modified MMP, such as a modified MMP-1 polypeptide, contains replacement of a neutral amino acid at a position corresponding to any of positions 157, 159, 161, 171, 173, and generally corresponding to position 159, with a hydrophobic amino acid residue that is a phenylalanine (F), methionine (M), tryptophan (W), isoleucine (I), valine (V), leucine (L), alanine (A) or proline (P).

Exemplary of modified MMP polypeptides provided herein containing at least one amino acid replacement at a position directly involved with calcium coordination, such as a modified MMP-1, are those that contain at least one amino acid replacement from among replacement with: A at a position corresponding to position 105; C at a position corresponding to position 105; F at a position corresponding to position 105; G at a position corresponding to position 105; I at a position corresponding to position 105; L at a position corresponding to position 105; M at a position corresponding to position 105; N at a position corresponding to position 105; P at a position corresponding to position 105; R at a position corresponding to position 105; S at a position corresponding to position 105; T at a position corresponding to position 105; V at a position corresponding to position 105; W at a position corresponding to position 105; H at a position corresponding to position 156; L at a position corresponding to position 156; A at a position corresponding to position 156; W at a position corresponding to position 156; C at a position corresponding to position 156; P at a position corresponding to position 156; V at a position corresponding to position 156; K at a position corresponding to position 156; S at a position corresponding to position 156; G at a position corresponding to position 156; T at a position corresponding to position 156; Y at a position corresponding to position 156; R at a position corresponding to position 156; M at a position corresponding to position 156; P at a position corresponding to position 159; V at a position corresponding to position 159; A at a position corresponding to position 159; M at a position corresponding to position 159; I at a position corresponding to position 159; W at a position corresponding to position 159; L at a position corresponding to position 159; P at a position corresponding to position 179; L at a position corresponding to position 179; G at a position corresponding to position 179; G at a Position corresponding to position 179; S at a position corresponding to position 179; A at a position corresponding to position 179; K at a position corresponding to position 179; T at a position corresponding to position 179; I at a position corresponding to position 179; R at a position corresponding to position 179; N at a position corresponding to position 179; W at a position corresponding to position 179; Q at a position corresponding to position 179; V at a position corresponding to position 179; C at a position corresponding to position 179; M at a position corresponding to position 180; P at a position corresponding to position 180; K at a position corresponding to position 180; Y at a position corresponding to position 180; Q at a position corresponding to position 180; R at a position corresponding to position 180; A at a position corresponding to position 180; T at a position corresponding to position 180; I at a position corresponding to position 180; F at a position corresponding to position 180; C at a position corresponding to position 180; G at a position corresponding to position 180; S at a position corresponding to position 180; N at a position corresponding to position 180; C at a position corresponding to position 182; P at a position corresponding to position 182; P at a position corresponding to position 182; S at a position corresponding to position 182; T at a position corresponding to position 182; R at a position corresponding to position 182; A at a position corresponding to position 182; F at a position corresponding to position 182; L at a position corresponding to position 182; I at a position corresponding to position 182; Y at a position corresponding to position 182; Q at a position corresponding to position 182; W at a position corresponding to position 182; M at a position corresponding to position 182; or G at a position corresponding to position 182, with reference to amino acid positions set forth in SEQ ID NO:2.

In particular, the amino acid replacement is replacement A at a position corresponding to position 105; I at a position corresponding to position 105; N at a position corresponding to position 105; L at a position corresponding to position 105; G at a position corresponding to position 105; R at a position corresponding to position 156; H at a position corresponding to position 156; K at a position corresponding to position 156; T at a position corresponding to position 156; V at a position corresponding to position 159; N at a position corresponding to position 179; T at a position corresponding to position 180; F at a position corresponding to position 180; and T at a position corresponding to position 182, with reference to amino acid positions set forth in SEQ ID NO:2. In particular examples, the amino acid replacement is replacement with threonine (T) at a position corresponding to position 156; replacement with valine (V) at a position corresponding to position 159; and/or replacement with Asparagine (N) at a position corresponding to position 179, with reference to positions set forth in SEQ ID NO:2.

Any of the above modifications, such as amino acid replacements, at an amino acid residue involved in calcium coordination can be made at a corresponding position of a MMP polypeptide, such as any set forth in SEQ ID NOS:17, 20, 23, 26, 29, 32, 35, 38, 41, 44, 47, 50, 53, 56, 59, 62, 65, 68, 71, 74, 76, 80, 83, or mature or catalytically active form thereof or variants of any of such forms, such as those that exhibit at least 60%, 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:17, 20, 23, 26, 29, 32, 35, 38, 41, 44, 47, 50, 53, 56, 59, 62, 65, 68, 71, 74, 76, 80, 83. For example, the modifications (e.g., amino acid replacements) can be made in a mature or catalytically active form of any of the above polypeptides, such as any MMP polypeptide set forth in any of SEQ ID NOS:5, 132, 133, 134, 135, 137, 138, 140, 143 or 145 or a catalytically active fragment thereof, or any form or variant thereof that has a sequence of amino acids that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:5, 132, 133, 134, 135, 137, 138, 140, 143 or 145 or a catalytically active fragment thereof, so long as the modified MMP exhibits increased activity at a calcium concentration greater than physiological levels compared to activity at physiological concentrations of calcium. Table 6 depicts exemplary corresponding amino acid replacements with reference to exemplary zymogen forms of modified MMPs (e.g., FIGS. 2A-2C). For use in the methods herein, the modified MMP includes any active or catalytically active form of the modified zymogen lacking the propeptide domain and including the corresponding amino acid replacement(s).

TABLE 6
Exemplary modifications in MMPs

| | SEQ | |
|---|---|---|
| MMP | ID NO | Amino Acid Modifications |
| MMP-1 | 2 | D105A; D105I; D105N; D105L; D105G; D156R; |
| (zymogen) | | D156K; D156H; D156T; G159V; D179N; E180T; |
| | | E180F; E182T |
| MMP-1 | 5 | D25A; D25I; D25N; D25L; D25G; D76R; D76K; |
| (mature) | | D76H; D76T; G79V; D99N; E100T; E100F; E102T |
| MMP-8 | 17 | Q103A; Q103I; Q103N; Q103L; Q105G; D154R; |
| | | D154K; D154H; D154T; N157V; D177N; A180T; |
| | | A180F; E182T |
| MMP-13 | 20 | D109A; D109I; D109N; D109L; D109G; D160R; |
| | | D160K; D160H; D160T; S163V; D183N; D184T; |
| | | D184F; E186T |
| MMP-18 | 23 | D107A; D107I; D107N; D107L; D107G; D158R; |
| | | D158K; D158H; D158T; G161V; D181N; E182T; |
| | | E182F; E184T |

In particular examples, the amino acid replacement or replacements is in a MMP-1 polypeptide, such as in a precursor MMP-1 polypeptide set forth in SEQ ID NO:1, an inactive pro-enzyme MMP-1 containing the propeptide (zymogen form) set forth in SEQ ID NO:2, a mature MMP-1 polypeptide lacking the propeptide set forth in SEQ ID NO:5, or any variant (e.g., species, allelic or modified variant) or active fragment thereof that has 40%, 50%, 60%, 70%, 80%, 90%, 91%, 92%, 93%, 94%, 95%, 96%, 97%, 98%, or 99% sequence identity to any of the MMP-1 polypeptides set forth in SEQ ID NOS:1, 2 or 5, so long as the modified MMP polypeptide retains increased calcium dependency for enzymatic activity. For example, provided herein are modified MMP-1 polypeptides: containing an amino acid replacement corresponding to G159V having the sequence of amino acids set forth in SEQ ID NO:158 (zymogen form) or in an SEQ ID NO:162 (mature form); containing an amino acid replacement corresponding to D156T having the sequence of amino acids set forth in SEQ ID NO:160 (zymogen form) or SEQ ID NO:164 (mature form); or containing an amino acid replacement corresponding to D179N having the sequence of amino acids set forth in SEQ ID NO:161 (zymogen form) or SEQ ID NO:165 (mature form). Also provided herein are modified MMP-1 polypeptides having a sequence of amino acids that exhibits at least 70%, 80%, 90%, 91%, 92%, 93%, 94%, 95%, 96%, 97%, 98%, or 99% sequence identity to any of the MMP-1 polypeptides set forth in SEQ ID NOS:158, 160, 161, 162, 164 or 165, so long as the modified MMP polypeptide contains the corresponding amino acid replacement and retains increased calcium dependency for enzymatic activity.

In particular, provided herein is a modified MMP polypeptide, such as a modified MMP-1 polypeptide, that contains an amino acid replacement of valine (V) at a position corresponding to position 159, and in particular in a position corresponding to Gly159. G159 is highly conserved among MMP proteins (see e.g., Maskos et al. (2005) Biochimie 87:249-263). Exemplary of such a modified MMP is one that exhibits at least 40%, 50%, 60%, 70%, 75%, 80%, 85%, 86%, 87%, 88%, 89%, 90%, 91%, 92%, 93%, 94%, 95%, 96%, 97%, 98%, 99% or more sequence identity to SEQ ID NO:2 or SEQ ID NO:5, and contains the amino acid replacement corresponding to G159V. For example, the modified MMP polypeptide is a MMP-1 polypeptide that contains the amino acid replacement G159V with reference to SEQ ID NO:2, which corresponds to G79V with reference to SEQ ID NO:5. Such modified polypeptides include those that have the sequence of amino acids set forth in SEQ ID NO:158 (zymogen form) or SEQ ID NO:162 (mature form), or that have a sequence of amino acids that exhibits at least 70%, 80%, 90%, 91%, 92%, 93%, 94%, 95%, 96%, 97%, 98%, or 99% sequence identity to any of the MMP-1 polypeptides set forth in SEQ ID NOS:158 or 162, so long as the modified MMP polypeptide retains increased calcium dependency for enzymatic activity. G159 is located between n-sheet strands III and IV in the S-loop, and coordinating the calcium 3 calcium molecule along with five other amino acids (156, 157, 161, 179 and 182) facilitates the stabilization of the S-loop to the underlying β-sheet strand IV, which, in turn, is involved in tertiary structure stabilization. Hence, position 159, such as G159, plays a role in Ca²⁺ binding. In particular, substitution of the larger, hydrophobic side chain of valine for the hydrogen molecule of glycine, or other neutral amino acid, disrupts the structure of the Ca²⁺ binding pocket such that its affinity for Ca²⁺ is altered. At higher concentrations of Ca²⁺, the tertiary structure is stabilized but as the concentration decreases, Ca²⁺ does not bind as efficiently. Concomitant with the decreased Ca²⁺ binding also is the loss of tertiary structure and the subsequent inactivation and autolysis of the protein.

2. Modification of a Residue Corresponding to Position 227

Also among the modified csMMP polypeptides provided herein for use in the methods herein are csMMP polypeptides containing an amino acid modification in a starting, unmodified MMP at an amino acid residue that corresponds to position 227, with reference to SEQ ID NO:2. Typically, the modification is an amino acid replacement. The amino acid replacement can be to any other amino acid so long as the resulting modified MMP polypeptide exhibits calcium sensitivity at concentrations of calcium greater than the physiological concentration, and reduced activity at physiological concentrations of calcium. For example, amino acid replacements include replacement of amino acids to an acidic (D or E); basic (H, K or R); neutral (C, N, Q, T, Y, S, G) or hydrophobic (F, M, W, I, V, L A, P) amino acid residue. For example, amino acid replacements at the noted position include replacement by amino acid residues E, H, R, C, Q, T, S, G, M, W, I, V, L, A, P, N, F, D, Y or K. In particular examples herein, the amino acid replacement is to a glutamic acid (E).

The modification (e.g., amino acid replacement) can be made at a corresponding position in a MMP polypeptide, such as any set forth in SEQ ID NOS:17, 20, 23, 26, 29, 32, 35, 38, 41, 44, 47, 50, 53, 56, 59, 62, 65, 68, 71, 74, 76, 80, 83, or mature or catalytically active form thereof or variants of any of such forms, such as those that exhibit at least 60%, 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:17, 20, 23, 26, 29, 32, 35, 38, 41, 44, 47, 50, 53, 56, 59, 62, 65, 68, 71, 74, 76, 80, 83. For example, the modification (e.g., amino acid replacement) can be made in a mature or catalytically active form of any of the above polypeptides, such as any MMP polypeptide set forth in any of SEQ ID NOS:5, 132, 133, 134, 135, 137, 138, 140, 143 or 145 or a catalytically active fragment thereof, or any form or variant thereof that has a sequence of amino acids that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:5, 132, 133, 134, 135, 137, 138, 140, 143 or 145 or a catalytically active fragment thereof, so long as the modified MMP exhibits increased activity at a calcium concentration greater than physiological levels compared to activity at physiological concentrations of calcium.

For example, provided herein are modified MMP polypeptides, such as modified MMP-1 polypeptides, having an amino acid replacement corresponding to V227E, with reference to positions set forth in SEQ ID NO:2. Such modified polypeptides include those that have the sequence of amino acids set forth in SEQ ID NO:157 (zymogen form) or SEQ ID NO:167 (mature form), or that have a sequence of amino acids that exhibits at least 70%, 80%, 90%, 91%, 92%, 93%, 94%, 95%, 96%, 97%, 98%, or 99% sequence identity to any of the MMP-1 polypeptides set forth in SEQ ID NOS:157 or 167, so long as the modified MMP polypeptide retains increased calcium dependency for enzymatic activity.

3. Combination Mutants

Modified MMP polypeptides, such as modified MMP-1 polypeptides, used in the methods provided herein, can contain any two or more modifications described above, whereby the resulting modified MMP exhibits calcium-sensitive activity in the presence of high calcium concentrations greater than physiological levels, and decreased activity in the presence of physiological levels of calcium. For example, MMP-1 polypeptides can contain 2, 3, 4, 5, 6, 7, 8, 9, 10, 11, 12, 13, 14, 15, 16, 17, 18, 19, 20 or more modifications compared to a starting or reference MMP-1 polypeptide. Such combination mutants are calcium sensitive and can exhibit the same, more, or less calcium dependence as compared to a csMMP-1 polypeptide containing a single modified residue or fewer modified residues. Typically, combination mutants retain activity at high concentrations of calcium (e.g., 10 mM Ca²⁺) compared to the single mutant MMP-1 polypeptides alone or compared to an unmodified MMP-1 polypeptide not containing the amino acid changes (e.g., a wild-type MMP-1 polypeptide set forth in SEQ ID NO:2 or active forms or other forms thereof) in the presence of high calcium concentrations (e.g., 10 mM Ca²⁺).

For example, a modified MMP polypeptide, such as a modified MMP-1 polypeptide, provided herein for use in the methods include polypeptides with any two or more modifications at positions as described above. In particular examples, the modified MMP polypeptide, such as a modified MMP-1 polypeptide, contains two or more modifications at positions described above that are at or near metal-binding sites. For example, modified MMP-1 polypeptides provided herein contain amino acid replacements, such as any described above, at any two or more positions corresponding to any of the following positions: 102, 103, 104, 105, 106, 107, 108, 136, 137, 138, 139, 140, 141, 142, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 196, 197, 198, 199, 201, 202, 203, 204, 205, 206, 207, 208, 209, 210, 211, 212, 263, 264, 265, 266, 267, 268, 269, 307, 308, 309, 310, 311, 312, 313, 356, 357, 358, 359, 360, 361, 362, 405, 406, 407, 408, 409, 410, 411, with reference to positions set forth in SEQ ID NO:2. The two or more modifications (e.g., amino acid replacements) can be made at a corresponding position of a MMP polypeptide, such as any set forth in SEQ ID NOS:17, 20, 23, 26, 29, 32, 35, 38, 41, 44, 47, 50, 53, 56, 59, 62, 65, 68, 71, 74, 76, 80, 83, or mature or catalytically active form thereof or variants of any of such forms, such as those that exhibit at least 60%, 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:17, 20, 23, 26, 29, 32, 35, 38, 41, 44, 47, 50, 53, 56, 59, 62, 65, 68, 71, 74, 76, 80, 83. For example, the two or more modifications (e.g., amino acid replacements) can be made in a mature or catalytically active form of any of the above polypeptides, such as any MMP polypeptide set forth in any of SEQ ID NOS:5, 132, 133, 134, 135, 137, 138, 140, 143 or 145 or a catalytically active fragment thereof, or any form or variant thereof that has a sequence of amino acids that exhibits at least 70%, 75%, 80%, 85%, 86%, 87%, 88%, 89%, 90%, 91%, 92%, 93%, 94%, 95%, 96%, 97%, 98%, 99% or more sequence identity to any of SEQ ID NOS:5, 132, 133, 134, 135, 137, 138, 140, 143 or 145 or a catalytically active fragment thereof, so long as the modified MMP exhibits increased activity at a calcium concentration greater than physiological levels compared to activity at physiological concentrations of calcium.

For example, among the modified MMP polypeptides provided herein are polypeptides that contain a modification at a position corresponding to position 159 (e.g., G159) with reference to positions set forth in SEQ ID NO:2, and another modification at another position at or near a metal binding site. Exemplary of such an additional replacement is replacement with a basic amino acid, for example, lysine (K), at a position corresponding to position 208, with reference to amino acid positions set forth in SEQ ID NO:2. For example, provided herein are modified MMP polypeptides, such as modified MMP-1 polypeptides, having amino acid replacements corresponding to G159V and S208K, with reference to positions set forth in SEQ ID NO:2. Such modified polypeptides include those that have the sequence of amino acids set forth in SEQ ID NO:159 (zymogen form) or SEQ ID NO:163 (mature form), or that have a sequence of amino acids that exhibits at least 70%, 80%, 90%, 91%, 92%, 93%, 94%, 95%, 96%, 97%, 98%, or 99% sequence identity to any of the MMP-1 polypeptides set forth in SEQ ID NOS:159 or 163, so long as the modified MMP polypeptide retains increased calcium dependency for enzymatic activity.

In particular, modified MMP polypeptides, such as modified MMP-1 polypeptides, provided herein can include any two or more modifications (e.g., amino acid replacements) at positions involved in calcium coordination from among any of the following positions: 102, 103, 104, 105, 106, 107, 108, 136, 137, 138, 139, 140, 141, 142, 153, 154, 155, 156, 157, 158, 159, 160, 161, 162, 163, 164, 168, 169, 170, 171, 172, 173, 174, 175, 176, 177, 178, 179, 180, 181, 182, 183, 184, 185, 263, 264, 265, 266, 267, 268, 269, 307, 308, 309, 310, 311, 312, 313, 356, 357, 358, 359, 360, 361, 362, 405, 406, 407, 408, 409, 410, 411 with reference to positions set forth in SEQ ID NO:2. Exemplary modified MMP polypeptides contain two or more modifications at any of positions 105, 156, 159, 179, 180 or 182, and generally two or more modifications at positions 156, 159 or 179, each with reference to positions set forth in SEQ ID NO:2. Typically, the modification is an amino acid replacement, such as any replacement at the noted positions as described above. For example, provided herein is a modified MMP polypeptide, such as a modified MMP-1 polypeptide, having amino acid replacements corresponding to D156T and D179N, with reference to positions set forth in SEQ ID NO:2. Such modified polypeptides include those that have the sequence of amino acids set forth in SEQ ID NO:156 (zymogen form) or SEQ ID NO:166 (mature form), or that have a sequence of amino acids that exhibits at least 70%, 80%, 90%, 91%, 92%, 93%, 94%, 95%, 96%, 97%, 98%, or 99% sequence identity to any of the MMP-1 polypeptides set forth in SEQ ID NOS:156 or 166, so long as the modified MMP polypeptide retains increased calcium dependency for enzymatic activity.

4. Additional Modifications

Any modified MMP polypeptide, such as any modified MMP-1 polypeptide, provided for use in the methods also can contain one or more other modifications described in the art, so long as the modified polypeptide retains increased calcium dependency for enzymatic activity. In addition to containing one or more modification(s) described above in Sections D.1-D.3, any modified MMP-1 polypeptide provided herein can contain 1, 2, 3, 4, 5, 6, 7, 8, 9, 10, 11, 12, 13, 14, 15, 16, 17, 18, 19, 20 or more additional modifications. The additional modifications can include modifications of the primary sequence and modifications not in the primary sequence of the polypeptide (e.g., post-translational modifications, conjugations or fusions), including any known in the art. For example, any amino acid substitution, deletion or insertion known in the art can be included. The additional modifications can confer additional properties to the enzyme, for example, increased stability, increased half-life and/or increased resistance to inhibitors, for example, TIMP.

The additional modification can be any one or more of the modifications set forth in Table 7 corresponding to amino acid positions in human MMP-1 as set forth in SEQ ID NO:2: In other examples, modified MMP polypeptides, including modified MMP-1 polypeptides, for use in the methods herein can contain only one, or more than one, of the amino acid replacements set forth in Table 7, whereby the resulting modified polypeptide exhibits calcium-sensitive activity in the presence of high calcium concentrations greater than physiological levels, and decreased activity in the presence of physiological levels of calcium.

TABLE 7
Additional Modifications

| | Corresponding | |
|---|---|---|
| | hMMP-1 | |
| Posi- | Residue (SEQ | |
| tion | ID NO: 2) | Amino Acid Substitutions |
| 81 | F | E; H; R; C; Q; T; S; G; M; W; I; V; L; A; P |
| 82 | V | R; C; N; Q; T; Y; S; G; F; M; W; I; L; A; P |
| 83 | L | D; E; H; R; C; Q; T; Y; S; G; M; W; I; A; P |
| 84 | T | D; E; H; R; C; Q; Y; S; G; F; I; V; L; A; P |
| 85 | E | K; R; C; N; Q; T; Y; S; G; F; M; V; L; A; P |
| 86 | G | D; H; K; C; N; T; Y; S; F; M; W; I; V; L; P |
| 87 | N | E; H; R; C; Q; Y; S; G; F; M; I; V; L; A; P |
| 88 | P | D; E; H; K; R; C; Q; T; Y; G; W; I; V; L; A |
| 89 | R | E; H; K; N; T; Y; S; G; F; M; W; V; L; A; P |
| 90 | W | E; H; R; N; Q; T; S; G; F; M; I; V; L; A; P |
| 91 | E | D; H; R; C; N; T; Y; S; G; F; W; I; V; L; A |
| 92 | Q | E; K; R; N; T; Y; S; G; F; W; I; V; L; A; P |
| 93 | T | D; E; K; R; N; S; G; F; M; W; I; V; L; A; P |
| 94 | H | D; E; R; N; T; S; G; F; M; W; I; V; L; A; P |
| 95 | L | D; E; H; K; R; C; T; Y; S; G; W; I; V; A; P |
| 96 | T | E; H; R; C; N; Q; S; G; F; W; I; V; L; A; P |
| 97 | Y | D; E; H; K; R; N; Q; T; S; G; W; V; L; A; P |
| 98 | R | D; E; H; K; C; Y; S; G; F; M; W; V; L; A; P |
| 99 | I | E; H; R; C; N; Q; T; Y; S; G; F; W; V; L; A; P |
| 100 | E | D; H; R; N; T; Y; S; G; F; M; W; I; V; L; P |
| 101 | N | D; H; K; R; C; T; Y; S; F; M; W; V; L; A; P |
| 102 | Y | D; E; K; R; C; N; Q; S; G; F; M; V; L; A; P |
| 103 | T | D; E; K; R; C; N; Q; Y; S; G; W; V; L; A; P |
| 104 | P | D; E; H; R; C; Q; T; Y; S; G; F; M; V; L; A |
| 105 | D | E; R; C; N; T; S; G; F; M; W; I; V; L; A; P |
| 106 | L | D; H; R; C; N; T; Y; S; G; F; M; I; V; A; P |
| 107 | P | D; K; R; C; T; Y; S; G; F; M; W; I; V; L; A |
| 108 | R | E; K; C; N; T; Y; S; G; F; W; I; V; L; A; P |
| 109 | A | D; E; H; R; N; Q; T; Y; S; G; M; W; I; V; L; |
| 110 | D | H; R; C; Q; T; Y; S; G; F; M; I; V; L; A; P |
| 111 | V | D; E; K; R; C; Q; T; Y; S; G; W; I; L; A; P |
| 112 | D | H; K; R; C; Q; T; Y; S; G; F; M; W; I; V; L; A; P |
| 113 | H | D; E; R; N; T; Y; S; G; F; M; W; V; L; A; P |
| 114 | A | E; R; C; N; Q; T; S; G; F; M; W; I; V; L; P |
| 115 | I | D; E; H; K; R; C; Q; T; S; G; F; W; V; L; P |
| 116 | E | D; H; K; R; C; N; Q; S; G; F; M; I; L; A; P |
| 117 | K | D; E; H; R; N; Q; T; Y; S; G; F; W; L; A; P |
| 118 | A | D; E; H; K; R; Q; T; S; G; F; W; I; V; L; P |
| 119 | F | E; H; K; R; C; N; T; Y; S; G; W; V; L; A; P |
| 120 | Q | D; E; H; K; R; C; N; T; Y; G; M; W; V; A; P |
| 121 | L | E; H; K; R; C; N; Q; T; S; G; F; I; V; A; P |
| 122 | W | E; H; K; R; N; Q; T; Y; S; G; F; V; L; A; P |
| 123 | S | D; H; K; R; C; N; Q; T; Y; G; F; M; W; I; V; L; |
| | | A; P |
| 124 | N | D; K; R; C; T; S; G; F; M; W; I; V; L; A; P |
| 125 | V | D; E; H; R; C; Q; T; Y; S; G; F; M; W; A; P |
| 126 | T | E; H; K; R; N; Q; S; G; F; M; W; V; L; A; P |
| 127 | P | E; H; K; R; C; Q; T; S; F; M; W; I; V; L; A |
| 128 | L | D; K; R; C; Q; T; S; G; F; M; W; I; V; A; P |
| 129 | T | E; H; K; R; C; Y; S; G; F; M; I; V; L; A; P |
| 130 | F | E; H; K; R; C; N; T; Y; S; G; I; V; L; A; P |
| 131 | T | D; E; H; R; C; Q; Y; S; G; F; M; I; L; A; P |
| 132 | K | D; E; H; R; T; Y; S; G; F; M; I; V; L; A; P |
| 133 | V | D; E; H; K; R; C; N; T; S; G; M; W; L; A; P |
| 134 | S | D; E; H; K; R; C; N; Q; T; Y; G; V; L; A; P |
| 135 | E | D; H; R; N; Q; T; S; F; M; W; I; V; L; A; P |
| 136 | G | D; E; H; R; C; N; T; S; M; W; I; V; L; A; P |
| 137 | Q | E; H; K; R; C; N; T; Y; S; G; F; W; L; A; P |
| 138 | A | D; E; H; R; C; Q; T; S; G; M; W; I; V; L; P |
| 139 | D | E; H; R; C; N; Y; S; G; F; M; W; I; V; L; A; P |
| 140 | I | D; E; H; K; R; C; T; Y; G; F; M; W; V; L; A |
| 141 | M | D; E; H; R; C; N; T; Y; S; G; W; I; L; A; P |
| 142 | I | K; R; N; Q; T; Y; S; G; F; M; W; V; L; A; P |
| 143 | S | E; H; R; C; N; Q; T; Y; G; M; W; I; L; A; P |
| 144 | F | E; H; K; R; C; N; Q; T; S; G; M; W; V; L; P |
| 145 | V | D; E; H; K; R; C; N; Q; T; S; G; W; L; A; P |
| 146 | R | D; E; H; K; C; N; Q; T; Y; S; F; V; L; A; P |
| 147 | G | E; H; R; C; Q; T; S; F; M; W; I; V; L; A; P |
| 148 | D | E; K; R; C; N; T; S; G; M; W; I; V; L; A; P |
| 149 | H | E; R; C; N; Q; T; Y; S; G; W; I; V; L; A; P |
| 150 | R | D; E; H; K; N; T; S; G; M; W; I; V; L; A; P |
| 151 | D | K; R; N; Q; T; Y; S; G; F; M; W; V; L; A; P |
| 152 | N | D; H; K; R; C; T; Y; S; G; F; W; I; L; A; P |
| 153 | S | D; H; K; R; C; Q; T; Y; G; F; I; V; L; A; P |
| 154 | P | H; K; R; C; N; Q; T; Y; S; F; W; I; V; L; A |
| 155 | F | E; H; R; N; Q; T; Y; S; G; M; W; V; L; A; P |
| 156 | D | E; H; K; R; C; T; Y; S; G; M; W; V; L; A; P |
| 157 | G | D; H; K; R; N; Q; T; Y; S; F; M; V; L; A; P |
| 158 | P | D; K; R; C; N; Q; T; Y; S; G; F; W; I; V; L; A |
| 159 | G | E; K; R; C; Q; T; Y; S; M; W; I; V; L; A; P |
| 160 | G | E; H; R; C; N; Q; T; S; M; W; I; V; L; A; P |
| 161 | N | E; H; R; C; Q; T; Y; S; G; F; W; I; V; L; P |
| 162 | L | D; E; R; C; Q; T; Y; S; G; F; M; W; I; A; P |
| 163 | A | E; K; R; C; N; Q; T; Y; S; G; F; I; V; L; P |
| 164 | H | E; K; R; C; N; Q; Y; S; G; F; M; V; L; A; P |
| 165 | A | D; H; K; R; N; Q; T; S; G; F; M; W; V; L; P |
| 166 | F | E; H; K; R; C; N; S; G; M; W; I; V; L; A; P |
| 167 | Q | D; E; K; R; N; T; Y; S; G; F; M; V; L; A; P |
| 168 | P | D; H; R; C; N; T; S; G; F; M; W; I; V; L; A |
| 169 | G | D; E; H; R; C; Q; T; S; M; W; I; V; L; A; P |
| 170 | P | D; H; K; R; C; Q; T; S; G; F; M; W; I; L; A |
| 171 | G | D; E; H; K; R; C; N; Q; Y; S; M; W; L; A; P |
| 172 | I | D; E; R; C; N; Q; T; Y; G; M; W; V; L; A; P |
| 173 | G | D; K; R; C; N; T; Y; S; F; M; W; V; L; A; P |
| 174 | G | D; E; H; R; N; T; Y; S; F; M; W; V; L; A; P |
| 175 | D | E; H; R; C; N; Q; T; Y; S; G; F; I; V; L; A; P |
| 176 | A | D; E; K; R; C; N; Q; T; S; G; F; W; V; L; P |
| 177 | H | D; R; C; N; Q; T; Y; S; G; W; I; V; L; A; P |
| 178 | F | E; H; K; R; C; Q; T; Y; S; G; W; I; V; L; A; P |
| 179 | D | E; K; R; C; N; Q; T; S; G; W; I; V; L; A; P |
| 180 | E | D; K; R; C; N; Q; T; Y; S; G; F; M; I; A; P |
| 181 | D | E; K; R; C; Q; T; Y; S; G; F; M; V; L; A; P |
| 182 | E | D; R; C; Q; T; Y; S; G; F; M; W; I; L; A; P |
| 183 | R | E; H; K; C; N; T; S; G; M; W; I; V; L; A; P |
| 184 | W | E; H; R; N; Q; T; S; G; F; M; I; V; L; A; P |
| 185 | T | D; E; H; R; C; N; Q; Y; S; G; W; V; L; A; P |
| 186 | N | D; E; H; R; C; Q; T; Y; S; G; F; V; L; A; P |
| 187 | N | D; H; K; R; C; T; S; G; F; M; W; I; L; A; P |
| 188 | F | D; E; H; K; R; N; Q; S; G; W; I; V; L; A; P |
| 189 | R | D; E; H; K; C; N; Q; T; Y; G; W; V; L; A; P |
| 190 | E | D; H; K; R; C; T; Y; S; G; M; I; V; L; A; P |
| 191 | Y | D; E; H; K; R; C; Q; T; S; G; W; V; L; A; P |
| 192 | N | D; H; K; R; C; Q; T; S; G; M; W; V; L; A; P |
| 193 | L | D; E; K; R; N; Q; T; Y; S; G; F; W; I; A; P |
| 194 | H | E; K; Q; T; Y; S; G; F; M; W; I; V; L; A; P |
| 195 | R | D; E; K; C; Q; T; Y; S; G; F; W; V; L; A; P |
| 196 | V | D; E; H; K; R; Q; T; Y; S; G; M; I; L; A; P |
| 197 | A | E; H; R; C; N; Q; T; Y; S; G; W; I; V; L; P |
| 198 | A | D; E; H; K; R; T; Y; S; G; F; M; W; V; L; P |
| 199 | H | E; K; R; C; N; T; S; G; M; W; I; V; L; A; P |
| 200 | E | D; R; C; N; T; Y; S; G; F; M; W; I; V; A; P |
| 201 | L | D; E; K; R; N; Q; T; S; G; M; W; I; V; A; P |
| 202 | G | D; E; H; K; R; C; T; Y; S; M; I; V; L; A; P |
| 203 | H | D; E; R; C; N; Q; T; Y; S; G; I; V; L; A; P |
| 204 | S | D; H; K; R; N; Q; T; Y; G; W; I; V; L; A; P |
| 205 | L | D; E; R; C; N; Q; T; S; G; M; W; I; V; A; P |
| 206 | G | D; E; H; R; C; Q; T; S; M; W; I; V; L; A; P |
| 207 | L | D; H; K; R; N; Q; Y; S; G; M; W; I; V; A; P |
| 208 | S | D; E; K; R; C; N; Q; T; G; F; W; V; L; A; P |
| 209 | H | D; R; C; N; Q; T; Y; S; G; F; W; V; L; A; P |
| 210 | S | H; K; R; C; N; Q; T; G; F; W; I; V; L; A; P |
| 211 | T | D; H; K; R; N; Q; S; G; F; M; W; V; L; A; P |
| 212 | D | E; H; K; R; N; Q; T; Y; S; G; F; V; L; A; P |
| 213 | I | D; E; H; K; R; C; N; Q; T; S; G; F; M; V; L; A; P |
| 214 | G | D; E; R; C; Q; T; Y; S; F; M; I; V; L; A; P |
| 215 | A | D; H; K; R; C; N; Q; T; S; G; M; W; I; V; L; P |
| 216 | L | D; E; K; R; C; Q; T; S; G; M; W; I; V; A; P |
| 217 | M | D; H; K; R; C; N; Q; T; Y; S; G; I; L; A; P |
| 218 | Y | D; E; R; C; N; Q; S; G; F; W; I; V; L; A; P |
| 219 | P | D; E; H; K; R; C; Q; T; S; G; F; W; V; L; A |
| 220 | S | E; H; K; R; N; Q; T; G; F; M; I; V; L; A; P |
| 221 | Y | E; K; R; C; N; Q; T; S; G; M; W; V; L; A; P |
| 222 | T | D; H; R; C; N; Y; S; G; F; M; W; I; V; L; A; P |
| 223 | F | E; H; K; R; C; N; Q; T; Y; S; G; M; L; A; P |
| 224 | S | D; H; K; R; C; Q; T; G; M; W; I; V; L; A; P |
| 225 | G | D; E; H; K; R; C; N; Q; T; S; M; W; V; A; P |
| 226 | D | E; H; R; C; N; T; S; G; M; W; I; V; L; A; P |
| 227 | V | D; E; H; K; R; C; Q; T; Y; S; G; W; L; A; P |
| 228 | Q | D; E; H; K; R; N; T; Y; S; G; M; W; L; A; P |
| 229 | L | D; E; H; R; C; Q; T; Y; G; M; W; I; V; A; P |
| 230 | A | D; H; R; C; N; T; Y; S; G; M; W; I; V; L; P |
| 231 | Q | D; H; R; C; Y; S; G; F; M; W; I; V; L; A; P |
| 232 | D | E; H; K; R; N; Q; T; Y; S; G; F; W; V; L; P |
| 233 | D | E; K; R; N; Q; T; S; G; M; W; I; V; L; A; P |
| 234 | I | D; E; H; C; N; Q; T; Y; G; M; W; V; L; A; P |
| 235 | D | E; H; R; C; N; Q; T; Y; S; G; I; V; L; A; P |
| 236 | G | D; E; K; R; C; N; T; Y; S; F; M; I; V; L; P |
| 237 | I | D; E; K; R; C; N; Q; T; Y; S; G; W; L; A; P |
| 238 | Q | E; H; K; R; C; N; T; Y; S; G; F; W; I; L; P |
| 239 | A | D; H; K; R; C; Q; T; Y; S; G; F; W; I; V; L; P |
| 240 | I | D; K; R; C; Q; T; Y; S; G; F; M; V; L; A; P |
| 241 | Y | D; H; R; N; Q; T; S; G; M; W; I; V; L; A; P |
| 242 | G | E; H; K; R; N; T; Y; S; F; W; I; V; L; A; P |
| 243 | R | D; H; K; C; N; Q; T; Y; S; G; I; V; L; A; P |
| 244 | S | D; E; H; R; Q; T; Y; G; F; M; W; V; L; A; P |
| 245 | Q | E; H; K; R; C; T; S; G; F; M; W; I; V; L; P |
| 246 | N | D; K; R; C; Q; T; Y; S; G; F; W; I; V; L; A; P |
| 247 | P | D; E; H; K; R; N; Q; T; S; G; F; I; V; L; A |
| 248 | V | E; H; K; R; C; Q; T; Y; S; G; F; M; W; I; L; A |
| 249 | Q | E; H; K; R; C; N; T; Y; G; W; I; V; L; A; P |
| 250 | P | D; K; R; N; Q; T; Y; S; G; F; M; W; V; L; A |
| 251 | I | D; E; K; R; C; Q; T; Y; S; G; W; V; L; A; P |
| 252 | G | D; E; H; K; R; C; T; S; F; M; W; I; V; L; A; P |
| 253 | P | E; K; R; C; N; Q; T; Y; G; M; W; I; V; L; A |
| 254 | Q | D; E; R; C; T; Y; S; G; F; W; I; V; L; A; P |
| 255 | T | E; H; K; R; C; N; Q; S; G; F; I; V; L; A; P |
| 256 | P | E; K; R; C; N; Q; Y; S; G; F; M; I; V; L; A |
| 257 | K | E; R; C; N; T; S; G; F; M; W; I; V; L; A; P |
| 258 | A | D; E; R; N; Q; T; Y; G; F; M; W; I; V; L; P |

The additional modification can be an allelic variant or other variant known in the art. Exemplary modifications that can be included in a polypeptide provided herein include, but are not limited to, amino acid replacements corresponding to T4P, Q10P, R30M, R30S, T96R, A114V, F166C, I172V, D181H, R189T, H199A; E200A, G214E, D232N, D233G, R243S, Q254P, I271A, R272A, T286A, 1298T, E314G, F315S, V374M, R386Q, S387T, G391S, and T432A, with reference to positions set forth in SEQ ID NO:2.

In some examples, additional modifications can confer increased sensitivity to temperature, pH, or other ions, such as monovalent cations (e.g., Na⁺ or K⁺). In particular examples, additional modifications can include modifications that confer temperature sensitivity. For example, MMP polypeptides that are modified to be temperature sensitive exhibit increased activity at a permissive temperature (e.g., 25° C.) compared to non-permissive temperatures (e.g., 34° C. or 37° C.). Exemplary modifications which can render a MMP-1 polypeptide temperature sensitive are set forth in U.S. Publication No. 2010/0284995. For example, modified MMP polypeptides, such as modified MMP-1 polypeptides, that exhibit at least 1.5 times or more activity at a temperature of 25° C. compared to at 34° C. or 37° C. include those that have an amino acid replacement corresponding to any one or more of modifications L95K, D105A, D105F, D105G, D1051, D105L, D105N, D105R, D105S, D105T, D105W, R150P, D151G, F155A, D156K, D156T, D156L, D156A, D156W, D156V, D156H, D156R, G159V, G159T, A176F, D179N, E180Y, E180T, E180F, D181L, D181K, E182T, E182Q, T185R, T185H, T185Q, T185A, T185E, N187R, N187M, N187F, N187K, N1871, R195V, A198L, A198M, G206A, G206S, S210V, Y218S, F223E, V227C, V227E, V227W, Q228P, L229T, L229I, D233E, I234A, I234T, I234E, 1240S, 1240C and C259Q, with reference to positions set forth in SEQ ID NO:2.

In other examples, additional modifications can confer increased activity of the modified MMP polypeptide compared to the unmodified polypeptide not containing the amino acid replacement. Exemplary modifications which can render a MMP-1 polypeptide temperature sensitive are set forth in U.S. Publication No. 2010/0284995. Exemplary modifications which can render a MMP-1 polypeptide temperature sensitive are set forth in U.S. Publication No. 2010/0284995. For example, modified MMP polypeptides, such as modified MMP-1 polypeptides, having increased activity include those that have an amino acid replacement corresponding to any one or more of modifications N161I, S208K, I213G, G214E, Q228A, Q228D, Q228E, Q228G, Q228H, Q228K, Q228L, Q228M, Q228N, Q228R, Q228S, Q228W, Q228Y, L229V, A230G, A230D, A230S, A230C, A230T, A230M, A230N, A230H, Q231A, Q231D, Q231G, Q231V, Q231S, D232H, D232G, D232P, D232V, D232K, D232W, D232Q, D232E, or D232T, with reference to positions in SEQ ID NO:2

Other modifications that are or are not in the primary sequence of the polypeptide also can be included in a modified MMP polypeptide, such as a modified MMP-1 polypeptide provided herein. Such additional modifications include posttranslational modifications, such as acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, or sulfation.

The modified MMP polypeptides also can be provided for use in the methods as conjugates, whereby the polypeptide is linked, directly or indirectly, to another moiety. The other moiety can be a peptide, protein, polymer, sugar, lipid or toxin. For example, the polymer can a polyethylene glycol (PEG) moiety. In other cases, the polypeptides are linked, directly or indirectly, to a multimerization domain such as an F_{c} domain. For example, such additional modifications can be made to increase the stability or half-life of the protein.

### E. METHODS OF PRODUCING NUCLEIC ACIDS ENCODING csMMPs AND POLYPEPTIDES THEREOF

Modified csMMP polypeptides set forth herein can be obtained by methods well known in the art for protein purification and recombinant protein expression. Any method known to those of skill in the art for identification of nucleic acids that encode desired genes can be used. Any method available in the art can be used to obtain a full length (i.e., encompassing the entire coding region) cDNA or genomic DNA clone encoding a desired MMP, such as from a cell or tissue source. Modified or modified variant csMMPs, can be engineered from a wild-type polypeptide, such as by site-directed mutagenesis.

Polypeptides can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a desired polypeptide, including for example, polymerase chain reaction (PCR) methods. A nucleic acid-containing material can be used as a starting material from which a desired polypeptide-encoding nucleic acid molecule can be isolated. For example, DNA and mRNA preparations, cell extracts, tissue extracts, fluid samples (e.g., blood, serum, saliva), or samples from healthy and/or diseased subjects can be used in amplification methods. Nucleic acid libraries also can be used as a source of starting material. Primers can be designed to amplify a desired polypeptide. For example, primers can be designed based on expressed sequences from which a desired polypeptide is generated. Primers can be designed based on back-translation of a polypeptide amino acid sequence. Nucleic acid molecules generated by amplification can be sequenced and confirmed to encode a desired polypeptide.

Additional nucleotide sequences can be joined to a polypeptide-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a polypeptide-encoding nucleic acid molecule. Examples of such sequences include, but are not limited to, promoter sequences designed to facilitate intracellular protein expression, and secretion sequences, for example heterologous signal sequences, designed to facilitate protein secretion. Such sequences are known to those of skill in the art. For example, exemplary heterologous signal sequences include, but are not limited to, human kappa IgG heterologous signal sequence set forth in SEQ ID NO:92. For bacterial expression, and exemplary heterologous signal sequence is the pelB leader sequence, for example, as set forth in SEQ ID NO:130. Additional nucleotide residues sequences, such as sequences of bases specifying protein binding regions, also can be linked to enzyme-encoding nucleic acid molecules. Such regions include, but are not limited to, sequences of residues that facilitate or encode proteins that facilitate uptake of an enzyme into specific target cells, or otherwise alter pharmacokinetics of a product of a synthetic gene. For example, enzymes can be linked to PEG moieties.

In addition, tags or other moieties can be added, for example, to aid in detection or affinity purification of the polypeptide. For example, additional nucleotide residues sequences, such as sequences of bases specifying an epitope tag or other detectable marker, also can be linked to enzyme-encoding nucleic acid molecules. Exemplary of such sequences include nucleic acid sequences encoding a His tag (e.g., 6×His, HHHHHH; SEQ ID NO:89) or Flag Tag (DYKDDDDK; SEQ ID NO:91).

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pCMV4, pBR322 or pUC plasmid derivatives or the Bluescript vector (Stratagene, La Jolla, Calif.). Other expression vectors include the pET303CTHis (SEQ ID NO:90; Invitrogen, CA) or pET-26B (SEQ ID NO:131) expression vector exemplified herein. The insertion into a cloning vector can, for example, be accomplished by ligating the DNA fragment into a cloning vector which has complementary cohesive termini. Insertion can be effected using TOPO cloning vectors (Invitrogen, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and protein gene can be modified by homopolymeric tailing. Recombinant molecules can be introduced into host cells via, for example, transformation, transfection, infection, electroporation and sonoporation, so that many copies of the gene sequence are generated.

In specific embodiments, transformation of host cells with recombinant DNA molecules that incorporate the isolated protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

1. Vectors and Cells

For recombinant expression of one or more of the desired proteins, such as any described herein, the nucleic acid containing all or a portion of the nucleotide sequence encoding the protein can be inserted into an appropriate expression vector, i.e., a vector that contains the necessary elements for the transcription and translation of the inserted protein coding sequence. The necessary transcriptional and translational signals also can be supplied by the native promoter for enzyme genes, and/or their flanking regions.

Vectors containing nucleic acid encoding a modified csMMP polypeptide can be introduced into a cell, including a prokaryotic or eukaryotic cell for protein production. Such cells include bacterial cells, yeast cells, fungal cells, Archea, plant cells, insect cells and animal cells. For example, the expression vector can be expressed in an animal cell that is an endothelial cell. The cells are used to produce a protein thereof by growing the above-described cells under conditions whereby the encoded protein is expressed by the cell, and recovering the expressed protein. Vectors can be selected for expression of the enzyme protein in a cell such that the enzyme protein is retained within the cell or secreted into the culture medium. For purposes herein, for example, the enzyme can be secreted into the medium.

The proenzyme (i.e., zymogen) form of the enzyme can be purified for use as a calcium-dependent, conditionally-active enzyme. Alternatively, upon secretion, the propeptide can be cleaved by chemical agents or catalytically or autocatalytically to generate a mature enzyme by the use of a processing agent. This processing step can be performed during the purification step and/or immediately before use of the enzyme. If desired, the processing agent can be dialyzed away or otherwise purified away from the purified protein before use. Additionally, if necessary, the enzyme can be purified such that the cleaved propeptide is removed from the preparation.

A variety of host-vector systems can be used to express the protein coding sequence. These include, but are not limited to, mammalian cell systems transfected with plasmid DNA or infected with virus (e.g., vaccinia virus, adenovirus and other viruses); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding protein, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for a desired protein. Promoters which can be used include, but are not limited to, the SV40 early promoter (Bemoist and Chambon (1981) Nature 290:304-310), the promoter contained in the 3′ long terminal repeat of Rous sarcoma virus (Yamamoto et al. (1980) Cell 22:787-797), the herpes thymidine kinase promoter (Wagner et al. (1981) Proc. Natl. Acad. Sci. U.S.A. 78:1441-1445), the regulatory sequences of the metallothionein gene (Brinster et al. (1982) Nature 296:39-42); prokaryotic expression vectors such as the β-lactamase promoter (Jay et al. (1981) Proc. Natl. Acad. Sci. U.S.A. 78:5543) or the tac promoter (DeBoer et al. (1983) Proc. Natl. Acad. Sci. U.S.A. 80:21-25); see also Gilbert and VIIIa-Komaroff, “Useful Proteins from Recombinant Bacteria,” Sci. Am. (1980) 242:74-94; plant expression vectors containing the nopaline synthase promoter (Herrera-Estrella et al. (1984) Nature 303:209-213) or the cauliflower mosaic virus 35S RNA promoter (Gardner et al. (1981) Nucleic Acids Res. 9:2871), and the promoter of the photosynthetic enzyme ribulose bisphosphate carboxylase (Herrera-Estrella et al. (1984) Nature 310:115-120); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al. (1984) Cell 38:639-646; Ornitz et al. (1986) Cold Spring Harbor Symp. Quant. Biol. 50:399-409; MacDonald (1987) Hepatol. 7:425-515); insulin gene control region which is active in pancreatic beta cells (Hanahan et al. (1985) Nature 315:115-122), immunoglobulin gene control region which is active in lymphoid cells (Grosschedl et al. (1984) Cell 38:647-658; Adams et al. (1985) Nature 318:533-538; Alexander et al. (1987) Mol. Cell. Biol. 7:1436-1444), mouse mammary tumor virus control region which is active in testicular, breast, lymphoid and mast cells (Leder et al. (1986) Cell 45:485-495), albumin gene control region which is active in liver (Pinkert et al. (1987) Genes Dev. 1:268-276), alpha-fetoprotein gene control region which is active in liver (Krumlauf et al. (1985) Mol. Cell. Biol. 5:1639-1648; Hammer et al. (1987) Science 235:53-58), alpha-1 antitrypsin gene control region which is active in liver (Kelsey et al. (1987) Genes Dev. 1:161-171), beta globin gene control region which is active in myeloid cells (Magram et al. (1985) Nature 315:338-340; Kollias et al. (1986) Cell 46:89-94), myelin basic protein gene control region which is active in oligodendrocyte cells of the brain (Readhead et al. (1987) Cell 48:703-712), myosin light chain-2 gene control region which is active in skeletal muscle (Shani (1985) Nature 314:283-286), and gonadotrophic releasing hormone gene control region which is active in gonadotrophs of the hypothalamus (Mason et al. (1986) Science 234:1372-1378).

In a specific embodiment, a vector is used that contains a promoter operably linked to nucleic acids encoding a desired protein, or a domain, fragment, derivative or homolog thereof, one or more origins of replication, and optionally, one or more selectable markers (e.g., an antibiotic resistance gene). Exemplary plasmid vectors for transformation of E. coli cells include, for example, the pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, t₀ and T1 transcriptional terminators, ColE1 origin of replication, and a beta-lactamase gene for conferring ampicillin resistance. The pQE vectors enable placement of a 6×His tag at either the N- or C-terminus of the recombinant protein. Such plasmids include pQE 32, pQE 30, and pQE 31 which provide multiple cloning sites for all three reading frames and provide for the expression of N-terminally 6×His-tagged proteins. Other exemplary plasmid vectors for transformation of E. coli cells include, for example, the pET expression vectors (see e.g., U.S. Pat. No. 4,952,496; available from Novagen, Madison, Wis.; see also literature published by Novagen describing the system). Such plasmids include pET 11a, which contains the T7lac promoter, T7 terminator, the inducible E. coli lac operator, and the lac repressor gene; pET 12a-c, which contains the T7 promoter, T7 terminator, and the E. coli ompT secretion signal; and pET 15b and pET19b (Novagen, Madison, Wis.), which contain a His-Tag™ leader sequence for use in purification with a His column and a thrombin cleavage site that permits cleavage following purification over the column, the T7-lac promoter region and the T7 terminator, and pET-26B (SEQ ID NO:131). An additional pET vector is pET303CTHis (set forth in SEQ ID NO:90; Invitrogen, CA), which contains a T7lac promoter, T7 terminator, the inducible E. coli lac operator, a beta-lactamase gene for conferring ampicillin resistance, and also a His-Tag sequence for use in purification.

Exemplary of a vector for mammalian cell expression is the HZ24 expression vector. The HZ24 expression vector was derived from the pCI vector backbone (Promega). It contains DNA encoding the beta-lactamase resistance gene (AmpR), an F1 origin of replication, a cytomegalovirus immediate-early enhancer/promoter region (CMV), and an SV40 late polyadenylation signal (SV40). The expression vector also has an internal ribosome entry site (IRES) from the ECMV virus (Clontech) and the mouse dihydrofolate reductase (DHFR) gene.

2. Expression

Modified csMMP polypeptides can be produced by any method known to those of skill in the art including in vivo and in vitro methods. Desired proteins can be expressed in any organism suitable to produce the required amounts and forms of the proteins, such as for example, needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of proteins. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

Modified csMMP polypeptides also can be utilized or expressed as protein fusions. For example, an enzyme fusion can be generated to add additional functionality to an enzyme. Examples of enzyme fusion proteins include, but are not limited to, fusions of a signal sequence, a tag such as for localization, e.g., a His₆ tag or a myc tag, or a tag for purification, for example, a GST fusion, and a sequence for directing protein secretion and/or membrane association.

Generally, modified csMMP polypeptides are expressed in an inactive zymogen form. Zymogen conversion can be achieved by exposure to chemical agents, to other proteases or to autocatalysis to generate a mature enzyme as described elsewhere herein. Any form of an enzyme is contemplated herein. It is understood that, if provided and expressed in a zymogen form, that it is processed to yield a mature polypeptide prior to use by a processing agent.

a. Prokaryotic Cells

Prokaryotes, especially E. coli, provide a system for producing large amounts of proteins. Transformation of E. coli is a simple and rapid technique well known to those of skill in the art. Expression vectors for E. coli can contain inducible promoters. Such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Proteins, such as any provided herein, can be expressed in the cytoplasmic environment of E. coli. The cytoplasm is a reducing environment and for some molecules, this can result in the formation of insoluble inclusion bodies. Reducing agents such as dithiothreitol and β-mercaptoethanol and denaturants, such as guanidine-HCl and urea can be used to re-solubilize the proteins. An alternative approach is the expression of proteins in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader (SEQ ID NO:130) from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm—by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast Cells

Yeasts such as Saccharomyces cerevisae, Schizosaccharomyces pombe, Yarrowia lipolytica, Kluyveromyces lactis and Pichia pastoris are well known yeast expression hosts that can be used for production of proteins, such as any described herein. Yeast can be transformed with episomal replicating vectors or by stable chromosomal integration by homologous recombination. Typically, inducible promoters are used to regulate gene expression. Examples of such promoters include GAL1, GAL7 and GALS and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site, such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as matrix-degrading enzymes. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express proteins including csMMPs. Expression constructs can be transferred to mammalian cells by viral infection such as by adenovirus, or by direct DNA transfer such as by liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. IRES elements also can be added to permit bicistronic expression with another gene, such as a selectable marker. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase (DHFR) and thymidine kinase. For example, expression can be performed in the presence of methotrexate to select for only those cells expressing the DHFR gene. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available adapted to serum-free media which facilitates purification of secreted proteins from the cell culture media. Examples include CHO-S cells (Invitrogen, Carlsbad, Calif., cat #11619-012) and the serum free EBNA-1 cell line (Pham et al. (2003) Biotechnol. Bioeng. 84:332-42). Cell lines also are available that are adapted to grow in special mediums optimized for maximal expression. For example, DG44 CHO cells are adapted to grow in suspension culture in a chemically defined, animal product-free medium.

e. Plants

Transgenic plant cells and plants can be used to express proteins such as any described herein. Expression constructs are typically transferred to plants using direct DNA transfer such as microprojectile bombardment and PEG-mediated transfer into protoplasts, and with Agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthase promoter, the ribose bisphosphate carboxylase promoter and the ubiquitin and UBQ3 promoters.

Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce matrix-degrading enzymes. Because plants have different glycosylation patterns than mammalian cells, this can influence the choice of protein produced in these hosts.

3. Purification Techniques

Methods for purification of polypeptides, including modified csMMP polypeptides, from host cells will depend on the chosen host cells and expression systems. For secreted molecules, proteins generally are purified from the culture media after removing the cells. For intracellular expression, cells can be lysed and the proteins purified from the extract. When transgenic organisms such as transgenic plants and animals are used for expression, tissues or organs can be used as starting material to make a lysed cell extract. Additionally, transgenic animal production can include the production of polypeptides in milk or eggs, which can be collected, and if necessary, the proteins can be extracted and further purified using standard methods in the art. If there are free cysteines, these can be replaced with other amino acids, such as serine. Replacement of free cysteines can prevent unwanted aggregation.

Generally, modified csMMP polypeptides are expressed and purified to be in an inactive form (zymogen form) for subsequent activation as described in the systems and methods provided herein. Hence, following expression, mature forms can be generated by the use of a processing agent and chemical modification, catalysis and/or autocatalysis to remove the inactivating propeptide. Generally, a processing agent is chosen that is acceptable for administration to a subject. If necessary, additional purification steps can be performed to remove the processing agent from the purified preparation. In addition, if necessary, additional purification steps can be performed to remove the propeptide from the purified preparation. Activation can be monitored by SDS-PAGE (e.g., a 3 kilodalton shift) and by enzyme activity (cleavage of a fluorogenic substrate). Where an active enzyme is desired, typically, an enzyme is allowed to achieve >75% activation before purification. Typically, MMPs are rendered active by activation cleavage removing the propeptide or prosegment to generate a mature enzyme from a zymogen form. In some applications, for example in the presence of low calcium concentrations, csMMPs are inactive in their mature form until exposure to the requisite calcium concentration as described herein. For example, many MMPs provided herein are not active or are substantially inactive at low calcium concentrations (e.g., 1 mM Ca²⁺).

Proteins, such as modified csMMP polypeptides, can be purified using standard protein purification techniques known in the art including, but not limited to, SDS-PAGE, size fraction and size exclusion chromatography, ammonium sulfate precipitation and ionic exchange chromatography, such as anion exchange. Affinity purification techniques also can be utilized to improve the efficiency and purity of the preparations. For example, antibodies, receptors and other molecules that bind MMPs can be used in affinity purification. Expression constructs also can be engineered to add an affinity tag to a protein such as a myc epitope, GST fusion or His₆ and affinity purified with myc antibody, glutathione resin and Ni-resin, respectively. Purity can be assessed by any method known in the art including gel electrophoresis and staining and spectrophotometric techniques.

4. Methods of Activation

MMPs require processing for activation. Generally, processing involves removal of the propeptide and/or conformational changes of the enzyme to generate a processed mature form. Processing of the enzyme by removal of the propeptide is required for activity of MMPs. For normal MMPs (e.g., wild-type) that are not conditionally active as provided herein, the processed mature form is an active enzyme. Thus, it is understood that wild-type MMPs in their processed mature form are enzymatically active, and thus for these enzymes this is the active form. csMMPs provided herein, however, also additionally require the presence of sufficient calcium to be fully active.

Processing (and thereby activation) can be induced by processing agents such as proteases, including other previously activated MMPs; by chemical activation, such as thiol-modifying agents (4-aminophenylmercuric acetate, HgCl₂ and N-ethylmaleimide), oxidized glutathione, SDS, chaotropic agents and reactive oxygens; and by low pH or heat treatment. For example, Table 8 below lists exemplary processing agents (see also Visse et al. (2003) Circ. Res. 92:827-839; Khan et al. (1998) Protein Science 7:815-836; Okada et al. (1988) Biochem. J. 254:731-741; Okada & Nakanashi (1989) FEBS Lett. 249:353-356; Nagase et al. (1990) Biochemistry 29:5783-5789; Koklitis et al. (1991) Biochem. J. 276:217-221; Springman et al. (1990) PNAS 87:364-368; Murphy et al. (1997) Matrix Biol. 15:511-518).

TABLE 8
Zymogen Activators (i.e., processing agents)
Proteolytic Compounds

| | |
|---|---|
| Proteases | Plasmin |
| | Plasma kallikrein |
| | Trypsin-1 (Trypsin I) |
| | Trypsin-2 (Trypsin II) |
| | Neutrophil elastase |
| | Cathepsin G |
| | Tryptase |
| | Chymase |
| | Proteinase-3 |
| | Furin |
| | uPA |
| | MMPs, including MMP-1, MMP-2, MMP-3, |
| | MMP-7, MMP-10, MMP-26, and MT1-MMP |

Non-Proteolytic Compounds

| | |
|---|---|
| Thiol-modifying Agents | 4-aminophenylmercuric acetate (APMA) |
| | HgCl₂ |
| | N-ethylmaleimide |
| Conformational | Sodium dodecyl sulfate (SDS) |
| Perturbants | Chaotropic agents |
| Other Chemical Agents | Oxidized glutathione (GSSG) |
| | Reactive oxygen |
| | Au(I) salts |

Other Activating Conditions

Acidic pH
Heat

MMP activation occurs in a stepwise manner. For example, activation by proteases involves a first proteolytic attack of a bait region (corresponding to amino acids 32-38 of proMMP-1 (SEQ ID NO:2)), an exposed loop region found between the first and second helices of the pro-peptide. The sequence of the bait region confers cleavage specificity. Following initial cleavage, the remaining propeptide is destabilized allowing for intermolecular processing by other partially active MMP intermediates or active MMPs. For example, the protease plasmin activates both proMMP-1 and proMMP-3. Once activated, MMP-3 effects the final activation of proMMP-1. Alternatively, activation by chemicals, for example APMA, initially causes the modification of the propeptide cysteine residue, which in turn causes partial activation and intramolecular cleavage of the propeptide. The remaining segment of propeptide is then processed by other proteases or MMPs.

### F. PHARMACEUTICAL COMPOSITIONS, DOSAGES AND FORMULATIONS

The methods provided herein utilize pharmaceutical compositions that contain csMMP polypeptides, such as any described in Section D above. Pharmaceutically acceptable compositions are prepared in view of approvals for a regulatory agency or other agency prepared in accordance with generally recognized pharmacopeia for use in animals and in humans. Typically, the compounds are formulated into pharmaceutical compositions using techniques and procedures well known in the art (see e.g., Ansel, Introduction to Pharmaceutical Dosage Forms, Fourth Edition, 1985, p. 126).

1. Compositions for Conditional Activity

The calcium-sensitive MMPs used in the methods provided herein require the presence of sufficient calcium concentrations for activity. Thus, compositions containing a modified MMP polypeptide, such as any of the csMMP polypeptides described herein in Section D, can be provided that expose the csMMP to the calcium concentrations required for activation. Exposure of the csMMP to calcium can take place in vitro or in vivo. The csMMPs can be exposed to calcium prior to, simultaneously with, subsequent to, or intermittently upon in vivo administration. For example, the modified MMP polypeptide can be administered as a composition that contains higher than physiological calcium concentrations. In other examples, a calcium-containing formulation that contains higher than physiological calcium concentrations can be administered separately from the modified MMP polypeptide, such as prior to, simultaneously with, subsequent to, or intermittently upon in vivo administration of the modified MMP polypeptide.

The calcium ions in the modified MMP compositions or calcium-containing formulations can be any calcium-containing salt that is well-tolerated upon administration to a patient. For example, the modified MMP compositions or calcium-containing formulation can include calcium chloride, calcium phosphate, calcium pyruvate, calcium gluconate, calcium lactate, calcium citrate, calcium acetate, calcium disodium versenate, or other sources of calcium ions, or combinations thereof. In some examples, a calcium salt is dissolved in a buffer or other liquid. Typically, calcium chloride is used to increase the calcium concentration in a composition containing a modified MMP or other calcium-containing formulations.

The effected duration of csMMP activation depends on the calcium concentration local to the enzyme. Thus, csMMP activation is flexible and can be adapted to the particular enzyme that is used, the disease or condition being treated, the site of administration, or other factors. For example, csMMP activity can be regulated by the concentration of calcium in the csMMP-containing formulation that is initially administered, the concentration of calcium in a formulation that is administered simultaneously with or subsequent to csMMP administration, and the frequency and concentration of calcium of additionally administered calcium-containing formulations. It is within the level of the skilled artisan to determine the amount of calcium in a MMP composition and/or the amount or frequency of administration of a calcium-containing formulation to regulate the activity of the csMMP.

For example, the modified MMP compositions or calcium-containing formulations can contain calcium in a concentration that is greater than physiological calcium levels (e.g., 1-1.3 mM). In particular, the concentration of calcium in the compositions or formulations is greater than 1.5 mM and generally between or about between 2 mM to 100 mM, and typically 5 mM to 20 mM. For example, the concentration of calcium is at least or about at least 2 mM, 3 mM, 4 mM, 5 mM, 6 mM, 7 mM, 8 mM, 9 mM, 10 mM, 15 mM, 20 mM, 25 mM, 50 mM, 100 mM or greater than 100 mM. The calcium-containing formulation, for example, a buffer or liquid, can be provided in the same composition as the csMMP or in a separate composition. When provided separately, it can be administered prior to, simultaneously with, subsequent to, or intermittently with the csMMP.

Hence, where the csMMP requires calcium concentrations that are greater than physiological calcium levels (e.g., 1-1.3 mM), the csMMP is provided in and/or exposed to a calcium-containing formulation that, upon administration of the modified MMP, results in an extracellular calcium concentration at the local site of administration of at least or about at least 2 mM, 3 mM, 4 mM, 5 mM, 6 mM, 7 mM, 8 mM, 9 mM, 10 mM, 15 mM, 20 mM, 25 mM, 50 mM, 100 mM or greater than 100 mM. Upon administration in vivo where the physiologic extracellular Ca²⁺ concentration is at or about 1-1.3 mM Ca²⁺, the Ca²⁺ concentration will decrease in the local environment of the csMMP as the Ca²⁺ ions dissipate, thereby resulting in inactivation, or substantial inactivation, of the csMMP and temporal control thereof (which could occur immediately or almost immediately, depending on the starting calcium concentration of the composition administered). One of skill in the art can empirically determine the length of time required for application depending on the particular target depth of the tissue that is being treated, the particular enzyme that is being used, and other factors based on known testing protocols or extrapolation from in vivo or in vitro test data. In addition, one of skill in the art can empirically determine the amount of Ca²⁺ to administer to achieve csMMP activation for a predetermined length of time.

The csMMP polypeptides for use in the methods provided herein also can be prepared in compositions containing other requisite metals required for activity. For example, MMP polypeptides are Zn-dependent in addition to being Ca-dependent. It is within the level of one of skill in the art to empirically determine the optimal concentration of zinc and calcium required for activity. The optimal concentration of zinc and calcium is a concentration that maintains the calcium-sensitive phenotype of the csMMP. For example, the optimal concentration of ZnCl₂ in the MMP compositions provided herein is typically less than 0.01 mM, for example, 0.0005 mM to 0.009 mM, and in particular 0.0005 mM to 0.005 mM, for example 0.001 mM. Other metals also can be included in the compositions as required for activity.

The compositions and formulations generally also contain salt (e.g., NaCl), which can provide stabilizing effects on the enzyme. The pharmaceutical compositions or formulations provided herein are prepared in accordance with the requirements of the active agent(s). It is within the level of one of skill in the art to assess the stability of the active agent(s) in the formulation. In particular examples herein, the pharmaceutical compositions contain NaCl at a concentration of between or about between 10 mM to 200 mM, such as 10 mM to 50 mM, 50 mM to 200 mM, 50 mM to 120 mM, 50 mM to 100 mM, 50 mM to 90 mM, 120 mM to 160 mM, 130 mM to 150 mM, 80 mM to 140 mM, 80 mM to 120 mM, 80 mM to 100 mM, 80 mM to 160 mM, 100 mM to 140 mM, 120 mM to 120 mM or 140 mM to 180 mM.

The pharmaceutical compositions also are prepared at a pH of between or about between 6.0 to 8.0, such as between or about between 6.5 to 7.8, 6.5 to 7.2, 7.0 to 7.8, 7.0 to 7.6 or 7.2 to 7.4, and generally at a pH of about 7.5. Reference to pH herein is based on measurement of pH at room temperature. It is understood that the pH can change based on temperature, exposure to other components and other conditions. For example, the pH can vary by ±0.1, 0.2, 0.3, 0.4, 0.5, 0.6, 0.7, 0.8, 0.9, 1.0, 1.2, 1.3, 1.4, 1.5 or more. If necessary, pH can be adjusted using acidifying agents to lower the pH or alkalizing agents to increase the pH. Exemplary acidifying agents include, but are not limited to, acetic acid, citric acid, sulfuric acid, hydrochloric acid, monobasic sodium phosphate solution, and phosphoric acid. Exemplary alkalizing agents include, but are not limited to, dibasic sodium phosphate solution, sodium carbonate, or sodium hydroxide. The compositions are generally prepared using a buffering agent that maintains the pH range.

The compositions are generally prepared using a buffering agent that does not interfere or interact with the calcium ions. For example, generally phosphate buffers are not utilized, since calcium precipitates as calcium phosphate in phosphate buffers. In addition, citrate is a known calcium chelator and thus citrate buffers generally are not utilized as buffers in compositions herein. Examples of particularly suitable buffers include Tris, succinate, acetate, aconitate, malate and carbonate. Those of skill in the art, however, will recognize that formulations provided herein are not limited to a particular buffer, so long as the buffer maintains the calcium concentration and provides an acceptable degree of pH stability, or “buffer capacity” in the range indicated. Generally, a buffer has an adequate buffer capacity within about 1 pH unit of its pK (Lachman et al. in: The Theory and Practice of Industrial Pharmacy, 3rd Edn. (Lachman, L., Lieberman, HA. and Kanig, J. L., Eds.), Lea and Febiger, Philadelphia, p. 458-460, 1986). Buffer suitability can be estimated based on published pK tabulations or can be determined empirically by methods well known in the art. The pH of the solution can be adjusted to the desired endpoint using any acceptable acid or base.

Buffers that can be included in the co-formulations provided herein include, but are not limited to, Tris (Tromethamine) or histidine buffers. Such buffering agents can be present in the compositions or formulations at concentrations between or about between 1 mM to 100 mM, such as at least or about at least 10 mM to 50 mM or 20 mM to 40 mM, such as at or about or at least 50 mM. For example, such buffering agents can be present in the compositions or formulations in a concentration of at least or about at least 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, or more.

An exemplary composition for use in the methods herein contains a therapeutically effective amount of a modified MMP (e.g., any described in Section D); a calcium concentration to render the enzyme conditionally active upon administration (e.g., greater than physiological levels of calcium as described above, such as at least or about 5 mM or 10 mM); a buffer (e.g., Tris) at a concentration of about or at least 50 mM; a salt (e.g., NaCl) at a concentration of about or at least 150 mM; and is prepared at a pH of about or at least 7.5.

The composition containing the csMMP polypeptide can include a pharmaceutically acceptable carrier. Pharmaceutical compositions can include carriers such as a diluent, adjuvant, excipient, or vehicle with which an enzyme is administered. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent, such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents.

2. Formulations

The compositions used in the methods provided herein can be formulated into suitable pharmaceutical preparations such as solutions, suspensions, tablets, dispersible tablets, pills, capsules, powders, sustained release formulations, or elixirs, for oral administration, as well as transdermal patch preparation and dry powder inhalers. A composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, and other such agents. The formulation should suit the mode of administration. Typically, the compounds are formulated into pharmaceutical compositions using techniques and procedures well known in the art (see e.g., Ansel, Introduction to Pharmaceutical Dosage Forms, Fourth Edition, 1985, 126).

Formulations are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof.

Compositions can be formulated for administration by any route known to those of skill in the art including intramuscular, intravenous, intradermal, intralesional, intraperitoneal injection, subcutaneous, epidural, nasal, oral, vaginal, rectal, topical, local, otic, inhalational, buccal (e.g., sublingual), and transdermal administration or any route. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Topical applications of formulations can be facilitated by methods such as iontophoresis or sonophoresis. Compositions also can be administered with other biologically active agents, either sequentially, intermittently or in the same composition. Administration also can include controlled release systems including controlled release formulations and device controlled release, such as by means of a pump.

The most suitable route in any given case depends on a variety of factors, such as the nature of the disease, the progress of the disease, the severity of the disease the particular composition which is used. For purposes herein, it is desired that csMMP polypeptides are administered so that they reach the interstitium of skin or tissues. Thus, direct administration under the skin, such as by sub-epidermal administration methods, is contemplated. These include, for example, subcutaneous, intradermal and intramuscular routes of administration. Thus, in one example, local administration can be achieved by injection, such as from a syringe or other article of manufacture containing an injection device such as a needle. Other modes of administration also are contemplated. Pharmaceutical compositions can be formulated in dosage forms appropriate for each route of administration. Typically, the compositions herein are formulated for parenteral administration, generally characterized by injection, either subcutaneously, intramuscularly or intradermally, or for topical administration. Injectable and topically administrable formulations are described below.

In one example, the pharmaceutical preparation can be in liquid form, for example, solutions, syrups or suspensions. If provided in liquid form, the pharmaceutical preparation of a csMMP can be provided as a concentrated preparation to be diluted to a therapeutically effective concentration with a concentrated calcium-containing buffer such that the final calcium concentration is sufficient to render the csMMP active. A calcium-containing buffer can be added to the csMMP preparation prior to administration, or a calcium-containing buffer can be administered simultaneously, intermittently or sequentially with the csMMP preparation. Liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

In another example, pharmaceutical preparations can be presented in lyophilized form for reconstitution with water, calcium-containing buffer or other suitable vehicle before use. For example, the pharmaceutical preparations of csMMPs can be reconstituted with a solution containing activating concentrations of calcium. Alternatively, once reconstituted, the preparation can be mixed with a solution or buffer containing calcium in order to achieve a desired calcium concentration so as to render the csMMP active prior to use. For example, the final, reconstituted liquid preparation can contain a calcium concentration of 2 mM to 100 mM.

a. Injectables, Solutions and Emulsions

Parenteral administration, generally characterized by injection, either subcutaneously, intramuscularly or intradermally is contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. The pharmaceutical compositions also may contain other minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see e.g., U.S. Pat. No. 3,710,795) also is contemplated herein. The percentage of active compound contained in such parenteral compositions is highly dependent on the specific nature thereof, as well as the activity of the compound and the needs of the subject.

Parenteral administration of the compositions generally includes sub-epidermal routes of administration such as intradermal, subcutaneous and intramuscular administrations. If desired, intravenous administration also is contemplated. Injectables are designed for local and systemic administration. For purposes herein, local administration is desired for direct administration to the affected interstitium. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include polysorbate 80 (TWEEN 80). Sequestering or chelating agents of metal ions include ethylenediaminetetraacetic acid (EDTA) and ethylene glycol tetraacetic acid (EGTA). Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art. The unit-dose parenteral preparations are packaged in an ampoule, a vial or a syringe with a needle. The volume of liquid solution or reconstituted powder preparation, containing the pharmaceutically active compound, is a function of the disease to be treated and the particular article of manufacture chosen for package. For example, for the treatment of cellulite, it is contemplated that for parenteral injection the injected volume is or is about 10 to 50 milliliters. All preparations for parenteral administration must be sterile, as is known and practiced in the art.

Lyophilized Powders

Of interest herein are lyophilized powders, which can be reconstituted for administration as solutions, emulsions and other mixtures. They can also be reconstituted and formulated as solids or gels. A sterile, lyophilized powder is prepared by dissolving a compound of inactive enzyme in a buffer solution. The buffer solution may contain an excipient which improves the stability or other pharmacological component of the powder or reconstituted solution, prepared from the powder. Subsequent sterile filtration of the solution followed by lyophilization under standard conditions known to those of skill in the art provides the desired formulation. Briefly, the lyophilized powder is prepared by dissolving an excipient, such as dextrose, sorbitol, fructose, corn syrup, xylitol, glycerin, glucose, sucrose or other suitable agent, in a suitable buffer, such as citrate, sodium or potassium phosphate or other such buffer known to those of skill in the art. Then, a selected enzyme is added to the resulting mixture, and stirred until it dissolves. The resulting mixture is sterile filtered or treated to remove particulates and to insure sterility, and apportioned into vials for lyophilization. Each prepared vial will contain a single dosage (1 mg to 1 g, generally 1 to 100 mg, such as 1 to 5 mg) or multiple dosages of the compound. The lyophilized powder can be stored under appropriate conditions, such as at about 4° C. to room temperature.

Reconstitution of this lyophilized powder with a buffer solution provides a formulation for use in parenteral administration. The solution chosen for reconstitution can be any buffer. For reconstitution about 1 μg-20 mg, preferably 10 μg-1 mg, more preferably about 100 μg is added per mL of buffer or other suitable carrier. The precise amount depends upon the indication treated and selected compound. Such amount can be empirically determined.

b. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see e.g., U.S. Pat. Nos. 4,044,126; 4,414,209; and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically diameters of less than 50 microns, preferably less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients also can be administered.

Formulations suitable for transdermal administration are provided. They can be provided in any suitable format, such as discrete patches adapted to remain in intimate contact with the epidermis of the recipient for a prolonged period of time. Such patches contain the active compound in optionally buffered aqueous solution of, for example, 0.1 to 0.2 M concentration with respect to the active compound. Formulations suitable for transdermal administration also can be delivered by iontophoresis (see, e.g., Tyle, P. (1986) Pharm. Res. 3(6):318-326) or sonophoresis (Soroha et al. (2011) Int. Curr. Pharm. Res. 3(3):89-97) and typically take the form of an optionally buffered aqueous solution of the active compound.

c. Compositions for Other Routes of Administration

Depending upon the condition treated other routes of administration, such as topical application, transdermal patches, oral and rectal administration are also contemplated herein. For example, pharmaceutical dosage forms for rectal administration are rectal suppositories, capsules and tablets for systemic effect. Rectal suppositories include solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 g. Tablets and capsules for rectal administration are manufactured using the same pharmaceutically acceptable substance and by the same methods as for formulations for oral administration. Formulations suitable for rectal administration can be provided as unit dose suppositories. These can be prepared by admixing the active compound with one or more conventional solid carriers, for example, cocoa butter, and then shaping the resulting mixture.

For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions also can be administered by controlled release formulations and/or delivery devices (see e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533; and 5,733,566).

Various delivery systems are known and can be used to administer selected csMMPs, such as but not limited to, encapsulation in liposomes, microparticles, microcapsules, recombinant cells capable of expressing the compound, receptor mediated endocytosis, and delivery of nucleic acid molecules encoding selected matrix-degrading enzymes such as retrovirus delivery systems.

Hence, in certain embodiments, liposomes and/or nanoparticles also can be employed with administration of matrix-degrading enzymes. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 angstroms containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can demonstrate low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use herein, and such particles can be easily made.

3. Dosages and Administration

Pharmaceutical compositions containing a modified MMP, such as any described herein, are typically formulated and administered in a therapeutically effective amount. In particular, a modified MMP, such as any selected csMMP polypeptide described herein, is administered in an amount sufficient that when activated to a mature form and exposed to high calcium concentrations (e.g., 10 mM Ca²⁺), exerts a therapeutically useful effect in the absence of undesirable side effects on the subject receiving treatment. Therapeutically effective concentrations can be determined empirically by testing the compounds in known in vitro and in vivo systems, such as the assays provided herein. The concentration of a selected csMMP polypeptide in the composition depends on absorption, inactivation and excretion rates of the complex, the physicochemical characteristics of the complex, the dosage schedule, and amount administered as well as other factors known to those of skill in the art. For example, it is understood that the precise dosage and duration of treatment is a function of the tissue being treated and may be determined empirically using known testing protocols or by extrapolation from in vivo or in vitro test data. It is to be noted that concentrations and dosage values may also vary with the age or gender of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope thereof.

The amount of a selected csMMP polypeptide to be administered for the treatment of an ECM-mediated disease or condition, for example a collagen-mediated disease or condition, such as cellulite or lymphedema, can be determined by standard clinical techniques. In addition, in vitro assays and animal models can be employed to help identify optimal dosage ranges. The precise dosage, which can be determined empirically, can depend on the particular enzyme, the route of administration, the type of disease to be treated and the seriousness of the disease. Dosage levels also can be determined based on a variety of factors, such as body weight of the individual, general health, age, the activity of the specific compound employed, sex, diet, time of administration, rate of excretion, drug combination, the severity and course of the disease, and the patient's disposition to the disease and the judgment of the treating physician. The amount of active ingredient that can be combined with the carrier materials to produce a single dosage form will vary depending upon the particular matrix-degrading enzyme, the host treated, the particular mode of administration, and the calcium concentration required for activation, and/or the predetermined or length of time in which activation is desired.

Exemplary dosages range from or about 10 μg to 100 mg, particularly 50 μg to 75 mg, 100 μg to 50 mg, 250 μg to 25 mg, 500 μg to 10 mg, 1 mg to 5 mg, or 2 mg to 4 mg. The particular dosage and formulation thereof depends upon the indication and individual. If necessary, dosage can be empirically determined. Typically the dosage is administered for indications described herein in a volume of 1-100 mL, particularly, 1-50 mL, 10-50 mL, 10-30 mL, 1-20 mL, or 1-10 mL volumes following reconstitution, for example, in a buffer containing high concentrations of calcium greater than physiological levels of calcium (e.g., 2 mM to 100 mM, such as 5 mM to 50 mM, and generally at least or about at least 10 mM Ca²⁺) so as to render the csMMP active. Typically, such dosages are from at or about 100 μg to 50 mg, generally 1 mg to 5 mg, in a final volume of 10 to 50 mL. The pharmaceutical compositions typically should provide a dosage of from about 1 μg/mL to about 20 mg/mL. Generally, dosages are from or about 10 μg/mL to 1 mg/mL, typically about 100 μg/mL, per single dosage administration.

A csMMP can be formulated in compositions to be administered at once, or can be divided into a number of smaller doses to be administered at intervals of time. Selected csMMP polypeptides can be administered in one or more doses over the course of a treatment time for example over several hours, days, weeks, or months. In some cases, continuous administration is useful. It is understood that the precise dosage and course of administration depends on the methods of calcium-dependent activation contemplated.

Pharmaceutically therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging. Generally, dosage forms or compositions containing active ingredient in the range of 0.005% to 100% with the balance made up from non-toxic carrier can be prepared.

Also, it is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them. The compositions can be administered hourly, daily, weekly, monthly, yearly or once. Generally, dosage regimens are chosen to limit toxicity. It should be noted that the attending physician would know how to and when to terminate, interrupt or adjust therapy to lower dosage due to toxicity, or bone marrow, liver or kidney or other tissue dysfunctions. Conversely, the attending physician would also know how to and when to adjust treatment to higher levels if the clinical response is not adequate (provided toxic side effects do not preclude a higher dose).

Administration methods can be employed to decrease the exposure of modified MMP polypeptides to degradative processes, such as proteolytic degradation and immunological intervention via antigenic and immunogenic responses. Examples of such methods include local administration at the site of treatment. PEGylation of therapeutics has been reported to increase resistance to proteolysis, increase plasma half-life, and decrease antigenicity and immunogenicity. Examples of PEGylation methodologies are known in the art (see for example, Lu and Felix (1994) Int. J. Peptide Protein Res. 43:127-138; Lu and Felix (1993) Peptide Res. 6:140-146; Felix et al. (1995) Int. J. Peptide Res. 46:253-264; Benhar et al. (1994) J. Biol. Chem. 269:13398-13404; Brumeanu et al. (1995) J. Immunol. 154:3088-3095; see also, Caliceti et al. (2003) Adv. Drug Deliv. Rev. 55(10):1261-1277 and Molineux (2003) Pharmacotherapy 23 (8 Pt 2):3S-8S). PEGylation also can be used in the delivery of nucleic acid molecules in vivo. For example, PEGylation of adenovirus can increase stability and gene transfer (see e.g., Cheng et al. (2003) Pharm. Res. 20(9):1444-1451).

### G. METHODS OF ASSESSING csMMP ACTIVITY

Modified MMPs, including csMMPs, can be tested for their enzymatic activity against known substrates. Activity assessment can be performed at varying calcium concentrations or at varying concentrations combined with variations in other parameters, such as varying temperatures. Activity assessments can be performed on conditioned medium or other supernatants or on purified protein.

1. Methods of Assessing Enzymatic Activity

Enzymatic activity can be assessed by assaying for substrate cleavage using known substrates of the enzyme. The substrates can be in the form of a purified protein or provided as peptide substrates. For example, enzymatic activity of a MMP can be assessed by cleavage of collagen. Cleavage of a purified protein by an enzyme can be assessed using any method of protein detection, including, but not limited to, HPLC, SDS-PAGE analysis, ELISA, Western blotting, immunohistochemistry, immunoprecipitation, N-terminal sequencing, protein labeling and fluorometric methods. For example, Example 6 describes an assay to assess enzymatic activity for cleavage of a collagen that is FITC-labeled. Fluorescence of the supernatant is an indication of the enzymatic activity of the protein and can be normalized to protein concentration and a standard curve for specific activity assessment.

In addition, enzymatic activity can be assessed on tetrapeptide substrates. The use of fluorogenic groups on the substrates facilitates detection of cleavage. For example, substrates can be provided as fluorogenically tagged tetrapeptides of the peptide substrate, such as an ACC- or 7-amino-4-methylcoumarin (AMC)-tetrapeptide. Other fluorogenic groups are known and can be used and coupled to protein or peptide substrates. These include, for example, 7-amino-4-methyl-2-quinolinone (AMeq), 2-naphthylamine (NHNap) and 7 amino-4-methylcoumarin (NHMec) (Sarath et al., “Protease Assay Methods,” in Proteolytic Enzymes: A Practical Approach., Ed. Robert J. Beynon and Judith S. Bond; Oxford University Press, 2001. pp. 45-76). Peptide substrates are known to one of skill in the art, as are exemplary fluorogenic peptide substrates. For example, exemplary substrates for MMP include peptide IX, designated as Mca-K-P-L-G-L-Dpa-A-R-NH₂ (SEQ ID NO:88; Mca=(7-methoxycoumarin-4-yl)acetyl; Dpa=N-3-(2,4,-dinitrophenyl)-L-2,3-diaminopropionyl; R&D Systems, Minneapolis, Minn., Cat#ES010) and variations thereof such as with different fluorogenic groups. Enzyme assays to measure enzymatic activity by fluorescence intensity are standard and are typically performed as a function of incubation time of the enzyme and substrate (see e.g., Dehrmann et al. (1995) Arch. Biochem. Biophys. 324:93-98; Barrett et al. (1981) Methods Enzymol. 80:536-561). Exemplary assays using fluorescence substrates are described in Example 2.C herein.

While detection of fluorogenic compounds can be accomplished using a fluorometer, detection can be accomplished by a variety of other methods well known to those of skill in the art. Thus, for example, when the fluorophores emit in the visible wavelengths, detection can be simply by visual inspection of fluorescence in response to excitation by a light source. Detection also can be by means of an image analysis system utilizing a video camera interfaced to a digitizer or other image acquisition system. Detection also can be by visualization through a filter, as under a fluorescence microscope. The microscope can provide a signal that is simply visualized by the operator. Alternatively, the signal can be recorded on photographic film or using a video analysis system. The signal also can simply be quantified in real-time using either an image analysis system or a photometer.

Thus, for example, a basic assay for enzyme activity of a sample involves suspending or dissolving the sample in a buffer (at the pH optima of the particular protease being assayed) adding to the buffer a fluorogenic enzyme peptide indicator, and monitoring the resulting change in fluorescence using a spectrofluorometer as shown in e.g., Harris et al., (1998) J. Biol. Chem. 273(42):27364-27373. The spectrofluorometer is set to excite the fluorophore at the excitation wavelength of the fluorophore. The fluorogenic enzyme indicator is a substrate sequence of an enzyme (e.g., of a protease) that changes in fluorescence due to a protease cleaving the indicator.

2. Methods of Assessing Degradation of ECM Component

The degradation of extracellular matrix proteins by modified csMMPs including, but not limited to, those described above, such as csMMP-1, can be assessed in vitro or in vivo. Assays for such assessment are known to those of skill in the art, and can be used to test the activities of a variety of csMMPs on a variety of extracellular matrix proteins, including, but not limited to collagen (I, II, III and IV), fibronectin, vitronectin and proteoglycans. Assays can be performed at high (e.g., 10 mM) and low (e.g., 1-1.3 mM) concentrations of Ca²⁺. Experiments also can be performed in the presence of a MMP that is not modified to be calcium sensitive. It is understood that assays for enzymatic activity are performed subsequent to activation of the enzyme by a processing agent. As a further control, activity of the zymogen enzyme also can be assessed.

a. In Vitro Assays

Exemplary in vitro assays include assays to assess the degradation products of extracellular matrix proteins following incubation with a csMMP. In some examples, the assays detect a single, specific degradation product. In other examples, the assays detect multiple degradation products, the identity of which may or may not be known. Assessment of degradation products can be performed using methods well known in the art including, but not limited to, HPLC, CE, Mass spectrometry, SDS-PAGE analysis, ELISA, Western blotting, immunohistochemistry, immunoprecipitation, N-terminal sequencing, and protein labeling. Extracellular matrix degradation products can be visualized, for example, by SDS-PAGE analysis following incubation with a csMMPs for an appropriate amount of time in the presence of the appropriate concentration of Ca²⁺. For example, collagen can be incubated with a mature csMMP and subjected to SDS-PAGE using, for example, a 4-20% Tris/glycine gel to separate the products. Coomassie staining of the gel facilitates visualization of smaller degradation products, or disappearance of collagen bands, compared to intact collagen. Immunoblotting using, for example, a polyclonal Ig specific to the extracellular matrix protein also can be used to visualize the degradation products following separation with SDS-PAGE.

Assays that specifically detect a single product following degradation of an extracellular matrix protein also are known in the art and can be used to assess the ability of a csMMP to degrade an extracellular matrix protein. For example, the hydroxyproline (HP) assay can be used to measure degradation of collagen. 4-hydroxyproline is a modified imino acid that makes up approximately 12% of the weight of collagen. HP assays measure the amount of solubilized collagen by determining the amount of HP in the supernatant following incubation with a matrix-degrading enzyme (see e.g., Reddy and Enwemeka (1996) Clin. Biochem. 29:225-229). Measurement of HP can be effected by, for example, colorimetric methods, high performance liquid chromatography, mass spectrometry and enzymatic methods (see e.g., Edwards et al. (1980) Clin. Chim. Acta 104:161-167; Green (1992) Anal. Biochem. 201:265-269; Tredget et al. (1990) Anal. Biochem. 190:259-265; Ito et al. (1985) Anal. Biochem. 151:510-514; Garnero et al. (1998) J Biol. Chem. 273:32347-32352).

The collagen source used in such in vitro assays can include, but is not limited to, commercially available purified collagen, bone particles, skin, cartilage and rat tail tendon. Collagenolytic activity of a csMMP, such as csMMP-1, can be assessed by incubating the activated enzyme with an insoluble collagen suspension, followed by hydrolysis, such as with HCl. The amount of hydroxyproline derived from the solubilized (degraded) collagen can be determined by spectrophotometric methods, such as measuring the absorbance at 550 nm following incubation with Ehrlich's reagent. In some examples, the collagen source is rat or pig skin explant that is surgically removed from anesthetized animals and then perfused with the csMMP, for example, csMMP-1, prior to, subsequent to, simultaneously with or intermittently with a calcium-containing formulation. HP levels in the perfusates can then be assessed. In a modification of this method, the effect on the fibrous septae in the explants also can be assessed. Briefly, following perfusion with the enzyme, the explants are cut into small pieces and embedded in paraffin and analyzed by microscopy following Masson's Trichrome staining for visualization of collagen. The number of collagen fibrous septae can be visualized and compared to tissue that has not been treated with an enzyme.

Assays to detect degradation of specific collagens also are known in the art. Such assays can employ immunological methods to detect a degradation product unique to the specific collagen. For example, the degradation of collagen I by some MMPs releases telopeptides with different epitopes that can be detected using immunoassays. Such assays detect the cross-linked N-telopeptides (NTx) and/or the cross-linked C-telopeptides (CTx and ICTP), each of which contain unique epitopes. Typically, CTx assays utilize the CrossLaps (Nordic Biosciences) antibodies that recognize the 8-amino acid sequence EKAHD-β-GGR octapeptide (SEQ ID NO:77), where the aspartic acid is in β-isomerized configuration, in the C-terminal telopeptide region of the α1 chain (Eastell (2001) Bone Markers: Biochemical and Clinical Perspectives, pg 40). Immunoassays to detect ICTP also are known in the art and can be used to detect degradation of collagen I (U.S. Pat. No. 5,538,853). In other examples, immunoassays, such as, for example, ELISAs, can be used to detect NTx following incubation of collagen type I with proteases such as a MMP (Atley et al. (2000) Bone 26:241-247). Other antibodies and assays specific for degraded collagens are known in the art and can be used to detect degradation by matrix-degrading enzymes. These include antibodies and assays specific for degraded collagen I (Hartmann et al. (1990) Clin. Chem. 36:421-426), collagen II (Hollander et al. (1994) J. Clin. Invest. 93:1722-1732), collagen III (U.S. Pat. No. 5,342,756), and collagen IV (Wilkinson et al. (1990) Anal. Biochem. 185:294-296).

b. In Vivo Assays

Assays to detect the in vivo degradation of ECM also are known in the art. Such assays can utilize the methods described above to detect, for example, hydroxyproline and N- and C-telopeptides and degraded collagens or other ECM in biological samples such as urine, blood, serum and tissue. Detection of degraded ECM can be performed following administration to the patient of one or more enzymes. Detection of pyridinoline (PYD) and deoxypyridinoline (DPYD) also can be used to assess degradation of collagen. Also known as hydroxylysylpyridinoline and lysylpyridinoline, respectively, PYD and DPYD are the two nonreducible trivalent cross-links that stabilize type I collagen chains and are released during the degradation of mature collagen fibrils. Pyridinoline is abundant in bone and cartilage, whereas deoxypyridinoline is largely confined to bone. Type III collagen also contains pyridinoline cross-links at the amino terminus. Total PYD and DPYD can be measured, for example, in hydrolyzed urine samples or serum by fluorometric detection after reverse-phase HPLC (Hata et al. (1995) Clin. Chimica. Acta. 235:221-227).

c. Non-Human Animal Models

Non-human animal models can be used to assess the activity of matrix-degrading enzymes. For example, non-human animals can be used as models for a disease or condition. Non-human animals can be injected with disease and/or phenotype-inducing substances prior to administration of enzymes. Genetic models also are useful. Animals, such as mice, can be generated which mimic a disease or condition by the overexpression, underexpression or knock-out of one or more genes. For example, animal models are known in the art for conditions including, but not limited to, Peyronie's Disease (Davila et al. (2004) Biol. Reprod 71:1568-1577), tendinosis (Warden et al. (2006) Br. J. Sports Med. 41:232-240) and scleroderma (Yamamoto (2005) Cur. Rheum. Rev. 1:105-109).

Non-human animals also can be used to test the activity of enzymes in vivo in a non-diseased animal. For example, enzymes can be administered to non-human animals, such as, a mouse, rat or pig, and the level of ECM degradation can be determined. In some examples, the animals are used to obtain explants for ex vivo assessment of ECM degradation. In other examples, ECM degradation is assessed in vivo. For example, collagen degradation of the skin of anesthetized animals can be assessed. Briefly, a csMMP, such as a csMMP-1, is perfused prior to, simultaneously, subsequently or intermittently with administration of calcium-containing formulation via insertion of a needle into the dermal layer of the skin of the tail. Perfusate fractions are collected from the tail skin and analyzed for collagen degradation by hydroxyproline analysis. Other methods can be used to detect degradation including, but not limited to, any of the assays described above, such as immunoassays to detect specific degradation products.

### H. METHODS OF CONDITIONAL ACTIVATION FOR TREATING DISEASES OR DEFECTS OF THE ECM

The methods provided herein use modified MMP polypeptides, such as any of the csMMPs described above, for treatment of any fibrotic disease or condition mediated by any one or more ECM components, particularly collagen-mediated conditions. In particular examples herein, modified MMP-1 polypeptides or catalytically active fragments thereof are employed in methods of treatment of fibrotic diseases or condition. Excessive deposition of ECM components is the predominant characteristic of many fibrotic diseases. The range of affected organs and tissues is large and includes a host of diseases that include, but are not limited to, pulmonary diseases, liver diseases, kidney fibrosis, localized scleroderma, Peyronie's Disease, Dupuytren's contracture, hypertrophic scarring, keloids, frozen shoulder, lipoma, cellulite, scarred tendons, glaucoma, herniated intervertebral discs and others. Fibrotic diseases and conditions are principally caused by the production or overproduction of collagens and other extracellular matrix proteins, which can occur due to tissue damage or other injury. The production or overproduction of collagen or other ECM components can be caused by TGF-β signaling in fibroblasts associated with the tissue fibrosis. Concomitant with the increase in ECM components is the decrease in the synthesis of MMPs and the increase in their inhibitors (e.g., TIMPS), which is a contributing factor for the establishment and progression of fibrosis.

This section provides exemplary methods, uses and applications for the csMMPs that are modified MMPs that exhibit calcium-regulated activity. These methods of therapy described herein are exemplary and do not limit the applications of the methods provided. Such methods include, but are not limited to, methods of treatment of any fibrotic disease or condition that is caused by excess, aberrant or accumulated expression of any one or more ECM component. Exemplary of diseases or conditions to be treated are any mediated by collagen, elastin, fibronectin, or a glycosaminoglycan such as a proteoglycan. In particular, the diseases and conditions are those that involve or are associated with aberrant or accumulated production of collagen type I or collagen type II. Exemplary of such fibrotic diseases or conditions that are collagen-mediated diseases or disorders include, but are not limited to, cellulite, Dupuytren's disease (also called Dupuytren's contracture), Peyronie's disease, frozen shoulder, chronic tendinosis or scar tissue of the tendons, localized scleroderma, lipoma, lymphedema, keloids, hypertrophic scars. Also included are methods of using the modified MMPs in uses and applications for the treatment of glaucoma or herniated invertebral discs or as an adjunct to vitrectomy. It is within the skill of a treating physician to identify such diseases or conditions.

1. Selecting Modified MMP

The particular disease or condition to be treated dictates the enzyme that is selected. For example, treatment of a collagen-mediated disease or disorder can be effected by administration of a csMMP that cleaves collagen. In particular, the modified MMP is generally selected that has a range of substrate specificity that matches the particular disease or condition. For example, generally collagen-mediated diseases or the condition of the ECM are mediated or associated with type I or type III collagen, and hence a MMP is selected that cleaves type I and/or type III collagen. For example, if the disease or condition is mediated or associated with a type I collagen, a modified MMP is selected that is a modified MMP-1, MMP-2, MMP-7, MMP-8, MMP-12, MMP-13, MMP-14 and MMP-18, or catalytically active fragment thereof. If the disease or condition is mediated or associated with a type III collagen, a modified MMP is selected that is a modified MMP-1, MMP-2, MMP-3, MMP-8, MMP-10, MMP-13, MMP-14 and MMP-16. In other cases, if the disease or condition is mediated or associated with a type I collagen and a type III collagen, a modified MMP is selected that is a MMP-1, MMP-2, MMP-8, MMP-13 and MMP-14. In particular examples herein, the modified MMP for use in the methods and treatments herein is a modified MMP-1 or catalytically active fragment thereof. In particular, any of the modified MMP polypeptides, such as modified MMP-1 polypeptides, described herein above can be used. Particular csMMPs, and systems and methods for activation, can be chosen accordingly to treat a particular disease or condition.

Treatment of diseases and conditions with csMMPs can be effected by any suitable route of administration using suitable formulations as described herein including, but not limited to, subcutaneous injection, intramuscular, intradermal, oral, and topical and transdermal administration. As described above, a route of administration of csMMPs typically is chosen that results in administration to the ECM directly to the affected site, such as administration under the skin. Exemplary of such routes of administration include, but are not limited to, subcutaneous, intramuscular, or intradermal injection.

If necessary, a particular dosage and duration and treatment protocol can be empirically determined or extrapolated. For example, exemplary doses of csMMPs, such as any described above, can be used as a starting point to determine appropriate dosages. It is understood that the amount to administer will be a function of the csMMP and the calcium concentration to be administered, the indication treated, and possibly side effects that will be tolerated. Dosages can be empirically determined using recognized models for each disorder. Also, as described elsewhere herein, csMMPs, can be administered in combination with other agents sequentially, simultaneously or intermittently. Exemplary of such agents include, but are not limited to, lidocaine, epinephrine, a dispersing agent such as hyaluronidase and combinations thereof.

Upon improvement of a patient's condition, a maintenance dose of an activating formulation, additional compound for treatment, or additional or different csMMP compositions can be administered if necessary; and the dosage, the dosage form, or frequency of administration, or a combination thereof, can be modified. In some cases, a subject can require intermittent treatment on a long-term basis upon any recurrence of disease symptoms.

2. Methods of Conditional Activation

Bacterial collagenases have been used in the treatment of fibrotic diseases and conditions (see e.g., U.S. Pat. Nos. RE39,941; 5,589,171; 6,086,872; 7,811,560; 6,074,664; and 7,641,900; and U.S. Published Application Nos. US 2007/0224184; US 2003/0170225; US 2006/0222639; and US 2008/0145357). MMP-1 also has been used for the treatment of fibrotic diseases or conditions (Limuro et al. (2003) Gastroenterol. 124:445-458; Bedair et al. (2007) J. Appl. Physiol. 102:2338-2345); Kaar et a (2008) Acta Biomaterialia 4:1411-1420). Such methods have shown that the use of MMPs can decrease fibrosis by degrading or cleaving a collagen component, such as collagen (e.g., type I or type III). Existing methods, however, are limited by the prolonged activation of the enzyme in vivo. For example, diffusion of the administered enzyme at the site of administration would result in degradation of the ECM component in unwanted areas that are peripheral to the desired treatment area.

In contrast to these methods, the instant methods are directed to methods of treatment using conditionally activated MMP polypeptides, including conditionally active MMP-1 polypeptides, that are regulated through the use of calcium. Hence, through the use of high concentrations of calcium greater than physiological concentrations, the modified MMP can be used to temporally or conditionally cleave a component of the ECM (e.g., collagen, such as type I or type III collagen) to thereby treat the fibrotic disease or condition. In some examples of the methods herein, conditional activation of the administered MMP polypeptide also can be achieved by temperature regulation. U.S. Published Application No. 2010/0284995 describes temperature sensitive mutants of MMPS that exhibit reduced activity at the temperature of the physiological environment (e.g., 37° C.) and greater activity at lower temperatures (e.g., 25° C.), and the use thereof for treatment of fibrotic diseases and conditions, including collagen-mediated diseases and conditions. Hence, one more modified MMP polypeptide can be employed to permit conditional regulation of the MMP activity based on both calcium and temperature.

a. Calcium

The condition-dependent (i.e., calcium-dependent) nature of the csMMP polypeptide activity, used in the methods provided herein, permits regulation by administration of formulations that contain calcium. Hence, the activity of the csMMP polypeptide can be conditionally controlled or regulated by calcium in the methods provided herein. In the methods provided herein, the modified MMP polypeptide is co-administered with a high concentration of calcium greater than physiological levels (e.g., 2 mM to 100 mM, such as 5 mM to 50 mM or 2 mM to 20 mM, for example at least 10 mM) to a physiological locus. For example, the modified MMP is co-administered with the high concentration of calcium to the ECM of a subject, such as by injection. The modified MMP and calcium can be administered together as a single composition. In other cases, compositions containing a modified MMP and high concentrations of calcium can be administered separately, whereby the calcium is administered prior to, subsequently with, simultaneously with or intermittently with the modified MMP at or near the same site or locus of administration of the MMP. For example, the separately administered calcium-containing formulation is administered to the site containing the csMMP and/or the affected site where csMMP activity is desired. For example, in instances where csMMP activity is desired in the upper epidermis, transdermal delivery of Ca²⁺-containing formulations can be effected by sonophoresis (Menon et al. (1994) J. Invest. Dermatol. 102(5):789-795). In any of such examples, when administered to a physiologic locus of a subject in vivo that normally contains a physiological level of calcium (e.g., less than 2 mM, such as about 1 mM to 1.3 mM), the activity of the modified enzyme will be restricted to the desired treatment area regulated to contain the higher calcium concentration, since diffusion of the enzyme from this area would cause it to encounter the lower, physiological calcium concentration and be inactivated.

In aspects of the methods herein, the csMMP activity can be restored after the activity expires in vivo at the site of administration. For example, after the activity of previously activated csMMP (e.g., csMMP administered in the presence of high Ca²⁺, such as 10 mM Ca²⁺) becomes inactive or exhibits substantially decreased activity, following dispersion or cellular uptake of the extracellular Ca²⁺ in the local environment of the csMMP, the csMMP can be reactivated, in other words the activity of the csMMP can be restored, upon administration of a calcium-containing formulation that returns the calcium concentration in the local environment to a sufficient concentration to reinstate csMMP activity. For example, a calcium-containing buffer or fluid can be administered to reinstate csMMP activity after the activity has lapsed. The calcium-containing formulation formulated for subsequent administration can contain concentrations of Ca²⁺ of at least or about at least 2 mM, 3 mM, 4 mM, 5 mM, 6 mM, 7 mM, 8 mM, 9 mM, 10 mM, 15 mM, 20 mM, 25 mM, 50 mM, 100 mM or greater than 100 mM Ca²⁺. In examples where the particular enzyme is reversibly active, the calcium-containing formulation can be administered intermittently over a course of hours or days. Thus, the time duration of csMMP activity renewal also can be controlled by continued or recurring exposure to high calcium concentrations (e.g., 10 mM Ca²⁺) for a predetermined length of time.

If the treating physician determines that deactivation, or reduced activity, of the administered csMMP is necessary, for example upon completion of treatment or to cease or prevent undesired side-effects, a deactivating formulation also can be administered. Due to the sensitivity of the csMMP polypeptides used in the methods provided herein to calcium concentrations, the methods provided herein optionally include the use of formulations capable of sequestering calcium ions thereby effecting deactivation of csMMP polypeptides rendered active by calcium. Calcium chelating agents, such as ethylenediaminetetraacetic acid (EDTA), ethylene glycol tetraacetic acid (EGTA), 1,2-bis(o-aminophenoxy)ethane-N,N,N′,N′-tetraacetic acid (BAPTA), derivatives thereof (e.g., diesters of BAPTA), or other synthetic calcium chelating agents (see e.g., U.S. Pat. No. 7,799,831; Woessner (1999) Ann. N.Y. Acad. Sci. 878:388-403), sequester calcium, resulting in a decrease in the availability of Ca²⁺ ions. Hence, deactivating formulations can include calcium-chelating agents, such as BAPTA, EGTA and/or EDTA, which sequester Ca²⁺ ions, thereby reducing csMMP activity and/or rendering the administered csMMP inactive or substantially inactive. Deactivating formulations can be administered by any suitable route of administration using suitable formulations as described herein including, but not limited to, subcutaneous injection, intramuscular, intradermal, oral, and topical and transdermal administration. A route of administration of a deactivating formulation typically is chosen that results in administration under the skin directly to the site containing the csMMP. Exemplary of such routes of administration include, but are not limited to, subcutaneous, intramuscular, or intradermal injection.

Thus, calcium chelating agents can be included in formulations, used in the methods provided herein, to sequester calcium in the environment of an active csMMP, resulting in a local decrease in calcium concentration and inactivation or decreased activity of the csMMP. The calcium chelator compositions can be formulated into any suitable pharmaceutical preparation such as a solution, suspension, tablet, dispersible tablet, pill, capsule, powder, sustained release formulation, or elixir, for oral administration. Typically, the calcium chelator-containing composition is formulated for parenteral administration, generally characterized by either subcutaneous, intramuscular or intradermal injection. Thus, formulations described in Section F above can include a calcium chelating agent. Calcium chelator-containing formulations typically do not include csMMP polypeptides.

In one example, a composition containing an effective amount of calcium chelating agent is administered to a location containing an active csMMP to inactivate or reduce the activity of the csMMP. The effective amount of calcium chelating agent in the formulation will vary depending on the particular chelating agent, the particular csMMP, the particular patient, the tissue to which the formulation is administered, the disease to be treated, the route of administration, and other considerations, including the urgency of cessation of csMMP activity. Thus, dosages can be empirically determined based on known testing protocols or extrapolation from in vivo or in vitro test data.

Typically, a calcium chelating formulation contains a calcium chelating agent, such as BAPTA, EGTA or EDTA at a concentration of 1 μM to 100 mM, such as 10 μM to 20 mM. Volumes administered can be adjusted depending on the disease to be treated and the route of administration. It is contemplated herein that 1-100 mL, 1-50 mL, 10-50 mL, 10-30 mL, 1-20 mL, or 1-10 mL, typically 1-20 mL of a calcium chelating formulation can be administered subcutaneously to regulate the activity of a csMMP used for the treatment of an ECM-mediated disease or condition, such as cellulite. The administration can be subsequent to or intermittent with administration of a calcium-containing formulation. Thus, one of skill in the art can modulate csMMP activity using both calcium-containing and calcium-chelating formulations to achieve the desired duration of activity.

b. Temperature

The procedures used to activate csMMP polypeptides, which employ co-formulation or co-administration of calcium, can be combined with other procedures which can modulate csMMP activity. In particular examples, among the csMMPs described herein are those that contain modifications that confer temperature sensitivity in addition to calcium sensitivity. For example, csMMP polypeptides that are modified to be temperature sensitive exhibit increased activity at a permissive temperature (e.g., 25° C.) compared to non-permissive temperatures (e.g., 34° C. or 37° C.). Exemplary modifications which can render a MMP, such as a MMP-1 polypeptide, temperature sensitive are set forth in U.S. Publication No. 2010/0284995. Thus, where the additional feature is temperature sensitivity, activation of the csMMP can be achieved by increasing the local calcium concentration and by altering the temperature at the site of administration. In other words, a temperature-sensitive csMMP can be activated by exogenous application of a temperature condition and administration of sufficient calcium.

The temperature of the site of injection can be modulated by any method known in the art, for example methods disclosed in U.S. Publication No. 2010/0284995. For example, the temperature of the temperature-sensitive csMMP can be adjusted by changing the temperature of one or more injection buffers or by applying a heating or cooling pack. For example, where the permissive temperature of the temperature-sensitive csMMP is 25° C. and the non-permissive temperature is body temperature (e.g., 34° C. or 37° C.), a buffer or other liquid diluent that is less than or less than about 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C. or less can be injected to cool the site of treatment. In other words, the temperature-sensitive csMMP is provided and/or exposed to a buffer or other liquid diluent that is less than or less than about 25° C., 24° C., 23° C., 22° C., 21° C., 20° C., 19° C., 18° C., 17° C., 16° C., 15° C., 14° C., 13° C., 12° C.-11° C., 10° C., 9° C., 8° C., 7° C., 6° C., 5° C. or less. The buffer or liquid can be provided in the same composition as the temperature-sensitive csMMP or in a separate composition. When provided separately, it can be administered prior to, simultaneously with, subsequent to or intermittently with the temperature-sensitive csMMP. Upon administration in vivo where the physiologic temperature is at or about 37° C., the temperature of the buffer warms by heat transfer from the body of the subject to a temperature providing the permissive temperature for activation of the temperature-sensitive csMMP (which could occur immediately or almost immediately depending on the temperature of the liquid).

In another example, the temperature at the site of temperature-sensitive csMMP administration can be altered by a cold pack or a hot pack, depending on the particular enzyme and the permissive temperature required. For example, ice wraps, gel ice packs, cold therapy, ice packs, cold compress, ice blankets, or other similar items can be used to apply exogenous cooling and achieve permissive temperatures at the site of treatment. Hot packs or heating pads can be used to apply exogenous heating to achieve permissive temperatures at the site of treatment. In other words, the locus of administration of the temperature-sensitive csMMP can be exposed to the cold pack or hot pack in order to cool or warm the site of administration below or above the physiological temperature of the body, respectively, prior to, concurrently with or subsequent to administration of the temperature-sensitive csMMP to the same locus. For example, the cold pack can be frozen (e.g., ice pack), or can be a liquid cold pack maintained at a temperature that is less than physiological temperatures, and generally up to 4° C., 5° C., 6° C., 7° C., 8° C., 9° C., 10° C., 11° C., 12° C., 13° C., 14° C., 15° C. or more. A cold or hot pack can be applied directly to the locus of treatment, and generally is applied locally to the skin at the site of administration of the temperature-sensitive csMMP. One of skill in the art can empirically determine the length of time required for application depending of the particular target depth of the tissue that is being treated, the particular enzyme that is being used, and other factors based on known testing protocols or extrapolation from in vivo or in vitro test data. The hot pack or cold pack can be applied prior to, subsequent to, simultaneously with or intermittently with the temperature-sensitive csMMP. For example, if the particular enzyme is reversibly active, the cold pack can be applied intermittently over a course of hours or days in combination with administration of calcium. It is understood that it is customary for a subject to feel cold, aching and burning and numbness upon administration of a cold pack, and such symptoms can be monitored by the subject or a treating physician.

In aspects of the methods herein, exposure of the temperature-sensitive csMMP to the permissive temperature is generally not sufficient to render the temperature-sensitive csMMP sufficiently active in the absence of sufficient calcium to satisfy the increased calcium dependency of the enzyme. Thus, the calcium concentration at the site of temperature-sensitive csMMP administration is generally greater than the physiological calcium concentration (e.g., 10 mM), in addition to conditions providing the permissive temperature to activate the temperature-sensitive csMMP. Due to the physiologic temperature and calcium conditions in vivo, the temperature will eventually warm to the non-permissive temperature, and the calcium concentration will reduce to physiological levels following dissipation, sequestration, or uptake of Ca²⁺ ions, thereby resulting in inactivation of the enzyme and temporal control thereof.

In particular embodiments, the temperature-sensitive csMMP is exposed to a temperature that is at or below the permissive temperature of the body immediately before administration. For example, the temperature-sensitive csMMP is stored at a cold temperature and/or is reconstituted in a cold buffer. A permissive concentration of calcium (e.g., 10 mM Ca²⁺) also can be included in the reconstitution buffer or the calcium-containing formulation can be administered separate from the chilled temperature-sensitive csMMP-containing buffer. In some examples, the locus of administration of the temperature-sensitive csMMP also is exposed cold by exposure to a cold pack to cool the site of administration below the physiologic temperature of the body. In some examples, the locus of administration can be pre-treated with a calcium containing formulation that increases the calcium concentration at the locus of administration to a concentration that is above physiological levels. Upon administration of the temperature-sensitive csMMP, the temperature-sensitive csMMP is exposed to the permissive temperature (and calcium concentration), which will steadily return to the nonpermissive physiological interstitial calcium concentration and temperature of the body (e.g., about 37° C.). Where the temperature reaches the nonpermissive temperature or the calcium concentration decreases to concentrations below permissive conditions, the temperature-sensitive csMMP is rendered inactive or substantially inactive. Hence, activation of the temperature-sensitive csMMP is conditionally controlled. The duration of time of exposure to a permissive temperature below the physiological temperature of the body can be controlled by continued exposure to a cold pack, and calcium-containing formulations also can be administered as described elsewhere herein, to achieve activity at the site of administration for the predetermined length of time.

3. Exemplary Fibrotic Diseases and Conditions (e.g., Collagen-Mediated Diseases and Conditions)

Among fibrotic diseases and conditions are those that are mediated by or associated with production or accumulation of collagen, and in particular type I or type II collagen. Type I collagen is generally found in tendon, bone, ligaments, scar tissue and Dupuytren's tissue. Type III collagen is generally found in tendon, scar, Dupuytren's tissue and granulation tissue. In many diseases and conditions, an underlying effect of tissue damage or other trauma is the production of collagen, including type I and type III collagen, that can result in the irregular formation of collagen fibers. These irregularly-formed collagen fibers can include fibrous septae, fibrous scars, fibrous plaques, cords, nodules and other fibrous structure. In the methods herein, the modified MMP conditionally or temporally severs the fibers, for example by cleavage or degradation of the collagen components therein, and thereby treats the disease or condition.

Descriptions of the involvement of collagen to collagen-mediated diseases or conditions is provided below as an example of the role of ECM components in diverse disease and conditions. Such descriptions are meant to be exemplary only and are not intended to limit the application of the methods provided herein to particular ECM-mediated diseases or conditions. One of skill in the art can select a method using a selected csMMP to treat any desired fibrotic or ECM-mediated disease, based on the ability of the selected enzyme to cleave or degrade the ECM component involved in the particular disease or condition. For example, as described herein, MMP-1 cleaves type I and type III collagens, such as those abundant in the skin. Hence, a csMMP-1 can be used for treatments, uses and processes for treating a collagen-mediated disease or condition. The particular treatment and dosage can be determined by one of skill in the art. Considerations in assessing treatment include, for example, the disease to be treated, the ECM component involved in the disease, the severity and course of the disease, whether the csMMP is administered for preventive or therapeutic purposes, previous therapy, the patient's clinical history and response to therapy, and the discretion of the attending physician.

Collagen is a major structural constituent of mammalian organisms and makes up a large portion of the total protein content of the skin and other parts of the animal body. Numerous diseases and conditions are associated with excess collagen deposition, for example, due to erratic accumulation of fibrous tissue rich in collagen or other causes. Collagen-mediated diseases or conditions (also referred to as fibrotic tissue disorders) are known to one of skill in the art (see e.g., published U.S. Application No. 2007/0224183; U.S. Pat. Nos. 6,353,028; 6,060,474; 6,566,331; and 6,294,350). Excess collagen has been associated with diseases and conditions, such as, but not limited to, fibrotic diseases or conditions resulting in scar formation, cellulite, Dupuytren's syndrome, Peyronie's disease, frozen shoulder, localized scleroderma, lymphedema, interstitial cystitis (IC), telangiectasia, Barrett's metaplasia, Pneumatosis cytoides intestinalis, collagenous colitis. For example, disfiguring conditions of the skin, such as wrinkling, cellulite formation and neoplastic fibrosis result from excessive collagen deposition, which produces unwanted binding and distortion of normal tissue architecture.

The methods provided herein use csMMPs, including but not limited to csMMP-1, to treat collagen-mediated diseases or conditions. The csMMPs used in the methods of treating collagen-mediated diseases are substantially active only when in the presence of sufficient concentrations of calcium, for example calcium concentrations that exceed physiological calcium concentrations, such as calcium concentrations at or greater than 2 mM Ca²⁺, for example 10 mM Ca²⁺. For example, temporary activation of a csMMP administered to the extracellular matrix, such as the skin interstitium, can be achieved by infusing or injecting a calcium-containing (i.e., activating) buffered solution or other liquid directly to the affected site and/or the locus of csMMP administration. In one example, a calcium-containing buffer can be administered via sub-epidermal administration, i.e., under the skin, such that administration is effected directly at the site where ECM components are present and accumulated. Other methods of calcium-based regulation of csMMP activity can be employed, and are known to one of skill in the art in view of the descriptions herein.

a. Cellulite

Modified MMP polypeptides, such as any of the csMMPs (e.g., csMMP-1) described herein, can be used in methods to treat cellulite (also known as edematous fibrosclerotic panniculophaty). In particular examples, the modified MMP is selected that cleaves or degrades a type I or type III collagen, which are the collagen types most abundant in the fibrous septae that cause dimpling associated with cellulite.

In normal adipose tissues, a fine mesh of blood vessels and lymph vessels supplies the tissue with necessary nutrients and oxygen, and takes care of the removal of metabolized products. For example, triglycerides are stored in individual adipocytes that are grouped into capillary rich lobules. Each fat lobule is composed of adipocytes. Vertical strands of collagen fibers named fibrous septae separate the fat lobules and tether the overlying superficial fascia to the underlying muscle.

Cellulite is typically characterized by dermal deterioration due to a breakdown in blood vessel integrity and a loss of capillary networks in the dermal and subdermal levels of the skin. The vascular deterioration tends to decrease the dermal metabolism. This decreased metabolism hinders protein synthesis and repair processes, which results in dermal thinning. The condition is further characterized by fat cells becoming engorged with lipids, swelling and clumping together, as well as excess fluid retention in the dermal and subdermal regions of the skin. The accumulation of fat globules or adipose cells creates a need for a bigger blood supply to provide extra nourishment. To provide the blood to tissues, new capillaries are formed, which release more filtrate resulting in a saturation of tissues with interstitial fluid causing edema in the adipose tissues. Abundant reticular fibers in the interstitial tissues accumulate and thicken around the aggregated adipose cells; they form capsules or septa, which gradually transform into collagen fibers and are felt as nodules. The formation of these septa further occludes fat cells. Collagen fibers are also laid down in the interstitial tissue spaces, rendering the connective tissue sclerotic (hard). Ultimately, cellulite presents by skin dimpling that occurs mainly on the pelvic region of women. The dimpling is caused when the fibrous septae, made of types I and III collagen, in the subcutaneous tissue connect the dermis to the deeper hypodermis. In cellulite, subcutaneous fat cells swell and push upwards while the septae act as an anchor to pull the epidermis downward to form the classic cellulite dimple lesion.

Cellulite is more prevalent among females than males. The prevalence of cellulite is estimated between 60% and 80% of the female population and its severity tends to worsen with obesity. One published study showed by in vivo magnetic resonance imaging that women with cellulite have a higher percentage of perpendicular fibrous septae than women without cellulite or men (Querleux et al. (2002) Skin Res. Technol. 8:118-124). Cellulite occurs most often on the hips, thighs and upper arms. For example, premenopausal females tend to accumulate fat subcutaneously, primarily in the gluteal/thigh areas where cellulite is most common. Clinically, cellulite is accompanied by symptoms that include thinning of the epidermis, reduction and breakdown of the microvasculature leading to subdermal accumulations of fluids, and subdermal agglomerations of fatty tissues.

b. Dupuytren's Disease

Modified MMP polypeptides, such as any of the csMMPs (e.g., csMMP-1) described herein, can be used in to treat Dupuytren's syndrome (also called Dupuytren's contracture). In particular examples, the modified MMP is selected that cleaves or degrades type I or type III collagen, which are the collagen types most abundant in the fibrous cords or nodules associated with this disease.

Dupuytren's contracture (also known as Morbus Dupuytren) is a fixed flexion contracture of the hand where the fingers bend towards the palm and cannot be fully extended. A similar lesion sometimes occurs in the foot. The connective tissue within the hand becomes abnormally thick and is accompanied by the presence of nodules containing fibroblasts and collagen, particularly type III collagen. The fibrous cord of collagen is often interspersed with a septa-like arrangement of adipose tissue. These present clinically as mattress-type “lumps” of varying sized and in Dupuytren's disease are termed nodules. This can cause the fingers to curl, and can result in impaired function of the fingers, especially the small and ring fingers. Dupuytren's disease occurs predominantly in men. It is generally found in middle aged and elderly persons, those of Northern European ancestry, and in those with certain chronic illnesses such as diabetes, alcoholism and smoking.

Dupuytren's disease is a slowly progressive disease that occurs over many years causing fixed flexion deformities in the metacarpophalangeal (MP) and proximal interphalangeal (PIP) joints of the fingers. The small and ring fingers are the most often affected. The disease progresses through three stages (Luck et al. (1959) J. Bone Joint Surg. 41A:635-664). The initial proliferative stage is characterized by nodule formation in the palmar fascia in which a cell known as the myofibroblast appears and begins to proliferate. The involutional or mid-disease stage involves myofibroblast proliferation and active type III collagen formation. In the last or residual phase, the nodule disappears leaving acellular tissue and thick bands of collagen. The ratio of type III collagen to type I collagen increases.

Treatment of Dupuytren's disease with a modified MMP, such as any csMMP provided herein, is typically in the mid-disease and residual disease stages. The modified MMP is generally injected directly into the cord. The injection can be repeated, if necessary. Also, the treatment can optionally include a finger extension procedure in order to further break up the cord. Finger extension exercises also can be employed following injection.

c. Peyronie's Disease

Modified MMP polypeptides, such as any of the csMMPs (e.g., csMMP-1) described herein, can be used in methods to treat Peyronie's disease. In particular examples, the modified MMP is selected that cleaves or degrades type I or type III collagen, which are the collagen types most abundant in the fibrous plaques associated with this disease.

Peyronie's disease is a connective tissue disorder involving the growth of fibrous plaques in the soft tissue of the penis affecting as many as 1-4% of men. Collagen, and principally type I and type III collagen fibers, are the major component of the plaque in Peyronie's disease (see e.g., Bivalacqua et al. (2000) Curr. Urol. Reports 1:297-301). Specifically, the fibrosing process occurs in the tunica albuginea, a fibrous envelope surrounding the penile corpora cavernosa. The pain and disfigurement associated with Peyronie's disease relate to the physical structure of the penis in which is found two erectile rods, called the corpora cavernosa, a conduit (the urethra) through which urine flows from the bladder, and the tunica which separates the cavernosa from the outer layers of skin of the penis. A person exhibiting Peyronie's disease will have formation(s) of plaque or scar tissue between the tunica and these outer layers of the skin (referred to as “sub-dermal” in this application). The scarring or plaque accumulation of the tunica reduces its elasticity causes such that, in the affected area, it will not stretch to the same degree (if at all) as the surrounding, unaffected tissues. Thus, the erect penis bends in the direction of the scar or plaque accumulation, often with associated pain of some degree. In all but minor manifestations of Peyronie's disease, the patient has some degree of sexual dysfunction. In more severe cases, sexual intercourse is either impossible, or is so painful as to be effectively prohibitive.

Empirical evidence indicates an incidence of Peyronie's disease in approximately one percent of the male population. Although the disease occurs mostly in middle-aged men, younger and older men can acquire it. About 30 percent of men with Peyronie's disease also develop fibrosis (hardened cells) in other elastic tissues of the body, such as on the hand or foot. Common examples of such other conditions include Dupuytren's contracture of the hand and Ledderhose Fibrosis of the foot.

d. Ledderhose Fibrosis

Modified MMP polypeptides, such as any of the csMMPs (e.g., csMMP-1) described herein, can be used in methods to treat Ledderhose fibrosis. In particular examples, the modified MMP is selected that cleaves or degrades type I or type III collagen, which are the collagen types most abundant in the plantar fibrosis associated with this disease. Ledderhose fibrosis is similar to Dupuytren's disease and Peyronie's disease, except that the fibrosis due to fibroblast proliferation and collagen deposition, principally type I or type III collagen, occurs in the foot. Ledderhose disease is characterized by plantar fibrosis over the medial sole of the foot, and is sometimes referred to as plantar fibrosis.

e. Stiff Joints

Modified MMP polypeptides, such as any of the csMMPs (e.g., csMMP-1) described herein, can be used in methods to treat stiff joints, for example, frozen shoulder. In particular examples, the modified MMP is selected that cleaves or degrades type I or type III collagen, which are the collagen types most abundant in the fibrous capsule associated with this disease.

Frozen shoulder (adhesive capsulitis) is a chronic fibrozing condition of the capsule of the joint characterized by pain and loss of motion or stiffness in the shoulder. It affects about 2% of the general population. Frozen shoulder results from increased fibroblast matrix synthesis. The synthesis is caused by an excessive inflammatory response resulting in the overproduction of cytokines and growth factors. Fibroblasts and myofibroblasts lay down a dense matrix of collagen in particular, type-I and type-III collagen within the capsule of the shoulder. This results in a scarred contracted shoulder capsule and causes joint stiffness.

Other examples of stiff joints include, but are not limited to, those caused by capsular contractures, adhesive capsulitis and arthrofibrosis, which result from musculoskeletal surgery. Such stiff joints can occur in joints, including, for example, joints of the knees, shoulders, elbows, ankles and hips. Like frozen shoulder, such joint diseases are caused by increased matrix synthesis and scar formation. The stiff joints inevitably can cause abnormally high forces to be transmitted to the articular cartilage of the affected area. Over time, these forces result in the development of degenerative joint disease and arthritis. For example, in arthrofibrosis and capsular contracture, fibroblasts form excessive amounts of matrix in response to local trauma, such as joint dislocation.

f. Existing Scars

Modified MMP polypeptides, such as any of the csMMPs (e.g., csMMP-1) described herein, can be used in methods to treat existing scars. In particular examples, the modified MMP is selected that cleaves or degrades type I or type III collagen, which are the collagen types found in scar tissue.

Collagen is particularly important in the wound healing process and in the process of natural aging, where it is produced by fibroblast cells. In some cases, however, an exaggerated healing response can result in the production of copious amounts of healing tissue (ground substance), also termed scar tissue. For example, various skin traumas such as burns, surgery, infection, wounds and accident are often characterized by the erratic accumulation of fibrous tissue rich in collagen, and particular type I or type III collagen. There also is often an increased proteoglycan content. In addition to the replacement of the normal tissue that has been damaged or destroyed, excessive and disfiguring deposits of new tissue sometimes form during the healing process. The excess collagen deposition has been attributed to a disturbance in the balance between collagen synthesis and collagen degradation. Including among scars are, for example, chronic tendinosis or scar tissue of the tendons, surgical adhesions, keloids, hypertrophic scars, and depressed scars.

i. Surgical Adhesions

Modified MMP polypeptides, such as any of the csMMPs (e.g., csMMP-1) described herein, can be used in methods to treat surgical adhesions. In particular examples, the modified MMP is selected that cleaves or degrades type I or type III collagen, which are the collagen types found in surgical scars.

Surgical adhesions are attachments of organs or tissues to each other through scar formation, which can cause severe clinical problems. The adhesions are fibrous bands that form between tissues and organs. The formation of some scar tissue after surgery or tissue injury is normal. In some cases, however, the scar tissue overgrows the region of injury and creates surgical adhesions, which tend to restrict the normal mobility and function of affected body parts. As the fibrous adhesions persist, tissue repair cells (e.g., macrophages or fibroblasts), lay down collagen (e.g., type I or type III collagen) and other matrix substances to form a permanent fibrous adhesion. In particular, fibroblast proliferation and matrix synthesis is increased locally following such soft tissue injury. Adhesions then form when the body attempts to repair tissue by inducing a healing response. For example, this healing process can occur between two or more otherwise healthy separate structures (such as between loops of bowel following abdominal surgery). Alternately, following local trauma to a peripheral nerve, fibrous adhesions can form, resulting in severe pain during normal movement.

ii. Keloids

Modified MMP polypeptides, such as any of the csMMPs (e.g., csMMP-1) described herein, can be used in methods to treat keloids. In particular examples, the modified MMP is selected that cleaves or degrades type I or type III collagen, which are the collagen types found in the fibrous nodules of keloids.

Keloids are scars of connective tissue containing hyperplastic masses that occur in the dermis and adjacent subcutaneous tissue, most commonly following trauma. Keloids generally are fibrous nodules that can vary in color from pink or red to dark brown. Keloids form in scar tissue as a result of overgrowth of collagen, which participates in wound repair. Keloids are composed mainly of type III or type I collagen. Keloid lesions are formed when local skin fibroblasts undergo vigorous hyperplasia and proliferation in response to local stimuli. The resulting lesion can result in a lump many times larger than the original scar. In addition to occur as a result of wound or other trauma, keloids also can form from piercing, pimples, a scratch, severe acne, chickenpox scarring, infection at a wound site, repeated trauma to an area, or excessive skin tension during wound closure.

iii. Hypertrophic Scars

Modified MMP polypeptides, such as any of the csMMPs (e.g., csMMP-1) described herein, can be used in methods to treat hypertrophic scars. In particular examples, the modified MMP is selected that cleaves or degrades type III collagen, which is the collagen types found in the hypertrophic scars.

Hypertrophic scars are similar to keloids, except that they do not generally extend beyond the initial site of injury. For example, hypertrophic scars are generally raised scars that form at the site of wounds. They generally do not grow beyond the boundaries of the original wound. Like keloid scars, hypertrophic scars are a result of the body overproducing collagen. They generally form within 4 to 8 weeks following. wound infection or other traumatic injury. Like keloid scars, hypertrophic scars contain an overabundance of dermal collagen, primarily type III collagen.

g. Scleroderma

Modified MMP polypeptides, such as any of the csMMPs (e.g., csMMP-1) described herein, can be used in methods to treat scleroderma. In particular examples, the modified MMP is selected that cleaves or degrades type I collagen, which is the collagen types found the thickened skin associated with scleroderma.

Scleroderma is characterized by a thickening of the collagen. In particular, increased deposition of type I collagen is evident in the skin and involved internal organs. The more common form of the disease, localized scleroderma, affects only the skin, usually in just a few places, and sometimes the face. It is sometimes referred to as CREST syndrome. Symptoms include hardening of the skin and associated scarring. The skin also appears reddish or scaly, and blood vessels can be more visible. In more serious cases, scleroderma can affect the blood vessels and internal organs. Diffuse scleroderma can be fatal as a result of heart, kidney lung or intestinal damage, due to musculoskeletal, pulmonary, gastrointestinal, renal and other complications.

The condition is characterized by collagen buildup leading to loss of elasticity. The overproduction of collagen has been attributed to autoimmune dysfunction, resulting in accumulation of T cells and production of cytokines and other proteins that stimulate collagen deposition from fibroblasts.

h. Lymphedema

Modified MMP polypeptides, such as any of the csMMPs (e.g., csMMP-1) described herein, can be used in methods to treat lymphedema. In particular examples, the modified MMP is selected that cleaves or degrades type I or type III collagen, which is the collagen types found the skin of subjects with lymphedema.

Lymphedema is an accumulation of lymphatic fluid that causes swelling in the arms and legs. Lymphedema can progress to include skin changes such as, for example, lymphostatic fibrosis, sclerosis and papillomas (benign skin tumors) and swelling. Tissue changes associated with lymphedema include proliferation of connective tissue cells, such as fibroblasts, production of collagen fibers, an increase in fatty deposits and fibrotic changes. These changes occur first at the lower extremities, i.e. the fingers and toes. Lymphedema can be identified based on the degree of enlargement of the extremities. For example, one method to assess lymphedema is based on identification of 2-cm or 3-cm difference between four comparative points of the involved and uninvolved extremities.

i. Collagenous colitis

Calcium-sensitive MMPs, such as csMMP-1, can be used in methods to treat collagenous colitis. Collagenous colitis was first described as chronic watery diarrhea (Lindstrom et al. (1976) Pathol. Eur. 11:87-89). Collagenous colitis is characterized by collagen deposition, likely resulting from an imbalance between collagen production by mucosal fibroblasts and collagen degradation. It results in secretory diarrhea. The incidence of collagenous colitis is similar to primary biliary cirrhosis. The disease has an annual incidence of 1.8 per 100,000 and a prevalence of 15.7 per 100,000, which is similar to primary biliary cirrhosis (12.8 per 100,000) and lower than ulcerative colitis (234 per 100,000), Crohn's disease (146 per 100,000) or celiac disease (5 per 100,000). In patients with chronic diarrhea, about 0.3 to 5% have collagenous colitis. Collagenous colitis is an inflammatory disease resulting in increased production of cytokines and other agents that stimulate the proliferation of fibroblasts, resulting in increased collagen accumulation.

2. Spinal Pathologies

As described herein, the methods using csMMPs provided herein can be used to treat diseases and conditions of the ECM or involving the ECM. These include spinal pathologies, typically referred to as herniated disc or bulging discs, that can be treated by methods of administering a csMMP, and enabling temporary activation thereof, by regulating the concentration of calcium available to the administered csMMP, as described herein. Herniated discs that can be treated using the methods provided herein include protruded and extruded discs. A protruded disc is one that is intact but bulging. In an extruded disk, the fibrous wrapper has torn and nucleus pulposus (NP) has oozed out, but is still connected to the disk. While the NP is not the cause of the herniation, the NP contributes to pressure on the nerves, causing pain. The NP contains hyaluronic acid, chondrocytes, collagen fibrils, and proteoglycan aggrecans that have hyaluronic long chains which attract water. Attached to each hyaluronic chain are side chains of chondroitin sulfate and keratan sulfate.

Herniated discs have been treated with chemonucleolytic drugs, such as chymopapain and a collagenase, typically by local introduction of the drug into the disc. A chemonucleolytic drug degrades one or more components of the NP, thereby relieving pressure. Chemonucleolysis is effective on protruded and extruded disks. Chemonucleolysis has been used treat lumbar (lower) spine and cervical (upper spine) hernias. Hence, the csMMPs provided herein can be used as chemonucleolytic drugs and administered, such as by injection, to the affected disc, under conditions that activate the csMMP.

### I. COMBINATION THERAPIES

Any of the modified MMP polypeptides described herein can be further co-formulated or co-administered together with, prior to, intermittently with, or subsequent to, other therapeutic or pharmacologic agents or procedures. Therapeutic or pharmacologic agents that can be co-formulated or co-administered with csMMP polypeptides include, but are not limited to, other biologics, small molecule compounds, dispersing agents, anesthetics, vasoconstrictors and surgery, and combinations thereof. For example, for any disease or condition, including all those exemplified above, for which other agents and treatments are available, selected csMMPs for such diseases and conditions can be used in combination therewith. In another example, a local anesthetic, for example, lidocaine can be administered to provide pain relief. In some examples, the anesthetic can be provided in combination with a vasoconstrictor to increase the duration of the anesthetic effects. Any of the pharmacological agents provided herein can be combined with a dispersion agent that facilitates access into the tissue of pharmacologic agents, for example, following subcutaneous administration. Such substances are known in the art and include, for example, soluble glycosaminoglycanase enzymes such as members of the hyaluronidase glycoprotein family (US 2005/0260186, US 2006/0104968).

1. Anesthesia and Vasoconstrictors

Compositions of modified csMMPs, used in the methods provided herein, can be co-formulated or co-administered with a local anesthesia. Anesthetics include short-acting and long-lasting local anesthetic drug formulations. Short-acting local anesthetic drug formulations contain lidocaine or a related local anesthetic drug dissolved in saline or other suitable injection vehicle. Typically, local anesthesia with short-acting local anesthetics last approximately 20-30 minutes. Exemplary anesthetics include, for example, non-inhalation local anesthetics such as ambucaines; amoxecaines; amylocalnes; aptocaines; articaines; benoxinates; benzyl alcohols; benzocaines; betoxycaines; biphenamines; bucricaines; bumecaines; bupivacaines; butacaines; butambens; butanilicaines; carbizocaines; chloroprocaine; clibucaines; clodacaines; cocaines; dexivacaines; diamocaines; dibucaines; dyclonines; elucaines; etidocaines; euprocins; fexicaines; fomocaines; heptacaines; hexylcaines; hydroxyprocaines; hydroxytetracaines; isobutambens; ketocaines; leucinocaines; lidocaines; mepivacaines; meprylcaines; octocaines; orthocaines; oxethacaines; oxybuprocaines; phenacaines; pinolcaines; piperocaines; piridocaines; polidocanols; pramocaines; prilocalnes; procaines; propanocaines; propipocaines; propoxycaines; proxymetacaines; pyrrocaines; quatacaines; quinisocaines; risocaines; rodocaines; ropivacaines; salicyl alcohols; suicaines; tetracaines; trapencaines; and trimecaines; as well as various other non-inhalation anesthetics such as alfaxalones; amolanones; etoxadrols; fentanyls; ketamines; levoxadrols; methiturals; methohexitals; midazolams; minaxolones; propanidids; propoxates; pramoxines; propofols; remifentanyls; sufentanyls; tiletamines; and zolamine. The effective amount in the formulation will vary depending on the particular patient, disease to be treated, route of administration and other considerations. Such dosages can be determined empirically.

Due to the short half-life of local anesthetics, it is often desirable to co-administer or co-formulate such anesthetics with a vasoconstrictor. Examples of vasoconstrictors include alpha adrenergic receptor agonists including catecholamines and catecholamine derivatives. Particular examples include, but are not limited to, levonordefrin, epinephrine and norepinephrine. For example, a local anesthetic formulation, such as lidocaine, can be formulated to contain low concentrations of epinephrine or another adrenergic receptor agonist such as levonordefrin. Combining local anesthetics with adrenergic receptor agonists is common in pharmaceutical preparations (see e.g., U.S. Pat. Nos. 7,261,889 and 5,976,556). The vasoconstrictor is necessary to increase the half-life of anesthetics. The vasoconstrictor, such as epinephrine, stimulates alpha-adrenergic receptors on the blood vessels in the injected tissue. This has the effect of constriction the blood vessels in the tissue. The blood vessel constriction causes the local anesthetic to stay in the tissue much longer, resulting in a large increase in the duration of the anesthetic effect.

Generally, a vasoconstrictor is used herein in combination with an anesthetic. The anesthetic agent and vasoconstrictor can be administered together as part of a single pharmaceutical composition or as part of separate pharmaceutical compositions acting together to prolong the effect of the anesthesia, so long as the vasoconstrictor acts to constrict the blood vessels in the vicinity of the administered anesthetic agent. In one example, the anesthetic agent and vasoconstrictor are administered together in solution. In addition, the anesthetic agent and vasoconstrictor can be formulated together or separate from the modified MMP and/or calcium-containing compositions. Single formulations are preferred. The anesthetic agent and vasoconstrictor can be administered by injection, by infiltration or by topical administration, e.g., as part of a gel or paste. Typically, the anesthetic agent and vasoconstrictor are administered by injection directly into the site to be anesthetized, for example, by subcutaneous administration. The effective amount in the formulation will vary depending on the particular patient, disease to be treated, route of administration and other considerations. Such dosages can be determined empirically. For example, exemplary amounts of lidocaine are or are about 10 mg to 1000 mg, 100 mg to 500 mg, 200 mg to 400 mg, 20 mg to 60 mg, or 30 mg to 50 mg. The dosage of lidocaine administered will vary depending on the individual and the route of administration. Epinephrine can be administered in amounts such as, for example, 10 μg to 5 mg, 50 μg to 1 mg, 50 μg to 500 μg, 50 μg to 250 μg, 100 μg to 500 μg, 200 μg to 400 μg, 1 mg to 5 mg or 2 mg to 4 mg. Typically, epinephrine can be combined with lidocaine in a 1:100,000 to 1:200,000 dilution, which means that 100 mL of anesthetic contains 0.5 to 1 mg of epinephrine. Volumes administered can be adjusted depending on the disease to be treated and the route of administration. It is contemplated herein that 1-100 mL, 1-50 mL, 10-50 mL, 10-30 mL, 1-20 mL, or 1-10 mL, typically 10-50 mL of an anesthetic/vasoconstrictor formulation can be administered subcutaneously for the treatment of an ECM-mediated disease or condition, such as cellulite. The administration can be subsequent, simultaneous or intermittent with administration of an modified MMP and/or high-calcium containing composition.

2. Dispersion Agent

Compositions of modified MMP polypeptides, for example csMMPs, provided herein also can be co-formulated or co-administered with a dispersion agent. The dispersion agent also can be co-formulated or co-administered with other pharmacological agents, such as anesthetics, vasoconstrictors, or other biologic agents. Exemplary of dispersion agents are glycosaminoglycanases that open channels in the interstitial space through degradation of glycosaminoglycans. These channels can remain relatively open for a period of 24-48 hours depending on dose and formulation. Such channels can be used to facilitate the diffusion of exogenously added molecules such as fluids, small molecules, proteins (such as matrix degrading enzymes), nucleic acids and gene therapy vectors and other molecules less than about 500 nm in size. In addition, it is thought that the formation of such channels can facilitate bulk fluid flow within an interstitial space, which can in turn promote the dispersion or movement of a solute (such as a detectable molecule or other diagnostic agent, an anesthetic or other tissue-modifying agent, a pharmacologic or pharmaceutically effective agent, or a cosmetic or other esthetic agent) that is effectively carried by the fluid in a process sometimes referred to as “convective transport” or simply convection. Such convective transport can substantially exceed the rate and cumulative effects of molecular diffusion and can thus cause the therapeutic or other administered molecule to more rapidly and effectively perfuse a tissue. Furthermore, when an agent, such as a csMMP, anesthetic or other agent, is co-formulated or co-administered with a glycosaminoglycanase and both are injected into a relatively confined local site, such as a site of non-intravenous parenteral administration (e.g., intradermal, subcutaneous, intramuscular, or into or around other internal tissues, organs or other relatively confined spaces within the body), then the fluid associated with the administered dose can both provide a local driving force (i.e. hydrostatic pressure) as well as lower impedance to flow (by opening channels within the interstitial matrix), both of which could increase fluid flow, and with it convective transport of the therapeutic agent or other molecule contained within the fluid. As a result, the use of glycosaminoglycanases can have substantial utility for improving the bioavailability as well as manipulating other pharmacokinetic and/or pharmacodynamic characteristics of co-formulated or co-administered agents, such as matrix degrading enzymes.

Hyaluronidases

Exemplary of glycosaminoglycanases are hyaluronidases. Hyaluronidases are a family of enzymes that degrade hyaluronic acid. By catalyzing the hydrolysis of hyaluronic acid, a major constituent of the interstitial barrier, hyaluronidase lowers the viscosity of hyaluronic acid, thereby increasing tissue permeability. There are three general classes of hyaluronidases: Mammalian-type hyaluronidases, (EC 3.2.1.35; e.g., SEQ ID NOS:93-97, 106-121) which are endo-beta-N-acetylhexosaminidases with both hydrolytic and transglycosidase activities, and can degrade hyaluronan and chondroitin sulfates (CS), generally C4-S and C6-S, with tetrasaccharides and hexasaccharides as the major end products; Bacterial hyaluronidases (EC 4.2.99.1; e.g., SEQ ID NOS:122-129), which are endo-beta-N-acetylhexosaminidases that operate by a beta elimination reaction to degrade hyaluronan and to various extents, CS and DS, to yield primarily disaccharide end products; and Hyaluronidases (EC 3.2.1.36) from leeches, other parasites, and crustaceans that are endo-beta-glucuronidases that generate tetrasaccharide and hexasaccharide end products through hydrolysis of the beta 1-3 linkage.

There are six hyaluronidase-like genes in the human genome, HYAL1 (SEQ ID NO:93), HYAL2 (SEQ ID NO:94), HYAL3 (SEQ ID NO:95), HYAL4 (SEQ ID NO:96), PH20/SPAM1 (SEQ ID NO:97) and one expressed pseudogene, HYALP1. Among hyaluronidases, PH20 is the prototypical neutral active enzyme, while the others exhibit no catalytic activity towards hyaluronan or any known substrates, or are active only under acidic pH conditions. The hyaluronidase-like enzymes can also be characterized by those which are generally locked to the plasma membrane via a glycosylphosphatidyl inositol anchor such as human HYAL2 and human PH20 (Danilkovitch-Miagkova et al. (2003) Proc. Natl. Acad. Sci. U.S.A. 100(8):4580-4585), and those which are generally soluble such as human HYAL1 (Frost et al. (1997) Biochem. Biophys. Res. Commun. 236(1):10-15). N-linked glycosylation of some hyaluronidases can be very important for their catalytic activity and stability. While altering the type of glycan modifying a glycoprotein can have dramatic effects on a protein's antigenicity, structural folding, solubility, and stability, many enzymes are not thought to require glycosylation for optimal enzyme activity. Hyaluronidases are, therefore, unique in this regard, in that removal of N-linked glycosylation can result in near complete inactivation of the hyaluronidase activity. For such hyaluronidases, the presence of N-linked glycans is critical for generating an active enzyme.

Human PH20 (also known as sperm surface protein PH20) is naturally involved in sperm-egg adhesion and aids penetration by sperm of the layer of cumulus cells by digesting hyaluronic acid. The PH20 mRNA transcript (corresponding to nucleotides 1058-2503 of the sequence set forth in SEQ ID NO:98) is normally translated to generate a 509 amino acid precursor protein containing a 35 amino acid signal sequence at the N-terminus (amino acid residue positions 1-35) and a 19 amino acid GPI anchor at the C-terminus (corresponding to amino acid residues 491-509). The precursor sequence is set forth in SEQ ID NO:97. An mRNA transcript containing a mutation of C to T at nucleotide position 2188 of the sequence of nucleic acids set forth in SEQ ID NO:98 also exists and is a silent mutation resulting in the translated product set forth in SEQ ID NO:97. The mature PH20 is, therefore, a 474 amino acid polypeptide corresponding to amino acids 36-509 of the sequence of amino acids set forth in SEQ ID NO:97. There are potential N-linked glycosylation sites required for hyaluronidases activity at N82, N166, N235, N254, N368, N393, N490 of human PH20 exemplified in SEQ ID NO:97. Disulfide bonds form between the cysteine residues C60 and C351 and between C224 and C238 (corresponding to amino acids set forth in SEQ ID NO:97) to form the core hyaluronidase domain. Additional cysteine residues are required in the carboxy terminus for neutral enzyme catalytic activity such that amino acids 36 to 464 of SEQ ID NO:97 contain the minimally active human PH20 hyaluronidase domain.

Soluble forms of recombinant human PH20 have been produced and can be used in the methods described herein for co-administration or co-formulation with csMMPs, activating or deactivating formulations, anesthetics, vasoconstrictors, other pharmacologic or therapeutic agents, or combinations thereof, to permit the diffusion into tissues. The production of such soluble forms of PH20 is described in related application Ser. No. 11/065,716 (now U.S. Pat. No. 7,871,607) and Ser. No. 11/238,171. Soluble forms include, but are not limited to, any having C-terminal truncations to generate polypeptides containing amino acid 1 to amino acid 442, 443, 444, 445, 446 and 447 of the sequence of amino acids set forth in SEQ ID NOS:100-105. Examples of such polypeptides are those generated from a nucleic acid molecule encoding amino acids 1-482 set forth in SEQ ID NO:99. Resulting purified rHuPH20 can be heterogenous due to peptidases present in the culture medium upon production and purification. Generally, soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g., DG44 CHO cells).

The soluble PH20 can be administered by any suitable route as described elsewhere herein. Typically, administration is by parenteral administration, such as by intradermal, intramuscular, subcutaneous or intravascular administration. The compounds provided herein can be formulated for parenteral administration by injection, e.g., by bolus injection or continuous infusion. Formulations for injection can be presented in unit dosage form, e.g., in ampoules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use. For example, provided herein are parenteral formulations containing an effective amount of soluble PH20, such as 10 Units to 500,000 Units, 100 Units to 100,000 Units, 500 Units to 50,000 Units, 1000 Units to 10,000 Units, 5000 Units to 7500 Units, 5000 Units to 50,000 Units, or 1,000 Units to 10,000 Units, generally 10,000 to 50,000 Units, in a stabilized solution or suspension or a lyophilized from. The formulations can be provided in unit-dose forms such as, but not limited to, ampoules, syringes and individually packaged tablets or capsules. The dispersing agent can be administered alone, or with other pharmacologically effective agents in a total volume of 1-100 mL, 1-50 mL, 10-50 mL, 10-30 mL, 1-20 mL, or 1-10 mL, typically 10-50 mL.

In one example of a combination therapy, it is contemplated herein that an anesthetic, vasoconstrictor and dispersion agent are co-administered or co-formulated with a csMMP to be administered subsequently, simultaneously or intermittently therewith. An exemplary formulation is one containing lidocaine, epinephrine and a soluble PH20, for example, a soluble PH20 set forth in SEQ ID NO:100. Soluble PH20 can be mixed directly with lidocaine (Xylocalne®), and optionally with epinephrine. The formulation can be prepared in a unit dosage form, such as in a syringe. For example, the lidocaine/epinephrine/soluble PH20 formulation can be provided in a volume such as 1-100 mL, 1-50 mL, 10-50 mL, 10-30 mL, 1-20 mL, or 1-10 mL, typically 10-50 mL, prepackaged in a syringe for use.

In the combination therapies, the other pharmacologic agents, such as a lidocaine/epinephrine/soluble PH20 formulation, can be co-administered together with or in close temporal proximity to the administration of a modified MMP and/or calcium-containing composition. Typically, it is preferred that an anesthetic and/or dispersion agent be administered shortly before (e.g., 5 to 60 minutes before) or, for maximal convenience, together with the pharmacologic agent. As will be appreciated by those of skill in the art, the desired proximity of co-administration depends in significant part on the effective half-lives of the agents in the particular tissue setting, and the particular disease being treated, and can be readily optimized by testing the effects of administering the agents at varying times in suitable models, such as in suitable animal models.

### J. EXAMPLES

The following examples are included for illustrative purposes only and are not intended to limit the scope of the invention.

### Example 1

### Generation and Expression of hMMP-1 and hMMP-1 Mutant Library

A. Generation of MMP-1 Library of Single Amino Acid Variants

A human matrix metalloprotease 1 (hMMP-1) library was created by cloning DNA encoding human MMP-1 into a plasmid followed by mutagenesis, transformation and protein expression/isolation. The library was created by introducing mutations in a parent human MMP-1 DNA sequence having the sequence of nucleotides set forth in SEQ ID NO:3, which encodes the inactive zymogen proMMP-1 (set forth in SEQ ID NO:2), to generate single amino acid variants of MMP-1 across the catalytic domain and proline rich linker domain of the polypeptide. The hMMP-1 library was designed to contain at least 15 amino acid substitutions (replacements) at each of 178 amino acids positions within the catalytic domain (amino acids 81-242 of SEQ ID NO:2) and the linker region (amino acids 243-258 of SEQ ID NO:2) of human MMP-1. The cDNA encoding each individual hMMP-1 mutant was generated by changing the wild-type codon encoding each of the 178 amino acids positions to a codon encoding the desired amino acid substitution. Table 9 depicts the codon change and the resulting amino acid substitutions (replacements) at each of the amino acid positions.

The mutant MMP-1s have a sequence of nucleotides that is substantially the same as set forth in SEQ ID NO:3, except that the respective wild-type codon is substituted as set forth in Table 9. The encoded amino acid sequence of the mutant MMPs also is substantially the same as set forth in SEQ ID NO:2), except that the polypeptide contains the single amino acid replacement compared to wild-type MMP-1 set forth in SEQ ID NO:2. The DNA encoding each individual library member was generated according to standard DNA synthesis protocols and protein was expressed using routine molecular biology techniques. Briefly, the DNA was ligated into vector pET303CTHis (Invitrogen, SEQ ID NO:90) using routine molecular biology techniques.

TABLE 9
Codons encoding each amino acid substitution

| Mutation | Codon |
|---|---|
| F81C | TGT |
| F81E | GAG |
| F81I | ATT |
| F81L | CTG |
| F81P | CCT |
| F81S | TCT |
| F81A | GCG |
| F81M | ATG |
| F81G | GGG |
| F81T | ACG |
| F81Q | CAG |
| F81R | CGT |
| F81W | TGG |
| F81H | CAT |
| F81V | GTG |
| V82I | ATT |
| V82C | TGT |
| V82A | GCG |
| V82P | CCG |
| V82Y | TAT |
| V82M | ATG |
| V82Q | CAG |
| V82F | TTT |
| V82W | TGG |
| V82N | AAT |
| V82R | CGT |
| V82G | GGT |
| V82S | TCG |
| V82L | TTG |
| V82T | ACT |
| L83A | GCG |
| L83C | TGT |
| L83D | GAT |
| L83E | GAG |
| L83G | GGT |
| L83H | CAT |
| L83I | ATT |
| L83M | ATG |
| L83P | CCG |
| L83Q | CAG |
| L83R | CGG |
| L83S | AGT |
| L83T | ACG |
| L83W | TGG |
| L83Y | TAT |
| T84V | GTT |
| T84E | GAG |
| T84H | CAT |
| T93G | GGG |
| T93K | AAG |
| T93E | GAG |
| H94L | CTG |
| H94S | TCG |
| H94M | ATG |
| H94R | CGG |
| H94E | GAG |
| H94I | ATT |
| H94D | GAT |
| H94P | CCG |
| H94A | GCG |
| H94N | AAT |
| H94F | TTT |
| H94G | GGG |
| H94T | ACT |
| H94V | GTG |
| H94W | TGG |
| L95E | GAG |
| L95Y | TAT |
| L95R | CGG |
| L95A | GCT |
| L95G | GGG |
| L95K | AAG |
| L95S | AGT |
| L95T | ACG |
| L95H | CAT |
| L95W | TGG |
| L95V | GTG |
| L95C | TGT |
| L95P | CCT |
| L95D | GAT |
| L95I | ATT |
| T96E | GAG |
| T96R | CGG |
| T96P | CCG |
| T96S | TCG |
| T96A | GCG |
| T96L | TTG |
| T96W | TGG |
| T96N | AAT |
| T96G | GGT |
| T96F | TTT |
| T96Q | CAG |
| T96H | CAT |
| T96V | GTT |
| T96I | ATT |
| T96C | TGT |
| L106T | ACG |
| L106V | GTG |
| L106H | CAT |
| L106F | TTT |
| L106I | ATT |
| L106C | TGT |
| L106S | TCT |
| P107L | TTG |
| P107W | TGG |
| P107T | ACT |
| P107S | TCG |
| P107R | CGG |
| P107Y | TAT |
| P107M | ATG |
| P107V | GTG |
| P107D | GAT |
| P107A | GCG |
| P107C | TGT |
| P107K | AAG |
| P107F | TTT |
| P107I | ATT |
| P107G | GGT |
| R108P | CCT |
| R108G | GGT |
| R108T | ACG |
| R108E | GAG |
| R108A | GCG |
| R108Y | TAT |
| R108K | AAG |
| R108C | TGT |
| R108S | TCT |
| R108F | TTT |
| R108W | TGG |
| R108I | ATT |
| R108L | CTT |
| R108N | AAT |
| R108V | GTT |
| A109S | TCG |
| A109R | CGG |
| A109T | ACG |
| A109W | TGG |
| A109I | ATT |
| A109Q | CAG |
| A109N | AAT |
| A109Y | TAT |
| A109G | GGG |
| A109M | ATG |
| A109D | GAT |
| F119L | TTG |
| F119N | AAT |
| F119S | AGT |
| F119C | TGT |
| F119P | CCG |
| F119W | TGG |
| F119K | AAG |
| F119H | CAT |
| F119A | GCG |
| F119V | GTT |
| F119Y | TAT |
| F119E | GAG |
| Q120K | AAG |
| Q120N | AAT |
| Q120A | GCG |
| Q120V | GTG |
| Q120D | GAT |
| Q120R | CGG |
| Q120P | CCT |
| Q120W | TGG |
| Q120Y | TAT |
| Q120C | TGT |
| Q120H | CAT |
| Q120T | ACT |
| Q120M | ATG |
| Q120E | GAG |
| Q120G | GGT |
| L121E | GAG |
| L121Q | CAG |
| L121P | CCT |
| L121R | CGG |
| L121C | TGT |
| L121G | GGG |
| L121K | AAG |
| L121F | TTT |
| L121I | ATT |
| L121S | TCG |
| L121V | GTT |
| L121H | CAT |
| L121T | ACT |
| L121A | GCT |
| L121N | AAT |
| W122R | CGT |
| W122A | GCG |
| W122N | AAT |
| W122P | CCG |
| W122T | ACG |
| W122L | CTT |
| T131R | CGT |
| T131Y | TAT |
| T131M | ATG |
| K132G | GGT |
| K132V | GTG |
| K132L | TTG |
| K132A | GCT |
| K132P | CCG |
| K132F | TTT |
| K132R | CGG |
| K132I | ATT |
| K132H | CAT |
| K132S | TCT |
| K132M | ATG |
| K132D | GAT |
| K132T | ACT |
| K132Y | TAT |
| K132E | GAG |
| V133G | GGG |
| V133E | GAG |
| V133T | ACT |
| V133N | AAT |
| V133A | GCG |
| V133H | CAT |
| V133P | CCG |
| V133K | AAG |
| V133R | CGG |
| V133L | CTT |
| V133W | TGG |
| V133C | TGT |
| V133D | GAT |
| V133M | ATG |
| V133S | AGT |
| S134V | GTT |
| S134H | CAT |
| S134P | CCT |
| S134G | GGG |
| S134N | AAT |
| S134R | CGT |
| S134L | CTG |
| S134Q | CAG |
| S134E | GAG |
| S134Y | TAT |
| S134A | GCG |
| S134K | AAG |
| S134D | GAT |
| S134T | ACG |
| S134C | TGT |
| F144N | AAT |
| F144C | TGT |
| F144G | GGT |
| F144T | ACT |
| F144Q | CAG |
| F144H | CAT |
| F144V | GTG |
| V145A | GCG |
| V145T | ACG |
| V145L | CTG |
| V145P | CCG |
| V145K | AAG |
| V145N | AAT |
| V145D | GAT |
| V145H | CAT |
| V145R | CGG |
| V145Q | CAG |
| V145S | TCT |
| V145G | GGG |
| V145W | TGG |
| V145C | TGT |
| V145E | GAG |
| R146T | ACG |
| R146L | CTG |
| R146N | AAT |
| R146H | CAT |
| R146Q | CAG |
| R146K | AAG |
| R146C | TGT |
| R146S | AGT |
| R146D | GAT |
| R146A | GCT |
| R146Y | TAT |
| R146P | CCT |
| R146V | GTT |
| R146E | GAG |
| R146F | TTT |
| G147R | CGT |
| G147F | TTT |
| G147I | ATT |
| G147L | CTG |
| G147A | GCG |
| G147E | GAG |
| G147H | CAT |
| G147W | TGG |
| G147T | ACG |
| G147C | TGT |
| G147S | TCT |
| G157L | TTG |
| G157N | AAT |
| G157Y | TAT |
| G157S | TCG |
| G157T | ACG |
| G157A | GCT |
| G157Q | CAG |
| G157P | CCG |
| G157V | GTG |
| G157M | ATG |
| P158S | TCT |
| P158Y | TAT |
| P158R | CGG |
| P158L | CTT |
| P158V | GTG |
| P158C | TGT |
| P158A | GCG |
| P158W | TGG |
| P158I | ATT |
| P158F | TTT |
| P158Q | CAG |
| P158T | ACT |
| P158G | GGT |
| P158K | AAG |
| P158N | AAT |
| P158D | GAT |
| G159R | CGG |
| G159S | AGT |
| G159Q | CAG |
| G159P | CCT |
| G159V | GTG |
| G159K | AAG |
| G159A | GCG |
| G159Y | TAT |
| G159E | GAG |
| G159T | ACG |
| G159M | ATG |
| G159I | ATT |
| G159W | TGG |
| G159L | CTG |
| G159C | TGT |
| G160A | GCG |
| G160H | CAT |
| G160N | AAT |
| G160W | TGG |
| G160R | CGG |
| G160P | CCG |
| G160I | ATT |
| P170R | CGG |
| P170I | ATT |
| P170T | ACG |
| P170F | TTT |
| P170Q | CAG |
| P170G | GGG |
| P170S | TCT |
| P170H | CAT |
| P170C | TGT |
| P170M | ATG |
| P170K | AAG |
| P170W | TGG |
| P170D | GAT |
| P170A | GCG |
| G171S | TCT |
| G171M | ATG |
| G171N | AAT |
| G171P | CCT |
| G171R | CGG |
| G171Y | TAT |
| G171A | GCT |
| G171Q | CAG |
| G171H | CAT |
| G171L | CTT |
| G171W | TGG |
| G171C | TGT |
| G171K | AAG |
| G171E | GAG |
| G171D | GAT |
| I172Y | TAT |
| I172T | ACG |
| I172P | CCT |
| I172A | GCG |
| I172L | CTT |
| I172Q | CAG |
| I172E | GAG |
| I172C | TGT |
| I172M | ATG |
| I172D | GAT |
| I172V | GTT |
| I172R | CGT |
| I172G | GGG |
| I172W | TGG |
| I172N | AAT |
| G173C | TGT |
| G173L | CTG |
| G173K | AAG |
| G173W | TGG |
| E182Q | CAG |
| E182W | TGG |
| E182M | ATG |
| E182G | GGT |
| R183P | CCT |
| R183K | AAG |
| R183W | TGG |
| R183E | GAG |
| R183A | GCT |
| R183T | ACG |
| R183L | CTT |
| R183N | AAT |
| R183H | CAT |
| R183V | GTG |
| R183C | TGT |
| R183M | ATG |
| R183I | ATT |
| R183G | GGT |
| R183S | TCT |
| W184G | GGG |
| W184H | CAT |
| W184L | CTG |
| W184E | GAG |
| W184P | CCT |
| W184N | AAT |
| W184A | GCG |
| W184T | ACT |
| W184R | CGG |
| W184Q | CAG |
| W184V | GTG |
| W184S | TCT |
| W184M | ATG |
| W184I | A1T |
| W184F | TTT |
| T185R | CG1 |
| T185Y | TAT |
| T185W | TGG |
| T185H | CAT |
| T185G | GGG |
| T185P | CCT |
| T185S | TCG |
| T185V | GTT |
| T185Q | CAG |
| T185N | AAT |
| T185C | TGT |
| T185L | CTT |
| T185A | GCG |
| T185E | GAG |
| R195S | TCT |
| R195A | GCT |
| R195D | GAT |
| R195P | CCT |
| R195Y | TAT |
| R195E | GAG |
| R195V | GTG |
| V196T | ACG |
| V196D | GAT |
| V196G | GGG |
| V196E | GAG |
| V196A | GCG |
| V196S | AGT |
| V196Q | CAG |
| V196P | CCG |
| V196R | CGT |
| V196H | CAT |
| V196Y | TAT |
| V196I | ATT |
| V196L | CTG |
| V196K | AAG |
| V196M | ATG |
| A197G | GGT |
| A197S | AGT |
| A197L | CTT |
| A197P | CCG |
| A197V | GTG |
| A197Y | TAT |
| A197Q | CAG |
| A197R | CGG |
| A197T | ACT |
| A197I | ATT |
| A197H | CAT |
| A197E | GAG |
| A197W | TGG |
| A197N | AAT |
| A197C | TGT |
| A198T | ACG |
| A198K | AAG |
| A198S | TCG |
| A198H | CAT |
| A198G | GGT |
| A198E | GAG |
| A198P | CCG |
| A198L | TTG |
| A198R | CGT |
| A198V | GTT |
| A198M | ATG |
| S208K | AAG |
| S208N | AAT |
| S208F | TTT |
| S208Q | CAG |
| 5208W | TGG |
| S208T | ACG |
| S208E | GAG |
| S208C | TGT |
| S208R | CGT |
| S208L | CTT |
| H209T | ACG |
| H209Y | TAT |
| H209R | CGG |
| H209Q | CAG |
| H209A | GCT |
| H209G | GGG |
| H209N | AAT |
| H209P | CCT |
| H209W | TGG |
| H209V | GTT |
| H209D | GAT |
| H209S | AGT |
| H209F | TTT |
| H209L | CTG |
| H209C | TGT |
| S210C | TGT |
| S210G | GGT |
| S210I | ATT |
| S210R | CGT |
| S210L | CTG |
| S210V | GTG |
| S210H | CAT |
| S210N | AAT |
| S210F | TTT |
| S210P | CCG |
| S210W | TGG |
| S210Q | CAG |
| S210T | ACG |
| S210K | AAG |
| S210A | GCG |
| T211P | CCG |
| T211R | CGT |
| T211K | AAG |
| T211G | GGG |
| T211M | ATG |
| T211N | AAT |
| T211V | GTG |
| T211H | CAT |
| S220N | AAT |
| Y221W | TGG |
| Y221K | AAG |
| Y221Q | CAG |
| Y221C | TGT |
| Y221N | AAT |
| Y221P | CCT |
| Y221V | GTT |
| Y221A | GCG |
| Y221G | GGG |
| Y221R | CGG |
| Y221S | TCG |
| Y221M | ATG |
| Y221T | ACG |
| Y221L | CTT |
| Y221E | GAG |
| T222L | TTG |
| T222Y | TAT |
| T222R | CGT |
| T222V | GTT |
| T222P | CCT |
| T222S | AGT |
| T222A | GCT |
| T222H | CAT |
| T222G | GGG |
| T222M | ATG |
| T222F | TTT |
| T222C | TGT |
| T222I | ATT |
| T222N | AAT |
| T222W | TGG |
| T222D | GAT |
| F223L | TTG |
| F223T | ACG |
| F223C | TGT |
| F223R | CGT |
| F223N | AAT |
| F223P | CCT |
| F223E | GAG |
| F223G | GGG |
| F223Q | CAG |
| F223A | GCG |
| F223S | TCT |
| F223Y | TAT |
| F223H | CAT |
| F223K | AAG |
| F223M | ATG |
| S224G | GGG |
| D233V | GTG |
| D233M | ATG |
| D233L | CTG |
| D233K | AAG |
| D233I | ATT |
| I234A | GCT |
| I234T | ACG |
| I234V | GTT |
| I234W | TGG |
| I234E | GAG |
| I234G | GGT |
| I234L | CTT |
| I234H | CAT |
| I234M | ATG |
| I234N | AAT |
| I234Y | TAT |
| I234P | CCT |
| I234D | GAT |
| I234Q | CAG |
| I234C | TGT |
| D235H | CAT |
| D235G | GGG |
| D235A | GCG |
| D235P | CCG |
| D235L | CTT |
| D235V | GTG |
| D235E | GAG |
| D235R | CGT |
| D235Q | CAG |
| D235T | ACG |
| D235C | TGT |
| D235S | TCG |
| D235N | AAT |
| D235Y | TAT |
| D235I | ATT |
| G236M | ATG |
| G236R | CGG |
| G236D | GAT |
| G236S | TCT |
| G236T | ACT |
| G236C | TGT |
| G236K | AAG |
| G236E | GAG |
| G236P | CCG |
| G236I | ATT |
| G236Y | TAT |
| G236L | CTG |
| G236V | GTT |
| N246V | GTT |
| N246Q | CAG |
| N246Y | TAT |
| N246C | TGT |
| N246I | ATT |
| N246L | TTG |
| N246S | TCT |
| N246T | ACT |
| N246K | AAG |
| N246D | GAT |
| P247A | GCG |
| P247D | GAT |
| P247E | GAG |
| P247F | TTT |
| P247G | GGG |
| P247H | CAT |
| P247I | ATT |
| P247K | AAG |
| P247L | CTG |
| P247N | AAT |
| P247Q | CAG |
| P247R | CGT |
| P247S | TCG |
| P247T | ACG |
| P247V | GTT |
| V248W | TGG |
| V248L | CTG |
| V248Q | CAG |
| V248M | ATG |
| V248Y | TAT |
| V248G | GGG |
| V248C | TGT |
| V248R | CGG |
| V248A | GCG |
| V248H | CAT |
| V248I | ATT |
| V248T | ACT |
| V248K | AAG |
| V248S | TCG |
| V248F | TTT |
| V248E | GAG |
| Q249T | ACT |
| Q249W | TGG |
| Q249R | CGG |
| Q249E | GAG |
| Q249A | GCT |
| Q249P | CCG |
| Q249C | TGT |
| T84L | TTG |
| T84D | GAT |
| T84R | CGG |
| T84I | ATT |
| T84S | TCT |
| T84G | GGT |
| T84Q | CAG |
| T84P | CCT |
| T84A | GCG |
| T84C | TGT |
| T84Y | TAT |
| T84F | TTT |
| E85L | CTG |
| E85Q | CAG |
| E85P | CCT |
| E85T | ACT |
| E85K | AAG |
| E85M | ATG |
| E85G | GGT |
| E85R | CGT |
| E85S | TCT |
| E85C | TGT |
| E85Y | TAT |
| E85A | GCG |
| E85N | AAT |
| E85V | GTG |
| E85F | TTT |
| G86L | CTT |
| G86P | CCG |
| G86I | ATT |
| G86T | ACT |
| G86H | CAT |
| G86D | GAT |
| G86N | AAT |
| G86S | AGT |
| G86K | AAG |
| G86W | TGG |
| G86Y | TAT |
| G86V | GTT |
| G86C | TGT |
| G86M | ATG |
| G86F | TTT |
| N87M | ATG |
| N87L | CTG |
| N87P | CCG |
| N87V | GTT |
| N87R | CGT |
| N87F | TTT |
| Y97R | CGT |
| Y97V | GTG |
| Y97A | GCT |
| Y97P | CCT |
| Y97L | CTT |
| Y97T | ACG |
| Y97K | AAG |
| Y97W | TGG |
| Y97H | CAT |
| Y97S | TCG |
| Y97E | GAG |
| Y97D | GAT |
| Y97N | AAT |
| Y97G | GGT |
| Y97Q | CAG |
| R98H | CAT |
| R98K | AAG |
| R98C | TGT |
| R98L | CTG |
| R98M | ATG |
| R98F | TTT |
| R98W | TGG |
| R98Y | TAT |
| R98P | CCT |
| R98E | GAG |
| R98A | GCG |
| R98G | GGG |
| R98V | GTT |
| R98S | TCG |
| R98D | GAT |
| I99C | TGT |
| I99E | GAG |
| I99G | GGG |
| I99H | CAT |
| I99N | AAT |
| I99P | CCT |
| I99T | ACG |
| I99V | GTT |
| I99A | GCG |
| I99F | TTT |
| I99L | CTG |
| I99R | CGT |
| I99S | TCG |
| I99Q | CAG |
| I99W | TGG |
| I99Y | TAT |
| E100V | GTT |
| E100P | CCG |
| A109V | GTT |
| A109E | GAG |
| A109L | CTT |
| A109H | CAT |
| D110P | CCT |
| D110F | TTT |
| D110Q | CAG |
| D110R | CGG |
| D110M | ATG |
| D110H | CAT |
| D110I | ATT |
| D110L | CTT |
| D110V | GTG |
| D110T | ACG |
| D110S | TCG |
| D110Y | TAT |
| D110G | GGT |
| D110C | TGT |
| D110A | GCG |
| V111E | GAG |
| V111A | GCT |
| V111S | TCT |
| V111W | TGG |
| V111G | GGT |
| V111Y | TAT |
| V111P | CCG |
| V111L | CTG |
| V111D | GAT |
| V111K | AAG |
| V111T | ACT |
| V111Q | CAG |
| V111I | ATT |
| V111C | TGT |
| V111R | CGT |
| D112A | GCG |
| D112M | ATG |
| D112V | GTT |
| D112R | CGG |
| D112K | AAG |
| D112P | CCT |
| D112Q | CAG |
| D112F | TTT |
| D112G | GGG |
| D112C | TGT |
| D112W | TGG |
| D112T | ACT |
| D112H | CAT |
| D112S | TCT |
| W122G | GGG |
| W122S | TCG |
| W122V | GTT |
| W122H | CAT |
| W122F | TTT |
| W122Y | TAT |
| W122K | AAG |
| W122Q | CAG |
| W122E | GAG |
| S123D | GAT |
| S123L | TTG |
| S123A | GCT |
| S123C | TGT |
| S123I | ATT |
| S123K | AAG |
| S123N | AAT |
| S123F | TTT |
| S123Y | TAT |
| S123M | ATG |
| S123H | CAT |
| S123R | CGG |
| S123W | TGG |
| S123T | ACG |
| S123P | CCT |
| S123G | GGG |
| S123Q | CAG |
| S123V | GTT |
| N124G | GGT |
| N124C | TGT |
| N124V | GTG |
| N124L | CTT |
| N124T | ACG |
| N124R | CGT |
| N124M | ATG |
| N124S | TCG |
| N124P | CCT |
| N124A | GCG |
| N124K | AAG |
| N124F | TTT |
| N124W | TGG |
| N124I | ATT |
| N124D | GAT |
| V125G | GGG |
| V125Q | CAG |
| V125S | TCG |
| V125P | CCG |
| V125M | ATG |
| V125Y | TAT |
| E135V | GTT |
| E135M | ATG |
| E135S | TCG |
| E135D | GAT |
| E135T | ACG |
| E135L | CTG |
| E135A | GCG |
| E135W | TGG |
| E135F | TTT |
| E135P | CCG |
| E135R | CGG |
| E135N | AAT |
| E135H | CAT |
| E135Q | CAG |
| E135I | ATT |
| G136V | GTG |
| G136W | TGG |
| G136D | GAT |
| G136M | ATG |
| G136N | AAT |
| G136A | GCG |
| G136L | TTG |
| G136C | TGT |
| G136P | CCG |
| G136T | ACG |
| G136R | CGT |
| G136S | TCG |
| G136I | ATT |
| G136H | CAT |
| G136E | GAG |
| Q137A | GCT |
| Q137R | CGG |
| Q137G | GGG |
| Q137K | AAG |
| Q137H | CAT |
| Q137P | CCT |
| Q137S | TCG |
| Q137L | CTG |
| Q137W | TGG |
| Q137F | TTT |
| Q137T | ACG |
| Q137C | TGT |
| Q137Y | TAT |
| Q137N | AAT |
| Q137E | GAG |
| A138V | GTT |
| A138L | CTT |
| A138P | CCG |
| G147V | GTT |
| G147Q | CAG |
| G147M | ATG |
| G147P | CCT |
| D148R | CGG |
| D148I | ATT |
| D148T | ACG |
| D148G | GGT |
| D148L | CTG |
| D148V | GTT |
| D148A | GCG |
| D148W | TGG |
| D148P | CCG |
| D148S | TCG |
| D148K | AAG |
| D148E | GAG |
| D148M | ATG |
| D148N | AAT |
| D148C | TGT |
| H149W | TGG |
| H149A | GCG |
| H149L | TTG |
| H149C | TGT |
| H149Q | CAG |
| H149T | ACT |
| H149Y | TAT |
| H149P | CCG |
| H149V | GTT |
| H149R | CGG |
| H149G | GGT |
| H149E | GAG |
| H149S | AGT |
| H149I | ATT |
| H149N | AAT |
| R150S | TCG |
| R150E | GAG |
| R150G | GGG |
| R150M | ATG |
| R150P | CCG |
| R150T | ACG |
| R150W | TGG |
| R150A | GCG |
| R150N | AAT |
| R150K | AAG |
| R150L | TTG |
| R150V | GTT |
| R150D | GAT |
| R150I | ATT |
| G160M | ATG |
| G160C | TGT |
| G160Q | CAG |
| G160V | GTT |
| G160S | AGT |
| G160E | GAG |
| G160L | CTT |
| G160T | ACG |
| N161S | AGT |
| N161C | TGT |
| N161L | TTG |
| N161R | CGT |
| N161G | GGT |
| N161W | TGG |
| N161Y | TAT |
| N161E | GAG |
| N161P | CCT |
| N161T | ACG |
| N161H | CAT |
| N161I | ATT |
| N161V | GTG |
| N161F | TTT |
| N161Q | CAG |
| L162A | GCT |
| L162G | GGG |
| L162C | TGT |
| L162P | CCG |
| L162R | CGG |
| L162I | ATT |
| L162S | TCT |
| L162D | GAT |
| L162M | ATG |
| L162E | GAG |
| L162T | ACT |
| L162Y | TAT |
| L162F | TTT |
| L162W | TGG |
| L162Q | CAG |
| A163R | CGT |
| A163G | GGG |
| A163Y | TAT |
| A163P | CCT |
| A163S | AGT |
| A163L | CTT |
| A163C | TGT |
| A163K | AAG |
| A163V | GTG |
| A163F | TTT |
| G173S | AGT |
| G173A | GCG |
| G173R | AGG |
| G173N | AAT |
| G173T | ACG |
| G173D | GAT |
| G173V | GTT |
| G173F | TTT |
| G173M | ATG |
| G173Y | TAT |
| G173P | CCG |
| G174R | CGT |
| G174A | GCG |
| G174E | GAG |
| G174F | TTT |
| G174H | CAT |
| G174T | ACT |
| G174D | GAT |
| G174S | AGT |
| G174P | CCG |
| G174W | TGG |
| G174V | GTT |
| G174N | AAT |
| G174Y | TAT |
| G174M | ATG |
| G174L | CTT |
| D175I | ATT |
| D175T | ACG |
| D175N | AAT |
| D175V | GTT |
| D175S | TCG |
| D175R | CGG |
| D175G | GGG |
| D175A | GCG |
| D175F | TTT |
| D175C | TGT |
| D175Q | CAG |
| D175Y | TAT |
| D175L | CTG |
| D175H | CAT |
| D175P | CCG |
| D175E | GAG |
| A176F | TTT |
| A176Q | CAG |
| A176V | GTG |
| A176E | GAG |
| A176T | ACT |
| A176C | TGT |
| T185D | GAT |
| N186G | GGG |
| N186A | GCT |
| N186T | ACT |
| N186R | CGT |
| N186L | TTG |
| N186P | CCG |
| N186S | AGT |
| N186V | GTG |
| N186Q | CAG |
| N186H | CAT |
| N186C | TGT |
| N186E | GAG |
| N186F | TTT |
| N186Y | TAT |
| N186D | GAT |
| N187R | CGG |
| N187M | ATG |
| N187S | TCT |
| N187T | ACG |
| N187L | CTG |
| N187W | TGG |
| N187F | TTT |
| N187K | AAG |
| N187I | ATT |
| N187A | GCT |
| N187P | CCG |
| N187D | GAT |
| N187G | GGG |
| N187C | TGT |
| N187H | CAT |
| F188P | CCG |
| F188I | ATT |
| F188N | AAT |
| F188S | AGT |
| F188Q | CAG |
| F188K | AAG |
| F188G | GGG |
| F188W | TGG |
| F188E | GAG |
| F188H | CAT |
| F188D | GAT |
| F188A | GCG |
| F188L | CTT |
| F188R | CGT |
| F188V | GTT |
| R189L | TTG |
| R189G | GGG |
| A198F | TTT |
| A198W | TGG |
| A198Y | TAT |
| A198D | GAT |
| H199I | ATT |
| H199P | CCG |
| H199G | GGT |
| H199N | AAT |
| H199S | TCG |
| H199L | TTG |
| H199M | ATG |
| H199A | GCG |
| H199C | TGT |
| H199K | AAG |
| H199R | CGT |
| H199V | GTG |
| H199W | TGG |
| H199T | ACT |
| H199E | GAG |
| E200P | CCG |
| E200G | GGG |
| E200A | GCT |
| E200T | ACG |
| E200I | ATT |
| E200W | TGG |
| E200R | CGG |
| E200F | TTT |
| E200M | ATG |
| E200D | GAT |
| E200V | GTG |
| E200C | TGT |
| E200S | TCT |
| E200Y | TAT |
| E200N | AAT |
| L201A | GCG |
| L201R | CGG |
| L201E | GAG |
| L201P | CCT |
| L201G | GGT |
| L201V | GTT |
| L201T | ACG |
| L201I | ATT |
| L201S | TCT |
| L201W | TGG |
| L201Q | CAG |
| L201D | GAT |
| L201M | ATG |
| L201K | AAG |
| T211Q | CAG |
| T211S | TCG |
| T211A | GCG |
| T211F | TTT |
| T211D | GAT |
| T211W | TGG |
| T211L | CTG |
| D212E | GAG |
| D212A | GCG |
| D212K | AAG |
| D212R | CGG |
| D212T | ACG |
| D212N | AAT |
| D212G | GGG |
| D212S | TCT |
| D212P | CCG |
| D212Q | CAG |
| D212V | GTT |
| D212L | TTG |
| D212F | TTT |
| D212H | CAT |
| D212Y | TAT |
| I213Q | CAG |
| I213T | ACT |
| I213C | TGT |
| I213P | CCT |
| I213H | CAT |
| I213A | GCG |
| I213V | GTT |
| I213G | GGG |
| I213N | AAT |
| I213L | CTT |
| I213S | AGT |
| I213M | ATG |
| I213R | CGG |
| I213K | AAG |
| I213F | TTT |
| I213D | GAT |
| I213E | GAG |
| G214L | TTG |
| G214Q | CAG |
| G214S | TCT |
| G214T | ACT |
| G214V | GTG |
| G214I | ATT |
| G214R | CGT |
| G214P | CCG |
| G214E | GAG |
| S224T | ACG |
| S224Q | CAG |
| S224R | CGG |
| S224P | CCG |
| S224I | ATT |
| S224V | GTT |
| S224L | TTG |
| S224C | TGT |
| S224K | AAG |
| S224D | GAT |
| S224H | CAT |
| S224M | ATG |
| S224A | GCT |
| S224W | TGG |
| G225D | GAT |
| G225R | CGT |
| G225Q | CAG |
| G225M | ATG |
| G225P | CCT |
| G225W | TGG |
| G225S | TCT |
| G225E | GAG |
| G225V | GTT |
| G225T | ACG |
| G225K | AAG |
| G225N | AAT |
| G225C | TGT |
| G225H | CAT |
| G225A | GCG |
| D226S | TCT |
| D226W | TGG |
| D226R | CGG |
| D226A | GCT |
| D226N | AAT |
| D226T | ACT |
| D226E | GAG |
| D226L | CTT |
| D226P | CCT |
| D226H | CAT |
| D226G | GGT |
| D226I | ATT |
| D226M | ATG |
| D226V | GTG |
| D226C | TGT |
| V227A | GCT |
| V227C | TGT |
| V227D | GAT |
| V227E | GAG |
| G236N | AAT |
| G236F | TTT |
| I237S | TCG |
| I237L | CTG |
| I237R | CGT |
| I237Q | CAG |
| I237K | AAG |
| I237D | GAT |
| I237A | GCG |
| I237T | ACG |
| I237E | GAG |
| I237C | TGT |
| I237G | GGG |
| I237P | CCT |
| I237Y | TAT |
| I237W | TGG |
| I237N | AAT |
| Q238G | GGG |
| Q238H | CAT |
| Q238S | TCG |
| Q238Y | TAT |
| Q238F | TTT |
| Q238E | GAG |
| Q238L | TTG |
| Q238W | TGG |
| Q238P | CCG |
| Q238R | AGG |
| Q238C | TGT |
| Q238N | AAT |
| Q238I | ATT |
| Q238T | ACG |
| Q238K | AAG |
| A239S | TCT |
| A239Q | CAG |
| A239T | ACG |
| A239P | CCT |
| A239V | GTG |
| A239L | CTG |
| A239Y | TAT |
| A239I | ATT |
| A239C | TGT |
| A239G | GGG |
| A239W | TGG |
| A239F | TTT |
| A239K | AAG |
| A239H | CAT |
| A239R | CGT |
| A239D | GAT |
| Q249G | GGT |
| Q249N | AAT |
| Q249K | AAG |
| Q249I | ATT |
| Q249Y | TAT |
| Q249V | GTG |
| Q249L | TTG |
| Q249H | CAT |
| P250L | CTG |
| P250S | TCG |
| P250R | CGG |
| P250Y | TAT |
| P250M | ATG |
| P250F | TTT |
| P250A | GCT |
| P250K | AAG |
| P250G | GGT |
| P250N | AAT |
| P250T | ACT |
| P250W | TGG |
| P250D | GAT |
| P250V | GTG |
| P250Q | CAG |
| I251A | GCG |
| I251Q | CAG |
| I251G | GGG |
| I251L | CTG |
| I251K | AAG |
| I251R | CGT |
| I251E | GAG |
| I251D | GAT |
| I251T | ACG |
| I251C | TGT |
| I251Y | TAT |
| I251P | CCT |
| I251S | TCT |
| I251W | TGG |
| I251V | GTT |
| G252F | TTT |
| G252W | TGG |
| G252A | GCG |
| G252R | CGG |
| G252L | CTT |
| G252E | GAG |
| G252D | GAT |
| G252K | AAG |
| G252S | TCG |
| G252T | ACG |
| N87S | AGT |
| N87I | ATT |
| N87C | TGT |
| N87A | GCG |
| N87G | GGT |
| N87Y | TAT |
| N87E | GAG |
| N87H | CAT |
| N87Q | CAG |
| P88C | TGT |
| P88K | AAG |
| P88W | TGG |
| P88G | GGG |
| P88L | CTG |
| P88Q | CAG |
| P88A | GCG |
| P88T | ACG |
| P88Y | TAT |
| P88R | CGG |
| P88H | CAT |
| P88I | ATT |
| P88V | GTG |
| P88E | GAG |
| P88D | GAT |
| R89V | GTG |
| R89W | TGG |
| R89M | ATG |
| R89A | GCG |
| R89T | ACG |
| R89G | GGG |
| R89S | TCT |
| R89K | AAG |
| R89F | TTT |
| R89Y | TAT |
| R89N | AAT |
| R89H | CAT |
| R89L | TTG |
| R89E | GAG |
| R89P | CCT |
| W90L | TTG |
| W90G | GGG |
| W90P | CCG |
| W90T | ACT |
| W90S | TCG |
| W90V | GTG |
| W90I | ATT |
| W90A | GCT |
| W90F | TTT |
| E100L | CTG |
| E100H | CAT |
| E100D | GAT |
| E100M | ATG |
| E100G | GGT |
| E100W | TGG |
| E100Y | TAT |
| E100R | CGT |
| E100S | TCT |
| E100T | ACG |
| E100F | TTT |
| E100I | ATT |
| E100N | AAT |
| N101M | ATG |
| N101F | TTT |
| N101L | TTG |
| N101V | GTG |
| N101H | CAT |
| N101R | CGG |
| N101C | TGT |
| N101T | ACT |
| N101P | CCT |
| N101W | TGG |
| N101K | AAG |
| N101S | TCG |
| N101D | GAT |
| N101A | GCG |
| N101Y | TAT |
| Y102R | CGT |
| Y102K | AAG |
| Y102V | GTG |
| Y102M | ATG |
| Y102P | CCG |
| Y102N | AAT |
| Y102G | GGG |
| Y102L | CTG |
| Y102D | GAT |
| Y102S | TCG |
| Y102F | TTT |
| Y102A | GCT |
| Y102E | GAG |
| Y102Q | CAG |
| Y102C | TGT |
| T103E | GAG |
| T103D | GAT |
| T103S | AGT |
| T103L | CTG |
| T103V | GTT |
| D112I | ATT |
| D112Y | TAT |
| D112L | TTG |
| H113T | ACT |
| H113L | CTG |
| H113M | ATG |
| H113S | TCG |
| H113N | AAT |
| H113R | AGG |
| H113A | GCT |
| H113E | GAG |
| H113V | GTG |
| H113Y | TAT |
| H113F | TTT |
| H113D | GAT |
| H113W | TGG |
| H113G | GGG |
| H113P | CCG |
| A114E | GAG |
| A114S | TCG |
| A114I | ATT |
| A114P | CCT |
| A114N | AAT |
| A114L | C1T |
| A114T | ACT |
| A114F | TTT |
| A114V | GTT |
| A114G | GGT |
| A114C | TGT |
| A114M | ATG |
| A114R | AGG |
| A114W | TGG |
| A114Q | CAG |
| I115F | TTT |
| I115T | ACT |
| I115H | CAT |
| I115G | GGT |
| I115K | AAG |
| I115E | GAG |
| I115S | AGT |
| I115P | CCT |
| I115C | TGT |
| I115L | CTT |
| I115Q | CAG |
| I115R | CGG |
| I115W | TGG |
| I115V | GTT |
| I115D | GAT |
| V125T | ACG |
| V125A | GCT |
| V125C | TGT |
| V125D | GAT |
| V125W | TGG |
| V125R | CGG |
| V125E | GAA |
| V125F | TTT |
| V125H | CAT |
| T126K | AAG |
| T126V | GTG |
| T126G | GGG |
| T126R | CGG |
| T126L | TTG |
| T126H | CAT |
| T126M | ATG |
| T126P | CCG |
| T126A | GCG |
| T126N | AAT |
| T126E | GAG |
| T126F | TTT |
| T126W | TGG |
| T126Q | CAG |
| T126S | AGT |
| P127C | TGT |
| P127F | TTT |
| P127T | ACG |
| P127E | GAG |
| P127W | TGG |
| P127A | GCT |
| P127S | AGT |
| P127H | CAT |
| P127Q | CAG |
| P127K | AAG |
| P127R | CGG |
| P127I | ATT |
| P127V | GTG |
| P127L | CTG |
| P127M | ATG |
| L128F | TTT |
| L128M | ATG |
| L128T | ACT |
| L128R | CGT |
| L128S | TCG |
| L128G | GGT |
| L128I | ATT |
| L128Q | CAG |
| L128P | CCT |
| A138C | TGT |
| A138T | ACG |
| A138S | TCT |
| A138R | CGT |
| A138G | GGG |
| A138E | GAG |
| A138H | CAT |
| A138M | ATG |
| A138Q | CAG |
| A138I | ATT |
| A138D | GAT |
| A138W | TGG |
| D139R | CGT |
| D139V | GTT |
| D139M | ATG |
| D139C | TGT |
| D139P | CCT |
| D139S | TCT |
| D139L | CTT |
| D139I | ATT |
| D139H | CAT |
| D139A | GCG |
| D139G | GGG |
| D139F | TTT |
| D139N | AAT |
| D139W | TGG |
| D139Y | TAT |
| D139E | GAG |
| I140D | GAT |
| I140K | AAG |
| I140A | GCT |
| I140G | GGG |
| I140C | TGT |
| I140Y | TAT |
| I140V | GTT |
| I140W | TGG |
| I140F | TTT |
| I140H | CAT |
| I140L | CTG |
| I140R | CGG |
| I140E | GAG |
| I140M | ATG |
| I140T | ACT |
| M141E | GAG |
| M141I | ATT |
| M141R | CGG |
| M141T | ACG |
| M141P | CCG |
| R150H | CAT |
| D151R | CGT |
| D151F | TTT |
| D151P | CCG |
| D151W | TGG |
| D151Q | CAG |
| D151L | CTT |
| D151S | TCG |
| D151G | GGT |
| D151A | GCT |
| D151N | AAT |
| D151K | AAG |
| D151Y | TAT |
| D151V | GTT |
| D151T | ACT |
| D151M | ATG |
| N152G | GGG |
| N152C | TGT |
| N152F | TTT |
| N152L | TTG |
| N152P | CCG |
| N152R | CGG |
| N152H | CAT |
| N152T | ACG |
| N152Y | TAT |
| N152K | AAG |
| N152D | GAT |
| N152W | TGG |
| N152I | ATT |
| N152A | GCG |
| N152S | TCT |
| S153I | ATT |
| S153R | CGG |
| S153K | AAG |
| S153C | TGT |
| S153G | GGG |
| S153H | CAT |
| S153L | CTT |
| S153V | GTT |
| S153T | ACG |
| S153P | CCT |
| S153A | GCG |
| S153F | TTT |
| S153D | GAT |
| S153Q | CAG |
| S153Y | TAT |
| P154V | GTT |
| P154W | TGG |
| A163E | GAG |
| A163T | ACG |
| A163Q | CAG |
| A163I | ATT |
| A163N | AAT |
| H164L | CTT |
| H164M | ATG |
| H164K | AAG |
| H164P | CCG |
| H164C | TGT |
| H164R | CGT |
| H164A | GCG |
| H164V | GTG |
| H164S | TCG |
| H164N | AAT |
| H164G | GGG |
| H164F | TTT |
| H164Y | TAT |
| H164Q | CAG |
| H164E | GAG |
| A165W | TGG |
| A165V | GTT |
| A165G | GGG |
| A165K | AAG |
| A165L | TTG |
| A165P | CCT |
| A165Q | CAG |
| A165D | GAT |
| A165H | CAT |
| A165F | TTT |
| A165S | AGT |
| A165T | ACT |
| A165R | CGG |
| A165N | AAT |
| A165M | ATG |
| F166G | GGG |
| F166S | TCG |
| F166L | CTT |
| F166V | GTG |
| F166P | CCT |
| F166N | AAT |
| F166R | CGT |
| F166A | GCG |
| F166K | AAG |
| F166H | CAT |
| F166W | TGG |
| F166I | ATT |
| F166M | ATG |
| A176L | CTG |
| A176P | CCT |
| A176N | AAT |
| A176G | GGT |
| A176S | TCT |
| A176R | CGT |
| A176K | AAG |
| A176D | GAT |
| A176W | TGG |
| H177T | ACG |
| H177P | CCG |
| H177Q | CAG |
| H177A | GCG |
| H177S | TCG |
| H177G | GGG |
| H177W | TGG |
| H177L | CTG |
| H177V | GTT |
| H177I | ATT |
| H177R | CGG |
| H177N | AAT |
| H177Y | TAT |
| H177C | TGT |
| H177D | GAT |
| F178G | GGT |
| F178C | TGT |
| F178W | TGG |
| F178R | CGG |
| F178K | AAG |
| F178S | AGT |
| F178H | CAT |
| F178P | CCT |
| F178V | GTT |
| F178A | GCT |
| F178Q | CAG |
| F178Y | TAT |
| F178I | ATT |
| F178T | ACT |
| F178L | CTG |
| F178E | GAG |
| D179P | CCT |
| D179L | TTG |
| D179E | GAG |
| D179G | GGG |
| D179S | AGT |
| D179A | GCT |
| D179K | AAG |
| D179T | ACT |
| R189K | AAG |
| R189P | CCG |
| R189E | GAG |
| R189V | GTT |
| R189D | GAT |
| R189Y | TAT |
| R189C | TGT |
| R189A | GCT |
| R189H | CAT |
| R189W | TGG |
| R189N | AAT |
| R189T | ACT |
| R189Q | CAG |
| E190A | GCG |
| E190H | CAT |
| E190V | GTG |
| E190P | CCG |
| E190C | TGT |
| E190G | GGT |
| E190R | CGG |
| E190I | ATT |
| E190S | TCG |
| E190T | ACT |
| E190M | ATG |
| E190L | TTG |
| F190K | AAG |
| E190Y | TAT |
| E190D | GAT |
| Y191T | ACT |
| Y191H | CAT |
| Y191G | GGG |
| Y191L | TTG |
| Y191P | CCT |
| Y191Q | CAG |
| Y171K | AAG |
| Y191D | GAT |
| Y191A | GCG |
| Y191W | TGG |
| Y191S | TCT |
| Y191V | GTT |
| Y191E | GAG |
| Y191R | CGT |
| Y191C | TGT |
| N192R | CGG |
| N192L | CTG |
| N192Q | CAG |
| N192P | CCT |
| N192H | CAT |
| L201N | AAT |
| G202T | ACG |
| G202Y | TAT |
| G202E | GAG |
| G202V | GTG |
| G202S | TCT |
| G202L | CTG |
| G202I | ATT |
| G202M | ATG |
| G202H | CAT |
| G202C | TGT |
| G202R | CGT |
| G202P | CCT |
| G202A | GCT |
| G202K | AAG |
| G202D | GAT |
| H203Y | TAT |
| H203E | GAG |
| H203R | CGG |
| H203Q | CAG |
| H203P | CCG |
| H203G | GGG |
| H203T | ACT |
| H203D | GAT |
| H203L | TTG |
| H203N | AAT |
| H203A | GCT |
| H203S | TCT |
| H203V | GTT |
| H203I | ATT |
| H203C | TGT |
| S204R | CGG |
| S204N | AAT |
| S204A | GCG |
| S204T | ACT |
| S204Y | TAT |
| S204V | GTG |
| S204L | CTT |
| S204H | CAT |
| S204D | GAT |
| S204Q | CAG |
| S204G | GGG |
| S204W | TGG |
| S204I | ATT |
| S204K | AAG |
| S204P | CCT |
| L205T | ACG |
| L205D | GAT |
| G214A | GCT |
| G214D | GAT |
| G214F | TTT |
| G214Y | TAT |
| G214M | ATG |
| G214C | TGT |
| A215L | CTG |
| A215Q | CAG |
| A215M | ATG |
| A215G | GGT |
| A215W | TGG |
| A215S | AGT |
| A215T | ACG |
| A215V | GTT |
| A215N | AAT |
| A215P | CCG |
| A215H | CAT |
| A215K | AAG |
| A215I | ATT |
| A215R | CGT |
| A215C | TGT |
| A215D | GAT |
| L216A | GCT |
| L216C | TGT |
| L216D | GAT |
| L216E | GAG |
| L216G | GGG |
| L216I | ATT |
| L216K | AAG |
| L216M | ATG |
| L216P | CCT |
| L216Q | CAG |
| L216R | CGG |
| L216S | TCT |
| L216T | ACT |
| L216V | GTG |
| L216W | TGG |
| M217P | CCT |
| M217Y | TAT |
| M217T | ACG |
| M217C | TGT |
| M217S | AGT |
| M217L | CTG |
| M217N | AAT |
| M217R | CGG |
| M217Q | CAG |
| M217K | AAG |
| M217G | GGG |
| V227K | AAG |
| V227L | CTG |
| V227P | CCT |
| V227S | TCT |
| V227T | ACT |
| V227W | TGG |
| V227Y | TAT |
| V227G | GGG |
| V227H | CAT |
| V227Q | CAG |
| V227R | CGT |
| Q228A | GCT |
| Q228D | GAT |
| Q228E | GAG |
| Q228G | GGT |
| Q228H | CAT |
| Q228K | AAG |
| Q228L | CTG |
| Q228M | ATG |
| Q228N | AAT |
| Q228P | CCG |
| Q228R | CGG |
| Q228S | TCT |
| Q228T | ACG |
| Q228W | TGG |
| Q228Y | TAT |
| L229R | CGG |
| L229A | GCG |
| L229T | ACG |
| L229Q | CAG |
| L229P | CCT |
| L229E | GAG |
| L229W | TGG |
| L229M | ATG |
| L229I | ATT |
| L229G | GGT |
| L229C | TGT |
| L229Y | TAT |
| L229D | GAT |
| L229H | CAT |
| L229V | GTG |
| A230L | TTG |
| A230G | GGT |
| A230W | TGG |
| A230P | CCG |
| A230D | GAT |
| A230R | CGT |
| A230I | ATT |
| I240G | GGG |
| I240Q | CAG |
| I240P | CCG |
| I240R | CGG |
| I240S | TCG |
| I240K | AAG |
| I240V | GTG |
| I240D | GAT |
| I240A | GCG |
| I240C | TGT |
| I240L | CTT |
| I240F | TTT |
| I240Y | TAT |
| I240M | ATG |
| I240T | ACG |
| Y241V | GTT |
| Y241A | GCT |
| Y241G | GGG |
| Y241H | CAT |
| Y241R | CGG |
| Y241P | CCG |
| Y241Q | CAG |
| Y241L | TTG |
| Y241T | ACG |
| Y241S | AGT |
| Y241W | TGG |
| Y241N | AAT |
| Y241M | ATG |
| Y241I | ATT |
| Y241D | GAT |
| G242A | GCG |
| G242F | TTT |
| G242L | CTT |
| G242N | AAT |
| G242P | CCT |
| G242W | TGG |
| G242T | ACG |
| G242R | CGT |
| G242V | GTT |
| G242S | TCG |
| G242I | ATT |
| G242Y | TAT |
| G242H | CAT |
| G242E | GAG |
| G242K | AAG |
| R243P | CCG |
| R243K | AAG |
| R243T | ACG |
| G252P | CCT |
| G252H | CAT |
| G252C | TGT |
| G252V | GTT |
| G252I | ATT |
| P253C | TGT |
| P253G | GGT |
| P253Q | CAG |
| P253I | ATT |
| P253L | CTG |
| P253R | CGG |
| P253A | GCT |
| P253E | GAG |
| P253Y | TAT |
| P253W | TGG |
| P253M | ATG |
| P253V | GTG |
| P253T | ACT |
| P253K | AAG |
| P253N | AAT |
| Q254R | CGT |
| Q254G | GGG |
| Q254W | TGG |
| Q254T | ACT |
| Q254A | GCT |
| Q254F | TTT |
| Q254D | GAT |
| Q254P | CCG |
| Q254L | CTG |
| Q254C | TGT |
| Q254Y | TAT |
| Q254I | ATT |
| Q254E | GAG |
| Q254V | GTG |
| Q254S | TCT |
| T255I | ATT |
| T255Q | CAG |
| T255P | CCG |
| T255R | CGT |
| T255C | TGT |
| T255N | AAT |
| T255S | AGT |
| T255V | GTG |
| T255E | GAG |
| T255G | GGG |
| T255K | AAG |
| T255A | GCT |
| T255F | TTT |
| W90H | CAT |
| W90M | ATG |
| W90R | CGG |
| W90E | GAG |
| W90N | AAT |
| W90Q | CAG |
| E91N | AAT |
| E91R | CGG |
| E91W | TGG |
| E91G | GGG |
| E91V | GTG |
| E91Y | TAT |
| E91C | TGT |
| E91H | CAT |
| E91T | ACG |
| E91S | AGT |
| E91A | GCG |
| E91I | ATT |
| E91D | GAT |
| E91F | TTT |
| E91L | TTG |
| Q92V | GTT |
| Q92Y | TAT |
| Q92L | CTG |
| Q92N | AAT |
| Q92E | GAG |
| Q92I | ATT |
| Q92T | ACT |
| Q92G | GGT |
| Q92P | CCG |
| Q92W | TGG |
| Q92F | TTT |
| Q92S | TCG |
| Q92R | CGG |
| Q92K | AAG |
| Q92A | GCT |
| T93A | GCG |
| T93L | CTT |
| T93M | ATG |
| T93N | AAT |
| T93V | GTG |
| T93I | ATT |
| T93D | GAT |
| T93S | TCG |
| T93R | CGG |
| T93W | TGG |
| T93F | TTT |
| T93P | CCT |
| T103R | CGG |
| T103Y | TAT |
| T103N | AAT |
| T103C | TGT |
| T103Q | CAG |
| T103W | TGG |
| T103P | CCG |
| T103A | GCG |
| T103G | GGG |
| T103K | AAG |
| P104G | GGG |
| P104E | GAG |
| P104T | ACT |
| P104F | TTT |
| P104R | CGT |
| P104D | GAT |
| P104C | TGT |
| P104Q | CAG |
| P104V | GTG |
| P104Y | TAT |
| P104H | CAT |
| P104L | TTG |
| P104S | TCG |
| P104A | GCG |
| P104M | ATG |
| D105A | GCT |
| D105C | TGT |
| D105F | TTT |
| D105G | GGT |
| D105I | ATT |
| D105L | CTG |
| D105M | ATG |
| D105N | AAT |
| D105P | CCT |
| D105R | CGG |
| D105S | TCG |
| D105T | ACG |
| D105V | GTT |
| D105W | TGG |
| D105E | GAG |
| L106P | CCG |
| L106D | GAT |
| L106N | AAT |
| L106G | GGT |
| L106M | ATG |
| L106A | GCT |
| L106R | CGG |
| L106Y | TAT |
| E116A | GCG |
| E116C | TGT |
| E116D | GAT |
| E116F | TTT |
| E116G | GGT |
| E116H | CAT |
| E116I | ATT |
| E116K | AAG |
| E116L | CTG |
| E116M | ATG |
| E116N | AAT |
| E116P | CCG |
| E116Q | CAG |
| E116R | AGG |
| E116S | TCT |
| K117H | CAT |
| K117T | ACG |
| K117Q | CAG |
| K117E | GAG |
| K117A | GCG |
| K117F | TTT |
| K117D | GAT |
| K117N | AAT |
| K117G | GGT |
| K117W | TGG |
| K117Y | TAT |
| K117L | TTG |
| K117S | AGT |
| K117P | CCG |
| K117R | AGG |
| A118G | GGG |
| A118R | CGT |
| A118W | TGG |
| A118K | AAG |
| A118P | CCT |
| A118V | GTG |
| A118L | TTG |
| A118D | GAT |
| A118S | AGT |
| A118F | TTT |
| A118I | ATT |
| A118H | CAT |
| A118E | GAG |
| A118Q | CAG |
| A118T | ACT |
| F119G | GGG |
| F119T | ACT |
| F119R | CGG |
| L128A | GCG |
| L128D | GAT |
| L128V | GTG |
| L128W | TGG |
| L128C | TGT |
| L128K | AAG |
| T129G | GGT |
| T129A | GCT |
| T129C | TGT |
| T129K | AAG |
| T129F | TTT |
| T129Y | TAT |
| T129S | TCG |
| T129R | CGG |
| T129V | GTT |
| T129L | CTT |
| T129H | CAT |
| T129P | CCT |
| T129E | GAG |
| T129I | ATT |
| T129M | ATG |
| F130L | CTG |
| F130P | CCT |
| F130C | TGT |
| F130R | CGG |
| F130Y | TAT |
| F130H | CAT |
| F130I | ATT |
| F130V | GTT |
| F130K | AAG |
| F130T | ACT |
| F130E | GAG |
| F130A | GCG |
| F130N | AAT |
| F130G | GGT |
| F130S | AGT |
| T131F | TTT |
| T131P | CCG |
| T131A | GCG |
| T131S | TCT |
| T131G | GGT |
| T131I | ATT |
| T131L | CTT |
| T131H | CAT |
| T131Q | CAG |
| T131D | GAT |
| T131E | GAG |
| T131C | TGT |
| M141S | AGT |
| M141C | TGT |
| M141L | CTG |
| M141A | GCG |
| M141D | GAT |
| M141W | TGG |
| M141G | GGT |
| M141H | CAT |
| M141Y | TAT |
| M141N | AAT |
| I142L | CTG |
| I142M | ATG |
| I142G | GGT |
| I142K | AAG |
| I142A | GCT |
| I142N | AAT |
| I142W | TGG |
| I142P | CCG |
| I142Q | CAG |
| I142Y | TAT |
| I142V | GTG |
| I142T | ACT |
| I142R | CGG |
| I142S | AGT |
| I142F | TTT |
| S143P | CCG |
| S143C | TGT |
| S143E | GAG |
| S143G | GGT |
| S143H | CAT |
| S143R | CGT |
| S143L | TTG |
| S143Q | CAG |
| S143N | AAT |
| S143W | TGG |
| S143A | GCT |
| S143T | ACT |
| S143Y | TAT |
| S143M | ATG |
| S143I | ATT |
| F144K | AAG |
| F144M | ATG |
| F144E | GAG |
| F144S | AGT |
| F144L | CTG |
| F144W | TGG |
| F144P | CCG |
| F144R | CGG |
| P154L | CTT |
| P154C | TGT |
| P154S | TCT |
| P154K | AAG |
| P154I | ATT |
| P154A | GCT |
| P154T | ACG |
| P154H | CAT |
| P154Y | TAT |
| P154N | AAT |
| P154F | TTT |
| P154R | CGT |
| P154Q | CAG |
| F155S | TCT |
| F155T | ACT |
| F155G | GGT |
| F155N | AAT |
| F155R | CGG |
| F155W | TGG |
| F155L | CTG |
| F155Q | CAG |
| F155M | ATG |
| F155E | GAG |
| F155A | GCG |
| F155P | CCT |
| F155V | GTT |
| F155H | CAT |
| F155Y | TAT |
| D156H | CAT |
| D156L | CTT |
| D156E | GAG |
| D156A | GCT |
| D156W | TGG |
| D156C | TGT |
| D156P | CCT |
| D156V | GTT |
| D156K | AAG |
| D156S | TCT |
| D156G | GGG |
| D156T | ACT |
| D156Y | TAT |
| D156R | CGT |
| D156M | ATG |
| G157K | AAG |
| G157D | GAT |
| G157F | TTT |
| G157R | CGT |
| G157H | CAT |
| F166C | TGT |
| F166E | GAG |
| Q167D | GAT |
| Q167R | CGG |
| Q167A | GCG |
| Q167S | AGT |
| Q167F | TTT |
| Q167Y | TAT |
| Q167P | CCG |
| Q167T | ACT |
| Q167V | GTG |
| Q167L | CTG |
| Q167M | ATG |
| Q167N | AAT |
| Q167G | GGG |
| Q167K | AAG |
| Q167E | GAG |
| P168N | AAT |
| P168F | TTT |
| P168R | CGG |
| P168W | TGG |
| P168A | GCT |
| P168T | ACG |
| P168V | GTT |
| P168G | GGG |
| P168C | TGT |
| P168M | ATG |
| P168H | CAT |
| P168L | CTT |
| P168S | AGT |
| P168I | ATT |
| P168D | GAT |
| G169H | CAT |
| G169A | GCG |
| G169E | GAG |
| G169C | TGT |
| G169S | TCG |
| G169L | CTG |
| G169V | GTT |
| G169T | ACG |
| G169R | CGG |
| G169W | TGG |
| G169M | ATG |
| G169I | ATT |
| G169P | CCG |
| G169D | GAT |
| G169Q | CAG |
| P170L | CTT |
| D179I | ATT |
| D179R | CGT |
| D179N | AAT |
| D179W | TGG |
| D179Q | CAG |
| D179V | GTG |
| D179C | TGT |
| E180M | ATG |
| E180P | CCT |
| E180K | AAG |
| E180Y | TAT |
| E180Q | CAG |
| E180R | CGG |
| E180A | GCG |
| E180T | ACT |
| E180I | ATT |
| E180F | TTT |
| E180C | TGT |
| E180G | GGG |
| E180S | TCG |
| E180N | AAT |
| E180D | GAT |
| D181S | TCG |
| D181Q | CAG |
| D181P | CCT |
| D181Y | TAT |
| D181R | CGT |
| D181V | GTT |
| D181F | TTT |
| D181A | GCT |
| D181T | ACG |
| D181L | TTG |
| D181E | GAG |
| D181K | AAG |
| D181M | ATG |
| D181C | TGT |
| D181G | GGT |
| E182C | TGT |
| E182P | CCT |
| E182S | AGT |
| E182T | ACG |
| E182R | CGG |
| E182D | GAT |
| E182A | GCT |
| E182F | TTT |
| E182L | CTT |
| E182I | ATT |
| E182Y | TAT |
| N192S | TCG |
| N192W | TGG |
| N192G | GGG |
| N192D | GAT |
| N192V | GTG |
| N192A | GCT |
| N192T | ACT |
| N192K | AAG |
| N192C | TGT |
| N192M | ATG |
| L193P | CCG |
| L193G | GGG |
| L193F | TTT |
| L193S | TCG |
| L193W | TGG |
| L193A | GCT |
| L193R | CGT |
| L193Q | CAG |
| L193E | GAG |
| L193K | AAG |
| L193N | AAT |
| L193I | ATT |
| L193T | ACT |
| L193D | GAT |
| L193Y | TAT |
| H194S | AGT |
| H194E | GAG |
| H194K | AAG |
| H194Q | CAG |
| H194V | GTT |
| H194T | ACT |
| H194L | CTG |
| H194Y | TAT |
| H194F | TTT |
| H194G | GGT |
| H194I | ATT |
| H194W | TGG |
| H194M | ATG |
| H194A | GCT |
| H194P | CCT |
| R195C | TGT |
| R195F | TTT |
| R195W | TGG |
| R195T | ACT |
| R195L | CTG |
| R195G | GGT |
| R195Q | CAG |
| R195K | AAG |
| L205S | TCT |
| L205G | GGT |
| L205P | CCT |
| L205E | GAG |
| L205V | GTG |
| L205M | ATG |
| L205N | AAT |
| L205C | TGT |
| L205I | ATT |
| L205A | GCG |
| L205R | CGG |
| L205W | TGG |
| L205Q | CAG |
| G206I | ATT |
| G206V | GTG |
| G206A | GCG |
| G206C | TGT |
| G206S | TCG |
| G206P | CCG |
| G206L | TTG |
| G206D | GAT |
| G206M | ATG |
| G206R | CGG |
| G206Q | CAG |
| G206E | GAG |
| G206H | CAT |
| G206T | ACG |
| G206W | TGG |
| L207S | TCT |
| L207Y | TAT |
| L207A | GCG |
| L207R | CGT |
| L207P | CCG |
| L207Q | CAG |
| L207N | AAT |
| L207K | AAG |
| L207M | ATG |
| L207W | TGG |
| L207H | CAT |
| L207D | GAT |
| L207V | GTT |
| L207I | ATT |
| L207G | GGT |
| S208D | GAT |
| S208V | GTT |
| S208P | CCT |
| S208G | GGT |
| S208A | GCG |
| M217A | GCG |
| M217H | CAT |
| M217I | ATT |
| M217D | GAT |
| Y218C | TGT |
| Y218F | TTT |
| Y218W | TGG |
| Y218L | CTG |
| Y218A | GCG |
| Y218P | CCG |
| Y218R | CGG |
| Y218N | AAT |
| Y218V | GTG |
| Y218Q | CAG |
| Y218I | ATT |
| Y218D | GAT |
| Y218S | TCG |
| Y218G | GGG |
| Y218E | GAG |
| P219L | TTG |
| P219C | TGT |
| P219V | GTG |
| P219D | GAT |
| P219F | TTT |
| P219A | GCG |
| P219T | ACT |
| P219E | GAG |
| P219Q | CAG |
| P219R | CGG |
| P219H | CAT |
| P219G | GGG |
| P219K | AAG |
| P219S | TCG |
| P219W | TGG |
| S220R | CGT |
| S220A | GCG |
| S220Q | CAG |
| S220T | ACT |
| S220L | CTT |
| S220K | AAG |
| S220G | GGG |
| S220H | CAT |
| S220E | GAG |
| S220M | ATG |
| S220V | GTT |
| S220P | CCG |
| S220I | ATT |
| S220F | TTT |
| A230S | TCG |
| A230C | TGT |
| A230V | GTT |
| A230T | ACT |
| A230Y | TAT |
| A230M | ATG |
| A230N | AAT |
| A230H | CAT |
| Q231I | ATT |
| Q231A | GCT |
| Q231F | TTT |
| Q231P | CCT |
| Q231Y | TAT |
| Q231R | CGT |
| Q231L | CTG |
| Q231D | GAT |
| Q231G | GGT |
| Q231V | GTT |
| Q231W | TGG |
| Q231S | AGT |
| Q231H | CAT |
| Q231C | TGT |
| Q231M | ATG |
| D232H | CAT |
| D232G | GGG |
| D232R | CGT |
| D232P | CCT |
| D232Y | TAT |
| D232N | AAT |
| D232S | TCG |
| D232F | TTT |
| D232V | GTG |
| D232K | AAG |
| D232W | TGG |
| D232Q | CAG |
| D232E | GAG |
| D232T | ACT |
| D232L | CTG |
| D233Q | CAG |
| D233P | CCG |
| D233S | TCT |
| D233T | ACG |
| D233A | GCG |
| D233W | TGG |
| D233G | GGT |
| D233R | CGT |
| D233E | GAG |
| D233N | AAT |
| R243L | CTT |
| R243A | GCG |
| R243H | CAT |
| R243Q | CAG |
| R243S | AGT |
| R243I | ATT |
| R243C | TGT |
| R243N | AAT |
| R243Y | TAT |
| R243G | GGG |
| R243D | GAT |
| R243V | GTG |
| S244P | CCG |
| S244L | CTT |
| S244W | TGG |
| S244M | ATG |
| S244V | GTT |
| S244Q | CAG |
| S244D | GAT |
| S244E | GAG |
| S244T | ACG |
| S244H | CAT |
| S244G | GGT |
| S244A | GCT |
| S244F | TTT |
| S244Y | TAT |
| S244R | CGT |
| Q245P | CCT |
| Q245I | ATT |
| Q245F | TTT |
| Q245V | GTT |
| Q245M | ATG |
| Q245T | ACT |
| Q245E | GAG |
| Q245S | TCG |
| Q245R | CGG |
| Q245G | GGT |
| Q245H | CAT |
| Q245L | CTT |
| Q245K | AAG |
| Q245W | TGG |
| Q245C | TGT |
| N246W | TGG |
| N246R | CGG |
| N246A | GCG |
| N246F | TTT |
| N246G | GGT |
| N246P | CCT |
| T255L | TTG |
| T255H | CAT |
| P256S | AGT |
| P256V | GTG |
| P256F | TTT |
| P256Y | TAT |
| P256I | ATT |
| P256A | GCT |
| P256L | CTT |
| P256G | GGT |
| P256N | AAT |
| P256R | CGG |
| P256Q | CAG |
| P256E | GAG |
| P256K | AAG |
| P256M | ATG |
| P256C | TGT |
| K257C | TGT |
| K257M | ATG |
| K257V | GTT |
| K257A | GCT |
| K257E | GAG |
| K257S | TCT |
| K257L | CTT |
| K257I | ATT |
| K257G | GGG |
| K257N | AAT |
| K257F | TTT |
| K257W | TGG |
| K257R | CGG |
| K257P | CCG |
| K257T | ACT |
| A258Q | CAG |
| A258Y | TAT |
| A258W | TGG |
| A258G | GGG |
| A258L | TTG |
| A258F | TTT |
| A258M | ATG |
| A258N | AAT |
| A258V | GTG |
| A258T | ACG |
| A258I | ATT |
| A258D | GAT |
| A258R | CGT |
| A258E | GAG |
| A258P | CCG |
| T255L | TTG |

B. Screening

Mutant MMP-1 members of the library were screened against a fluorogenic peptide substrate IX (Mca-K-P-L-Gl-L-Dpa-A-R-NH₂; SEQ ID NO:88; R&D Systems, Minneapolis, Minn., Cat#ES010) for decreased catalytic activity at 37° C. relative to 25° C. and for sufficient protein expression as described in published U.S. Application No. 2010/0284995. Briefly, wild-type and mutant MMP-1 cDNAs derived from the library above were transformed in BL21 (DE3) cells (Stratagene or Tigen, Beiging, China) in 96 well plates. Protein expression was induced with 1 mM isopropyl-β-D-thiogalactoside (IPTG) and the cells were incubated at 25° C. with shaking. After 6 hours, the cells were pelleted by centrifugation at 6,000 g for 10 minutes and the supernatant was removed. The periplasmic protein fraction was then isolated by incubating the cells in OS buffer (200 mM Tris-HCl, pH 7.5, 20% sucrose, 1 mM EDTA) with 0.5 mg/mL of DNase, RNase, and lysozyme. The cells were centrifuged after the addition of cold water and supernatant collected.

The supernatant containing the periplasmic protein fraction were transferred to 96 well plates. MMP-1 protein in the supernatants was activated with 1 mM APMA (4-aminophenylmercuric acetate; Sigma) at either 25° C. or 37° C. Following activation, 1.6 μL of TCNB containing 620 μM Mca-K-P-L-G-L-Dpa-A-R-NH₂ fluorescent substrate was added to each well to a final concentration of 10 μM, at the indicated reaction temperature (either 25° C. or 37° C.) for 1 hour. Fluorescence was detected by measuring fluorescence in a fluorescent plate reader at 320 nm exitation/405 nm emission. Relative fluorescence units (RFU) were determined. Supernatant from wild-type hMMP-1 and plasmid/vector transformed cells were used as positive and negative controls. Duplicate reactions were performed for each sample, reaction temperature, and positive and negative control. The results are set forth in Table 10.

TABLE 10
Results of Initial Screen of MMP-1 Mutant Library

| | | | | Res. | Res. |
|---|---|---|---|---|---|
| | | | | Act. | Act. |
| hMMP-1 | Avg. RFU | Avg. RFU | Ratio | Mut/wt | Mut/wt |
| mutation | 25° C. | 37° C. | 25° C./37° C. | 25° C. | 37° C. |
| F81C | 1740.62 | 3123.63 | 0.56 | 0.35 | 0.46 |
| F81E | 871.51 | 1243.66 | 0.70 | 0.18 | 0.18 |
| F81I | 4100.22 | 5376.62 | 0.76 | 0.83 | 0.79 |
| F81L | 8890.68 | 7913.44 | 1.12 | 1.57 | 1.51 |
| F81P | 1102.23 | 1043.87 | 1.06 | 0.19 | 0.20 |
| F81S | 2527.30 | 2312.47 | 1.09 | 0.45 | 0.44 |
| F81A | 8780.53 | 7784.51 | 1.13 | 1.55 | 1.48 |
| F81M | 2545.25 | 3095.21 | 0.82 | 0.45 | 0.59 |
| F81G | 8979.05 | 7773.71 | 1.16 | 1.59 | 1.48 |
| F81T | 1564.49 | 1373.60 | 1.14 | 0.28 | 0.26 |
| F81Q | 9225.28 | 7923.69 | 1.16 | 1.63 | 1.51 |
| F81R | 8514.40 | 7454.74 | 1.14 | 1.50 | 1.42 |
| F81W | 6078.70 | 5909.04 | 1.03 | 1.07 | 1.12 |
| F81H | 8126.15 | 7360.21 | 1.10 | 1.44 | 1.40 |
| F81V | 7263.15 | 6614.17 | 1.10 | 1.28 | 1.26 |
| V82I | 535.78 | 548.02 | 0.98 | 0.06 | 0.06 |
| V82C | 4177.57 | 6476.29 | 0.65 | 0.50 | 0.72 |
| V82A | 9540.61 | 9240.92 | 1.03 | 1.14 | 1.03 |
| V82P | 599.23 | 634.69 | 0.94 | 0.07 | 0.07 |
| V82Y | 3295.59 | 6173.45 | 0.53 | 0.39 | 0.69 |
| V82M | 6824.39 | 8606.64 | 0.79 | 0.82 | 0.96 |
| V82Q | 581.51 | 652.74 | 0.89 | 0.07 | 0.07 |
| V82F | 7233.54 | 8739.45 | 0.83 | 0.87 | 0.98 |
| V82W | 6194.12 | 8397.19 | 0.74 | 0.74 | 0.94 |
| V82N | 9421.72 | 8759.51 | 1.08 | 1.13 | 0.98 |
| V82R | 603.22 | 781.77 | 0.77 | 0.07 | 0.09 |
| V82G | 8298.42 | 8911.04 | 0.93 | 0.99 | 0.99 |
| V82S | 8293.03 | 9022.13 | 0.92 | 0.99 | 1.01 |
| V82L | 6951.75 | 8694.05 | 0.80 | 0.83 | 0.97 |
| V82T | 7993.81 | 8975.05 | 0.89 | 0.96 | 1.00 |
| L83A | 8629.03 | 9023.51 | 0.96 | 1.03 | 1.01 |
| L83C | 554.26 | 567.87 | 0.98 | 0.07 | 0.06 |
| L83D | 8705.34 | 8957.38 | 0.97 | 1.04 | 1.00 |
| L83E | 9212.48 | 9265.02 | 0.99 | 1.10 | 1.03 |
| L83G | 7713.92 | 9073.74 | 0.85 | 0.92 | 1.01 |
| L83H | 6449.24 | 7800.76 | 0.83 | 0.77 | 0.87 |
| L83I | 4575.76 | 6963.24 | 0.66 | 0.55 | 0.78 |
| L83M | 5921.65 | 8064.61 | 0.73 | 0.71 | 0.90 |
| L83P | 7794.15 | 8608.36 | 0.91 | 0.93 | 0.96 |
| L83Q | 7291.24 | 8673.39 | 0.84 | 0.87 | 0.97 |
| L83R | 8509.58 | 8988.62 | 0.95 | 1.02 | 1.00 |
| L83S | 9261.79 | 9205.93 | 1.01 | 1.11 | 1.03 |
| L83T | 7549.73 | 8580.54 | 0.88 | 0.90 | 0.96 |
| L83W | 4193.18 | 6044.52 | 0.69 | 0.50 | 0.67 |
| L83Y | 7968.79 | 9051.39 | 0.88 | 0.95 | 1.01 |
| T84V | 3169.35 | 4931.29 | 0.64 | 0.64 | 0.72 |
| T84E | 498.18 | 627.84 | 0.79 | 0.10 | 0.09 |
| T84H | 7046.83 | 6974.20 | 1.01 | 1.24 | 1.33 |
| T84L | 7687.84 | 6946.59 | 1.11 | 1.36 | 1.32 |
| T84D | 7972.32 | 7331.43 | 1.09 | 1.41 | 1.39 |
| T84R | 7298.49 | 6880.17 | 1.06 | 1.29 | 1.31 |
| T84I | 6508.69 | 5860.75 | 1.11 | 1.15 | 1.11 |
| T84S | 6073.28 | 5981.85 | 1.02 | 1.07 | 1.14 |
| T84G | 8087.79 | 7200.99 | 1.12 | 1.43 | 1.37 |
| T84Q | 6275.12 | 6690.38 | 0.94 | 1.11 | 1.27 |
| T84P | 3528.37 | 3832.34 | 0.92 | 0.62 | 0.73 |
| T84A | 8718.27 | 7840.72 | 1.11 | 1.54 | 1.49 |
| T84C | 5177.89 | 5107.57 | 1.01 | 0.91 | 0.97 |
| T84Y | 4768.51 | 4818.30 | 0.99 | 0.84 | 0.92 |
| T84F | 6312.72 | 6453.46 | 0.98 | 1.10 | 1.27 |
| E85L | 1633.29 | 2148.43 | 0.76 | 0.33 | 0.31 |
| E85Q | 2834.50 | 4068.60 | 0.70 | 0.57 | 0.59 |
| E85P | 2855.52 | 3389.51 | 0.84 | 0.58 | 0.50 |
| E85T | 401.26 | 382.58 | 1.05 | 0.08 | 0.06 |
| E85K | 2293.84 | 3049.87 | 0.75 | 0.46 | 0.45 |
| E85M | 2158.30 | 2821.39 | 0.76 | 0.44 | 0.41 |
| E85G | 1767.69 | 1734.31 | 1.02 | 0.31 | 0.33 |
| E85R | 912.46 | 7286.41 | 0.13 | 0.16 | 1.39 |
| E85S | 7811.54 | 7488.09 | 1.04 | 1.38 | 1.42 |
| E85C | 6027.10 | 5938.05 | 1.01 | 1.06 | 1.13 |
| E85Y | 4449.33 | 3909.71 | 1.14 | 0.79 | 0.74 |
| E85A | 5552.19 | 5461.08 | 1.02 | 0.98 | 1.04 |
| E85N | 522.81 | 7634.45 | 0.07 | 0.09 | 1.45 |
| E85V | 7152.74 | 7011.60 | 1.02 | 1.26 | 1.33 |
| E85F | 6092.47 | 6362.37 | 0.96 | 1.06 | 1.26 |
| G86L | 2452.10 | 3232.22 | 0.76 | 0.50 | 0.47 |
| G86P | 2117.46 | 5219.90 | 0.41 | 0.43 | 0.76 |
| G86I | 1888.26 | 2293.71 | 0.82 | 0.38 | 0.34 |
| G86T | 363.85 | 380.61 | 0.96 | 0.07 | 0.06 |
| G86H | 389.15 | 372.78 | 1.04 | 0.08 | 0.05 |
| G86D | 415.45 | 406.81 | 1.02 | 0.08 | 0.06 |
| G86N | 2612.85 | 3755.02 | 0.70 | 0.53 | 0.55 |
| G86S | 8500.13 | 7717.19 | 1.10 | 1.50 | 1.47 |
| G86K | 1660.95 | 2002.39 | 0.83 | 0.29 | 0.38 |
| G86W | 1570.85 | 1690.05 | 0.93 | 0.28 | 0.32 |
| G86Y | 1829.24 | 2126.68 | 0.86 | 0.32 | 0.40 |
| G86V | 1830.80 | 2092.69 | 0.87 | 0.32 | 0.40 |
| G86C | 1784.05 | 2091.03 | 0.85 | 0.32 | 0.40 |
| G86M | 1687.28 | 2025.99 | 0.83 | 0.30 | 0.39 |
| G86F | 1897.87 | 1483.82 | 1.28 | 0.34 | 0.28 |
| N87M | 418.35 | 412.23 | 1.01 | 0.08 | 0.06 |
| N87L | 3385.42 | 4941.20 | 0.69 | 0.69 | 0.72 |
| N87P | 8762.48 | 8941.20 | 0.98 | 1.55 | 1.70 |
| N87V | 6199.21 | 7269.38 | 0.85 | 1.09 | 1.38 |
| N87R | 7761.00 | 8810.25 | 0.88 | 1.37 | 1.68 |
| N87F | 6882.19 | 4428.08 | 1.55 | 1.22 | 0.84 |
| N87S | 2083.05 | 3304.46 | 0.63 | 0.37 | 0.63 |
| N87I | 7572.66 | 8090.13 | 0.94 | 1.34 | 1.54 |
| N87C | 3291.22 | 3945.40 | 0.83 | 0.58 | 0.75 |
| N87A | 5482.33 | 6869.11 | 0.80 | 0.97 | 1.31 |
| N87G | 8060.01 | 8916.11 | 0.90 | 1.42 | 1.70 |
| N87Y | 4397.56 | 5611.87 | 0.78 | 0.78 | 1.07 |
| N87E | 5876.33 | 4763.86 | 1.23 | 1.04 | 0.91 |
| N87H | 5013.05 | 7306.33 | 0.69 | 0.89 | 1.39 |
| N87Q | 8559.37 | 9021.72 | 0.95 | 1.51 | 1.72 |
| P88C | 1255.12 | 2197.65 | 0.57 | 0.15 | 0.25 |
| P88K | 6857.61 | 8492.90 | 0.81 | 0.82 | 0.95 |
| P88W | 664.95 | 845.70 | 0.79 | 0.08 | 0.09 |
| P88G | 1694.96 | 3159.20 | 0.54 | 0.20 | 0.35 |
| P88L | 2562.59 | 3576.95 | 0.72 | 0.31 | 0.40 |
| P88Q | 4499.52 | 7270.91 | 0.62 | 0.54 | 0.81 |
| P88A | 6549.92 | 8130.83 | 0.81 | 0.78 | 0.91 |
| P88T | 6576.99 | 8126.45 | 0.81 | 0.79 | 0.91 |
| P88Y | 5515.19 | 7868.29 | 0.70 | 0.66 | 0.88 |
| P88R | 4209.25 | 6681.38 | 0.63 | 0.50 | 0.75 |
| P88H | 2580.97 | 4465.31 | 0.58 | 0.31 | 0.50 |
| P88I | 841.81 | 1249.17 | 0.67 | 0.10 | 0.14 |
| P88V | 1666.69 | 1915.49 | 0.87 | 0.20 | 0.21 |
| P88E | 971.61 | 1460.63 | 0.67 | 0.12 | 0.16 |
| P88D | 1300.22 | 1911.83 | 0.68 | 0.16 | 0.21 |
| R89V | 1163.86 | 2620.05 | 0.44 | 0.24 | 0.38 |
| R89W | 1252.89 | 1744.18 | 0.72 | 0.25 | 0.25 |
| R89M | 402.00 | 386.98 | 1.04 | 0.08 | 0.06 |
| R89A | 7883.15 | 8954.83 | 0.88 | 1.39 | 1.70 |
| R89T | 6791.27 | 6752.46 | 1.01 | 1.20 | 1.28 |
| R89G | 8957.06 | 8693.72 | 1.03 | 1.58 | 1.65 |
| R89S | 7342.24 | 4138.54 | 1.77 | 1.30 | 0.79 |
| R89K | 7679.02 | 8254.00 | 0.93 | 1.36 | 1.57 |
| R89F | 4764.35 | 5589.97 | 0.85 | 0.84 | 1.06 |
| R89Y | 5614.23 | 5949.31 | 0.94 | 0.99 | 1.13 |
| R89N | 3502.08 | 1995.86 | 1.75 | 0.62 | 0.38 |
| R89H | 3611.69 | 4222.47 | 0.86 | 0.64 | 0.80 |
| R89L | 3123.66 | 3332.30 | 0.94 | 0.55 | 0.63 |
| R89E | 1490.93 | 1265.89 | 1.18 | 0.26 | 0.24 |
| R89P | 2659.02 | 3342.56 | 0.80 | 0.47 | 0.64 |
| W90L | 394.24 | 411.82 | 0.96 | 0.08 | 0.06 |
| W90G | 448.08 | 427.28 | 1.05 | 0.09 | 0.06 |
| W90P | 444.72 | 442.65 | 1.00 | 0.09 | 0.06 |
| W90T | 397.42 | 365.04 | 1.09 | 0.08 | 0.05 |
| W90S | 443.43 | 442.72 | 1.00 | 0.09 | 0.06 |
| W90V | 384.57 | 385.18 | 1.00 | 0.08 | 0.06 |
| W90I | 443.81 | 432.28 | 1.03 | 0.09 | 0.06 |
| W90A | 497.94 | 554.07 | 0.90 | 0.10 | 0.08 |
| W90F | 730.98 | 656.84 | 1.11 | 0.15 | 0.10 |
| W90H | 498.51 | 493.15 | 1.01 | 0.10 | 0.07 |
| W90M | 512.18 | 508.03 | 1.01 | 0.10 | 0.07 |
| W90R | 1974.98 | 1695.11 | 1.17 | 0.23 | 0.19 |
| W90E | 1537.84 | 1076.32 | 1.43 | 0.18 | 0.12 |
| W90N | 1308.11 | 1001.91 | 1.31 | 0.15 | 0.11 |
| W90Q | 1392.58 | 1015.03 | 1.37 | 0.16 | 0.12 |
| E91N | 4746.43 | 6166.37 | 0.77 | 0.96 | 0.90 |
| E91R | 2760.48 | 3810.12 | 0.72 | 0.56 | 0.56 |
| E91W | 2595.35 | 5651.48 | 0.46 | 0.53 | 0.83 |
| E91G | 4826.02 | 6684.79 | 0.72 | 0.98 | 0.98 |
| E91V | 454.87 | 459.17 | 0.99 | 0.09 | 0.07 |
| E91Y | 4885.18 | 5469.16 | 0.89 | 0.99 | 0.80 |
| E91C | 3525.68 | 5567.75 | 0.63 | 0.71 | 0.81 |
| E91H | 5114.86 | 6610.88 | 0.77 | 1.04 | 0.97 |
| E91T | 442.21 | 427.42 | 1.03 | 0.09 | 0.06 |
| E91S | 8147.93 | 7696.77 | 1.06 | 0.94 | 0.87 |
| E91A | 1140.60 | 1252.34 | 0.91 | 0.13 | 0.14 |
| E91I | 8414.79 | 8744.30 | 0.96 | 0.97 | 0.99 |
| E91D | 8482.61 | 8681.73 | 0.98 | 0.98 | 0.98 |
| E91F | 1159.80 | 1117.15 | 1.04 | 0.13 | 0.13 |
| E91L | 2012.22 | 1956.07 | 1.03 | 0.23 | 0.22 |
| Q92V | 3748.94 | 5787.25 | 0.65 | 0.76 | 0.85 |
| Q92Y | 2141.40 | 5383.55 | 0.40 | 0.43 | 0.79 |
| Q92L | 2422.01 | 3765.30 | 0.64 | 0.49 | 0.55 |
| Q92N | 8685.91 | 8183.03 | 1.06 | 1.00 | 0.93 |
| Q92E | 8489.89 | 8972.33 | 0.95 | 0.98 | 1.02 |
| Q92I | 7791.35 | 8518.64 | 0.91 | 0.90 | 0.97 |
| Q92T | 8289.96 | 8916.74 | 0.93 | 0.96 | 1.01 |
| Q92G | 7218.56 | 8372.74 | 0.86 | 0.83 | 0.95 |
| Q92P | 3678.59 | 4021.57 | 0.91 | 0.43 | 0.46 |
| Q92W | 7277.76 | 8042.96 | 0.90 | 0.84 | 0.91 |
| Q92F | 8216.00 | 8989.59 | 0.91 | 0.95 | 1.02 |
| Q92S | 8760.81 | 9254.16 | 0.95 | 1.01 | 1.05 |
| Q92R | 8566.65 | 8894.65 | 0.96 | 0.99 | 1.01 |
| Q92K | 8790.93 | 9239.36 | 0.95 | 1.02 | 1.05 |
| Q92A | 8138.84 | 9037.58 | 0.90 | 0.94 | 1.02 |
| T93A | 2321.71 | 5447.47 | 0.43 | 0.47 | 0.80 |
| T93L | 541.85 | 545.89 | 0.99 | 0.11 | 0.08 |
| T93M | 5256.54 | 7088.24 | 0.74 | 1.07 | 1.04 |
| T93N | 5852.91 | 7141.52 | 0.82 | 1.19 | 1.04 |
| T93V | 7976.61 | 8668.81 | 0.92 | 0.92 | 0.98 |
| T93I | 9015.76 | 9426.26 | 0.96 | 1.04 | 1.07 |
| T93D | 8742.88 | 9032.61 | 0.97 | 1.01 | 1.02 |
| T93S | 8832.30 | 8978.09 | 0.98 | 1.02 | 1.02 |
| T93R | 8802.98 | 8782.70 | 1.00 | 1.02 | 1.00 |
| T93W | 7872.73 | 8474.20 | 0.93 | 0.91 | 0.96 |
| T93F | 4307.35 | 5656.46 | 0.76 | 0.50 | 0.64 |
| T93P | 8315.28 | 8629.67 | 0.96 | 0.96 | 0.98 |
| T93G | 4926.38 | 6453.11 | 0.76 | 0.57 | 0.73 |
| T93K | 8581.02 | 8663.14 | 0.99 | 0.99 | 0.98 |
| T93E | 8081.66 | 8373.46 | 0.97 | 0.93 | 0.95 |
| H94L | 509.44 | 507.83 | 1.00 | 0.10 | 0.07 |
| H94S | 3442.98 | 5184.16 | 0.66 | 0.70 | 0.76 |
| H94M | 7388.19 | 8302.74 | 0.89 | 0.85 | 0.94 |
| H94R | 7237.77 | 7718.22 | 0.94 | 0.84 | 0.87 |
| H94E | 8375.45 | 8466.04 | 0.99 | 0.97 | 0.96 |
| H94I | 6326.35 | 7655.54 | 0.83 | 0.73 | 0.87 |
| H94D | 7358.29 | 8057.05 | 0.91 | 0.85 | 0.91 |
| H94P | 2892.06 | 3183.37 | 0.91 | 0.33 | 0.36 |
| H94A | 8285.72 | 8772.41 | 0.94 | 0.96 | 0.99 |
| H94N | 8497.48 | 8732.16 | 0.97 | 0.98 | 0.99 |
| H94F | 6046.02 | 7839.76 | 0.77 | 0.70 | 0.89 |
| H94G | 7671.85 | 7912.46 | 0.97 | 0.89 | 0.90 |
| H94T | 7121.14 | 8100.48 | 0.88 | 0.82 | 0.92 |
| H94V | 7941.67 | 8381.81 | 0.95 | 0.92 | 0.95 |
| H94W | 6520.52 | 7583.08 | 0.86 | 0.75 | 0.86 |
| L95E | 4165.44 | 5381.52 | 0.77 | 0.48 | 0.61 |
| L95Y | 1044.63 | 1118.92 | 0.93 | 0.12 | 0.13 |
| L95R | 1328.79 | 1312.13 | 1.01 | 0.15 | 0.15 |
| L95A | 1262.99 | 1297.06 | 0.97 | 0.15 | 0.15 |
| L95G | 1090.24 | 1183.93 | 0.92 | 0.13 | 0.13 |
| L95K | 1333.28 | 1191.46 | 1.12 | 0.15 | 0.14 |
| L95S | 1077.02 | 1117.02 | 0.96 | 0.12 | 0.13 |
| L95T | 1407.58 | 1310.18 | 1.07 | 0.16 | 0.15 |
| L95H | 1270.21 | 1086.69 | 1.17 | 0.15 | 0.12 |
| L95W | 1133.63 | 1041.65 | 1.09 | 0.13 | 0.12 |
| L95V | 8390.57 | 8371.68 | 1.00 | 0.97 | 0.95 |
| L95C | 2189.50 | 2519.34 | 0.87 | 0.25 | 0.29 |
| L95P | 1084.88 | 1147.69 | 0.95 | 0.13 | 0.13 |
| L95D | 909.41 | 933.49 | 0.97 | 0.11 | 0.11 |
| L95I | 1707.98 | 2294.02 | 0.74 | 0.30 | 0.45 |
| T96E | 415.05 | 397.38 | 1.04 | 0.07 | 0.06 |
| T96R | 478.81 | 441.86 | 1.08 | 0.08 | 0.07 |
| T96P | 589.64 | 692.90 | 0.85 | 0.09 | 0.11 |
| T96S | 3055.53 | 4011.47 | 0.76 | 0.49 | 0.64 |
| T96A | 1873.45 | 2254.28 | 0.83 | 0.30 | 0.36 |
| T96L | 2337.67 | 3156.45 | 0.74 | 0.37 | 0.51 |
| T96W | 1194.79 | 1631.19 | 0.73 | 0.19 | 0.26 |
| T96N | 2674.35 | 3874.07 | 0.69 | 0.43 | 0.62 |
| T96G | 415.04 | 387.45 | 1.07 | 0.07 | 0.06 |
| T96F | 2640.74 | 3897.10 | 0.68 | 0.42 | 0.62 |
| T96Q | 1865.64 | 2509.48 | 0.74 | 0.30 | 0.40 |
| T96H | 1294.29 | 1620.22 | 0.80 | 0.21 | 0.26 |
| T96V | 1904.14 | 2730.27 | 0.70 | 0.30 | 0.44 |
| T96I | 1814.91 | 2921.26 | 0.62 | 0.29 | 0.47 |
| T96C | 701.03 | 774.48 | 0.91 | 0.11 | 0.12 |
| Y97R | 447.48 | 449.81 | 0.99 | 0.14 | 0.09 |
| Y97V | 637.70 | 789.90 | 0.81 | 0.20 | 0.16 |
| Y97A | 507.18 | 504.63 | 1.01 | 0.16 | 0.10 |
| Y97P | 488.40 | 452.67 | 1.08 | 0.15 | 0.09 |
| Y97L | 510.25 | 549.53 | 0.93 | 0.16 | 0.11 |
| Y97T | 538.83 | 600.97 | 0.90 | 0.17 | 0.12 |
| Y97K | 469.55 | 390.08 | 1.20 | 0.15 | 0.08 |
| Y97W | 3115.45 | 4974.99 | 0.63 | 0.98 | 1.01 |
| Y97H | 685.71 | 879.64 | 0.78 | 0.22 | 0.18 |
| Y97S | 482.94 | 471.85 | 1.02 | 0.15 | 0.10 |
| Y97E | 435.12 | 432.07 | 1.01 | 0.14 | 0.09 |
| Y97D | 466.89 | 455.39 | 1.03 | 0.15 | 0.09 |
| Y97N | 486.98 | 490.84 | 0.99 | 0.15 | 0.10 |
| Y97G | 521.34 | 516.64 | 1.01 | 0.11 | 0.08 |
| Y97Q | 567.66 | 575.73 | 0.99 | 0.12 | 0.08 |
| R98H | 1456.51 | 3257.97 | 0.45 | 0.46 | 0.66 |
| R98K | 2994.82 | 4670.17 | 0.64 | 0.95 | 0.95 |
| R98C | 761.34 | 938.43 | 0.81 | 0.24 | 0.19 |
| R98L | 3592.72 | 5087.24 | 0.71 | 1.14 | 1.03 |
| R98M | 3551.60 | 5834.31 | 0.61 | 1.12 | 1.18 |
| R98F | 2925.55 | 4988.31 | 0.59 | 0.92 | 1.01 |
| R98W | 833.68 | 1098.48 | 0.76 | 0.26 | 0.22 |
| R98Y | 505.91 | 479.02 | 1.06 | 0.16 | 0.10 |
| R98P | 2306.44 | 3388.72 | 0.68 | 0.73 | 0.69 |
| R98E | 1812.72 | 2769.07 | 0.65 | 0.57 | 0.56 |
| R98A | 3006.35 | 4371.72 | 0.69 | 0.95 | 0.89 |
| R98G | 1525.20 | 2367.66 | 0.64 | 0.48 | 0.48 |
| R98V | 1298.78 | 3330.10 | 0.39 | 0.26 | 0.49 |
| R98S | 4646.88 | 6142.58 | 0.76 | 0.94 | 0.90 |
| R98D | 2905.96 | 3867.31 | 0.75 | 0.33 | 0.47 |
| I99C | 514.61 | 514.66 | 1.00 | 0.16 | 0.10 |
| I99E | 550.80 | 548.33 | 1.00 | 0.17 | 0.11 |
| I99G | 588.17 | 598.60 | 0.98 | 0.19 | 0.12 |
| I99H | 749.01 | 834.15 | 0.90 | 0.24 | 0.17 |
| I99N | 691.55 | 805.73 | 0.86 | 0.22 | 0.16 |
| I99P | 567.03 | 526.02 | 1.08 | 0.18 | 0.11 |
| I99T | 1087.94 | 1583.58 | 0.69 | 0.34 | 0.32 |
| I99V | 2373.86 | 3390.37 | 0.70 | 0.75 | 0.69 |
| I99A | 654.22 | 809.57 | 0.81 | 0.13 | 0.12 |
| I99F | 2098.09 | 2958.45 | 0.71 | 0.43 | 0.43 |
| I99L | 2592.09 | 4336.89 | 0.60 | 0.53 | 0.63 |
| I99R | 561.16 | 555.21 | 1.01 | 0.11 | 0.08 |
| I99S | 616.13 | 673.46 | 0.91 | 0.12 | 0.10 |
| I99Q | 3318.21 | 4623.91 | 0.72 | 0.37 | 0.56 |
| I99W | 509.03 | 492.00 | 1.04 | 0.06 | 0.06 |
| I99Y | 690.55 | 700.48 | 0.99 | 0.08 | 0.09 |
| E100V | 3980.72 | 5009.20 | 0.79 | 1.26 | 1.01 |
| E100P | 727.82 | 785.14 | 0.93 | 0.23 | 0.16 |
| E100L | 3370.21 | 4726.28 | 0.71 | 1.06 | 0.96 |
| E100H | 1484.00 | 2354.50 | 0.63 | 0.47 | 0.48 |
| E100D | 1886.86 | 3049.67 | 0.62 | 0.60 | 0.62 |
| E100M | 3046.42 | 4566.62 | 0.67 | 0.96 | 0.92 |
| E100G | 541.78 | 567.31 | 0.95 | 0.11 | 0.08 |
| E100W | 1544.77 | 3766.06 | 0.41 | 0.31 | 0.55 |
| E100Y | 2885.60 | 4167.75 | 0.69 | 0.58 | 0.61 |
| E100R | 7410.11 | 7964.52 | 0.93 | 0.83 | 0.96 |
| E100S | 3768.09 | 4664.58 | 0.81 | 0.42 | 0.56 |
| E100T | 6985.28 | 7478.12 | 0.93 | 0.79 | 0.90 |
| E100F | 6709.27 | 7436.60 | 0.90 | 0.75 | 0.90 |
| E100I | 8824.19 | 8458.79 | 1.04 | 0.99 | 1.02 |
| E100N | 8809.68 | 8215.63 | 1.07 | 0.99 | 0.99 |
| N101M | 7907.75 | 7930.91 | 1.00 | 0.89 | 0.96 |
| N101F | 5045.54 | 5244.47 | 0.96 | 0.57 | 0.63 |
| N101L | 6427.09 | 6656.60 | 0.97 | 0.72 | 0.80 |
| N101V | 8153.10 | 7605.57 | 1.07 | 0.92 | 0.92 |
| N101H | 8863.48 | 8197.03 | 1.08 | 1.00 | 0.99 |
| N101R | 8050.92 | 7576.48 | 1.06 | 0.91 | 0.91 |
| N101C | 2651.70 | 3359.06 | 0.79 | 0.30 | 0.41 |
| N101T | 9660.15 | 8437.52 | 1.14 | 1.09 | 1.02 |
| N101P | 8232.08 | 7996.53 | 1.03 | 0.93 | 0.96 |
| N101W | 3302.54 | 3773.04 | 0.88 | 0.37 | 0.46 |
| N101K | 7396.60 | 7283.35 | 1.02 | 0.83 | 0.88 |
| N101S | 8913.81 | 8187.44 | 1.09 | 1.00 | 0.99 |
| N101D | 5424.50 | 5676.43 | 0.96 | 0.61 | 0.68 |
| N101A | 6371.04 | 6423.71 | 0.99 | 0.72 | 0.78 |
| N101Y | 3878.66 | 3898.52 | 0.99 | 0.44 | 0.47 |
| Y102R | 9027.37 | 8170.55 | 1.10 | 1.02 | 0.99 |
| Y102K | 5806.60 | 5157.24 | 1.13 | 0.65 | 0.62 |
| Y102V | 6412.64 | 6500.28 | 0.99 | 0.72 | 0.78 |
| Y102M | 6668.55 | 6964.91 | 0.96 | 0.75 | 0.84 |
| Y102P | 4670.45 | 4431.46 | 1.05 | 0.53 | 0.53 |
| Y102N | 4618.85 | 4579.81 | 1.01 | 0.52 | 0.55 |
| Y102G | 7272.09 | 6976.34 | 1.04 | 0.82 | 0.84 |
| Y102L | 3323.14 | 3802.15 | 0.87 | 0.37 | 0.46 |
| Y102D | 5174.42 | 4862.10 | 1.06 | 0.58 | 0.59 |
| Y102S | 8744.32 | 8244.11 | 1.06 | 0.98 | 0.99 |
| Y102F | 7629.25 | 8362.12 | 0.91 | 0.86 | 1.01 |
| Y102A | 7177.10 | 7304.92 | 0.98 | 0.81 | 0.88 |
| Y102E | 3375.30 | 3325.21 | 1.02 | 0.38 | 0.40 |
| Y102Q | 5644.96 | 5711.44 | 0.99 | 0.64 | 0.69 |
| Y102C | 1544.43 | 1842.83 | 0.84 | 0.17 | 0.22 |
| T103E | 619.06 | 617.97 | 1.00 | 0.20 | 0.13 |
| T103D | 848.44 | 877.46 | 0.97 | 0.27 | 0.18 |
| T103S | 761.49 | 855.80 | 0.89 | 0.24 | 0.17 |
| T103L | 855.65 | 650.61 | 1.32 | 0.27 | 0.13 |
| T103V | 822.60 | 1017.88 | 0.81 | 0.26 | 0.21 |
| T103R | 674.37 | 652.99 | 1.03 | 0.21 | 0.13 |
| T103Y | 1181.09 | 1423.76 | 0.83 | 0.37 | 0.29 |
| T103N | 3131.62 | 4822.91 | 0.65 | 0.99 | 0.98 |
| T103C | 628.62 | 604.98 | 1.04 | 0.20 | 0.12 |
| T103Q | 791.61 | 624.86 | 1.27 | 0.25 | 0.13 |
| T103W | 513.42 | 548.41 | 0.94 | 0.10 | 0.08 |
| T103P | 513.57 | 526.91 | 0.97 | 0.10 | 0.08 |
| T103A | 1058.92 | 950.05 | 1.11 | 0.21 | 0.14 |
| T103G | 749.67 | 656.69 | 1.14 | 0.15 | 0.10 |
| T103K | 884.09 | 777.94 | 1.14 | 0.18 | 0.11 |
| P104G | 602.57 | 620.78 | 0.97 | 0.19 | 0.13 |
| P104E | 4330.78 | 6029.01 | 0.72 | 1.37 | 1.22 |
| P104T | 3213.10 | 4681.67 | 0.69 | 1.02 | 0.95 |
| P104F | 2191.45 | 1923.19 | 1.14 | 0.69 | 0.39 |
| P104R | 591.46 | 5625.37 | 0.11 | 0.19 | 1.14 |
| P104D | 4022.87 | 5896.28 | 0.68 | 1.27 | 1.19 |
| P104C | 779.25 | 879.87 | 0.89 | 0.25 | 0.18 |
| P104Q | 4140.44 | 5971.62 | 0.69 | 1.31 | 1.21 |
| P104V | 2675.96 | 4161.77 | 0.64 | 0.85 | 0.84 |
| P104Y | 1907.52 | 2912.52 | 0.65 | 0.60 | 0.59 |
| P104H | 3404.74 | 5009.27 | 0.68 | 1.08 | 1.01 |
| P104L | 2981.52 | 4000.85 | 0.75 | 0.60 | 0.58 |
| P104S | 1205.11 | 2392.05 | 0.50 | 0.24 | 0.35 |
| P104A | 8861.30 | 8360.82 | 1.06 | 1.00 | 1.01 |
| P104M | 6709.44 | 7118.65 | 0.94 | 0.88 | 0.75 |
| D105A | 2674.16 | 1227.06 | 2.18 | 0.65 | 0.24 |
| D105C | 871.16 | 737.92 | 1.18 | 0.21 | 0.15 |
| D105F | 2009.56 | 1221.58 | 1.65 | 0.49 | 0.24 |
| D105G | 2407.89 | 1686.68 | 1.43 | 0.58 | 0.34 |
| D105I | 1732.38 | 1105.99 | 1.57 | 0.42 | 0.22 |
| D105L | 1563.61 | 859.56 | 1.82 | 0.38 | 0.17 |
| D105M | 2703.51 | 2920.93 | 0.93 | 0.65 | 0.58 |
| D105N | 3766.72 | 1475.08 | 2.55 | 0.91 | 0.29 |
| D105P | 856.02 | 604.56 | 1.42 | 0.21 | 0.12 |
| D105R | 3892.02 | 2016.90 | 1.93 | 0.94 | 0.40 |
| D105S | 3646.49 | 2727.22 | 1.34 | 0.88 | 0.54 |
| D105T | 2513.64 | 1729.46 | 1.45 | 0.61 | 0.34 |
| D105V | 5824.43 | 6784.65 | 0.86 | 1.41 | 1.35 |
| D105W | 2565.93 | 1855.05 | 1.38 | 0.62 | 0.37 |
| D105E | 4000.92 | 3366.64 | 1.19 | 0.59 | 0.45 |
| L106P | 793.18 | 480.45 | 1.65 | 0.16 | 0.10 |
| L106D | 455.97 | 436.46 | 1.04 | 0.09 | 0.09 |
| L106N | 579.84 | 499.77 | 1.16 | 0.12 | 0.10 |
| L106G | 778.24 | 578.12 | 1.35 | 0.16 | 0.12 |
| L106M | 2299.74 | 3704.96 | 0.62 | 0.48 | 0.74 |
| L106A | 3604.47 | 5633.39 | 0.64 | 0.75 | 1.12 |
| L106R | 658.60 | 552.82 | 1.19 | 0.14 | 0.11 |
| L106Y | 4761.33 | 5769.09 | 0.83 | 0.99 | 1.15 |
| L106T | 1604.22 | 1508.31 | 1.06 | 0.33 | 0.30 |
| L106V | 8561.50 | 8230.68 | 1.04 | 1.77 | 1.64 |
| L106H | 644.13 | 641.84 | 1.00 | 0.13 | 0.13 |
| L106F | 1776.88 | 2525.65 | 0.70 | 0.36 | 0.37 |
| L106I | 2787.16 | 4408.75 | 0.63 | 0.56 | 0.64 |
| L106C | 2995.56 | 3678.33 | 0.81 | 0.34 | 0.44 |
| L106S | 2730.64 | 2899.36 | 0.94 | 0.31 | 0.35 |
| P107L | 3183.54 | 3874.49 | 0.82 | 0.77 | 0.75 |
| P107W | 1255.79 | 1303.70 | 0.96 | 0.30 | 0.25 |
| P107T | 5673.07 | 6084.28 | 0.93 | 1.37 | 1.18 |
| P107S | 5865.31 | 6191.65 | 0.95 | 1.42 | 1.20 |
| P107R | 2981.87 | 3300.34 | 0.90 | 0.72 | 0.64 |
| P107Y | 2005.11 | 2383.15 | 0.84 | 0.48 | 0.46 |
| P107M | 3551.42 | 4031.55 | 0.88 | 0.86 | 0.78 |
| P107V | 3499.60 | 4142.87 | 0.84 | 0.85 | 0.80 |
| P107D | 3531.02 | 4095.17 | 0.86 | 0.85 | 0.80 |
| P107A | 5661.84 | 6316.88 | 0.90 | 1.37 | 1.23 |
| P107C | 786.68 | 776.20 | 1.01 | 0.19 | 0.15 |
| P107K | 3176.89 | 3653.27 | 0.87 | 0.77 | 0.71 |
| P107F | 1603.40 | 1832.50 | 0.87 | 0.39 | 0.36 |
| P107I | 2003.91 | 2369.36 | 0.85 | 0.48 | 0.46 |
| P107G | 2694.02 | 3272.63 | 0.82 | 0.65 | 0.64 |
| R108P | 4652.14 | 3388.90 | 1.37 | 0.96 | 0.67 |
| R108G | 4168.56 | 6204.10 | 0.67 | 0.86 | 1.24 |
| R108T | 1360.40 | 1652.92 | 0.82 | 0.28 | 0.33 |
| R108E | 5311.31 | 6829.34 | 0.78 | 1.10 | 1.36 |
| R108A | 5676.42 | 7183.19 | 0.79 | 1.18 | 1.43 |
| R108Y | 1527.69 | 2690.78 | 0.57 | 0.32 | 0.54 |
| R108K | 7212.78 | 9049.80 | 0.80 | 1.49 | 1.80 |
| R108C | 2092.15 | 2852.47 | 0.73 | 0.43 | 0.57 |
| R108S | 8515.31 | 8202.68 | 1.04 | 1.76 | 1.63 |
| R108F | 4264.07 | 5199.96 | 0.82 | 0.88 | 1.04 |
| R108W | 1522.39 | 2152.20 | 0.71 | 0.31 | 0.31 |
| R108I | 2968.84 | 4628.28 | 0.64 | 0.60 | 0.68 |
| R108L | 2200.90 | 3462.10 | 0.64 | 0.45 | 0.51 |
| R108N | 2820.25 | 4415.19 | 0.64 | 0.57 | 0.65 |
| R108V | 571.77 | 618.30 | 0.92 | 0.12 | 0.09 |
| A109S | 6193.70 | 7627.42 | 0.81 | 1.28 | 1.52 |
| A109R | 4933.84 | 9751.06 | 0.51 | 1.02 | 1.94 |
| A109T | 4678.95 | 6089.37 | 0.77 | 0.97 | 1.21 |
| A109W | 5152.58 | 6447.41 | 0.80 | 1.07 | 1.28 |
| A109I | 2587.03 | 4255.55 | 0.61 | 0.54 | 0.85 |
| A109Q | 3475.21 | 4698.87 | 0.74 | 0.72 | 0.94 |
| A109N | 6266.66 | 4399.73 | 1.42 | 1.30 | 0.88 |
| A109Y | 1880.37 | 1444.85 | 1.30 | 0.39 | 0.29 |
| A109G | 5864.62 | 12111.28 | 0.48 | 1.21 | 2.41 |
| A109M | 7784.21 | 8628.31 | 0.90 | 1.61 | 1.72 |
| A109D | 4410.30 | 6431.60 | 0.69 | 0.91 | 1.28 |
| A109V | 8073.90 | 8388.34 | 0.96 | 1.67 | 1.67 |
| A109E | 2859.74 | 7453.25 | 0.38 | 0.58 | 1.09 |
| A109L | 3649.92 | 5241.27 | 0.70 | 0.74 | 0.77 |
| A109H | 7206.01 | 7536.96 | 0.96 | 0.81 | 0.91 |
| D110P | 691.78 | 937.18 | 0.74 | 0.14 | 0.19 |
| D110F | 2469.89 | 3158.71 | 0.78 | 0.51 | 0.63 |
| D110Q | 3028.40 | 4201.99 | 0.72 | 0.63 | 0.84 |
| D110R | 756.25 | 1109.97 | 0.68 | 0.16 | 0.22 |
| D110M | 1094.79 | 917.81 | 1.19 | 0.23 | 0.18 |
| D110H | 3327.99 | 6569.83 | 0.51 | 0.69 | 1.31 |
| D110I | 1457.92 | 2219.69 | 0.66 | 0.30 | 0.44 |
| D110L | 1494.01 | 1991.44 | 0.75 | 0.31 | 0.40 |
| D110V | 2494.40 | 3413.88 | 0.73 | 0.52 | 0.68 |
| D110T | 2731.23 | 4170.98 | 0.65 | 0.57 | 0.83 |
| D110S | 1262.77 | 1714.94 | 0.74 | 0.26 | 0.34 |
| D110Y | 2764.78 | 5378.21 | 0.51 | 0.57 | 1.07 |
| D110G | 510.14 | 537.48 | 0.95 | 0.10 | 0.08 |
| D110C | 827.23 | 996.83 | 0.83 | 0.17 | 0.15 |
| D110A | 4179.59 | 5112.44 | 0.82 | 0.47 | 0.62 |
| V111E | 779.81 | 1134.36 | 0.69 | 0.16 | 0.23 |
| V111A | 1964.87 | 2890.23 | 0.68 | 0.41 | 0.58 |
| V111S | 2947.29 | 4188.33 | 0.70 | 0.61 | 0.83 |
| V111W | 601.19 | 580.46 | 1.04 | 0.12 | 0.12 |
| V111G | 833.15 | 974.76 | 0.85 | 0.17 | 0.19 |
| V111Y | 813.12 | 942.64 | 0.86 | 0.17 | 0.19 |
| V111P | 923.36 | 696.55 | 1.33 | 0.19 | 0.14 |
| V111L | 1070.50 | 1565.39 | 0.68 | 0.22 | 0.31 |
| V111D | 591.79 | 576.36 | 1.03 | 0.12 | 0.11 |
| V111K | 1017.96 | 1328.59 | 0.77 | 0.21 | 0.26 |
| V111T | 3551.97 | 4859.95 | 0.73 | 0.74 | 0.97 |
| V111Q | 1546.82 | 2061.98 | 0.75 | 0.32 | 0.41 |
| V111I | 4959.51 | 6699.66 | 0.74 | 1.03 | 1.33 |
| V111C | 843.17 | 943.89 | 0.89 | 0.17 | 0.14 |
| V111R | 2401.69 | 2925.16 | 0.82 | 0.27 | 0.35 |
| D112A | 1419.86 | 2167.48 | 0.66 | 0.29 | 0.43 |
| D112M | 1668.58 | 2249.91 | 0.74 | 0.35 | 0.45 |
| D112V | 2683.45 | 3699.41 | 0.73 | 0.56 | 0.74 |
| D112R | 1072.27 | 1395.54 | 0.77 | 0.22 | 0.28 |
| D112K | 967.53 | 1261.79 | 0.77 | 0.20 | 0.25 |
| D112P | 565.23 | 589.06 | 0.96 | 0.12 | 0.12 |
| D112Q | 4681.31 | 8975.21 | 0.52 | 0.97 | 1.79 |
| D112F | 1148.89 | 1477.74 | 0.78 | 0.24 | 0.29 |
| D112G | 1824.01 | 2601.95 | 0.70 | 0.38 | 0.52 |
| D112C | 866.83 | 1034.64 | 0.84 | 0.18 | 0.21 |
| D112W | 937.80 | 1277.50 | 0.73 | 0.19 | 0.25 |
| D112T | 2538.82 | 2941.38 | 0.86 | 0.53 | 0.59 |
| D112H | 480.11 | 467.40 | 1.03 | 0.10 | 0.07 |
| D112S | 7203.69 | 7600.93 | 0.95 | 0.81 | 0.92 |
| D112I | 4020.53 | 5498.90 | 0.73 | 0.44 | 0.72 |
| D112Y | 2132.97 | 2869.86 | 0.74 | 0.23 | 0.38 |
| D112L | 2626.71 | 4159.92 | 0.63 | 0.29 | 0.55 |
| H113T | 9107.72 | 8278.01 | 1.10 | 1.03 | 1.00 |
| H113L | 9479.59 | 8454.16 | 1.12 | 1.07 | 1.02 |
| H113M | 9463.40 | 8759.43 | 1.08 | 1.07 | 1.06 |
| H113S | 9278.22 | 9159.47 | 1.01 | 1.04 | 1.11 |
| H113N | 8609.35 | 8502.46 | 1.01 | 0.97 | 1.03 |
| H113R | 7702.30 | 7852.46 | 0.98 | 0.87 | 0.95 |
| H113A | 8505.43 | 8090.18 | 1.05 | 0.96 | 0.98 |
| H113E | 9118.02 | 8443.69 | 1.08 | 1.03 | 1.02 |
| H113V | 9183.53 | 8450.30 | 1.09 | 1.03 | 1.02 |
| H113Y | 9688.60 | 8548.83 | 1.13 | 1.09 | 1.03 |
| H113F | 9472.51 | 8729.41 | 1.09 | 1.07 | 1.05 |
| H113D | 9304.42 | 4925.78 | 1.89 | 1.05 | 0.59 |
| H113W | 8683.10 | 5775.24 | 1.50 | 0.98 | 0.70 |
| H113G | 8953.60 | 8320.09 | 1.08 | 1.01 | 1.00 |
| H113P | 2987.12 | 3102.32 | 0.96 | 0.34 | 0.37 |
| A114E | 7136.25 | 7924.97 | 0.90 | 0.80 | 0.96 |
| A114S | 9211.05 | 8794.50 | 1.05 | 1.04 | 1.06 |
| A114I | 7073.18 | 7475.79 | 0.95 | 0.80 | 0.90 |
| A114P | 1691.05 | 1357.51 | 1.25 | 0.19 | 0.16 |
| A114N | 9250.51 | 8746.70 | 1.06 | 1.04 | 1.06 |
| A114L | 7749.61 | 8007.88 | 0.97 | 0.87 | 0.97 |
| A114T | 6242.22 | 6974.59 | 0.89 | 0.70 | 0.84 |
| A114F | 605.35 | 675.10 | 0.90 | 0.07 | 0.08 |
| A114V | 5527.85 | 6054.48 | 0.91 | 0.62 | 0.73 |
| A114G | 7663.26 | 7892.13 | 0.97 | 0.86 | 0.95 |
| A114C | 2412.52 | 3005.83 | 0.80 | 0.27 | 0.36 |
| A114M | 5287.05 | 5931.99 | 0.89 | 0.60 | 0.72 |
| A114R | 4454.65 | 3915.86 | 1.14 | 0.50 | 0.47 |
| A114W | 4654.58 | 5477.95 | 0.85 | 0.52 | 0.66 |
| A114Q | 8094.57 | 8337.94 | 0.97 | 0.91 | 1.01 |
| I115F | 9634.62 | 9011.34 | 1.07 | 1.08 | 1.09 |
| I115T | 1935.83 | 2379.92 | 0.81 | 0.22 | 0.29 |
| I115H | 805.66 | 825.35 | 0.98 | 0.09 | 0.10 |
| I115G | 725.85 | 626.12 | 1.16 | 0.08 | 0.08 |
| I115K | 642.87 | 920.32 | 0.70 | 0.07 | 0.11 |
| I115E | 1276.09 | 1211.16 | 1.05 | 0.14 | 0.15 |
| I115S | 796.93 | 780.25 | 1.02 | 0.09 | 0.09 |
| I115P | 626.77 | 597.01 | 1.05 | 0.07 | 0.07 |
| I115C | 1021.21 | 982.43 | 1.04 | 0.11 | 0.12 |
| I115L | 8869.57 | 8467.55 | 1.05 | 1.00 | 1.02 |
| I115Q | 732.25 | 652.35 | 1.12 | 0.08 | 0.08 |
| I115R | 750.11 | 575.36 | 1.30 | 0.08 | 0.07 |
| I115W | 2203.68 | 2304.27 | 0.96 | 0.25 | 0.28 |
| I115V | 9365.90 | 8785.30 | 1.07 | 1.05 | 1.06 |
| I115D | 694.17 | 641.97 | 1.08 | 0.08 | 0.08 |
| E116A | 9273.62 | 9051.39 | 1.02 | 1.04 | 1.09 |
| E116C | 5022.73 | 5732.42 | 0.88 | 0.57 | 0.69 |
| E116D | 9114.14 | 8594.45 | 1.06 | 1.03 | 1.04 |
| E116F | 8569.56 | 8473.84 | 1.01 | 0.96 | 1.02 |
| E116G | 8305.07 | 8358.04 | 0.99 | 0.94 | 1.01 |
| E116H | 8630.15 | 8386.63 | 1.03 | 0.97 | 1.01 |
| E116I | 9386.17 | 8740.84 | 1.07 | 1.06 | 1.05 |
| E116K | 9320.21 | 8760.44 | 1.06 | 1.05 | 1.06 |
| E116L | 8997.58 | 8736.18 | 1.03 | 1.01 | 1.05 |
| E116M | 9046.33 | 8478.98 | 1.07 | 1.02 | 1.02 |
| E116N | 8629.15 | 8503.39 | 1.01 | 0.97 | 1.03 |
| E116P | 852.91 | 806.00 | 1.06 | 0.10 | 0.10 |
| E116Q | 9480.67 | 8716.72 | 1.09 | 1.07 | 1.05 |
| E116R | 8871.32 | 8479.40 | 1.05 | 1.00 | 1.02 |
| E116S | 9714.89 | 8843.17 | 1.10 | 1.09 | 1.07 |
| K117H | 4516.51 | 4612.42 | 0.98 | 0.52 | 0.55 |
| K117T | 6149.94 | 6317.74 | 0.97 | 0.71 | 0.75 |
| K117Q | 6602.62 | 6024.00 | 1.10 | 0.77 | 0.72 |
| K117E | 668.03 | 667.32 | 1.00 | 0.08 | 0.08 |
| K117A | 7727.36 | 7375.30 | 1.05 | 0.90 | 0.88 |
| K117F | 4020.90 | 4038.83 | 1.00 | 0.47 | 0.48 |
| K117D | 5330.37 | 5924.02 | 0.90 | 0.62 | 0.70 |
| K117N | 4666.40 | 7745.69 | 0.60 | 0.54 | 0.92 |
| K117G | 7619.16 | 7218.94 | 1.06 | 0.88 | 0.86 |
| K117W | 5440.86 | 4780.56 | 1.14 | 0.63 | 0.57 |
| K117Y | 5047.23 | 4760.05 | 1.06 | 0.59 | 0.57 |
| K117L | 5277.39 | 5328.70 | 0.99 | 0.61 | 0.63 |
| K117S | 7278.89 | 6995.65 | 1.04 | 0.85 | 0.83 |
| K117P | 737.96 | 1153.03 | 0.64 | 0.09 | 0.14 |
| K117R | 8236.16 | 7677.40 | 1.07 | 0.96 | 0.91 |
| A118G | 2782.31 | 6427.69 | 0.43 | 0.32 | 0.76 |
| A118R | 4889.61 | 5639.79 | 0.87 | 0.57 | 0.67 |
| A118W | 652.55 | 465.07 | 1.40 | 0.08 | 0.06 |
| A118K | 584.59 | 543.84 | 1.07 | 0.07 | 0.06 |
| A118P | 883.04 | 810.72 | 1.09 | 0.10 | 0.10 |
| A118V | 869.06 | 754.10 | 1.15 | 0.10 | 0.09 |
| A118L | 543.99 | 523.84 | 1.04 | 0.06 | 0.06 |
| A118D | 617.40 | 468.39 | 1.32 | 0.07 | 0.06 |
| A118S | 5502.11 | 8251.39 | 0.67 | 0.64 | 0.98 |
| A118F | 7092.17 | 7315.30 | 0.97 | 0.82 | 0.87 |
| A118I | 556.24 | 456.62 | 1.22 | 0.06 | 0.05 |
| A118H | 482.33 | 466.40 | 1.03 | 0.06 | 0.06 |
| A118E | 560.55 | 406.52 | 1.38 | 0.07 | 0.05 |
| A118Q | 517.05 | 477.18 | 1.08 | 0.06 | 0.06 |
| A118T | 745.83 | 665.63 | 1.12 | 0.13 | 0.13 |
| F119G | 2058.01 | 3284.43 | 0.63 | 0.24 | 0.39 |
| F119T | 4492.83 | 8234.56 | 0.55 | 0.52 | 0.98 |
| F119R | 648.01 | 665.21 | 0.97 | 0.08 | 0.08 |
| F119L | 8529.66 | 7666.52 | 1.11 | 0.99 | 0.91 |
| F119N | 1298.98 | 1614.30 | 0.80 | 0.15 | 0.19 |
| F119S | 3021.31 | 4383.36 | 0.69 | 0.35 | 0.52 |
| F119C | 2921.13 | 3375.91 | 0.87 | 0.34 | 0.40 |
| F119P | 567.10 | 665.80 | 0.85 | 0.07 | 0.08 |
| F119W | 4474.41 | 4610.60 | 0.97 | 0.52 | 0.55 |
| F119K | 679.32 | 762.81 | 0.89 | 0.08 | 0.09 |
| F119H | 2479.46 | 3939.67 | 0.63 | 0.29 | 0.47 |
| F119A | 7345.58 | 7881.39 | 0.93 | 0.85 | 0.94 |
| F119V | 7388.01 | 7712.75 | 0.96 | 0.86 | 0.92 |
| F119Y | 5832.62 | 6222.88 | 0.94 | 0.68 | 0.74 |
| F119E | 1044.34 | 1357.80 | 0.77 | 0.12 | 0.16 |
| Q120K | 8732.08 | 8385.78 | 1.04 | 1.01 | 1.00 |
| Q120N | 9186.34 | 8785.94 | 1.05 | 1.07 | 1.05 |
| Q120A | 613.72 | 979.68 | 0.63 | 0.07 | 0.12 |
| Q120V | 8711.16 | 8484.49 | 1.03 | 1.01 | 1.01 |
| Q120D | 5912.84 | 8887.98 | 0.67 | 0.69 | 1.06 |
| Q120R | 8845.48 | 8351.59 | 1.06 | 1.03 | 0.99 |
| Q120P | 1083.78 | 1186.17 | 0.91 | 0.13 | 0.14 |
| Q120W | 9339.94 | 7899.14 | 1.18 | 1.08 | 0.94 |
| Q120Y | 4891.24 | 8236.87 | 0.59 | 0.57 | 0.98 |
| Q120C | 5241.83 | 5502.66 | 0.95 | 0.61 | 0.65 |
| Q120H | 9155.83 | 8431.63 | 1.09 | 1.06 | 1.00 |
| Q120T | 9413.75 | 8645.01 | 1.09 | 1.09 | 1.03 |
| Q120M | 5740.33 | 8861.16 | 0.65 | 0.67 | 1.05 |
| Q120E | 8896.83 | 8424.76 | 1.06 | 1.03 | 1.00 |
| Q120G | 9176.97 | 8435.97 | 1.09 | 1.07 | 1.00 |
| L121E | 3183.74 | 2155.16 | 1.48 | 0.37 | 0.26 |
| L121Q | 2122.18 | 3128.11 | 0.68 | 0.25 | 0.37 |
| L121P | 1446.80 | 1342.92 | 1.08 | 0.17 | 0.16 |
| L121R | 1129.37 | 875.68 | 1.29 | 0.13 | 0.10 |
| L121C | 1592.13 | 1145.83 | 1.39 | 0.18 | 0.14 |
| L121G | 2613.21 | 4720.78 | 0.55 | 0.30 | 0.56 |
| L121K | 4678.64 | 4882.50 | 0.96 | 0.54 | 0.58 |
| L121F | 1227.52 | 957.08 | 1.28 | 0.14 | 0.11 |
| L121I | 7406.57 | 6937.72 | 1.07 | 0.86 | 0.83 |
| L121S | 2463.24 | 3614.68 | 0.68 | 0.29 | 0.43 |
| L121V | 7973.31 | 7244.01 | 1.10 | 0.93 | 0.86 |
| L121H | 3156.18 | 2605.48 | 1.21 | 0.37 | 0.31 |
| L121T | 7283.06 | 7372.13 | 0.99 | 0.85 | 0.88 |
| L121A | 3311.41 | 4989.77 | 0.66 | 0.38 | 0.59 |
| L121N | 6619.84 | 6504.79 | 1.02 | 0.77 | 0.77 |
| W122R | 651.20 | 598.41 | 1.09 | 0.08 | 0.07 |
| W122A | 699.90 | 617.84 | 1.13 | 0.08 | 0.07 |
| W122N | 484.17 | 598.30 | 0.81 | 0.06 | 0.07 |
| W122P | 619.39 | 605.42 | 1.02 | 0.07 | 0.07 |
| W122T | 621.86 | 570.65 | 1.09 | 0.07 | 0.07 |
| W122L | 580.35 | 563.09 | 1.03 | 0.07 | 0.07 |
| W122G | 602.75 | 646.94 | 0.93 | 0.07 | 0.08 |
| W122S | 602.28 | 564.94 | 1.07 | 0.07 | 0.07 |
| W122V | 607.75 | 532.36 | 1.14 | 0.07 | 0.06 |
| W122H | 596.81 | 545.92 | 1.09 | 0.07 | 0.06 |
| W122F | 2018.83 | 3056.56 | 0.66 | 0.23 | 0.36 |
| W122Y | 667.50 | 661.98 | 1.01 | 0.08 | 0.08 |
| W122K | 2724.60 | 2334.11 | 1.17 | 0.32 | 0.28 |
| W122Q | 576.75 | 528.48 | 1.09 | 0.07 | 0.06 |
| W122E | 564.38 | 580.16 | 0.97 | 0.07 | 0.07 |
| S123D | 9453.37 | 8830.71 | 1.07 | 0.92 | 0.94 |
| S123L | 9912.51 | 9431.98 | 1.05 | 0.97 | 1.01 |
| S123A | 9881.07 | 9237.14 | 1.07 | 0.97 | 0.99 |
| S123C | 10654.40 | 8973.60 | 1.19 | 1.04 | 0.96 |
| S123I | 9679.91 | 8521.19 | 1.14 | 0.95 | 0.91 |
| S123K | 10567.78 | 9024.26 | 1.17 | 1.03 | 0.96 |
| S123N | 6481.00 | 5911.32 | 1.10 | 0.63 | 0.63 |
| S123F | 7485.79 | 8458.67 | 0.88 | 0.73 | 0.90 |
| S123Y | 7667.20 | 8806.19 | 0.87 | 0.75 | 0.94 |
| S123M | 9800.43 | 9159.15 | 1.07 | 0.96 | 0.98 |
| S123H | 10038.71 | 9099.05 | 1.10 | 0.98 | 0.97 |
| S123R | 5290.53 | 9248.50 | 0.57 | 0.52 | 0.99 |
| S123W | 2039.75 | 5970.03 | 0.34 | 0.20 | 0.64 |
| S123T | 5042.33 | 9146.80 | 0.55 | 0.49 | 0.98 |
| S123P | 884.66 | 799.56 | 1.11 | 0.09 | 0.09 |
| S123G | 10847.89 | 9512.32 | 1.14 | 1.06 | 1.02 |
| S123Q | 10841.56 | 9551.30 | 1.14 | 1.06 | 1.02 |
| S123V | 3220.29 | 4504.25 | 0.71 | 0.41 | 0.60 |
| N124G | 5601.70 | 6396.41 | 0.88 | 1.35 | 1.27 |
| N124C | 2241.39 | 2691.13 | 0.83 | 0.54 | 0.53 |
| N124V | 2966.25 | 3399.34 | 0.87 | 0.72 | 0.68 |
| N124L | 2342.72 | 2849.98 | 0.82 | 0.57 | 0.57 |
| N124T | 3872.37 | 4747.39 | 0.82 | 0.94 | 0.94 |
| N124R | 3795.95 | 4479.74 | 0.85 | 0.92 | 0.89 |
| N124M | 2818.81 | 3511.81 | 0.80 | 0.68 | 0.70 |
| N124S | 4245.94 | 5151.63 | 0.82 | 1.03 | 1.02 |
| N124P | 3825.40 | 5084.71 | 0.75 | 0.92 | 1.01 |
| N124A | 4174.53 | 4857.21 | 0.86 | 1.01 | 0.96 |
| N124K | 5006.93 | 5514.55 | 0.91 | 1.21 | 1.10 |
| N124F | 3681.53 | 4406.27 | 0.84 | 0.89 | 0.88 |
| N124W | 1506.21 | 1714.90 | 0.88 | 0.36 | 0.34 |
| N124I | 1663.57 | 1830.11 | 0.91 | 0.40 | 0.36 |
| N124D | 6218.73 | 6620.92 | 0.94 | 0.92 | 0.88 |
| V125G | 532.18 | 540.26 | 0.99 | 0.09 | 0.07 |
| V125Q | 1480.08 | 1883.56 | 0.79 | 0.26 | 0.25 |
| V125S | 2153.87 | 2966.73 | 0.73 | 0.38 | 0.39 |
| V125P | 1410.46 | 1873.09 | 0.75 | 0.25 | 0.24 |
| V125M | 1056.84 | 1118.42 | 0.94 | 0.19 | 0.15 |
| V125Y | 1484.83 | 2214.89 | 0.67 | 0.26 | 0.29 |
| V125T | 1444.16 | 1850.94 | 0.78 | 0.25 | 0.24 |
| V125A | 3246.01 | 5558.20 | 0.58 | 0.57 | 0.73 |
| V125C | 892.38 | 690.11 | 1.29 | 0.16 | 0.09 |
| V125D | 727.50 | 723.91 | 1.00 | 0.13 | 0.09 |
| V125W | 1537.82 | 1638.09 | 0.94 | 0.27 | 0.21 |
| V125R | 1087.69 | 1057.82 | 1.03 | 0.19 | 0.14 |
| V125E | 1324.10 | 1545.82 | 0.86 | 0.23 | 0.20 |
| V125F | 1360.07 | 2068.15 | 0.66 | 0.24 | 0.27 |
| V125H | 2227.75 | 2720.50 | 0.82 | 0.39 | 0.36 |
| T126K | 646.69 | 546.31 | 1.18 | 0.16 | 0.11 |
| T126V | 3034.58 | 4559.28 | 0.67 | 0.73 | 0.91 |
| T126G | 970.67 | 820.16 | 1.18 | 0.23 | 0.16 |
| T126R | 692.68 | 612.62 | 1.13 | 0.17 | 0.12 |
| T126L | 1084.98 | 970.83 | 1.12 | 0.26 | 0.19 |
| T126H | 648.90 | 592.08 | 1.10 | 0.16 | 0.12 |
| T126M | 1168.66 | 1078.26 | 1.08 | 0.28 | 0.21 |
| T126P | 684.23 | 614.07 | 1.11 | 0.17 | 0.12 |
| T126A | 2433.37 | 2923.43 | 0.83 | 0.59 | 0.58 |
| T126N | 1449.19 | 1384.47 | 1.05 | 0.35 | 0.28 |
| T126E | 697.86 | 580.78 | 1.20 | 0.17 | 0.12 |
| T126F | 642.61 | 550.97 | 1.17 | 0.16 | 0.11 |
| T126W | 632.89 | 564.16 | 1.12 | 0.15 | 0.11 |
| T126Q | 664.00 | 591.91 | 1.12 | 0.16 | 0.12 |
| T126S | 7114.42 | 6856.69 | 1.04 | 1.06 | 0.91 |
| P127C | 1713.51 | 1846.56 | 0.93 | 0.41 | 0.37 |
| P127F | 1444.31 | 1603.37 | 0.90 | 0.35 | 0.32 |
| P127T | 2193.26 | 2519.16 | 0.87 | 0.53 | 0.50 |
| P127E | 2480.57 | 3177.56 | 0.78 | 0.60 | 0.63 |
| P127W | 1399.71 | 1476.35 | 0.95 | 0.34 | 0.29 |
| P127A | 1751.82 | 1662.47 | 1.05 | 0.42 | 0.33 |
| P127S | 2842.19 | 3070.41 | 0.93 | 0.69 | 0.61 |
| P127H | 2151.26 | 1693.77 | 1.27 | 0.52 | 0.34 |
| P127Q | 1729.40 | 1882.54 | 0.92 | 0.42 | 0.37 |
| P127K | 729.23 | 657.19 | 1.11 | 0.18 | 0.13 |
| P127R | 1590.44 | 1491.10 | 1.07 | 0.38 | 0.30 |
| P127I | 1432.03 | 1464.78 | 0.98 | 0.35 | 0.29 |
| P127V | 1214.79 | 1401.27 | 0.87 | 0.29 | 0.28 |
| P127L | 1536.18 | 1604.60 | 0.96 | 0.37 | 0.32 |
| P127M | 2950.98 | 3052.79 | 0.97 | 0.71 | 0.61 |
| L128F | 1165.63 | 1269.01 | 0.92 | 0.28 | 0.25 |
| L128M | 1898.38 | 2135.63 | 0.89 | 0.46 | 0.42 |
| L128T | 756.63 | 698.21 | 1.08 | 0.18 | 0.14 |
| L128R | 919.42 | 960.28 | 0.96 | 0.22 | 0.19 |
| L128S | 764.28 | 672.98 | 1.14 | 0.18 | 0.13 |
| L128G | 738.26 | 694.65 | 1.06 | 0.18 | 0.14 |
| L128I | 1482.67 | 1715.03 | 0.86 | 0.36 | 0.34 |
| L128Q | 1042.55 | 936.43 | 1.11 | 0.25 | 0.19 |
| L128P | 792.57 | 760.45 | 1.04 | 0.19 | 0.15 |
| L128A | 769.15 | 712.50 | 1.08 | 0.19 | 0.14 |
| L128D | 682.02 | 642.58 | 1.06 | 0.16 | 0.13 |
| L128V | 1285.36 | 1696.88 | 0.76 | 0.31 | 0.34 |
| L128W | 776.89 | 664.61 | 1.17 | 0.19 | 0.13 |
| L128C | 856.43 | 770.10 | 1.11 | 0.21 | 0.15 |
| L128K | 858.27 | 846.02 | 1.01 | 0.21 | 0.17 |
| T129G | 4435.98 | 5356.41 | 0.83 | 1.07 | 1.06 |
| T129A | 2021.18 | 2774.35 | 0.73 | 0.49 | 0.55 |
| T129C | 1057.55 | 1033.96 | 1.02 | 0.26 | 0.21 |
| T129K | 3686.95 | 4446.15 | 0.83 | 0.89 | 0.88 |
| T129F | 2980.80 | 3803.12 | 0.78 | 0.72 | 0.76 |
| T129Y | 2527.88 | 2885.14 | 0.88 | 0.61 | 0.57 |
| T129S | 1649.34 | 1529.13 | 1.08 | 0.40 | 0.30 |
| T129R | 3334.95 | 3827.40 | 0.87 | 0.81 | 0.76 |
| T129V | 4967.86 | 5698.42 | 0.87 | 1.20 | 1.13 |
| T129L | 1649.57 | 1692.51 | 0.97 | 0.40 | 0.34 |
| T129H | 3019.81 | 3803.29 | 0.79 | 0.73 | 0.76 |
| T129P | 647.52 | 619.28 | 1.05 | 0.16 | 0.12 |
| T129E | 3205.16 | 3919.94 | 0.82 | 0.77 | 0.78 |
| T129I | 3967.14 | 4452.39 | 0.89 | 0.96 | 0.88 |
| T129M | 4118.98 | 5214.21 | 0.79 | 1.00 | 1.04 |
| F130L | 1452.17 | 1651.58 | 0.88 | 0.25 | 0.23 |
| F130P | 703.36 | 797.39 | 0.88 | 0.12 | 0.11 |
| F130C | 803.88 | 939.50 | 0.86 | 0.14 | 0.13 |
| F130R | 613.42 | 687.58 | 0.89 | 0.10 | 0.09 |
| F130Y | 2355.45 | 3604.50 | 0.65 | 0.40 | 0.49 |
| F130H | 1209.72 | 1960.40 | 0.62 | 0.21 | 0.27 |
| F130I | 4480.99 | 5648.72 | 0.79 | 0.76 | 0.77 |
| F130V | 3403.91 | 4744.33 | 0.72 | 0.58 | 0.65 |
| F130K | 581.97 | 670.87 | 0.87 | 0.10 | 0.09 |
| F130T | 1529.21 | 2157.46 | 0.71 | 0.26 | 0.30 |
| F130E | 571.41 | 648.15 | 0.88 | 0.10 | 0.09 |
| F130A | 1414.89 | 1990.16 | 0.71 | 0.24 | 0.27 |
| F130N | 616.91 | 710.45 | 0.87 | 0.10 | 0.10 |
| F130G | 1553.35 | 1726.90 | 0.90 | 0.26 | 0.24 |
| F130S | 793.40 | 1055.93 | 0.75 | 0.13 | 0.14 |
| T131F | 2738.64 | 4500.49 | 0.61 | 0.48 | 0.59 |
| T131P | 540.49 | 640.19 | 0.84 | 0.10 | 0.08 |
| T131A | 3622.28 | 6028.39 | 0.60 | 0.64 | 0.79 |
| T131S | 3644.14 | 5779.25 | 0.63 | 0.64 | 0.75 |
| T131G | 3345.71 | 5523.72 | 0.61 | 0.59 | 0.72 |
| T131I | 2987.26 | 4570.78 | 0.65 | 0.53 | 0.60 |
| T131L | 3081.92 | 4518.80 | 0.68 | 0.54 | 0.59 |
| T131H | 4201.01 | 5298.03 | 0.79 | 0.74 | 0.69 |
| T131Q | 6169.43 | 8400.64 | 0.73 | 1.08 | 1.10 |
| T131D | 8629.30 | 9616.48 | 0.90 | 1.52 | 1.26 |
| T131E | 4396.59 | 6846.70 | 0.64 | 0.77 | 0.89 |
| T131C | 2232.15 | 3514.32 | 0.64 | 0.39 | 0.46 |
| T131R | 4325.73 | 6209.92 | 0.70 | 0.76 | 0.81 |
| T131Y | 2684.82 | 3916.43 | 0.69 | 0.47 | 0.51 |
| T131M | 3101.25 | 4674.29 | 0.66 | 0.55 | 0.61 |
| K132G | 3779.04 | 5835.32 | 0.65 | 0.64 | 0.80 |
| K132V | 3181.94 | 4834.70 | 0.66 | 0.54 | 0.66 |
| K132L | 2407.98 | 3744.64 | 0.64 | 0.41 | 0.51 |
| K132A | 5397.96 | 7468.39 | 0.72 | 0.92 | 1.02 |
| K132P | 4062.71 | 5742.05 | 0.71 | 0.69 | 0.79 |
| K132F | 2012.87 | 2934.12 | 0.69 | 0.34 | 0.40 |
| K132R | 7317.48 | 8467.31 | 0.86 | 1.24 | 1.16 |
| K132I | 1811.13 | 2747.29 | 0.66 | 0.31 | 0.38 |
| K132H | 3291.99 | 4588.07 | 0.72 | 0.56 | 0.63 |
| K132S | 4947.26 | 4913.96 | 1.01 | 0.84 | 0.67 |
| K132M | 4521.82 | 6773.06 | 0.67 | 0.77 | 0.93 |
| K132D | 2079.75 | 3166.80 | 0.66 | 0.35 | 0.43 |
| K132T | 2515.58 | 4096.35 | 0.61 | 0.43 | 0.56 |
| K132Y | 2363.32 | 3794.19 | 0.62 | 0.40 | 0.52 |
| K132E | 3617.16 | 5597.32 | 0.65 | 0.61 | 0.77 |
| V133G | 3203.88 | 5198.66 | 0.62 | 0.54 | 0.71 |
| V133E | 3621.55 | 5211.22 | 0.69 | 0.62 | 0.71 |
| V133T | 7931.49 | 8920.49 | 0.89 | 1.35 | 1.22 |
| V133N | 4321.90 | 6145.40 | 0.70 | 0.73 | 0.84 |
| V133A | 4764.29 | 6847.44 | 0.70 | 0.81 | 0.94 |
| V133H | 3351.37 | 4739.59 | 0.71 | 0.57 | 0.65 |
| V133P | 1405.33 | 2047.83 | 0.69 | 0.24 | 0.28 |
| V133K | 5737.27 | 7514.38 | 0.76 | 0.97 | 1.03 |
| V133R | 5773.82 | 7252.24 | 0.80 | 0.98 | 0.99 |
| V133L | 7039.08 | 8445.43 | 0.83 | 1.20 | 1.16 |
| V133W | 2475.35 | 3564.76 | 0.69 | 0.42 | 0.49 |
| V133C | 1863.63 | 2666.24 | 0.70 | 0.32 | 0.37 |
| V133D | 1792.46 | 2630.40 | 0.68 | 0.30 | 0.36 |
| V133M | 4618.45 | 6302.34 | 0.73 | 0.78 | 0.86 |
| V133S | 3077.53 | 4401.10 | 0.70 | 0.52 | 0.60 |
| S134V | 4041.51 | 5701.67 | 0.71 | 0.69 | 0.78 |
| S134H | 6079.44 | 7343.68 | 0.83 | 1.03 | 1.01 |
| S134P | 4779.98 | 6326.78 | 0.76 | 0.81 | 0.87 |
| S134G | 5540.33 | 7442.86 | 0.74 | 0.94 | 1.02 |
| S134N | 6292.89 | 7595.95 | 0.83 | 1.07 | 1.04 |
| S134R | 5129.73 | 6824.16 | 0.75 | 0.87 | 0.94 |
| S134L | 6015.18 | 8101.31 | 0.74 | 1.02 | 1.11 |
| S134Q | 4325.14 | 6159.02 | 0.70 | 0.73 | 0.84 |
| S134E | 7105.19 | 8577.53 | 0.83 | 1.21 | 1.18 |
| S134Y | 5061.86 | 6645.94 | 0.76 | 0.86 | 0.91 |
| S134A | 5179.47 | 6920.72 | 0.75 | 0.88 | 0.95 |
| S134K | 5768.02 | 7876.85 | 0.73 | 0.98 | 1.08 |
| S134D | 6958.26 | 8316.41 | 0.84 | 1.18 | 1.14 |
| S134T | 5585.98 | 7301.80 | 0.77 | 0.95 | 1.00 |
| S134C | 1950.58 | 2625.09 | 0.74 | 0.33 | 0.36 |
| E135V | 4036.77 | 5545.23 | 0.73 | 0.69 | 0.76 |
| E135M | 8700.42 | 9297.63 | 0.94 | 1.48 | 1.27 |
| E135S | 3895.80 | 5128.41 | 0.76 | 0.66 | 0.70 |
| E135D | 4858.77 | 6640.34 | 0.73 | 0.83 | 0.91 |
| E135T | 4870.41 | 6518.41 | 0.75 | 0.83 | 0.89 |
| E135L | 3276.24 | 4342.02 | 0.75 | 0.56 | 0.59 |
| E135A | 5143.68 | 7429.20 | 0.69 | 0.87 | 1.02 |
| E135W | 3407.93 | 4761.19 | 0.72 | 0.58 | 0.65 |
| E135F | 3206.26 | 4561.41 | 0.70 | 0.54 | 0.63 |
| E135P | 1077.62 | 1567.43 | 0.69 | 0.18 | 0.21 |
| E135R | 815.91 | 921.41 | 0.89 | 0.14 | 0.13 |
| E135N | 4626.44 | 6661.57 | 0.69 | 0.79 | 0.91 |
| E135H | 6074.22 | 7339.12 | 0.83 | 1.03 | 1.01 |
| E135Q | 5656.70 | 7144.49 | 0.79 | 0.96 | 0.98 |
| E135I | 2140.04 | 5232.08 | 0.41 | 0.36 | 0.72 |
| G136V | 1813.26 | 2616.78 | 0.69 | 0.31 | 0.36 |
| G136W | 993.10 | 1243.43 | 0.80 | 0.17 | 0.17 |
| G136D | 3591.52 | 5274.69 | 0.68 | 0.61 | 0.72 |
| G136M | 3515.40 | 5367.37 | 0.65 | 0.60 | 0.74 |
| G136N | 3503.65 | 5155.57 | 0.68 | 0.60 | 0.71 |
| G136A | 3559.58 | 5813.34 | 0.61 | 0.60 | 0.80 |
| G136L | 2187.68 | 3866.01 | 0.57 | 0.37 | 0.53 |
| G136C | 905.15 | 1515.22 | 0.60 | 0.15 | 0.21 |
| G136P | 3234.60 | 4934.89 | 0.66 | 0.55 | 0.68 |
| G136T | 2555.79 | 3746.36 | 0.68 | 0.43 | 0.51 |
| G136R | 2716.62 | 4398.06 | 0.62 | 0.46 | 0.60 |
| G136S | 3375.11 | 4670.21 | 0.72 | 0.57 | 0.64 |
| G136I | 2006.39 | 3604.58 | 0.56 | 0.34 | 0.49 |
| G136H | 3564.72 | 4804.54 | 0.74 | 0.61 | 0.66 |
| G136E | 5583.49 | 7289.45 | 0.77 | 0.95 | 1.00 |
| Q137A | 3966.25 | 6312.83 | 0.63 | 0.75 | 0.85 |
| Q137R | 3671.71 | 6256.44 | 0.59 | 0.69 | 0.84 |
| Q137G | 4573.46 | 6739.55 | 0.68 | 0.86 | 0.91 |
| Q137K | 6317.49 | 8133.50 | 0.78 | 1.19 | 1.09 |
| Q137H | 5645.75 | 7063.96 | 0.80 | 1.06 | 0.95 |
| Q137P | 5676.99 | 7744.50 | 0.73 | 1.07 | 1.04 |
| Q137S | 5384.46 | 7395.46 | 0.73 | 1.01 | 1.00 |
| Q137L | 5870.03 | 8003.53 | 0.73 | 1.10 | 1.08 |
| Q137W | 3200.77 | 5519.86 | 0.58 | 0.60 | 0.74 |
| Q137F | 3505.51 | 5883.52 | 0.60 | 0.66 | 0.79 |
| Q137T | 6636.95 | 8394.25 | 0.79 | 1.25 | 1.13 |
| Q137C | 1924.03 | 2898.49 | 0.66 | 0.36 | 0.39 |
| Q137Y | 5307.87 | 7091.79 | 0.75 | 1.00 | 0.95 |
| Q137N | 5369.70 | 7661.25 | 0.70 | 1.01 | 1.03 |
| Q137E | 5683.74 | 7333.42 | 0.78 | 1.07 | 0.99 |
| A138V | 1926.65 | 3043.75 | 0.63 | 0.36 | 0.41 |
| A138L | 594.07 | 675.23 | 0.88 | 0.11 | 0.09 |
| A138P | 1481.25 | 2478.85 | 0.60 | 0.28 | 0.33 |
| A138C | 1603.91 | 2981.97 | 0.54 | 0.30 | 0.40 |
| A138T | 1740.56 | 2785.40 | 0.62 | 0.33 | 0.37 |
| A138S | 2042.67 | 2909.34 | 0.70 | 0.38 | 0.39 |
| A138R | 759.61 | 962.30 | 0.79 | 0.14 | 0.13 |
| A138G | 3108.95 | 4692.85 | 0.66 | 0.58 | 0.63 |
| A138E | 1450.57 | 2644.44 | 0.55 | 0.27 | 0.36 |
| A138H | 667.58 | 839.37 | 0.80 | 0.13 | 0.11 |
| A138M | 626.50 | 749.79 | 0.84 | 0.12 | 0.10 |
| A138Q | 1747.09 | 2754.75 | 0.63 | 0.33 | 0.37 |
| A138I | 1984.57 | 3124.09 | 0.64 | 0.37 | 0.42 |
| A138D | 601.71 | 673.39 | 0.89 | 0.11 | 0.09 |
| A138W | 595.15 | 681.04 | 0.87 | 0.11 | 0.09 |
| D139R | 639.90 | 725.74 | 0.88 | 0.12 | 0.10 |
| D139V | 733.94 | 879.96 | 0.83 | 0.14 | 0.12 |
| D139M | 820.44 | 1093.19 | 0.75 | 0.15 | 0.15 |
| D139C | 763.17 | 886.67 | 0.86 | 0.14 | 0.12 |
| D139P | 794.89 | 1048.41 | 0.76 | 0.15 | 0.14 |
| D139S | 1060.53 | 1350.12 | 0.79 | 0.20 | 0.18 |
| D139L | 802.45 | 923.25 | 0.87 | 0.15 | 0.12 |
| D139I | 759.45 | 884.92 | 0.86 | 0.14 | 0.12 |
| D139H | 1442.05 | 1944.83 | 0.74 | 0.27 | 0.26 |
| D139A | 899.50 | 1179.38 | 0.76 | 0.17 | 0.16 |
| D139G | 667.47 | 801.55 | 0.83 | 0.13 | 0.11 |
| D139F | 670.14 | 828.73 | 0.81 | 0.13 | 0.11 |
| D139N | 1743.46 | 2795.27 | 0.62 | 0.33 | 0.38 |
| D139W | 641.04 | 769.42 | 0.83 | 0.12 | 0.10 |
| D139Y | 643.83 | 701.07 | 0.92 | 0.12 | 0.09 |
| D139E | 4365.22 | 7664.89 | 0.57 | 0.48 | 1.01 |
| I140D | 447.31 | 470.26 | 0.95 | 0.08 | 0.06 |
| I140K | 470.80 | 510.28 | 0.92 | 0.08 | 0.07 |
| I140A | 521.91 | 583.80 | 0.89 | 0.09 | 0.08 |
| I140G | 514.54 | 519.88 | 0.99 | 0.09 | 0.07 |
| I140C | 552.10 | 550.46 | 1.00 | 0.10 | 0.07 |
| I140Y | 476.67 | 511.05 | 0.93 | 0.08 | 0.07 |
| I140V | 1483.60 | 2240.10 | 0.66 | 0.26 | 0.29 |
| I140W | 541.30 | 540.55 | 1.00 | 0.10 | 0.07 |
| I140F | 671.22 | 710.61 | 0.94 | 0.12 | 0.09 |
| I140H | 568.54 | 584.67 | 0.97 | 0.10 | 0.08 |
| I140L | 4551.18 | 6862.42 | 0.66 | 0.80 | 0.90 |
| I140R | 479.35 | 467.38 | 1.03 | 0.08 | 0.06 |
| I140E | 480.00 | 481.92 | 1.00 | 0.08 | 0.06 |
| I140M | 1888.65 | 2695.29 | 0.70 | 0.33 | 0.35 |
| I140T | 493.59 | 505.63 | 0.98 | 0.09 | 0.07 |
| M141E | 2661.78 | 3381.08 | 0.79 | 0.64 | 0.66 |
| M141I | 3206.64 | 3834.28 | 0.84 | 0.77 | 0.74 |
| M141R | 3645.85 | 4050.91 | 0.90 | 0.88 | 0.79 |
| M141T | 1916.97 | 2186.65 | 0.88 | 0.46 | 0.42 |
| M141P | 957.33 | 1027.91 | 0.93 | 0.23 | 0.20 |
| M141S | 2578.82 | 3190.11 | 0.81 | 0.62 | 0.62 |
| M141C | 1162.41 | 1355.08 | 0.86 | 0.28 | 0.26 |
| M141L | 3257.69 | 4218.46 | 0.77 | 0.79 | 0.82 |
| M141A | 2798.52 | 3561.95 | 0.79 | 0.68 | 0.69 |
| M141D | 1943.58 | 2586.12 | 0.75 | 0.47 | 0.50 |
| M141W | 3860.87 | 4822.22 | 0.80 | 0.93 | 0.94 |
| M141G | 1252.97 | 1525.59 | 0.82 | 0.30 | 0.30 |
| M141H | 2221.73 | 2624.41 | 0.85 | 0.54 | 0.51 |
| M141Y | 2117.90 | 2326.92 | 0.91 | 0.51 | 0.45 |
| M141N | 3446.02 | 4175.18 | 0.83 | 0.83 | 0.81 |
| I142L | 4079.65 | 6338.98 | 0.64 | 0.72 | 0.83 |
| I142M | 2514.92 | 3872.66 | 0.65 | 0.44 | 0.51 |
| I142G | 590.45 | 573.21 | 1.03 | 0.10 | 0.07 |
| I142K | 567.58 | 566.13 | 1.00 | 0.10 | 0.07 |
| I142A | 1102.77 | 1776.73 | 0.62 | 0.19 | 0.23 |
| I142N | 544.78 | 582.84 | 0.93 | 0.10 | 0.08 |
| I142W | 614.88 | 660.20 | 0.93 | 0.11 | 0.09 |
| I142P | 517.98 | 553.16 | 0.94 | 0.09 | 0.07 |
| I142Q | 561.05 | 579.03 | 0.97 | 0.10 | 0.08 |
| I142Y | 535.36 | 568.63 | 0.94 | 0.09 | 0.07 |
| I142V | 2412.99 | 3835.99 | 0.63 | 0.42 | 0.50 |
| I142T | 619.92 | 700.51 | 0.88 | 0.11 | 0.09 |
| I142R | 592.22 | 631.35 | 0.94 | 0.10 | 0.08 |
| I142S | 560.11 | 608.47 | 0.92 | 0.10 | 0.08 |
| I142F | 988.49 | 1616.93 | 0.61 | 0.17 | 0.21 |
| S143P | 681.44 | 714.91 | 0.95 | 0.13 | 0.10 |
| S143C | 1242.25 | 1638.96 | 0.76 | 0.23 | 0.22 |
| S143E | 679.14 | 698.14 | 0.97 | 0.13 | 0.09 |
| S143G | 2178.31 | 3221.72 | 0.68 | 0.41 | 0.43 |
| S143H | 1946.43 | 3055.74 | 0.64 | 0.37 | 0.41 |
| S143R | 5284.60 | 7026.38 | 0.75 | 0.99 | 0.95 |
| S143L | 1855.11 | 3143.09 | 0.59 | 0.35 | 0.42 |
| S143Q | 4008.07 | 5922.69 | 0.68 | 0.75 | 0.80 |
| S143N | 3447.12 | 4827.78 | 0.71 | 0.65 | 0.65 |
| S143W | 1164.49 | 1414.47 | 0.82 | 0.22 | 0.19 |
| S143A | 4862.16 | 6797.05 | 0.72 | 0.91 | 0.91 |
| S143T | 3510.83 | 4873.23 | 0.72 | 0.66 | 0.66 |
| S143Y | 2566.36 | 3755.42 | 0.68 | 0.48 | 0.51 |
| S143M | 3680.60 | 6112.93 | 0.60 | 0.69 | 0.82 |
| S143I | 6798.46 | 8447.06 | 0.80 | 1.28 | 1.14 |
| F144K | 683.92 | 723.08 | 0.95 | 0.13 | 0.10 |
| F144M | 727.58 | 785.61 | 0.93 | 0.14 | 0.11 |
| F144E | 684.55 | 697.38 | 0.98 | 0.13 | 0.09 |
| F144S | 711.27 | 779.76 | 0.91 | 0.13 | 0.10 |
| F144L | 668.14 | 725.88 | 0.92 | 0.13 | 0.10 |
| F144W | 3272.56 | 4375.61 | 0.75 | 0.62 | 0.59 |
| F144P | 658.07 | 729.61 | 0.90 | 0.12 | 0.10 |
| F144R | 633.36 | 704.49 | 0.90 | 0.12 | 0.09 |
| F144N | 648.28 | 686.56 | 0.94 | 0.12 | 0.09 |
| F144C | 656.21 | 698.34 | 0.94 | 0.12 | 0.09 |
| F144G | 641.29 | 662.20 | 0.97 | 0.12 | 0.09 |
| F144T | 704.60 | 804.24 | 0.88 | 0.13 | 0.11 |
| F144Q | 679.28 | 759.34 | 0.89 | 0.13 | 0.10 |
| F144H | 766.54 | 861.04 | 0.89 | 0.14 | 0.12 |
| F144V | 664.73 | 737.66 | 0.90 | 0.12 | 0.10 |
| V145A | 5042.62 | 7103.17 | 0.71 | 0.89 | 0.93 |
| V145T | 3518.22 | 5408.09 | 0.65 | 0.62 | 0.71 |
| V145L | 4048.83 | 6522.67 | 0.62 | 0.71 | 0.85 |
| V145P | 2148.04 | 3271.70 | 0.66 | 0.38 | 0.43 |
| V145K | 4566.52 | 6542.14 | 0.70 | 0.80 | 0.85 |
| V145N | 5756.42 | 8553.91 | 0.67 | 1.01 | 1.12 |
| V145D | 3249.52 | 5915.18 | 0.55 | 0.57 | 0.77 |
| V145H | 3868.79 | 6370.16 | 0.61 | 0.68 | 0.83 |
| V145R | 5093.69 | 7494.19 | 0.68 | 0.90 | 0.98 |
| V145Q | 4550.79 | 6385.09 | 0.71 | 0.80 | 0.83 |
| V145S | 5229.00 | 7486.54 | 0.70 | 0.92 | 0.98 |
| V145G | 2139.70 | 3072.06 | 0.70 | 0.38 | 0.40 |
| V145W | 1735.30 | 3046.73 | 0.57 | 0.31 | 0.40 |
| V145C | 1652.16 | 3231.89 | 0.51 | 0.29 | 0.42 |
| V145E | 4086.60 | 6893.09 | 0.59 | 0.72 | 0.90 |
| R146T | 4145.84 | 6737.53 | 0.62 | 0.78 | 0.91 |
| R146L | 2149.16 | 3444.38 | 0.62 | 0.40 | 0.46 |
| R146N | 4441.83 | 6346.03 | 0.70 | 0.83 | 0.85 |
| R146H | 2791.26 | 3298.03 | 0.85 | 0.52 | 0.44 |
| R146Q | 4232.35 | 6620.08 | 0.64 | 0.80 | 0.89 |
| R146K | 5360.91 | 7104.88 | 0.75 | 1.01 | 0.96 |
| R146C | 776.54 | 868.97 | 0.89 | 0.15 | 0.12 |
| R146S | 8627.00 | 9288.66 | 0.93 | 1.62 | 1.25 |
| R146D | 3803.07 | 5389.79 | 0.71 | 0.71 | 0.73 |
| R146A | 4939.79 | 6859.12 | 0.72 | 0.93 | 0.92 |
| R146Y | 3175.81 | 5447.89 | 0.58 | 0.60 | 0.73 |
| R146P | 3019.88 | 2923.15 | 1.03 | 0.57 | 0.39 |
| R146V | 3662.01 | 6193.78 | 0.59 | 0.69 | 0.83 |
| R146E | 2538.71 | 3832.21 | 0.66 | 0.48 | 0.52 |
| R146F | 1272.11 | 2074.69 | 0.61 | 0.24 | 0.28 |
| G147R | 7744.82 | 8412.88 | 0.92 | 1.45 | 1.25 |
| G147F | 7821.91 | 7899.45 | 0.99 | 1.47 | 1.18 |
| G147I | 1451.75 | 1461.30 | 0.99 | 0.27 | 0.22 |
| G147L | 1325.33 | 1787.45 | 0.74 | 0.25 | 0.27 |
| G147A | 2288.45 | 2655.58 | 0.86 | 0.43 | 0.40 |
| G147E | 1802.97 | 2340.36 | 0.77 | 0.34 | 0.35 |
| G147H | 6234.09 | 7432.44 | 0.84 | 1.17 | 1.11 |
| G147W | 5140.21 | 6807.71 | 0.76 | 0.96 | 1.01 |
| G147T | 5531.13 | 7240.25 | 0.76 | 1.04 | 1.08 |
| G147C | 6950.51 | 7385.23 | 0.94 | 1.30 | 1.10 |
| G147S | 3071.28 | 3887.91 | 0.79 | 0.58 | 0.58 |
| G147V | 4516.19 | 5576.27 | 0.81 | 0.85 | 0.83 |
| G147Q | 6879.67 | 7686.98 | 0.89 | 1.29 | 1.14 |
| G147M | 6059.54 | 7381.69 | 0.82 | 1.14 | 1.10 |
| G147P | 494.94 | 392.93 | 1.26 | 0.07 | 0.05 |
| D148R | 5874.44 | 7069.98 | 0.83 | 1.10 | 1.05 |
| D148I | 4934.66 | 6621.95 | 0.75 | 0.93 | 0.99 |
| D148T | 5534.68 | 6527.74 | 0.85 | 1.04 | 0.97 |
| D148G | 5545.91 | 6487.49 | 0.85 | 1.04 | 0.97 |
| D148L | 1738.87 | 2136.70 | 0.81 | 0.33 | 0.32 |
| D148V | 4521.62 | 5307.74 | 0.85 | 0.85 | 0.79 |
| D148A | 7276.18 | 7955.06 | 0.91 | 1.36 | 1.18 |
| D148W | 3622.09 | 4894.24 | 0.74 | 0.68 | 0.73 |
| D148P | 7311.56 | 7404.67 | 0.99 | 1.37 | 1.10 |
| D148S | 3190.82 | 3936.89 | 0.81 | 0.60 | 0.59 |
| D148K | 2414.59 | 3115.25 | 0.78 | 0.45 | 0.46 |
| D148E | 2457.68 | 3171.30 | 0.77 | 0.46 | 0.47 |
| D148M | 928.12 | 1156.79 | 0.80 | 0.17 | 0.17 |
| D148N | 5136.96 | 5810.99 | 0.88 | 0.96 | 0.87 |
| D148C | 2617.98 | 3259.70 | 0.80 | 0.49 | 0.49 |
| H149W | 578.88 | 610.12 | 0.95 | 0.10 | 0.08 |
| H149A | 574.51 | 606.50 | 0.95 | 0.10 | 0.08 |
| H149L | 562.23 | 585.57 | 0.96 | 0.10 | 0.08 |
| H149C | 532.13 | 536.84 | 0.99 | 0.09 | 0.07 |
| H149Q | 547.46 | 565.60 | 0.97 | 0.10 | 0.07 |
| H149T | 545.99 | 567.99 | 0.96 | 0.10 | 0.07 |
| H149Y | 553.52 | 575.93 | 0.96 | 0.10 | 0.08 |
| H149P | 502.45 | 522.17 | 0.96 | 0.09 | 0.07 |
| H149V | 515.00 | 521.68 | 0.99 | 0.09 | 0.07 |
| H149R | 481.87 | 534.48 | 0.90 | 0.08 | 0.07 |
| H149G | 492.47 | 525.75 | 0.94 | 0.09 | 0.07 |
| H149E | 476.14 | 472.99 | 1.01 | 0.08 | 0.06 |
| H149S | 481.76 | 508.54 | 0.95 | 0.08 | 0.07 |
| H149I | 510.38 | 533.47 | 0.96 | 0.09 | 0.07 |
| H149N | 542.00 | 555.29 | 0.98 | 0.10 | 0.07 |
| R150S | 4221.17 | 4687.08 | 0.90 | 0.58 | 0.66 |
| R150E | 9557.47 | 8282.03 | 1.15 | 1.31 | 1.17 |
| R150G | 10002.15 | 8470.68 | 1.18 | 1.37 | 1.19 |
| R150M | 8614.46 | 8306.99 | 1.04 | 1.18 | 1.17 |
| R150P | 2291.14 | 828.28 | 2.77 | 0.31 | 0.12 |
| R150T | 9808.17 | 8294.42 | 1.18 | 1.35 | 1.17 |
| R150W | 8373.53 | 7574.51 | 1.11 | 1.15 | 1.07 |
| R150A | 10175.13 | 8554.82 | 1.19 | 1.40 | 1.20 |
| R150N | 10191.05 | 8571.32 | 1.19 | 1.40 | 1.21 |
| R150K | 9471.29 | 8346.99 | 1.13 | 1.30 | 1.18 |
| R150L | 9751.98 | 8444.63 | 1.15 | 1.34 | 1.19 |
| R150V | 6869.28 | 6604.61 | 1.04 | 1.20 | 1.30 |
| R150D | 7230.41 | 6033.28 | 1.20 | 1.26 | 1.19 |
| R150I | 3120.05 | 4082.34 | 0.76 | 0.39 | 0.55 |
| R150H | 8281.04 | 8056.17 | 1.03 | 1.05 | 1.08 |
| D151R | 576.24 | 545.21 | 1.06 | 0.11 | 0.08 |
| D151F | 626.76 | 601.08 | 1.04 | 0.12 | 0.09 |
| D151P | 670.23 | 610.90 | 1.10 | 0.13 | 0.09 |
| D151W | 691.38 | 656.86 | 1.05 | 0.13 | 0.10 |
| D151Q | 634.58 | 619.91 | 1.02 | 0.12 | 0.09 |
| D151L | 638.24 | 627.06 | 1.02 | 0.12 | 0.09 |
| D151S | 612.74 | 579.48 | 1.06 | 0.11 | 0.09 |
| D151G | 1073.32 | 733.89 | 1.46 | 0.20 | 0.11 |
| D151A | 635.33 | 608.12 | 1.04 | 0.12 | 0.09 |
| D151N | 631.72 | 612.41 | 1.03 | 0.12 | 0.09 |
| D151K | 648.63 | 635.47 | 1.02 | 0.12 | 0.09 |
| D151Y | 744.90 | 724.43 | 1.03 | 0.14 | 0.11 |
| D151V | 586.23 | 585.19 | 1.00 | 0.11 | 0.09 |
| D151T | 589.61 | 587.04 | 1.00 | 0.11 | 0.09 |
| D151M | 2945.18 | 605.81 | 4.86 | 0.55 | 0.09 |
| N152G | 9852.70 | 8326.15 | 1.18 | 1.35 | 1.17 |
| N152C | 6322.44 | 7849.36 | 0.81 | 0.87 | 1.11 |
| N152F | 9762.67 | 8643.34 | 1.13 | 1.34 | 1.22 |
| N152L | 9176.14 | 8510.93 | 1.08 | 1.26 | 1.20 |
| N152P | 7767.98 | 7907.66 | 0.98 | 1.07 | 1.11 |
| N152R | 8348.81 | 7893.25 | 1.06 | 1.14 | 1.11 |
| N152H | 3318.01 | 4257.74 | 0.78 | 0.46 | 0.60 |
| N152T | 7155.46 | 7180.94 | 1.00 | 0.98 | 1.01 |
| N152Y | 8343.23 | 7992.79 | 1.04 | 1.14 | 1.13 |
| N152K | 7868.59 | 7956.82 | 0.99 | 1.08 | 1.12 |
| N152D | 10221.41 | 8616.74 | 1.19 | 1.40 | 1.21 |
| N152W | 5717.25 | 7086.82 | 0.81 | 0.78 | 1.00 |
| N152I | 10161.44 | 8648.89 | 1.17 | 1.39 | 1.22 |
| N152A | 6669.94 | 5660.16 | 1.18 | 1.17 | 1.12 |
| N152S | 4607.85 | 8096.31 | 0.57 | 0.58 | 1.08 |
| S153I | 2873.12 | 2619.74 | 1.10 | 0.39 | 0.37 |
| S153R | 4799.14 | 4905.35 | 0.98 | 0.66 | 0.69 |
| S153K | 1002.00 | 1199.78 | 0.84 | 0.14 | 0.17 |
| S153C | 1934.36 | 3181.56 | 0.61 | 0.27 | 0.45 |
| S153G | 6175.12 | 6148.70 | 1.00 | 0.85 | 0.87 |
| S153H | 9759.94 | 8837.02 | 1.10 | 1.34 | 1.24 |
| S153L | 1285.63 | 1575.63 | 0.82 | 0.18 | 0.22 |
| S153V | 8993.77 | 8047.48 | 1.12 | 1.23 | 1.13 |
| S153T | 10530.07 | 8798.72 | 1.20 | 1.44 | 1.24 |
| S153P | 9442.29 | 8513.31 | 1.11 | 1.29 | 1.20 |
| S153A | 644.02 | 569.42 | 1.13 | 0.09 | 0.08 |
| S153F | 10583.60 | 8979.56 | 1.18 | 1.45 | 1.26 |
| S153D | 8477.40 | 8662.71 | 0.98 | 1.16 | 1.22 |
| S153Q | 6654.12 | 7947.98 | 0.84 | 0.91 | 1.12 |
| S153Y | 10164.62 | 8758.66 | 1.16 | 1.39 | 1.23 |
| P154V | 1257.75 | 1273.72 | 0.99 | 0.24 | 0.19 |
| P154W | 3838.51 | 2992.41 | 1.28 | 0.72 | 0.45 |
| P154L | 5826.55 | 6782.07 | 0.86 | 1.09 | 1.01 |
| P154C | 3097.69 | 3692.51 | 0.84 | 0.58 | 0.55 |
| P154S | 7417.09 | 8143.14 | 0.91 | 1.39 | 1.21 |
| P154K | 2407.68 | 1639.28 | 1.47 | 0.45 | 0.24 |
| P154I | 7298.30 | 7549.63 | 0.97 | 1.37 | 1.12 |
| P154A | 2043.76 | 2680.62 | 0.76 | 0.38 | 0.40 |
| P154T | 1763.73 | 2075.38 | 0.85 | 0.33 | 0.31 |
| P154H | 1072.51 | 1021.04 | 1.05 | 0.20 | 0.15 |
| P154Y | 946.74 | 834.91 | 1.13 | 0.18 | 0.12 |
| P154N | 1122.04 | 1229.36 | 0.91 | 0.21 | 0.18 |
| P154F | 845.38 | 757.86 | 1.12 | 0.16 | 0.11 |
| P154R | 1975.77 | 1915.36 | 1.03 | 0.37 | 0.29 |
| P154Q | 2228.56 | 2374.11 | 0.94 | 0.42 | 0.35 |
| F155S | 894.19 | 833.57 | 1.07 | 0.17 | 0.12 |
| F155T | 1137.71 | 1084.01 | 1.05 | 0.21 | 0.16 |
| F155G | 807.68 | 718.82 | 1.12 | 0.15 | 0.11 |
| F155N | 715.78 | 688.72 | 1.04 | 0.13 | 0.10 |
| F155R | 702.49 | 695.27 | 1.01 | 0.13 | 0.10 |
| F155W | 715.40 | 693.53 | 1.03 | 0.13 | 0.10 |
| F155L | 1322.13 | 864.19 | 1.53 | 0.25 | 0.13 |
| F155Q | 731.28 | 738.56 | 0.99 | 0.14 | 0.11 |
| F155M | 8252.43 | 8163.55 | 1.01 | 1.55 | 1.22 |
| F155E | 685.90 | 683.12 | 1.00 | 0.13 | 0.10 |
| F155A | 1250.93 | 760.12 | 1.65 | 0.23 | 0.11 |
| F155P | 666.89 | 658.85 | 1.01 | 0.13 | 0.10 |
| F155V | 681.25 | 679.13 | 1.00 | 0.13 | 0.10 |
| F155H | 696.34 | 683.06 | 1.02 | 0.13 | 0.10 |
| F155Y | 676.73 | 629.34 | 1.08 | 0.13 | 0.09 |
| D156H | 2722.09 | 2081.55 | 1.31 | 0.51 | 0.31 |
| D156L | 2548.30 | 1597.53 | 1.60 | 0.48 | 0.24 |
| D156E | 6300.50 | 6871.25 | 0.92 | 1.18 | 1.02 |
| D156A | 2679.29 | 1734.45 | 1.54 | 0.50 | 0.26 |
| D156W | 1575.39 | 1268.36 | 1.24 | 0.30 | 0.19 |
| D156C | 2842.85 | 2704.37 | 1.05 | 0.53 | 0.40 |
| D156P | 1002.13 | 998.80 | 1.00 | 0.19 | 0.15 |
| D156V | 1400.88 | 766.80 | 1.83 | 0.26 | 0.11 |
| D156K | 1292.89 | 966.62 | 1.34 | 0.24 | 0.14 |
| D156S | 969.70 | 837.57 | 1.16 | 0.18 | 0.12 |
| D156G | 794.14 | 709.60 | 1.12 | 0.15 | 0.11 |
| D156T | 2871.09 | 1843.03 | 1.56 | 0.54 | 0.27 |
| D156Y | 3406.50 | 3113.95 | 1.09 | 0.64 | 0.46 |
| D156R | 2431.23 | 1545.89 | 1.57 | 0.46 | 0.23 |
| D156M | 817.96 | 502.82 | 1.63 | 0.12 | 0.07 |
| G157K | 677.09 | 562.66 | 1.20 | 0.09 | 0.08 |
| G157D | 603.28 | 513.64 | 1.17 | 0.08 | 0.07 |
| G157F | 9535.19 | 8450.24 | 1.13 | 1.31 | 1.19 |
| G157R | 704.56 | 540.98 | 1.30 | 0.10 | 0.08 |
| G157H | 608.42 | 567.38 | 1.07 | 0.08 | 0.08 |
| G157L | 582.39 | 476.09 | 1.22 | 0.08 | 0.07 |
| G157N | 721.55 | 534.46 | 1.35 | 0.10 | 0.08 |
| G157Y | 654.13 | 541.41 | 1.21 | 0.09 | 0.08 |
| G157S | 924.62 | 596.70 | 1.55 | 0.13 | 0.08 |
| G157T | 669.55 | 551.99 | 1.21 | 0.09 | 0.08 |
| G157A | 861.29 | 552.54 | 1.56 | 0.12 | 0.08 |
| G157Q | 655.49 | 522.72 | 1.25 | 0.09 | 0.07 |
| G157P | 635.63 | 591.49 | 1.07 | 0.09 | 0.08 |
| G157V | 654.45 | 573.19 | 1.14 | 0.09 | 0.08 |
| G157M | 716.52 | 615.65 | 1.16 | 0.10 | 0.09 |
| P158S | 7974.07 | 7118.62 | 1.12 | 1.09 | 1.00 |
| P158Y | 7544.63 | 6885.81 | 1.10 | 1.03 | 0.97 |
| P158R | 7142.54 | 6214.55 | 1.15 | 0.98 | 0.88 |
| P158L | 9290.77 | 6775.04 | 1.37 | 1.27 | 0.95 |
| P158V | 10642.99 | 8919.30 | 1.19 | 1.46 | 1.26 |
| P158C | 6284.97 | 4792.49 | 1.31 | 0.86 | 0.67 |
| P158A | 9579.29 | 8514.00 | 1.13 | 1.31 | 1.20 |
| P158W | 5175.38 | 3078.22 | 1.68 | 0.71 | 0.43 |
| P158I | 10312.96 | 8597.26 | 1.20 | 1.41 | 1.21 |
| P158F | 6595.54 | 4090.71 | 1.61 | 0.90 | 0.58 |
| P158Q | 10928.51 | 8709.20 | 1.25 | 1.50 | 1.23 |
| P158T | 4204.23 | 3507.76 | 1.20 | 0.53 | 0.47 |
| P158G | 6277.86 | 5496.27 | 1.14 | 0.79 | 0.73 |
| P158K | 6860.82 | 6680.30 | 1.03 | 0.87 | 0.89 |
| P158N | 3656.04 | 3874.48 | 0.94 | 0.46 | 0.52 |
| P158D | 8959.02 | 7355.10 | 1.22 | 0.98 | 0.96 |
| G159R | 6441.49 | 5914.02 | 1.09 | 0.88 | 0.83 |
| G159S | 6594.46 | 6573.14 | 1.00 | 0.90 | 0.93 |
| G159Q | 3996.96 | 4391.10 | 0.91 | 0.55 | 0.62 |
| G159P | 596.30 | 564.24 | 1.06 | 0.08 | 0.08 |
| G159V | 2453.98 | 732.46 | 3.35 | 0.34 | 0.10 |
| G159K | 554.74 | 515.44 | 1.08 | 0.08 | 0.07 |
| G159A | 5157.14 | 4685.20 | 1.10 | 0.71 | 0.66 |
| G159Y | 1029.19 | 752.76 | 1.37 | 0.14 | 0.11 |
| G159E | 4327.74 | 4027.23 | 1.07 | 0.59 | 0.57 |
| G159T | 5059.91 | 1734.12 | 2.92 | 0.69 | 0.24 |
| G159M | 5905.06 | 4874.00 | 1.21 | 0.75 | 0.65 |
| G159I | 5725.99 | 5357.20 | 1.07 | 0.72 | 0.72 |
| G159W | 6787.40 | 6287.71 | 1.08 | 0.86 | 0.84 |
| G159L | 8231.62 | 7638.64 | 1.08 | 1.04 | 1.02 |
| G159C | 2897.77 | 3053.86 | 0.95 | 0.37 | 0.41 |
| G160A | 2080.01 | 2823.12 | 0.74 | 0.60 | 0.58 |
| G160H | 2001.10 | 3085.22 | 0.65 | 0.57 | 0.63 |
| G160N | 3546.69 | 5339.11 | 0.66 | 1.02 | 1.09 |
| G160W | 4334.72 | 3946.12 | 1.10 | 1.24 | 0.80 |
| G160R | 2347.11 | 3791.36 | 0.62 | 0.67 | 0.77 |
| G160P | 1047.77 | 929.25 | 1.13 | 0.30 | 0.19 |
| G160I | 1794.48 | 1596.11 | 1.12 | 0.51 | 0.33 |
| G160M | 2506.95 | 3576.91 | 0.70 | 0.72 | 0.73 |
| G160C | 580.68 | 627.99 | 0.92 | 0.17 | 0.13 |
| G160Q | 4740.98 | 6839.68 | 0.69 | 1.36 | 1.39 |
| G160V | 3284.36 | 3030.37 | 1.08 | 0.94 | 0.62 |
| G160S | 2991.02 | 4281.08 | 0.70 | 0.86 | 0.87 |
| G160E | 3899.28 | 4071.63 | 0.96 | 1.12 | 0.83 |
| G160L | 3396.11 | 4411.61 | 0.77 | 0.97 | 0.90 |
| G160T | 3844.32 | 3943.21 | 0.97 | 1.10 | 0.80 |
| N161S | 1251.34 | 2417.50 | 0.52 | 0.36 | 0.49 |
| N161C | 1591.15 | 2710.58 | 0.59 | 0.46 | 0.55 |
| N161L | 4840.46 | 7275.68 | 0.67 | 1.39 | 1.48 |
| N161R | 6437.08 | 7915.53 | 0.81 | 1.85 | 1.61 |
| N161G | 3755.93 | 6728.88 | 0.56 | 1.08 | 1.37 |
| N161W | 3135.58 | 3994.07 | 0.79 | 0.90 | 0.81 |
| N161Y | 5507.52 | 7319.79 | 0.75 | 1.58 | 1.49 |
| N161E | 5410.44 | 8192.46 | 0.66 | 1.55 | 1.67 |
| N161P | 1231.02 | 1593.36 | 0.77 | 0.35 | 0.32 |
| N161T | 7464.29 | 9082.12 | 0.82 | 2.14 | 1.85 |
| N161H | 2727.71 | 5034.74 | 0.54 | 0.78 | 1.03 |
| N161I | 8070.21 | 9914.56 | 0.81 | 2.31 | 2.02 |
| N161V | 5238.40 | 7429.22 | 0.71 | 1.50 | 1.51 |
| N161F | 3890.45 | 6624.80 | 0.59 | 1.12 | 1.35 |
| N161Q | 4690.72 | 7412.73 | 0.63 | 1.35 | 1.51 |
| L162A | 584.17 | 505.94 | 1.15 | 0.17 | 0.10 |
| L162G | 582.48 | 602.57 | 0.97 | 0.17 | 0.12 |
| L162C | 475.91 | 466.85 | 1.02 | 0.14 | 0.10 |
| L162P | 514.26 | 519.71 | 0.99 | 0.15 | 0.11 |
| L162R | 492.19 | 498.99 | 0.99 | 0.14 | 0.10 |
| L162I | 2948.11 | 4018.68 | 0.73 | 0.85 | 0.82 |
| L162S | 473.63 | 459.28 | 1.03 | 0.14 | 0.09 |
| L162D | 512.72 | 487.18 | 1.05 | 0.15 | 0.10 |
| L162M | 1013.31 | 1138.86 | 0.89 | 0.29 | 0.23 |
| L162E | 563.63 | 631.85 | 0.89 | 0.16 | 0.13 |
| L162T | 473.46 | 477.00 | 0.99 | 0.14 | 0.10 |
| L162Y | 484.26 | 519.58 | 0.93 | 0.14 | 0.11 |
| L162F | 484.37 | 469.30 | 1.03 | 0.14 | 0.10 |
| L162W | 463.12 | 457.12 | 1.01 | 0.13 | 0.09 |
| L162Q | 480.75 | 481.03 | 1.00 | 0.14 | 0.10 |
| A163R | 562.68 | 563.06 | 1.00 | 0.16 | 0.11 |
| A163G | 819.22 | 999.88 | 0.82 | 0.23 | 0.20 |
| A163Y | 562.12 | 549.69 | 1.02 | 0.16 | 0.11 |
| A163P | 557.10 | 559.72 | 1.00 | 0.16 | 0.11 |
| A163S | 572.70 | 542.75 | 1.06 | 0.16 | 0.11 |
| A163L | 532.98 | 539.88 | 0.99 | 0.15 | 0.11 |
| A163C | 528.01 | 546.64 | 0.97 | 0.15 | 0.11 |
| A163K | 510.99 | 502.63 | 1.02 | 0.15 | 0.10 |
| A163V | 567.33 | 572.66 | 0.99 | 0.16 | 0.12 |
| A163F | 931.85 | 1182.48 | 0.79 | 0.27 | 0.24 |
| A163E | 560.80 | 539.21 | 1.04 | 0.16 | 0.11 |
| A163T | 538.98 | 537.66 | 1.00 | 0.15 | 0.11 |
| A163Q | 586.94 | 586.24 | 1.00 | 0.17 | 0.12 |
| A163I | 554.29 | 579.47 | 0.96 | 0.16 | 0.12 |
| A163N | 575.87 | 580.49 | 0.99 | 0.17 | 0.12 |
| H164L | 547.21 | 565.25 | 0.97 | 0.16 | 0.12 |
| H164M | 552.91 | 590.51 | 0.94 | 0.16 | 0.12 |
| H164K | 575.53 | 589.33 | 0.98 | 0.17 | 0.12 |
| H164P | 573.34 | 570.59 | 1.00 | 0.16 | 0.12 |
| H164C | 551.45 | 576.69 | 0.96 | 0.16 | 0.12 |
| H164R | 558.91 | 553.87 | 1.01 | 0.16 | 0.11 |
| H164A | 549.93 | 598.96 | 0.92 | 0.16 | 0.12 |
| H164V | 567.08 | 579.35 | 0.98 | 0.16 | 0.12 |
| H164S | 4849.81 | 6939.34 | 0.70 | 1.39 | 1.41 |
| H164N | 437.45 | 585.42 | 0.75 | 0.13 | 0.12 |
| H164G | 545.54 | 547.89 | 1.00 | 0.16 | 0.11 |
| H164F | 540.67 | 537.69 | 1.01 | 0.16 | 0.11 |
| H164Y | 558.66 | 548.15 | 1.02 | 0.16 | 0.11 |
| H164Q | 566.62 | 555.39 | 1.02 | 0.16 | 0.11 |
| H164E | 569.92 | 612.16 | 0.93 | 0.16 | 0.12 |
| A165W | 583.56 | 591.99 | 0.99 | 0.17 | 0.12 |
| A165V | 560.38 | 564.19 | 0.99 | 0.16 | 0.11 |
| A165G | 445.09 | 575.94 | 0.77 | 0.13 | 0.12 |
| A165K | 537.18 | 537.57 | 1.00 | 0.15 | 0.11 |
| A165L | 552.58 | 553.45 | 1.00 | 0.16 | 0.11 |
| A165P | 535.50 | 554.41 | 0.97 | 0.15 | 0.11 |
| A165Q | 983.84 | 1344.06 | 0.73 | 0.28 | 0.27 |
| A165D | 534.17 | 577.13 | 0.93 | 0.15 | 0.12 |
| A165H | 515.90 | 536.85 | 0.96 | 0.15 | 0.11 |
| A165F | 493.42 | 496.39 | 0.99 | 0.14 | 0.10 |
| A165S | 390.18 | 578.00 | 0.68 | 0.11 | 0.12 |
| A165T | 506.15 | 502.78 | 1.01 | 0.15 | 0.10 |
| A165R | 485.08 | 477.19 | 1.02 | 0.14 | 0.10 |
| A165N | 509.08 | 499.01 | 1.02 | 0.15 | 0.10 |
| A165M | 473.24 | 523.60 | 0.90 | 0.14 | 0.11 |
| F166G | 623.89 | 586.56 | 1.06 | 0.11 | 0.08 |
| F166S | 724.53 | 695.67 | 1.04 | 0.12 | 0.10 |
| F166L | 760.25 | 829.02 | 0.92 | 0.13 | 0.12 |
| F166V | 552.68 | 564.25 | 0.98 | 0.09 | 0.08 |
| F166P | 530.80 | 562.94 | 0.94 | 0.09 | 0.08 |
| F166N | 613.07 | 589.89 | 1.04 | 0.10 | 0.08 |
| F166R | 534.62 | 543.15 | 0.98 | 0.09 | 0.08 |
| F166A | 638.77 | 712.81 | 0.90 | 0.11 | 0.10 |
| F166K | 598.42 | 615.59 | 0.97 | 0.10 | 0.09 |
| F166H | 2770.43 | 2606.89 | 1.06 | 0.47 | 0.37 |
| F166W | 8234.80 | 8549.89 | 0.96 | 1.40 | 1.20 |
| F166I | 617.86 | 613.36 | 1.01 | 0.10 | 0.09 |
| F166M | 537.05 | 571.21 | 0.94 | 0.09 | 0.08 |
| F166C | 661.10 | 639.33 | 1.03 | 0.11 | 0.09 |
| F166E | 616.99 | 582.48 | 1.06 | 0.10 | 0.08 |
| Q167D | 4883.56 | 4579.11 | 1.07 | 0.83 | 0.64 |
| Q167R | 7660.88 | 8025.72 | 0.95 | 1.30 | 1.12 |
| Q167A | 8466.37 | 8182.70 | 1.03 | 1.44 | 1.15 |
| Q167S | 7915.08 | 8512.14 | 0.93 | 1.34 | 1.19 |
| Q167F | 8209.07 | 8535.93 | 0.96 | 1.39 | 1.20 |
| Q167Y | 5687.17 | 7642.43 | 0.74 | 0.96 | 1.07 |
| Q167P | 7513.70 | 8011.11 | 0.94 | 1.27 | 1.12 |
| Q167T | 7772.59 | 8173.09 | 0.95 | 1.32 | 1.15 |
| Q167V | 7867.97 | 8191.44 | 0.96 | 1.33 | 1.15 |
| Q167L | 7174.37 | 7937.04 | 0.90 | 1.22 | 1.11 |
| Q167M | 8005.59 | 8974.74 | 0.89 | 1.36 | 1.26 |
| Q167N | 3612.25 | 5004.93 | 0.72 | 0.61 | 0.70 |
| Q167G | 6671.99 | 7782.93 | 0.86 | 1.13 | 1.09 |
| Q167K | 6453.33 | 7646.48 | 0.84 | 1.09 | 1.07 |
| Q167E | 5009.36 | 6388.75 | 0.78 | 0.85 | 0.90 |
| P168N | 638.46 | 590.71 | 1.08 | 0.11 | 0.08 |
| P168F | 673.09 | 638.53 | 1.05 | 0.11 | 0.09 |
| P168R | 7038.51 | 7902.50 | 0.89 | 1.19 | 1.11 |
| P168W | 737.53 | 666.57 | 1.11 | 0.13 | 0.09 |
| P168A | 833.88 | 736.90 | 1.13 | 0.14 | 0.10 |
| P168T | 646.42 | 645.28 | 1.00 | 0.11 | 0.09 |
| P168V | 499.02 | 557.37 | 0.90 | 0.08 | 0.08 |
| P168G | 686.51 | 644.44 | 1.07 | 0.12 | 0.09 |
| P168C | 568.42 | 598.90 | 0.95 | 0.10 | 0.08 |
| P168M | 734.84 | 652.57 | 1.13 | 0.12 | 0.09 |
| P168H | 590.54 | 588.07 | 1.00 | 0.10 | 0.08 |
| P168L | 715.20 | 706.06 | 1.01 | 0.12 | 0.10 |
| P168S | 641.79 | 605.44 | 1.06 | 0.11 | 0.08 |
| P168I | 560.15 | 568.90 | 0.98 | 0.09 | 0.08 |
| P168D | 530.69 | 575.63 | 0.92 | 0.09 | 0.08 |
| G169H | 791.08 | 828.63 | 0.95 | 0.13 | 0.12 |
| G169A | 1556.29 | 2632.37 | 0.59 | 0.26 | 0.37 |
| G169E | 789.82 | 829.24 | 0.95 | 0.13 | 0.12 |
| G169C | 714.55 | 744.33 | 0.96 | 0.12 | 0.10 |
| G169S | 1196.93 | 1427.18 | 0.84 | 0.20 | 0.20 |
| G169L | 450.44 | 534.57 | 0.84 | 0.08 | 0.07 |
| G169V | 703.56 | 675.20 | 1.04 | 0.12 | 0.09 |
| G169T | 676.59 | 685.16 | 0.99 | 0.11 | 0.10 |
| G169R | 1119.16 | 1166.00 | 0.96 | 0.19 | 0.16 |
| G169W | 802.02 | 921.44 | 0.87 | 0.14 | 0.13 |
| G169M | 962.20 | 1133.87 | 0.85 | 0.16 | 0.16 |
| G169I | 671.79 | 677.10 | 0.99 | 0.11 | 0.09 |
| G169P | 671.60 | 683.22 | 0.98 | 0.11 | 0.10 |
| G169D | 714.59 | 766.96 | 0.93 | 0.12 | 0.11 |
| G169Q | 977.05 | 901.01 | 1.08 | 0.17 | 0.13 |
| P170L | 5969.84 | 7995.99 | 0.75 | 1.01 | 1.12 |
| P170R | 3566.07 | 5876.72 | 0.61 | 0.60 | 0.82 |
| P170I | 5073.27 | 7150.78 | 0.71 | 0.86 | 1.00 |
| P170T | 6734.46 | 8153.81 | 0.83 | 1.14 | 1.14 |
| P170F | 2114.36 | 3365.04 | 0.63 | 0.36 | 0.47 |
| P170Q | 4204.94 | 6162.63 | 0.68 | 0.71 | 0.86 |
| P170G | 5005.05 | 6924.03 | 0.72 | 0.85 | 0.97 |
| P170S | 4526.99 | 6064.79 | 0.75 | 0.77 | 0.85 |
| P170H | 4569.14 | 6879.10 | 0.66 | 0.77 | 0.96 |
| P170C | 931.84 | 2355.40 | 0.40 | 0.16 | 0.33 |
| P170M | 3323.56 | 6318.16 | 0.53 | 0.56 | 0.89 |
| P170K | 4379.75 | 6206.45 | 0.71 | 0.74 | 0.87 |
| P170W | 1794.33 | 2781.05 | 0.65 | 0.30 | 0.39 |
| P170D | 1434.38 | 1462.91 | 0.98 | 0.25 | 0.29 |
| P170A | 2733.72 | 2793.24 | 0.98 | 0.48 | 0.55 |
| G171S | 2129.39 | 3316.87 | 0.64 | 0.36 | 0.46 |
| G171M | 2104.33 | 3308.36 | 0.64 | 0.36 | 0.46 |
| G171N | 4674.81 | 6965.17 | 0.67 | 0.79 | 0.98 |
| G171P | 1570.74 | 1204.39 | 1.30 | 0.27 | 0.17 |
| G171R | 1604.06 | 2486.74 | 0.65 | 0.27 | 0.35 |
| G171Y | 1519.56 | 2342.05 | 0.65 | 0.26 | 0.33 |
| G171A | 3517.89 | 5269.99 | 0.67 | 0.60 | 0.74 |
| G171Q | 2361.29 | 3915.33 | 0.60 | 0.40 | 0.55 |
| G171H | 1662.65 | 2616.63 | 0.64 | 0.28 | 0.37 |
| G171L | 1551.95 | 2516.17 | 0.62 | 0.26 | 0.35 |
| G171W | 1068.10 | 1663.46 | 0.64 | 0.18 | 0.23 |
| G171C | 1982.45 | 3409.68 | 0.58 | 0.34 | 0.48 |
| G171K | 1324.98 | 1867.15 | 0.71 | 0.22 | 0.26 |
| G171E | 1154.96 | 1199.65 | 0.96 | 0.20 | 0.24 |
| G171D | 791.81 | 690.33 | 1.15 | 0.14 | 0.14 |
| I172Y | 7427.25 | 7893.50 | 0.94 | 0.93 | 0.95 |
| I172T | 5861.67 | 6776.91 | 0.86 | 0.73 | 0.81 |
| I172P | 6297.28 | 7073.49 | 0.89 | 0.79 | 0.85 |
| I172A | 4666.76 | 6048.64 | 0.77 | 0.58 | 0.73 |
| I172L | 9324.48 | 8876.38 | 1.05 | 1.17 | 1.07 |
| I172Q | 6906.03 | 7743.66 | 0.89 | 0.87 | 0.93 |
| I172E | 3517.33 | 4567.20 | 0.77 | 0.44 | 0.55 |
| I172C | 1784.43 | 2422.99 | 0.74 | 0.22 | 0.29 |
| I172M | 9859.60 | 9096.03 | 1.08 | 1.24 | 1.09 |
| I172D | 4276.25 | 4281.60 | 1.00 | 0.54 | 0.51 |
| I172V | 9541.02 | 9174.91 | 1.04 | 1.20 | 1.10 |
| I172R | 7010.86 | 7581.48 | 0.92 | 0.88 | 0.91 |
| I172G | 2169.59 | 2350.53 | 0.92 | 0.27 | 0.28 |
| I172W | 5870.25 | 7244.46 | 0.81 | 0.74 | 0.87 |
| I172N | 5235.67 | 6253.19 | 0.84 | 0.66 | 0.75 |
| G173C | 816.12 | 1115.07 | 0.73 | 0.10 | 0.13 |
| G173L | 454.21 | 401.73 | 1.13 | 0.06 | 0.05 |
| G173K | 741.76 | 685.79 | 1.08 | 0.09 | 0.08 |
| G173W | 1278.44 | 1132.78 | 1.13 | 0.16 | 0.14 |
| G173S | 865.76 | 793.10 | 1.09 | 0.11 | 0.10 |
| G173A | 1041.22 | 1038.66 | 1.00 | 0.13 | 0.12 |
| G173R | 973.70 | 877.71 | 1.11 | 0.12 | 0.11 |
| G173N | 818.38 | 931.59 | 0.88 | 0.10 | 0.11 |
| G173T | 481.41 | 485.39 | 0.99 | 0.06 | 0.06 |
| G173D | 474.45 | 424.47 | 1.12 | 0.06 | 0.05 |
| G173V | 505.39 | 476.04 | 1.06 | 0.06 | 0.06 |
| G173F | 670.10 | 610.91 | 1.10 | 0.08 | 0.07 |
| G173M | 1085.74 | 1324.87 | 0.82 | 0.14 | 0.16 |
| G173Y | 1390.14 | 1296.93 | 1.07 | 0.17 | 0.16 |
| G173P | 458.79 | 435.59 | 1.05 | 0.06 | 0.05 |
| G174R | 452.76 | 437.41 | 1.04 | 0.06 | 0.05 |
| G174A | 491.35 | 460.84 | 1.07 | 0.06 | 0.06 |
| G174E | 489.49 | 459.57 | 1.07 | 0.06 | 0.06 |
| G174F | 598.90 | 520.03 | 1.15 | 0.08 | 0.06 |
| G174H | 577.19 | 518.90 | 1.11 | 0.07 | 0.06 |
| G174T | 505.38 | 483.64 | 1.04 | 0.06 | 0.06 |
| G174D | 476.79 | 454.13 | 1.05 | 0.06 | 0.05 |
| G174S | 641.62 | 580.36 | 1.11 | 0.08 | 0.07 |
| G174P | 525.07 | 505.41 | 1.04 | 0.07 | 0.06 |
| G174W | 538.38 | 502.50 | 1.07 | 0.07 | 0.06 |
| G174V | 504.50 | 425.17 | 1.19 | 0.06 | 0.05 |
| G174N | 506.54 | 460.95 | 1.10 | 0.06 | 0.06 |
| G174Y | 722.10 | 525.97 | 1.37 | 0.09 | 0.06 |
| G174M | 516.58 | 464.32 | 1.11 | 0.06 | 0.06 |
| G174L | 474.83 | 436.08 | 1.09 | 0.06 | 0.05 |
| D175I | 697.29 | 708.65 | 0.98 | 0.09 | 0.09 |
| D175T | 601.82 | 560.11 | 1.07 | 0.08 | 0.07 |
| D175N | 1495.98 | 1413.58 | 1.06 | 0.19 | 0.17 |
| D175V | 694.28 | 652.82 | 1.06 | 0.09 | 0.08 |
| D175S | 664.04 | 579.78 | 1.15 | 0.08 | 0.07 |
| D175R | 593.04 | 505.98 | 1.17 | 0.07 | 0.06 |
| D175G | 3147.62 | 4207.02 | 0.75 | 0.39 | 0.51 |
| D175A | 633.65 | 519.61 | 1.22 | 0.08 | 0.06 |
| D175F | 768.41 | 804.63 | 0.95 | 0.10 | 0.10 |
| D175C | 535.75 | 498.44 | 1.07 | 0.07 | 0.06 |
| D175Q | 702.74 | 633.64 | 1.11 | 0.09 | 0.08 |
| D175Y | 574.71 | 539.79 | 1.06 | 0.07 | 0.06 |
| D175L | 591.05 | 549.49 | 1.08 | 0.07 | 0.07 |
| D175H | 830.09 | 564.37 | 1.47 | 0.10 | 0.07 |
| D175P | 635.08 | 610.89 | 1.04 | 0.08 | 0.07 |
| D175E | 959.14 | 495.19 | 1.94 | 0.10 | 0.07 |
| A176F | 10486.82 | 6516.31 | 1.61 | 1.31 | 0.78 |
| A176Q | 6410.52 | 6665.27 | 0.96 | 0.80 | 0.80 |
| A176V | 8890.53 | 8780.42 | 1.01 | 1.11 | 1.05 |
| A176E | 589.82 | 546.54 | 1.08 | 0.07 | 0.07 |
| A176T | 8471.98 | 8213.74 | 1.03 | 1.06 | 0.99 |
| A176C | 6777.92 | 5924.96 | 1.14 | 0.85 | 0.71 |
| A176L | 7190.01 | 6291.31 | 1.14 | 0.90 | 0.76 |
| A176P | 639.90 | 596.40 | 1.07 | 0.08 | 0.07 |
| A176N | 1351.92 | 1250.94 | 1.08 | 0.17 | 0.15 |
| A176G | 2185.25 | 2395.33 | 0.91 | 0.27 | 0.29 |
| A176S | 3003.29 | 3887.12 | 0.77 | 0.38 | 0.47 |
| A176R | 919.15 | 792.44 | 1.16 | 0.12 | 0.10 |
| A176K | 561.66 | 522.94 | 1.07 | 0.07 | 0.06 |
| A176D | 863.82 | 792.98 | 1.09 | 0.11 | 0.10 |
| A176W | 482.38 | 414.85 | 1.16 | 0.06 | 0.06 |
| H177T | 600.03 | 570.23 | 1.05 | 0.08 | 0.07 |
| H177P | 579.96 | 544.77 | 1.06 | 0.07 | 0.07 |
| H177Q | 593.68 | 549.35 | 1.08 | 0.07 | 0.07 |
| H177A | 536.01 | 523.32 | 1.02 | 0.07 | 0.06 |
| H177S | 561.60 | 524.64 | 1.07 | 0.07 | 0.06 |
| H177G | 559.31 | 519.85 | 1.08 | 0.07 | 0.06 |
| H177W | 547.21 | 520.18 | 1.05 | 0.07 | 0.06 |
| H177L | 486.50 | 433.60 | 1.12 | 0.06 | 0.05 |
| H177V | 508.58 | 447.50 | 1.14 | 0.06 | 0.05 |
| H177I | 489.45 | 455.90 | 1.07 | 0.06 | 0.05 |
| H177R | 1913.95 | 2460.77 | 0.78 | 0.24 | 0.30 |
| H177N | 504.44 | 478.07 | 1.06 | 0.06 | 0.06 |
| H177Y | 519.99 | 467.72 | 1.11 | 0.07 | 0.06 |
| H177C | 521.67 | 489.93 | 1.06 | 0.07 | 0.06 |
| H177D | 534.87 | 505.07 | 1.06 | 0.07 | 0.06 |
| F178G | 451.96 | 391.96 | 1.15 | 0.05 | 0.05 |
| F178C | 491.76 | 403.57 | 1.22 | 0.05 | 0.05 |
| F178W | 488.21 | 441.35 | 1.11 | 0.05 | 0.05 |
| F178R | 492.40 | 411.21 | 1.20 | 0.05 | 0.05 |
| F178K | 490.87 | 494.09 | 0.99 | 0.05 | 0.06 |
| F178S | 489.84 | 507.26 | 0.97 | 0.05 | 0.06 |
| F178H | 525.63 | 500.02 | 1.05 | 0.06 | 0.06 |
| F178P | 441.78 | 397.05 | 1.11 | 0.05 | 0.05 |
| F178V | 742.61 | 814.06 | 0.91 | 0.08 | 0.10 |
| F178A | 421.25 | 367.26 | 1.15 | 0.05 | 0.04 |
| F178Q | 409.62 | 360.29 | 1.14 | 0.04 | 0.04 |
| F178Y | 861.20 | 830.80 | 1.04 | 0.09 | 0.10 |
| F178I | 1118.23 | 1329.96 | 0.84 | 0.12 | 0.16 |
| F178T | 560.54 | 487.01 | 1.15 | 0.10 | 0.10 |
| F178L | 1788.95 | 1314.38 | 1.36 | 0.31 | 0.26 |
| F178E | 524.72 | 515.62 | 1.02 | 0.06 | 0.07 |
| D179P | 526.54 | 527.98 | 1.00 | 0.06 | 0.06 |
| D179L | 444.31 | 410.22 | 1.08 | 0.05 | 0.05 |
| D179E | 520.82 | 438.27 | 1.19 | 0.06 | 0.05 |
| D179G | 470.21 | 426.64 | 1.10 | 0.05 | 0.05 |
| D179S | 461.51 | 421.09 | 1.10 | 0.05 | 0.05 |
| D179A | 464.49 | 431.24 | 1.08 | 0.05 | 0.05 |
| D179K | 483.67 | 456.75 | 1.06 | 0.05 | 0.05 |
| D179T | 451.18 | 419.76 | 1.07 | 0.05 | 0.05 |
| D179I | 425.91 | 372.56 | 1.14 | 0.05 | 0.04 |
| D179R | 473.21 | 450.10 | 1.05 | 0.05 | 0.05 |
| D179N | 2433.73 | 812.01 | 3.00 | 0.26 | 0.10 |
| D179W | 465.31 | 423.56 | 1.10 | 0.05 | 0.05 |
| D179Q | 446.51 | 414.79 | 1.08 | 0.05 | 0.05 |
| D179V | 604.63 | 490.35 | 1.23 | 0.11 | 0.10 |
| D179C | 613.81 | 503.76 | 1.22 | 0.11 | 0.10 |
| E180M | 9630.23 | 8513.41 | 1.13 | 1.04 | 1.00 |
| E180P | 523.92 | 492.75 | 1.06 | 0.06 | 0.06 |
| E180K | 4017.43 | 3889.45 | 1.03 | 0.43 | 0.46 |
| E180Y | 6655.19 | 5379.42 | 1.24 | 0.72 | 0.63 |
| E180Q | 5146.93 | 4568.90 | 1.13 | 0.56 | 0.54 |
| E180R | 6932.51 | 6309.81 | 1.10 | 0.75 | 0.74 |
| E180A | 9562.37 | 8450.18 | 1.13 | 1.04 | 1.00 |
| E180T | 3718.16 | 2425.13 | 1.53 | 0.40 | 0.29 |
| E180I | 9126.95 | 7770.14 | 1.17 | 0.99 | 0.92 |
| E180F | 7014.78 | 5382.78 | 1.30 | 0.76 | 0.63 |
| E180C | 2926.15 | 2569.75 | 1.14 | 0.32 | 0.30 |
| E180G | 5952.65 | 4547.28 | 1.31 | 1.04 | 0.90 |
| E180S | 5217.80 | 3977.60 | 1.31 | 0.91 | 0.78 |
| E180N | 6534.65 | 4843.84 | 1.35 | 1.14 | 0.96 |
| E180D | 7738.70 | 6277.22 | 1.23 | 1.35 | 1.24 |
| D181S | 9064.64 | 8368.97 | 1.08 | 0.98 | 0.99 |
| D181Q | 7875.57 | 7127.19 | 1.11 | 0.85 | 0.84 |
| D181P | 753.20 | 639.76 | 1.18 | 0.08 | 0.08 |
| D181Y | 1137.94 | 716.86 | 1.59 | 0.12 | 0.08 |
| D181R | 997.11 | 712.77 | 1.40 | 0.11 | 0.08 |
| D181V | 945.65 | 721.77 | 1.31 | 0.10 | 0.09 |
| D181F | 933.48 | 670.40 | 1.39 | 0.10 | 0.08 |
| D181A | 7936.89 | 7854.96 | 1.01 | 0.86 | 0.93 |
| D181T | 6867.00 | 6057.09 | 1.13 | 0.74 | 0.71 |
| D181L | 1727.20 | 1274.09 | 1.36 | 0.19 | 0.15 |
| D181E | 8647.28 | 8246.36 | 1.05 | 0.94 | 0.97 |
| D181K | 1087.36 | 696.83 | 1.56 | 0.12 | 0.08 |
| D181M | 3805.65 | 2986.75 | 1.27 | 0.41 | 0.35 |
| D181C | 549.29 | 447.40 | 1.23 | 0.10 | 0.09 |
| D181G | 2764.20 | 2056.56 | 1.34 | 0.48 | 0.41 |
| E182C | 601.45 | 561.21 | 1.07 | 0.07 | 0.07 |
| E182P | 606.01 | 574.24 | 1.06 | 0.07 | 0.07 |
| E182S | 967.49 | 642.27 | 1.51 | 0.10 | 0.08 |
| E182T | 2995.97 | 1779.42 | 1.68 | 0.32 | 0.21 |
| E182R | 661.10 | 622.75 | 1.06 | 0.07 | 0.07 |
| E182D | 2078.47 | 2140.28 | 0.97 | 0.23 | 0.25 |
| E182A | 619.23 | 531.55 | 1.16 | 0.07 | 0.06 |
| E182F | 1484.85 | 1677.82 | 0.88 | 0.16 | 0.20 |
| E182L | 569.35 | 524.25 | 1.09 | 0.06 | 0.06 |
| E182I | 606.88 | 519.75 | 1.17 | 0.07 | 0.06 |
| E182Y | 593.61 | 561.88 | 1.06 | 0.06 | 0.07 |
| E182Q | 1393.28 | 804.84 | 1.73 | 0.15 | 0.09 |
| E182W | 556.78 | 536.32 | 1.04 | 0.06 | 0.06 |
| E182M | 649.73 | 524.43 | 1.24 | 0.11 | 0.10 |
| E182G | 604.92 | 543.78 | 1.11 | 0.11 | 0.11 |
| R183P | 9143.00 | 8148.29 | 1.12 | 0.99 | 0.96 |
| R183K | 9843.98 | 8685.25 | 1.13 | 1.07 | 1.02 |
| R183W | 8144.07 | 7669.02 | 1.06 | 0.88 | 0.90 |
| R183E | 9873.25 | 8403.44 | 1.17 | 1.07 | 0.99 |
| R183A | 9386.14 | 8368.29 | 1.12 | 1.02 | 0.99 |
| R183T | 4841.94 | 8385.09 | 0.58 | 0.52 | 0.99 |
| R183L | 517.07 | 532.72 | 0.97 | 0.06 | 0.06 |
| R183N | 10062.02 | 8456.13 | 1.19 | 1.09 | 1.00 |
| R183H | 9434.01 | 8295.55 | 1.14 | 1.02 | 0.98 |
| R183V | 9252.08 | 7954.42 | 1.16 | 1.00 | 0.94 |
| R183C | 6603.93 | 6597.30 | 1.00 | 0.72 | 0.78 |
| R183M | 9679.52 | 8250.27 | 1.17 | 1.05 | 0.97 |
| R183I | 495.34 | 8009.63 | 0.06 | 0.05 | 0.94 |
| R183G | 7326.36 | 6021.39 | 1.22 | 1.28 | 1.19 |
| R183S | 7896.17 | 6240.74 | 1.27 | 1.38 | 1.23 |
| W184G | 430.62 | 391.79 | 1.10 | 0.05 | 0.05 |
| W184H | 440.24 | 428.35 | 1.03 | 0.05 | 0.05 |
| W184L | 476.77 | 428.90 | 1.11 | 0.06 | 0.05 |
| W184E | 463.88 | 438.97 | 1.06 | 0.05 | 0.05 |
| W184P | 437.57 | 387.71 | 1.13 | 0.05 | 0.05 |
| W184N | 467.91 | 468.52 | 1.00 | 0.06 | 0.06 |
| W184A | 452.63 | 451.66 | 1.00 | 0.05 | 0.06 |
| W184T | 421.08 | 419.51 | 1.00 | 0.05 | 0.05 |
| W184R | 457.42 | 390.02 | 1.17 | 0.05 | 0.05 |
| W184Q | 450.92 | 448.33 | 1.01 | 0.05 | 0.05 |
| W184V | 454.60 | 407.30 | 1.12 | 0.05 | 0.05 |
| W184S | 486.70 | 485.16 | 1.00 | 0.06 | 0.06 |
| W184M | 447.30 | 395.61 | 1.13 | 0.05 | 0.05 |
| W184I | 478.17 | 503.24 | 0.95 | 0.06 | 0.06 |
| W184F | 455.86 | 427.51 | 1.07 | 0.05 | 0.05 |
| T185R | 1728.04 | 851.07 | 2.03 | 0.20 | 0.10 |
| T185Y | 937.75 | 540.66 | 1.73 | 0.11 | 0.07 |
| T185W | 577.54 | 501.10 | 1.15 | 0.07 | 0.06 |
| T185H | 1448.04 | 783.89 | 1.85 | 0.17 | 0.10 |
| T185G | 3922.30 | 1990.15 | 1.97 | 0.46 | 0.24 |
| T185P | 1773.27 | 1542.44 | 1.15 | 0.21 | 0.19 |
| T185S | 9554.77 | 8267.62 | 1.16 | 1.12 | 1.01 |
| T185V | 1648.14 | 897.66 | 1.84 | 0.19 | 0.11 |
| T185Q | 1594.81 | 583.93 | 2.73 | 0.19 | 0.07 |
| T185N | 790.61 | 546.44 | 1.45 | 0.09 | 0.07 |
| T185C | 1554.42 | 1248.58 | 1.24 | 0.18 | 0.15 |
| T185L | 483.25 | 463.52 | 1.04 | 0.06 | 0.06 |
| T185A | 1599.64 | 711.08 | 2.25 | 0.19 | 0.09 |
| T185E | 1324.02 | 703.76 | 1.88 | 0.16 | 0.09 |
| T185D | 485.86 | 418.67 | 1.16 | 0.06 | 0.06 |
| N186G | 7592.31 | 6944.43 | 1.09 | 0.89 | 0.85 |
| N186A | 7466.07 | 7519.13 | 0.99 | 0.88 | 0.92 |
| N186T | 8897.05 | 8063.02 | 1.10 | 1.05 | 0.98 |
| N186R | 3212.69 | 3085.21 | 1.04 | 0.38 | 0.38 |
| N186L | 8097.42 | 7286.32 | 1.11 | 0.95 | 0.89 |
| N186P | 2173.37 | 1948.86 | 1.12 | 0.26 | 0.24 |
| N186S | 6854.56 | 6735.79 | 1.02 | 0.81 | 0.82 |
| N186V | 6303.91 | 6575.96 | 0.96 | 0.74 | 0.80 |
| N186Q | 4834.56 | 4621.18 | 1.05 | 0.57 | 0.56 |
| N186H | 3390.53 | 3309.97 | 1.02 | 0.40 | 0.40 |
| N186C | 3139.47 | 3113.35 | 1.01 | 0.37 | 0.38 |
| N186E | 3801.36 | 3332.52 | 1.14 | 0.45 | 0.41 |
| N186F | 3794.65 | 3316.48 | 1.14 | 0.45 | 0.40 |
| N186Y | 6301.09 | 7570.59 | 0.83 | 0.74 | 0.92 |
| N186D | 6853.09 | 6333.37 | 1.08 | 0.81 | 0.77 |
| N187R | 1042.36 | 709.74 | 1.47 | 0.12 | 0.09 |
| N187M | 1731.67 | 995.07 | 1.74 | 0.20 | 0.12 |
| N187S | 9538.59 | 8971.12 | 1.06 | 1.12 | 1.10 |
| N187T | 9856.38 | 8855.58 | 1.11 | 1.16 | 1.08 |
| N187L | 505.93 | 464.62 | 1.09 | 0.06 | 0.06 |
| N187W | 1694.86 | 1425.68 | 1.19 | 0.20 | 0.17 |
| N187F | 1240.41 | 731.98 | 1.69 | 0.15 | 0.09 |
| N187K | 2331.93 | 1140.19 | 2.05 | 0.27 | 0.14 |
| N187I | 1444.98 | 683.03 | 2.12 | 0.17 | 0.08 |
| N187A | 4379.80 | 2616.49 | 1.67 | 0.52 | 0.32 |
| N187P | 644.27 | 572.98 | 1.12 | 0.08 | 0.07 |
| N187D | 9843.65 | 8801.57 | 1.12 | 1.16 | 1.07 |
| N187G | 535.06 | 514.10 | 1.04 | 0.07 | 0.07 |
| N187C | 1804.28 | 1860.67 | 0.97 | 0.23 | 0.25 |
| N187H | 1143.07 | 1071.67 | 1.07 | 0.14 | 0.14 |
| F188P | 10012.21 | 8943.91 | 1.12 | 1.18 | 1.09 |
| F188I | 7342.21 | 6782.40 | 1.08 | 0.86 | 0.83 |
| F188N | 10024.22 | 8961.63 | 1.12 | 1.18 | 1.09 |
| F188S | 9564.51 | 8841.98 | 1.08 | 1.13 | 1.08 |
| F188Q | 9591.39 | 8664.63 | 1.11 | 1.13 | 1.06 |
| F188K | 8347.12 | 7497.38 | 1.11 | 0.98 | 0.92 |
| F188G | 9891.61 | 9065.43 | 1.09 | 1.16 | 1.11 |
| F188W | 9389.97 | 8774.36 | 1.07 | 1.10 | 1.07 |
| F188E | 10235.38 | 8984.46 | 1.14 | 1.20 | 1.10 |
| F188H | 2065.12 | 1901.41 | 1.09 | 0.24 | 0.23 |
| F188D | 10087.61 | 8889.75 | 1.13 | 1.19 | 1.09 |
| F188A | 1502.70 | 1231.99 | 1.22 | 0.18 | 0.15 |
| F188L | 8309.64 | 7501.30 | 1.11 | 0.98 | 0.92 |
| F188R | 8182.64 | 7750.05 | 1.06 | 0.96 | 0.95 |
| F188V | 7116.29 | 5860.00 | 1.21 | 1.24 | 1.16 |
| R189L | 9236.08 | 8947.54 | 1.03 | 1.09 | 1.09 |
| R189G | 10307.88 | 9096.35 | 1.13 | 1.21 | 1.11 |
| R189K | 9365.15 | 9033.15 | 1.04 | 1.10 | 1.10 |
| R189P | 3200.68 | 3533.96 | 0.91 | 0.38 | 0.43 |
| R189E | 9552.57 | 8789.28 | 1.09 | 1.12 | 1.07 |
| R189V | 9150.17 | 8088.39 | 1.13 | 1.08 | 0.99 |
| R189D | 9506.16 | 8933.41 | 1.06 | 1.12 | 1.09 |
| R189Y | 9893.14 | 8946.52 | 1.11 | 1.16 | 1.09 |
| R189C | 5318.06 | 5457.78 | 0.97 | 0.63 | 0.67 |
| R189A | 9718.09 | 8798.13 | 1.10 | 1.14 | 1.07 |
| R189H | 1360.90 | 1350.33 | 1.01 | 0.16 | 0.16 |
| R189W | 7657.12 | 7070.92 | 1.08 | 0.90 | 0.86 |
| R189N | 7842.39 | 6675.36 | 1.17 | 1.37 | 1.32 |
| R189T | 7610.10 | 6459.94 | 1.18 | 1.33 | 1.27 |
| R189Q | 7465.37 | 6396.79 | 1.17 | 1.30 | 1.26 |
| E190A | 1510.06 | 2116.94 | 0.71 | 0.21 | 0.23 |
| E190H | 6276.13 | 7564.04 | 0.83 | 0.88 | 0.83 |
| E190V | 643.37 | 1658.11 | 0.39 | 0.09 | 0.18 |
| E190P | 2420.68 | 1767.43 | 1.37 | 0.34 | 0.19 |
| E190C | 1827.25 | 2083.88 | 0.88 | 0.26 | 0.23 |
| E190G | 5313.99 | 4365.93 | 1.22 | 0.75 | 0.48 |
| E190R | 1185.26 | 1810.53 | 0.65 | 0.17 | 0.20 |
| E190I | 1880.80 | 2886.28 | 0.65 | 0.27 | 0.32 |
| E190S | 4542.61 | 5987.33 | 0.76 | 0.64 | 0.66 |
| E190T | 2293.47 | 4444.68 | 0.52 | 0.32 | 0.49 |
| E190M | 2557.21 | 1317.73 | 1.94 | 0.36 | 0.15 |
| E190L | 2542.38 | 2986.91 | 0.85 | 0.36 | 0.33 |
| E190K | 2960.37 | 4343.12 | 0.68 | 0.42 | 0.48 |
| E190Y | 7243.54 | 5742.33 | 1.26 | 1.27 | 1.13 |
| E190D | 7910.21 | 6468.78 | 1.22 | 1.38 | 1.28 |
| Y191T | 611.75 | 535.95 | 1.14 | 0.07 | 0.06 |
| Y191H | 2333.85 | 2191.64 | 1.06 | 0.28 | 0.24 |
| Y191G | 428.17 | 432.65 | 0.99 | 0.05 | 0.05 |
| Y191L | 379.02 | 357.82 | 1.06 | 0.05 | 0.04 |
| Y191P | 1359.30 | 1046.33 | 1.30 | 0.16 | 0.12 |
| Y191Q | 451.92 | 403.46 | 1.12 | 0.05 | 0.05 |
| Y191K | 464.62 | 406.52 | 1.14 | 0.06 | 0.05 |
| Y191D | 392.24 | 370.67 | 1.06 | 0.05 | 0.04 |
| Y191A | 452.13 | 418.53 | 1.08 | 0.05 | 0.05 |
| Y191W | 395.63 | 411.91 | 0.96 | 0.05 | 0.05 |
| Y191S | 530.80 | 447.13 | 1.19 | 0.06 | 0.05 |
| Y191V | 1553.58 | 1254.11 | 1.24 | 0.19 | 0.14 |
| Y191E | 395.04 | 407.49 | 0.97 | 0.05 | 0.05 |
| Y191R | 652.95 | 725.68 | 0.90 | 0.08 | 0.08 |
| Y191C | 530.42 | 463.90 | 1.14 | 0.06 | 0.05 |
| N192R | 640.72 | 482.61 | 1.33 | 0.09 | 0.05 |
| N192L | 591.92 | 571.56 | 1.04 | 0.08 | 0.06 |
| N192Q | 1089.41 | 1020.23 | 1.07 | 0.15 | 0.11 |
| N192P | 685.62 | 856.11 | 0.80 | 0.10 | 0.09 |
| N192H | 2274.24 | 1058.80 | 2.15 | 0.32 | 0.12 |
| N192S | 2043.65 | 1630.74 | 1.25 | 0.29 | 0.18 |
| N192W | 548.30 | 538.86 | 1.02 | 0.08 | 0.06 |
| N192G | 899.47 | 659.29 | 1.36 | 0.13 | 0.07 |
| N192D | 4213.33 | 2216.40 | 1.90 | 0.59 | 0.24 |
| N192V | 588.02 | 537.64 | 1.09 | 0.08 | 0.06 |
| N192A | 574.26 | 543.66 | 1.06 | 0.08 | 0.06 |
| N192T | 536.50 | 576.21 | 0.93 | 0.08 | 0.06 |
| N192K | 685.26 | 633.89 | 1.08 | 0.10 | 0.07 |
| N192C | 1310.46 | 987.31 | 1.33 | 0.18 | 0.11 |
| N192M | 547.98 | 537.29 | 1.02 | 0.08 | 0.06 |
| L193P | 388.57 | 381.15 | 1.02 | 0.05 | 0.04 |
| L193G | 437.84 | 478.44 | 0.92 | 0.05 | 0.05 |
| L193F | 481.49 | 491.33 | 0.98 | 0.06 | 0.05 |
| L193S | 448.35 | 449.03 | 1.00 | 0.05 | 0.05 |
| L193W | 481.79 | 460.74 | 1.05 | 0.06 | 0.05 |
| L193A | 510.96 | 468.83 | 1.09 | 0.06 | 0.05 |
| L193R | 481.08 | 477.55 | 1.01 | 0.06 | 0.05 |
| L193Q | 417.53 | 412.01 | 1.01 | 0.05 | 0.05 |
| L193E | 401.70 | 409.23 | 0.98 | 0.05 | 0.05 |
| L193K | 417.39 | 426.26 | 0.98 | 0.05 | 0.05 |
| L193N | 432.35 | 434.95 | 0.99 | 0.05 | 0.05 |
| L193I | 2767.03 | 3467.03 | 0.80 | 0.33 | 0.39 |
| L193T | 679.48 | 638.88 | 1.06 | 0.08 | 0.07 |
| L193D | 419.37 | 424.41 | 0.99 | 0.05 | 0.05 |
| L193Y | 3022.00 | 2706.28 | 1.12 | 0.36 | 0.30 |
| H194S | 5518.27 | 6112.38 | 0.90 | 0.78 | 0.67 |
| H194E | 7667.53 | 8295.22 | 0.92 | 1.08 | 0.91 |
| H194K | 5130.62 | 6124.27 | 0.84 | 0.72 | 0.67 |
| H194Q | 6399.62 | 7113.56 | 0.90 | 0.90 | 0.78 |
| H194V | 1611.06 | 5696.43 | 0.28 | 0.23 | 0.63 |
| H194T | 3884.64 | 2598.12 | 1.50 | 0.55 | 0.29 |
| H194L | 5710.11 | 6872.56 | 0.83 | 0.80 | 0.76 |
| H194Y | 4922.31 | 5688.29 | 0.87 | 0.69 | 0.63 |
| H194F | 3787.65 | 5388.18 | 0.70 | 0.53 | 0.59 |
| H194G | 4636.22 | 5437.23 | 0.85 | 0.65 | 0.60 |
| H194I | 2901.13 | 3777.68 | 0.77 | 0.41 | 0.42 |
| H194W | 5434.60 | 7448.23 | 0.73 | 0.77 | 0.82 |
| H194M | 2941.85 | 9057.43 | 0.32 | 0.41 | 1.00 |
| H194A | 4681.45 | 2746.90 | 1.70 | 0.66 | 0.30 |
| H194P | 5264.79 | 5058.19 | 1.04 | 0.74 | 0.56 |
| R195C | 4231.32 | 1853.20 | 2.28 | 0.60 | 0.20 |
| R195F | 687.70 | 720.42 | 0.95 | 0.10 | 0.08 |
| R195W | 5099.23 | 4524.84 | 1.13 | 0.72 | 0.50 |
| R195T | 1101.98 | 1175.85 | 0.94 | 0.16 | 0.13 |
| R195L | 5073.57 | 4520.73 | 1.12 | 0.72 | 0.50 |
| R195G | 5269.21 | 3025.93 | 1.74 | 0.74 | 0.33 |
| R195Q | 1958.69 | 1361.83 | 1.44 | 0.28 | 0.15 |
| R195K | 3839.86 | 7080.78 | 0.54 | 0.54 | 0.78 |
| R195S | 642.14 | 649.21 | 0.99 | 0.09 | 0.07 |
| R195A | 5605.90 | 3852.81 | 1.46 | 0.79 | 0.42 |
| R195D | 2724.53 | 1907.81 | 1.43 | 0.38 | 0.21 |
| R195P | 571.50 | 615.21 | 0.93 | 0.08 | 0.07 |
| R195Y | 763.31 | 794.42 | 0.96 | 0.11 | 0.09 |
| R195E | 7597.55 | 8468.35 | 0.90 | 1.07 | 0.93 |
| R195V | 1711.48 | 1037.62 | 1.65 | 0.24 | 0.11 |
| V196T | 1040.90 | 1268.04 | 0.82 | 0.12 | 0.14 |
| V196D | 443.04 | 446.39 | 0.99 | 0.05 | 0.05 |
| V196G | 490.83 | 494.67 | 0.99 | 0.06 | 0.06 |
| V196E | 488.55 | 489.74 | 1.00 | 0.06 | 0.05 |
| V196A | 452.36 | 452.12 | 1.00 | 0.05 | 0.05 |
| V196S | 1186.52 | 949.52 | 1.25 | 0.14 | 0.11 |
| V196Q | 412.17 | 430.91 | 0.96 | 0.05 | 0.05 |
| V196P | 576.83 | 620.88 | 0.93 | 0.07 | 0.07 |
| V196R | 493.29 | 474.38 | 1.04 | 0.06 | 0.05 |
| V196H | 465.64 | 479.66 | 0.97 | 0.06 | 0.05 |
| V196Y | 462.28 | 474.94 | 0.97 | 0.06 | 0.05 |
| V196I | 1125.67 | 1229.87 | 0.92 | 0.13 | 0.14 |
| V196L | 464.80 | 491.01 | 0.95 | 0.06 | 0.05 |
| V196K | 455.84 | 482.44 | 0.94 | 0.05 | 0.05 |
| V196M | 479.36 | 518.00 | 0.93 | 0.06 | 0.06 |
| A197G | 1238.39 | 2552.91 | 0.49 | 0.17 | 0.28 |
| A197S | 3959.39 | 2633.91 | 1.50 | 0.56 | 0.29 |
| A197L | 1013.13 | 809.32 | 1.25 | 0.14 | 0.09 |
| A197P | 857.06 | 933.29 | 0.92 | 0.12 | 0.10 |
| A197V | 2549.12 | 4355.70 | 0.59 | 0.36 | 0.48 |
| A197Y | 650.21 | 722.49 | 0.90 | 0.09 | 0.08 |
| A197Q | 658.64 | 652.52 | 1.01 | 0.09 | 0.07 |
| A197R | 635.08 | 640.91 | 0.99 | 0.09 | 0.07 |
| A197T | 1933.94 | 4482.59 | 0.43 | 0.27 | 0.49 |
| A197I | 1440.69 | 1060.51 | 1.36 | 0.20 | 0.12 |
| A197H | 604.11 | 638.63 | 0.95 | 0.09 | 0.07 |
| A197E | 686.96 | 624.22 | 1.10 | 0.10 | 0.07 |
| A197W | 1448.83 | 2588.64 | 0.56 | 0.20 | 0.29 |
| A197N | 623.17 | 840.56 | 0.74 | 0.09 | 0.09 |
| A197C | 4012.80 | 3140.52 | 1.28 | 0.70 | 0.62 |
| A198T | 761.19 | 700.22 | 1.09 | 0.11 | 0.08 |
| A198K | 490.20 | 1179.92 | 0.42 | 0.07 | 0.13 |
| A198S | 4061.28 | 3136.65 | 1.29 | 0.57 | 0.35 |
| A198H | 581.41 | 575.73 | 1.01 | 0.08 | 0.06 |
| A198G | 2610.82 | 2368.26 | 1.10 | 0.37 | 0.26 |
| A198E | 485.45 | 662.62 | 0.73 | 0.07 | 0.07 |
| A198P | 656.48 | 580.71 | 1.13 | 0.09 | 0.06 |
| A198L | 1339.94 | 726.74 | 1.84 | 0.19 | 0.08 |
| A198R | 570.33 | 565.56 | 1.01 | 0.08 | 0.06 |
| A198V | 3026.36 | 7305.85 | 0.41 | 0.43 | 0.80 |
| A198M | 1384.46 | 999.55 | 1.39 | 0.20 | 0.11 |
| A198F | 572.48 | 559.57 | 1.02 | 0.08 | 0.06 |
| A198W | 560.48 | 547.72 | 1.02 | 0.08 | 0.06 |
| A198Y | 486.57 | 612.28 | 0.79 | 0.07 | 0.07 |
| A198D | 633.49 | 474.50 | 1.34 | 0.09 | 0.05 |
| H199I | 520.35 | 496.48 | 1.05 | 0.08 | 0.07 |
| H199P | 437.57 | 404.21 | 1.08 | 0.07 | 0.05 |
| H199G | 436.53 | 392.94 | 1.11 | 0.07 | 0.05 |
| H199N | 420.26 | 375.18 | 1.12 | 0.07 | 0.05 |
| H199S | 411.09 | 377.36 | 1.09 | 0.06 | 0.05 |
| H199L | 531.61 | 530.53 | 1.00 | 0.08 | 0.07 |
| H199M | 413.37 | 384.23 | 1.08 | 0.07 | 0.05 |
| H199A | 391.56 | 381.36 | 1.03 | 0.06 | 0.05 |
| H199C | 404.49 | 366.35 | 1.10 | 0.06 | 0.05 |
| H199K | 402.34 | 383.95 | 1.05 | 0.06 | 0.05 |
| H199R | 422.19 | 387.94 | 1.09 | 0.07 | 0.05 |
| H199V | 421.16 | 378.71 | 1.11 | 0.07 | 0.05 |
| H199W | 377.01 | 345.02 | 1.09 | 0.06 | 0.05 |
| H199T | 399.21 | 382.65 | 1.04 | 0.06 | 0.05 |
| H199E | 399.49 | 385.83 | 1.04 | 0.06 | 0.05 |
| E200P | 414.11 | 409.55 | 1.01 | 0.07 | 0.06 |
| E200G | 440.94 | 402.85 | 1.09 | 0.07 | 0.05 |
| E200A | 448.41 | 413.61 | 1.08 | 0.07 | 0.06 |
| E200T | 461.19 | 418.51 | 1.10 | 0.07 | 0.06 |
| E200I | 457.88 | 419.19 | 1.09 | 0.07 | 0.06 |
| E200W | 418.40 | 403.05 | 1.04 | 0.07 | 0.05 |
| E200R | 449.83 | 425.86 | 1.06 | 0.07 | 0.06 |
| E200F | 446.49 | 417.58 | 1.07 | 0.07 | 0.06 |
| E200M | 448.32 | 428.16 | 1.05 | 0.07 | 0.06 |
| E200D | 428.91 | 401.64 | 1.07 | 0.07 | 0.05 |
| E200V | 426.45 | 407.13 | 1.05 | 0.07 | 0.06 |
| E200C | 413.11 | 384.79 | 1.07 | 0.07 | 0.05 |
| E200S | 422.57 | 391.02 | 1.08 | 0.07 | 0.05 |
| E200Y | 412.07 | 393.97 | 1.05 | 0.07 | 0.05 |
| E200N | 430.94 | 412.07 | 1.05 | 0.07 | 0.06 |
| L201A | 754.66 | 957.77 | 0.79 | 0.12 | 0.13 |
| L201R | 442.35 | 442.57 | 1.00 | 0.07 | 0.06 |
| L201E | 464.22 | 443.89 | 1.05 | 0.07 | 0.06 |
| L201P | 494.97 | 471.92 | 1.05 | 0.08 | 0.06 |
| L201G | 574.82 | 590.26 | 0.97 | 0.09 | 0.08 |
| L201V | 3359.21 | 4623.67 | 0.73 | 0.53 | 0.62 |
| L201T | 1509.22 | 2175.97 | 0.69 | 0.24 | 0.29 |
| L201I | 2861.66 | 2231.87 | 1.28 | 0.45 | 0.30 |
| L201S | 859.79 | 964.65 | 0.89 | 0.14 | 0.13 |
| L201W | 1258.36 | 1335.56 | 0.94 | 0.20 | 0.18 |
| L201Q | 657.51 | 749.98 | 0.88 | 0.10 | 0.10 |
| L201D | 486.09 | 471.81 | 1.03 | 0.08 | 0.06 |
| L201M | 5637.84 | 7147.36 | 0.79 | 0.89 | 0.97 |
| L201K | 484.89 | 467.23 | 1.04 | 0.08 | 0.06 |
| L201N | 440.03 | 432.42 | 1.02 | 0.07 | 0.06 |
| G202T | 556.53 | 546.05 | 1.02 | 0.09 | 0.07 |
| G202Y | 533.64 | 530.73 | 1.01 | 0.08 | 0.07 |
| G202E | 558.69 | 543.68 | 1.03 | 0.09 | 0.07 |
| G202V | 569.22 | 572.58 | 0.99 | 0.09 | 0.08 |
| G202S | 512.82 | 503.35 | 1.02 | 0.08 | 0.07 |
| G202L | 513.71 | 508.34 | 1.01 | 0.08 | 0.07 |
| G202I | 535.96 | 516.37 | 1.04 | 0.08 | 0.07 |
| G202M | 507.94 | 500.04 | 1.02 | 0.08 | 0.07 |
| G202H | 567.88 | 547.25 | 1.04 | 0.09 | 0.07 |
| G202C | 508.19 | 499.05 | 1.02 | 0.08 | 0.07 |
| G202R | 537.10 | 511.28 | 1.05 | 0.08 | 0.07 |
| G202P | 544.39 | 535.24 | 1.02 | 0.09 | 0.07 |
| G202A | 580.75 | 571.95 | 1.02 | 0.09 | 0.08 |
| G202K | 531.07 | 520.45 | 1.02 | 0.08 | 0.07 |
| G202D | 559.64 | 544.50 | 1.03 | 0.09 | 0.07 |
| H203Y | 910.26 | 1218.02 | 0.75 | 0.14 | 0.16 |
| H203E | 7284.23 | 7937.91 | 0.92 | 1.15 | 1.07 |
| H203R | 545.70 | 545.36 | 1.00 | 0.09 | 0.07 |
| H203Q | 570.55 | 541.42 | 1.05 | 0.09 | 0.07 |
| H203P | 547.00 | 527.67 | 1.04 | 0.09 | 0.07 |
| H203G | 558.88 | 576.91 | 0.97 | 0.09 | 0.08 |
| H203T | 534.16 | 535.33 | 1.00 | 0.08 | 0.07 |
| H203D | 542.85 | 530.31 | 1.02 | 0.09 | 0.07 |
| H203L | 1224.67 | 1746.14 | 0.70 | 0.19 | 0.24 |
| H203N | 547.92 | 532.11 | 1.03 | 0.09 | 0.07 |
| H203A | 513.18 | 515.49 | 1.00 | 0.08 | 0.07 |
| H203S | 534.50 | 507.56 | 1.05 | 0.08 | 0.07 |
| H203V | 565.64 | 554.43 | 1.02 | 0.09 | 0.07 |
| H203I | 568.56 | 613.73 | 0.93 | 0.09 | 0.08 |
| H203C | 504.41 | 522.69 | 0.97 | 0.08 | 0.07 |
| S204R | 557.42 | 544.69 | 1.02 | 0.09 | 0.07 |
| S204N | 733.30 | 754.34 | 0.97 | 0.12 | 0.10 |
| S204A | 3654.83 | 3972.28 | 0.92 | 0.58 | 0.54 |
| S204T | 1697.49 | 3586.11 | 0.47 | 0.27 | 0.48 |
| S204Y | 550.01 | 538.07 | 1.02 | 0.09 | 0.07 |
| S204V | 3063.02 | 1827.71 | 1.68 | 0.48 | 0.25 |
| S204L | 501.10 | 594.44 | 0.84 | 0.08 | 0.08 |
| S204H | 486.78 | 508.13 | 0.96 | 0.08 | 0.07 |
| S204D | 507.05 | 489.81 | 1.04 | 0.08 | 0.07 |
| S204Q | 530.67 | 472.92 | 1.12 | 0.08 | 0.06 |
| S204G | 1483.41 | 1333.79 | 1.11 | 0.23 | 0.18 |
| S204W | 487.01 | 504.11 | 0.97 | 0.08 | 0.07 |
| S204I | 634.82 | 516.30 | 1.23 | 0.10 | 0.07 |
| S204K | 484.92 | 471.83 | 1.03 | 0.08 | 0.06 |
| S204P | 483.87 | 506.90 | 0.95 | 0.08 | 0.07 |
| L205T | 1304.89 | 1099.25 | 1.19 | 0.13 | 0.12 |
| L205D | 774.29 | 830.37 | 0.93 | 0.08 | 0.09 |
| L205S | 686.11 | 601.35 | 1.14 | 0.07 | 0.07 |
| L205G | 792.45 | 790.93 | 1.00 | 0.08 | 0.09 |
| L205P | 592.15 | 673.32 | 0.88 | 0.06 | 0.07 |
| L205E | 473.89 | 446.64 | 1.06 | 0.05 | 0.05 |
| L205V | 5589.64 | 7308.12 | 0.76 | 0.57 | 0.80 |
| L205M | 8334.85 | 8229.20 | 1.01 | 0.85 | 0.90 |
| L205N | 1426.11 | 1322.80 | 1.08 | 0.15 | 0.15 |
| L205C | 1903.14 | 2394.15 | 0.79 | 0.20 | 0.26 |
| L205I | 5644.28 | 7817.06 | 0.72 | 0.58 | 0.86 |
| L205A | 1796.22 | 1704.85 | 1.05 | 0.18 | 0.19 |
| L205R | 508.62 | 575.22 | 0.88 | 0.05 | 0.06 |
| L205W | 497.92 | 427.60 | 1.16 | 0.05 | 0.05 |
| L205Q | 2191.83 | 2399.54 | 0.91 | 0.22 | 0.26 |
| G206I | 467.21 | 460.72 | 1.01 | 0.05 | 0.05 |
| G206V | 619.10 | 682.58 | 0.91 | 0.06 | 0.07 |
| G206A | 4554.61 | 2702.11 | 1.69 | 0.47 | 0.30 |
| G206C | 491.44 | 469.90 | 1.05 | 0.05 | 0.05 |
| G206S | 1226.37 | 919.66 | 1.33 | 0.13 | 0.10 |
| G206P | 503.21 | 497.87 | 1.01 | 0.05 | 0.05 |
| G206L | 499.74 | 469.53 | 1.06 | 0.05 | 0.05 |
| G206D | 490.08 | 451.61 | 1.09 | 0.05 | 0.05 |
| G206M | 478.55 | 451.47 | 1.06 | 0.05 | 0.05 |
| G206R | 677.07 | 831.95 | 0.81 | 0.07 | 0.09 |
| G206Q | 805.32 | 851.38 | 0.95 | 0.08 | 0.09 |
| G206E | 469.86 | 447.60 | 1.05 | 0.05 | 0.05 |
| G206H | 463.25 | 437.73 | 1.06 | 0.05 | 0.05 |
| G206T | 475.20 | 491.10 | 0.97 | 0.05 | 0.05 |
| G206W | 472.91 | 437.66 | 1.08 | 0.05 | 0.05 |
| L207S | 657.07 | 501.03 | 1.31 | 0.07 | 0.05 |
| L207Y | 1032.96 | 1142.52 | 0.90 | 0.11 | 0.13 |
| L207A | 6302.90 | 5614.64 | 1.12 | 0.65 | 0.62 |
| L207R | 3476.88 | 1332.44 | 2.61 | 0.36 | 0.15 |
| L207P | 528.87 | 508.95 | 1.04 | 0.05 | 0.06 |
| L207Q | 671.72 | 518.36 | 1.30 | 0.07 | 0.06 |
| L207N | 551.03 | 476.05 | 1.16 | 0.06 | 0.05 |
| L207K | 860.90 | 594.92 | 1.45 | 0.09 | 0.07 |
| L207M | 11903.05 | 12984.69 | 0.92 | 1.22 | 1.42 |
| L207W | 509.40 | 470.26 | 1.08 | 0.05 | 0.05 |
| L207H | 620.20 | 595.55 | 1.04 | 0.06 | 0.07 |
| L207D | 523.82 | 473.80 | 1.11 | 0.05 | 0.05 |
| L207V | 656.95 | 550.54 | 1.19 | 0.08 | 0.07 |
| L207I | 645.37 | 550.32 | 1.17 | 0.08 | 0.07 |
| L207G | 610.01 | 484.35 | 1.26 | 0.08 | 0.06 |
| S208D | 10064.82 | 9325.26 | 1.08 | 1.03 | 1.02 |
| S208V | 10469.49 | 9334.16 | 1.12 | 1.07 | 1.02 |
| S208P | 9922.26 | 9236.91 | 1.07 | 1.02 | 1.01 |
| S208G | 10452.64 | 9295.93 | 1.12 | 1.07 | 1.02 |
| S208A | 10553.22 | 9517.15 | 1.11 | 1.08 | 1.04 |
| S208K | 22659.58 | 19984.18 | 1.13 | 2.32 | 2.19 |
| S208N | 9993.85 | 9327.07 | 1.07 | 1.02 | 1.02 |
| S208F | 8826.28 | 9040.21 | 0.98 | 0.91 | 0.99 |
| S208Q | 10196.89 | 9183.58 | 1.11 | 1.05 | 1.01 |
| S208W | 9229.04 | 9226.75 | 1.00 | 0.95 | 1.01 |
| S208T | 9241.73 | 8912.77 | 1.04 | 0.95 | 0.98 |
| S208E | 10198.81 | 9401.75 | 1.08 | 1.05 | 1.03 |
| S208C | 10497.72 | 16287.64 | 0.64 | 1.08 | 1.79 |
| S208R | 7639.06 | 6465.10 | 1.18 | 1.34 | 1.28 |
| S208L | 7811.78 | 6354.14 | 1.23 | 1.37 | 1.25 |
| H209T | 466.30 | 415.72 | 1.12 | 0.11 | 0.08 |
| H209Y | 471.70 | 455.15 | 1.04 | 0.11 | 0.09 |
| H209R | 489.49 | 463.09 | 1.06 | 0.12 | 0.09 |
| H209Q | 513.42 | 476.96 | 1.08 | 0.12 | 0.09 |
| H209A | 511.91 | 469.64 | 1.09 | 0.12 | 0.09 |
| H209G | 495.58 | 466.25 | 1.06 | 0.12 | 0.09 |
| H209N | 455.09 | 424.90 | 1.07 | 0.11 | 0.08 |
| H209P | 526.85 | 480.73 | 1.10 | 0.13 | 0.09 |
| H209W | 516.05 | 484.16 | 1.07 | 0.12 | 0.09 |
| H209V | 499.35 | 465.99 | 1.07 | 0.12 | 0.09 |
| H209D | 479.48 | 442.06 | 1.08 | 0.12 | 0.09 |
| H209S | 490.77 | 438.98 | 1.12 | 0.12 | 0.09 |
| H209F | 490.42 | 437.68 | 1.12 | 0.12 | 0.09 |
| H209L | 491.46 | 441.89 | 1.11 | 0.12 | 0.09 |
| H209C | 471.56 | 420.60 | 1.12 | 0.11 | 0.08 |
| S210C | 634.06 | 565.38 | 1.12 | 0.15 | 0.11 |
| S210G | 643.08 | 581.11 | 1.11 | 0.16 | 0.11 |
| S210I | 778.38 | 625.00 | 1.25 | 0.19 | 0.12 |
| S210R | 644.74 | 565.67 | 1.14 | 0.16 | 0.11 |
| S210L | 737.60 | 623.25 | 1.18 | 0.18 | 0.12 |
| S210V | 1190.35 | 856.63 | 1.39 | 0.29 | 0.17 |
| S210H | 605.43 | 521.90 | 1.16 | 0.15 | 0.10 |
| S210N | 615.29 | 556.38 | 1.11 | 0.15 | 0.11 |
| S210F | 529.93 | 487.42 | 1.09 | 0.13 | 0.09 |
| S210P | 544.94 | 513.59 | 1.06 | 0.13 | 0.10 |
| S210W | 527.32 | 486.97 | 1.08 | 0.13 | 0.09 |
| S210Q | 593.74 | 548.93 | 1.08 | 0.14 | 0.11 |
| S210T | 2977.61 | 3427.71 | 0.87 | 0.72 | 0.67 |
| S210K | 625.14 | 573.41 | 1.09 | 0.15 | 0.11 |
| S210A | 1682.05 | 1546.97 | 1.09 | 0.25 | 0.21 |
| T211P | 3493.13 | 3774.82 | 0.93 | 0.84 | 0.73 |
| T211R | 4636.24 | 5429.67 | 0.85 | 1.12 | 1.05 |
| T211K | 4457.25 | 5411.31 | 0.82 | 1.08 | 1.05 |
| T211G | 3443.93 | 3543.72 | 0.97 | 0.83 | 0.69 |
| T211M | 3806.80 | 4871.37 | 0.78 | 0.92 | 0.95 |
| T211N | 5924.95 | 6170.25 | 0.96 | 1.43 | 1.20 |
| T211V | 5095.76 | 5335.63 | 0.96 | 1.23 | 1.04 |
| T211H | 1885.69 | 1829.82 | 1.03 | 0.46 | 0.36 |
| T211Q | 4868.86 | 5772.70 | 0.84 | 1.18 | 1.12 |
| T211S | 4641.02 | 4565.80 | 1.02 | 1.12 | 0.89 |
| T211A | 2696.88 | 2830.43 | 0.95 | 0.65 | 0.55 |
| T211F | 1412.47 | 1277.53 | 1.11 | 0.34 | 0.25 |
| T211D | 2442.24 | 2154.48 | 1.13 | 0.59 | 0.42 |
| T211W | 1362.99 | 1207.40 | 1.13 | 0.33 | 0.23 |
| T211L | 2376.23 | 2102.07 | 1.13 | 0.35 | 0.28 |
| D212E | 4877.68 | 4473.75 | 1.09 | 1.18 | 0.87 |
| D212A | 2710.82 | 2417.65 | 1.12 | 0.66 | 0.47 |
| D212K | 2296.16 | 2049.97 | 1.12 | 0.55 | 0.40 |
| D212R | 2273.87 | 2004.06 | 1.13 | 0.55 | 0.39 |
| D212T | 2923.39 | 2699.75 | 1.08 | 0.71 | 0.52 |
| D212N | 4575.59 | 5229.75 | 0.87 | 1.11 | 1.02 |
| D212G | 1011.62 | 657.28 | 1.54 | 0.24 | 0.13 |
| D212S | 5035.28 | 4894.92 | 1.03 | 1.22 | 0.95 |
| D212P | 3270.81 | 2918.36 | 1.12 | 0.79 | 0.57 |
| D212Q | 2823.54 | 2576.63 | 1.10 | 0.68 | 0.50 |
| D212V | 2000.60 | 1876.86 | 1.07 | 0.48 | 0.36 |
| D212L | 517.72 | 497.60 | 1.04 | 0.13 | 0.10 |
| D212F | 2378.07 | 2185.27 | 1.09 | 0.57 | 0.42 |
| D212H | 4696.49 | 4001.41 | 1.17 | 0.70 | 0.53 |
| D212Y | 5489.99 | 5319.27 | 1.03 | 0.49 | 0.55 |
| I213Q | 9326.77 | 8702.80 | 1.07 | 0.96 | 0.95 |
| I213T | 9396.39 | 8742.82 | 1.07 | 0.96 | 0.96 |
| I213C | 9396.24 | 8859.11 | 1.06 | 0.96 | 0.97 |
| I213P | 10248.90 | 9319.88 | 1.10 | 1.05 | 1.02 |
| I213H | 9826.58 | 9076.23 | 1.08 | 1.01 | 1.00 |
| I213A | 10044.30 | 9249.07 | 1.09 | 1.03 | 1.01 |
| I213V | 10260.18 | 9459.80 | 1.08 | 1.05 | 1.04 |
| I213G | 21327.14 | 19706.88 | 1.08 | 2.19 | 2.16 |
| I213N | 8790.33 | 7995.03 | 1.10 | 0.90 | 0.88 |
| I213L | 9974.73 | 9208.92 | 1.08 | 1.02 | 1.01 |
| I213S | 9599.72 | 9004.65 | 1.07 | 0.98 | 0.99 |
| I213M | 9987.31 | 9083.77 | 1.10 | 1.02 | 1.00 |
| I213R | 9253.06 | 8997.34 | 1.03 | 0.95 | 0.99 |
| I213K | 9682.80 | 9286.32 | 1.04 | 0.99 | 1.02 |
| I213F | 9368.13 | 8940.38 | 1.05 | 0.96 | 0.98 |
| I213D | 7017.06 | 7368.43 | 0.95 | 0.77 | 0.97 |
| I213E | 8169.74 | 7234.77 | 1.13 | 0.90 | 0.95 |
| G214L | 13500.23 | 13135.46 | 1.03 | 1.38 | 1.44 |
| G214Q | 9914.48 | 9182.63 | 1.08 | 1.02 | 1.01 |
| G214S | 9503.68 | 9036.70 | 1.05 | 0.97 | 0.99 |
| G214T | 9940.86 | 9214.25 | 1.08 | 1.02 | 1.01 |
| G214V | 8185.36 | 7785.72 | 1.05 | 0.84 | 0.85 |
| G214I | 6068.79 | 5773.01 | 1.05 | 0.62 | 0.63 |
| G214R | 9720.43 | 9083.04 | 1.07 | 1.00 | 1.00 |
| G214P | 8763.31 | 8875.24 | 0.99 | 0.90 | 0.97 |
| G214E | 21602.30 | 19851.77 | 1.09 | 2.22 | 2.18 |
| G214A | 10063.30 | 9154.93 | 1.10 | 1.03 | 1.00 |
| G214D | 9967.49 | 9121.71 | 1.09 | 1.02 | 1.00 |
| G214F | 9750.30 | 9157.75 | 1.06 | 1.00 | 1.00 |
| G214Y | 9886.62 | 9025.45 | 1.10 | 1.01 | 0.99 |
| G214M | 9472.77 | 8919.05 | 1.06 | 0.97 | 0.98 |
| G214C | 6716.52 | 7097.60 | 0.95 | 0.69 | 0.78 |
| A215L | 454.59 | 428.41 | 1.06 | 0.07 | 0.06 |
| A215Q | 765.06 | 739.09 | 1.04 | 0.12 | 0.10 |
| A215M | 672.22 | 624.41 | 1.08 | 0.10 | 0.09 |
| A215G | 4240.44 | 6854.29 | 0.62 | 0.66 | 0.96 |
| A215W | 377.79 | 348.04 | 1.09 | 0.06 | 0.05 |
| A215S | 559.99 | 538.20 | 1.04 | 0.09 | 0.08 |
| A215T | 664.02 | 711.35 | 0.93 | 0.10 | 0.10 |
| A215V | 473.67 | 492.63 | 0.96 | 0.07 | 0.07 |
| A215N | 4328.77 | 4488.89 | 0.96 | 0.67 | 0.63 |
| A215P | 638.50 | 596.48 | 1.07 | 0.10 | 0.08 |
| A215H | 3954.04 | 4447.65 | 0.89 | 0.61 | 0.62 |
| A215K | 420.46 | 402.71 | 1.04 | 0.07 | 0.06 |
| A215I | 413.93 | 386.28 | 1.07 | 0.06 | 0.05 |
| A215R | 421.35 | 389.00 | 1.08 | 0.07 | 0.05 |
| A215C | 437.44 | 425.03 | 1.03 | 0.07 | 0.06 |
| A215D | 1031.48 | 913.25 | 1.13 | 0.11 | 0.12 |
| L216A | 808.93 | 759.54 | 1.07 | 0.13 | 0.12 |
| L216C | 473.05 | 462.23 | 1.02 | 0.08 | 0.07 |
| L216D | 497.61 | 457.15 | 1.09 | 0.08 | 0.07 |
| L216E | 480.72 | 458.21 | 1.05 | 0.08 | 0.07 |
| L216G | 473.61 | 452.00 | 1.05 | 0.08 | 0.07 |
| L216I | 7525.06 | 8586.88 | 0.88 | 1.20 | 1.37 |
| L216K | 478.52 | 460.66 | 1.04 | 0.08 | 0.07 |
| L216M | 4641.29 | 5160.67 | 0.90 | 0.74 | 0.83 |
| L216P | 466.46 | 475.96 | 0.98 | 0.07 | 0.08 |
| L216Q | 693.10 | 638.62 | 1.09 | 0.11 | 0.10 |
| L216R | 458.77 | 437.34 | 1.05 | 0.07 | 0.07 |
| L216S | 454.85 | 441.50 | 1.03 | 0.07 | 0.07 |
| L216T | 1484.74 | 1392.55 | 1.07 | 0.24 | 0.22 |
| L216V | 5022.20 | 6281.17 | 0.80 | 0.80 | 1.01 |
| L216W | 479.55 | 454.07 | 1.06 | 0.08 | 0.07 |
| M217P | 458.79 | 440.59 | 1.04 | 0.07 | 0.06 |
| M217Y | 459.96 | 427.87 | 1.07 | 0.07 | 0.06 |
| M217T | 699.27 | 663.16 | 1.05 | 0.11 | 0.09 |
| M217C | 5441.67 | 7486.86 | 0.73 | 0.85 | 1.05 |
| M217S | 470.92 | 424.03 | 1.11 | 0.07 | 0.06 |
| M217L | 443.33 | 403.49 | 1.10 | 0.07 | 0.06 |
| M217N | 462.12 | 424.21 | 1.09 | 0.07 | 0.06 |
| M217R | 454.58 | 442.20 | 1.03 | 0.07 | 0.06 |
| M217Q | 449.94 | 427.47 | 1.05 | 0.07 | 0.06 |
| M217K | 506.96 | 458.74 | 1.11 | 0.08 | 0.06 |
| M217G | 746.17 | 728.78 | 1.02 | 0.12 | 0.10 |
| M217A | 437.36 | 410.18 | 1.07 | 0.07 | 0.06 |
| M217H | 442.18 | 398.29 | 1.11 | 0.07 | 0.06 |
| M217I | 483.00 | 449.94 | 1.07 | 0.08 | 0.06 |
| M217D | 503.49 | 491.20 | 1.03 | 0.04 | 0.05 |
| Y218C | 511.72 | 486.65 | 1.05 | 0.08 | 0.07 |
| Y218F | 4555.92 | 6084.93 | 0.75 | 0.71 | 0.85 |
| Y218W | 8521.86 | 9311.36 | 0.92 | 1.32 | 1.30 |
| Y218L | 834.41 | 743.23 | 1.12 | 0.13 | 0.10 |
| Y218A | 1935.94 | 1652.76 | 1.17 | 0.30 | 0.23 |
| Y218P | 503.58 | 469.06 | 1.07 | 0.08 | 0.07 |
| Y218R | 508.52 | 465.38 | 1.09 | 0.08 | 0.07 |
| Y218N | 704.77 | 640.35 | 1.10 | 0.11 | 0.09 |
| Y218V | 527.03 | 480.30 | 1.10 | 0.08 | 0.07 |
| Y218Q | 513.25 | 468.66 | 1.10 | 0.08 | 0.07 |
| Y218I | 698.50 | 542.67 | 1.29 | 0.11 | 0.08 |
| Y218D | 835.29 | 885.84 | 0.94 | 0.13 | 0.12 |
| Y218S | 3702.49 | 3099.73 | 1.19 | 0.58 | 0.43 |
| Y218G | 504.65 | 460.32 | 1.10 | 0.08 | 0.06 |
| Y218E | 511.24 | 471.64 | 1.08 | 0.08 | 0.07 |
| P219L | 578.24 | 550.18 | 1.05 | 0.09 | 0.08 |
| P219C | 622.59 | 613.51 | 1.01 | 0.10 | 0.09 |
| P219V | 586.82 | 583.21 | 1.01 | 0.09 | 0.08 |
| P219D | 819.59 | 881.94 | 0.93 | 0.13 | 0.12 |
| P219F | 571.45 | 542.25 | 1.05 | 0.09 | 0.08 |
| P219A | 1749.52 | 1799.14 | 0.97 | 0.27 | 0.25 |
| P219T | 870.52 | 853.07 | 1.02 | 0.14 | 0.12 |
| P219E | 895.73 | 858.50 | 1.04 | 0.14 | 0.12 |
| P219Q | 601.64 | 557.23 | 1.08 | 0.09 | 0.08 |
| P219R | 580.05 | 533.83 | 1.09 | 0.09 | 0.07 |
| P219H | 595.81 | 592.49 | 1.01 | 0.09 | 0.08 |
| P219G | 625.62 | 619.20 | 1.01 | 0.10 | 0.09 |
| P219K | 647.47 | 633.20 | 1.02 | 0.10 | 0.09 |
| P219S | 1549.48 | 1669.93 | 0.93 | 0.24 | 0.23 |
| P219W | 929.41 | 912.72 | 1.02 | 0.14 | 0.13 |
| S220R | 7949.20 | 10460.71 | 0.76 | 1.23 | 1.46 |
| S220A | 9804.98 | 9347.41 | 1.05 | 1.52 | 1.31 |
| S220Q | 9804.83 | 9328.79 | 1.05 | 1.52 | 1.30 |
| S220T | 9371.43 | 9378.23 | 1.00 | 1.46 | 1.31 |
| S220L | 1688.62 | 2607.71 | 0.65 | 0.26 | 0.36 |
| S220K | 2607.58 | 3704.87 | 0.70 | 0.40 | 0.52 |
| S220G | 9916.14 | 9356.60 | 1.06 | 1.54 | 1.31 |
| S220H | 1496.17 | 1874.12 | 0.80 | 0.23 | 0.26 |
| S220E | 3553.14 | 3992.18 | 0.89 | 0.55 | 0.56 |
| S220M | 7913.94 | 8545.54 | 0.93 | 1.23 | 1.20 |
| S220V | 10179.81 | 9414.03 | 1.08 | 1.58 | 1.32 |
| S220P | 592.32 | 587.51 | 1.01 | 0.09 | 0.08 |
| S220I | 6596.23 | 8678.69 | 0.76 | 1.02 | 1.21 |
| S220F | 2458.37 | 3612.28 | 0.68 | 0.38 | 0.51 |
| S220N | 10548.94 | 9399.76 | 1.12 | 1.64 | 1.31 |
| Y221W | 1201.23 | 891.46 | 1.35 | 0.19 | 0.12 |
| Y221K | 595.72 | 575.31 | 1.04 | 0.09 | 0.08 |
| Y221Q | 592.45 | 568.96 | 1.04 | 0.09 | 0.08 |
| Y221C | 583.88 | 558.96 | 1.04 | 0.09 | 0.08 |
| Y221N | 607.96 | 599.09 | 1.01 | 0.09 | 0.08 |
| Y221P | 575.23 | 546.02 | 1.05 | 0.09 | 0.08 |
| Y221V | 600.84 | 608.45 | 0.99 | 0.09 | 0.09 |
| Y221A | 613.20 | 571.57 | 1.07 | 0.10 | 0.08 |
| Y221G | 558.30 | 544.78 | 1.02 | 0.09 | 0.08 |
| Y221R | 508.18 | 483.59 | 1.05 | 0.08 | 0.07 |
| Y221S | 551.66 | 511.82 | 1.08 | 0.09 | 0.07 |
| Y221M | 733.99 | 576.28 | 1.27 | 0.11 | 0.08 |
| Y221T | 552.92 | 554.37 | 1.00 | 0.09 | 0.08 |
| Y221L | 600.40 | 544.47 | 1.10 | 0.09 | 0.08 |
| Y221E | 585.19 | 609.28 | 0.96 | 0.09 | 0.09 |
| T222L | 1251.44 | 1749.83 | 0.72 | 0.21 | 0.25 |
| T222Y | 3088.86 | 4344.09 | 0.71 | 0.52 | 0.61 |
| T222R | 7857.83 | 8130.34 | 0.97 | 1.33 | 1.14 |
| T222V | 6050.08 | 7520.37 | 0.80 | 1.03 | 1.06 |
| T222P | 9566.57 | 8477.71 | 1.13 | 1.62 | 1.19 |
| T222S | 8669.64 | 8464.76 | 1.02 | 1.47 | 1.19 |
| T222A | 5927.34 | 6623.26 | 0.89 | 1.00 | 0.93 |
| T222H | 4207.02 | 5413.49 | 0.78 | 0.71 | 0.76 |
| T222G | 7265.81 | 7630.73 | 0.95 | 1.23 | 1.07 |
| T222M | 4765.98 | 5354.04 | 0.89 | 0.81 | 0.75 |
| T222F | 8084.32 | 8023.96 | 1.01 | 1.37 | 1.13 |
| T222C | 1134.10 | 1047.66 | 1.08 | 0.19 | 0.15 |
| T222I | 489.93 | 514.09 | 0.95 | 0.08 | 0.07 |
| T222N | 8082.92 | 8215.81 | 0.98 | 1.37 | 1.15 |
| T222W | 4390.84 | 5903.99 | 0.74 | 0.74 | 0.83 |
| T222D | 4859.28 | 5584.93 | 0.87 | 0.53 | 0.73 |
| F223L | 2776.43 | 3305.29 | 0.84 | 0.47 | 0.46 |
| F223T | 7801.97 | 7792.62 | 1.00 | 1.32 | 1.09 |
| F223C | 3115.11 | 2488.91 | 1.25 | 0.53 | 0.35 |
| F223R | 5508.50 | 5094.99 | 1.08 | 0.93 | 0.71 |
| F223N | 7434.46 | 6650.94 | 1.12 | 1.26 | 0.93 |
| F223P | 8466.83 | 7678.71 | 1.10 | 1.43 | 1.08 |
| F223E | 7194.34 | 5884.03 | 1.22 | 1.22 | 0.83 |
| F223G | 3236.56 | 2599.04 | 1.25 | 0.55 | 0.36 |
| F223Q | 8100.68 | 7468.16 | 1.08 | 1.37 | 1.05 |
| F223A | 5226.86 | 3982.92 | 1.31 | 0.89 | 0.56 |
| F223S | 6006.80 | 4916.07 | 1.22 | 1.02 | 0.69 |
| F223Y | 9072.25 | 8479.33 | 1.07 | 1.54 | 1.19 |
| F223H | 8573.59 | 8056.97 | 1.06 | 1.45 | 1.13 |
| F223K | 4021.97 | 3712.91 | 1.08 | 0.60 | 0.49 |
| F223M | 525.66 | 441.29 | 1.19 | 0.08 | 0.06 |
| S224G | 5580.59 | 6030.81 | 0.93 | 0.89 | 0.97 |
| S224T | 6189.79 | 7398.93 | 0.84 | 0.99 | 1.18 |
| S224Q | 7258.89 | 8221.79 | 0.88 | 1.16 | 1.32 |
| S224R | 4718.67 | 4984.94 | 0.95 | 0.76 | 0.80 |
| S224P | 475.19 | 459.57 | 1.03 | 0.08 | 0.07 |
| S224I | 5653.45 | 6319.33 | 0.89 | 0.90 | 1.01 |
| S224V | 4074.45 | 5042.87 | 0.81 | 0.65 | 0.81 |
| S224L | 4272.54 | 5590.35 | 0.76 | 0.68 | 0.89 |
| S224C | 4057.16 | 4912.59 | 0.83 | 0.65 | 0.79 |
| S224K | 7286.24 | 8122.32 | 0.90 | 1.17 | 1.30 |
| S224D | 7201.97 | 8490.41 | 0.85 | 1.15 | 1.36 |
| S224H | 5928.85 | 6787.33 | 0.87 | 0.95 | 1.09 |
| S224M | 5967.51 | 6770.07 | 0.88 | 0.95 | 1.08 |
| S224A | 469.39 | 427.21 | 1.10 | 0.08 | 0.07 |
| S224W | 4323.69 | 5971.55 | 0.72 | 0.69 | 0.96 |
| G225D | 4925.13 | 4615.89 | 1.07 | 0.83 | 0.65 |
| G225R | 6317.32 | 6775.84 | 0.93 | 1.07 | 0.95 |
| G225Q | 8693.50 | 8267.06 | 1.05 | 1.47 | 1.16 |
| G225M | 3626.70 | 3585.88 | 1.01 | 0.61 | 0.50 |
| G225P | 4775.00 | 4558.87 | 1.05 | 0.81 | 0.64 |
| G225W | 6452.91 | 7515.31 | 0.86 | 1.09 | 1.05 |
| G225S | 4811.54 | 4789.30 | 1.00 | 0.82 | 0.67 |
| G225E | 9174.21 | 8356.85 | 1.10 | 1.55 | 1.17 |
| G225V | 3525.03 | 3330.02 | 1.06 | 0.60 | 0.47 |
| G225T | 7463.15 | 7841.71 | 0.95 | 1.26 | 1.10 |
| G225K | 7135.01 | 7721.15 | 0.92 | 1.21 | 1.08 |
| G225N | 5858.96 | 5807.35 | 1.01 | 0.99 | 0.81 |
| G225C | 1631.86 | 1835.77 | 0.89 | 0.28 | 0.26 |
| G225H | 8719.92 | 8448.61 | 1.03 | 1.48 | 1.19 |
| G225A | 6048.29 | 5768.91 | 1.05 | 1.03 | 0.81 |
| D226S | 8608.26 | 8605.77 | 1.00 | 1.46 | 1.21 |
| D226W | 1817.34 | 2172.37 | 0.84 | 0.31 | 0.30 |
| D226R | 5584.63 | 7070.57 | 0.79 | 0.95 | 0.99 |
| D226A | 6987.67 | 7786.46 | 0.90 | 1.18 | 1.09 |
| D226N | 6464.01 | 7314.30 | 0.88 | 1.10 | 1.03 |
| D226T | 3450.45 | 4219.45 | 0.82 | 0.58 | 0.59 |
| D226E | 9308.62 | 8744.05 | 1.06 | 1.58 | 1.23 |
| D226L | 3411.80 | 4254.22 | 0.80 | 0.58 | 0.60 |
| D226P | 8574.77 | 8325.60 | 1.03 | 1.45 | 1.17 |
| D226H | 4217.71 | 4180.90 | 1.01 | 0.71 | 0.59 |
| D226G | 6320.31 | 7359.33 | 0.86 | 1.07 | 1.03 |
| D226I | 7753.45 | 8016.92 | 0.97 | 1.31 | 1.12 |
| D226M | 6501.53 | 7210.62 | 0.90 | 1.10 | 1.01 |
| D226V | 3680.55 | 4504.86 | 0.82 | 0.62 | 0.63 |
| D226C | 7227.15 | 7735.09 | 0.93 | 1.22 | 1.09 |
| V227A | 5109.18 | 5056.19 | 1.01 | 0.87 | 0.71 |
| V227C | 4040.96 | 3278.65 | 1.23 | 0.68 | 0.46 |
| V227D | 1190.09 | 731.34 | 1.63 | 0.20 | 0.10 |
| V227E | 5381.63 | 2605.20 | 2.07 | 0.91 | 0.37 |
| V227K | 580.24 | 550.24 | 1.05 | 0.10 | 0.08 |
| V227L | 4883.98 | 4000.68 | 1.22 | 0.83 | 0.56 |
| V227P | 3682.22 | 3644.94 | 1.01 | 0.62 | 0.51 |
| V227S | 3863.33 | 3131.47 | 1.23 | 0.65 | 0.44 |
| V227T | 9817.63 | 8523.33 | 1.15 | 1.66 | 1.20 |
| V227W | 1845.46 | 1374.06 | 1.34 | 0.31 | 0.19 |
| V227Y | 657.68 | 542.07 | 1.21 | 0.11 | 0.08 |
| V227G | 1040.74 | 883.01 | 1.18 | 0.15 | 0.12 |
| V227H | 689.20 | 504.65 | 1.37 | 0.10 | 0.07 |
| V227Q | 696.97 | 506.11 | 1.38 | 0.10 | 0.07 |
| V227R | 664.31 | 561.06 | 1.18 | 0.10 | 0.07 |
| Q228A | 9710.68 | 9175.62 | 1.06 | 4.50 | 3.13 |
| Q228D | 10931.89 | 9274.00 | 1.18 | 5.06 | 3.16 |
| Q228E | 9825.63 | 9396.31 | 1.05 | 4.55 | 3.20 |
| Q228G | 9400.23 | 9058.34 | 1.04 | 4.35 | 3.09 |
| Q228H | 9748.08 | 9288.74 | 1.05 | 4.51 | 3.17 |
| Q228K | 9999.23 | 9262.11 | 1.08 | 4.63 | 3.16 |
| Q228L | 9199.01 | 8900.02 | 1.03 | 4.26 | 3.03 |
| Q228M | 9510.07 | 8915.79 | 1.07 | 4.40 | 3.04 |
| Q228N | 8774.26 | 8679.09 | 1.01 | 4.06 | 2.96 |
| Q228P | 2862.74 | 1291.55 | 2.22 | 1.33 | 0.44 |
| Q228R | 7443.02 | 8091.71 | 0.92 | 3.45 | 2.76 |
| Q228S | 8188.30 | 8162.27 | 1.00 | 3.79 | 2.78 |
| Q228T | 4335.26 | 5179.47 | 0.84 | 2.01 | 1.77 |
| Q228W | 6169.39 | 6508.89 | 0.95 | 2.86 | 2.22 |
| Q228Y | 7426.87 | 7840.08 | 0.95 | 3.44 | 2.67 |
| L229R | 485.95 | 489.74 | 0.99 | 0.22 | 0.17 |
| L229A | 2627.78 | 2118.07 | 1.24 | 1.22 | 0.72 |
| L229T | 3780.54 | 1464.25 | 2.58 | 1.75 | 0.50 |
| L229Q | 5328.09 | 5303.89 | 1.00 | 2.47 | 1.81 |
| L229P | 4795.14 | 5009.73 | 0.96 | 2.22 | 1.71 |
| L229E | 737.30 | 657.34 | 1.12 | 0.34 | 0.22 |
| L229W | 577.28 | 520.84 | 1.11 | 0.27 | 0.18 |
| L229M | 3207.73 | 2829.20 | 1.13 | 1.49 | 0.96 |
| L229I | 1158.56 | 828.94 | 1.40 | 0.54 | 0.28 |
| L229G | 552.22 | 520.30 | 1.06 | 0.26 | 0.18 |
| L229C | 633.99 | 516.61 | 1.23 | 0.29 | 0.18 |
| L229Y | 549.90 | 504.60 | 1.09 | 0.25 | 0.17 |
| L229D | 498.06 | 485.50 | 1.03 | 0.23 | 0.17 |
| L229H | 551.54 | 501.77 | 1.10 | 0.26 | 0.17 |
| L229V | 6249.58 | 6487.57 | 0.96 | 2.89 | 2.21 |
| A230L | 3437.91 | 2154.62 | 1.60 | 1.59 | 0.73 |
| A230G | 6804.87 | 8304.63 | 0.82 | 3.15 | 2.83 |
| A230W | 4773.24 | 5118.69 | 0.93 | 2.21 | 1.74 |
| A230P | 699.78 | 568.62 | 1.23 | 0.32 | 0.19 |
| A230D | 7281.83 | 8033.59 | 0.91 | 3.37 | 2.74 |
| A230R | 2986.52 | 2304.10 | 1.30 | 1.38 | 0.79 |
| A230I | 4609.64 | 3490.44 | 1.32 | 2.13 | 1.19 |
| A230S | 9181.71 | 8982.15 | 1.02 | 4.25 | 3.06 |
| A230C | 5061.18 | 5781.86 | 0.88 | 2.34 | 1.97 |
| A230V | 5030.94 | 3433.18 | 1.47 | 2.33 | 1.17 |
| A230T | 8822.74 | 9169.52 | 0.96 | 4.08 | 3.13 |
| A230Y | 3327.47 | 2858.53 | 1.16 | 1.54 | 0.97 |
| A230M | 9543.01 | 9520.33 | 1.00 | 4.42 | 3.25 |
| A230N | 9217.40 | 9384.02 | 0.98 | 4.27 | 3.20 |
| A230H | 8514.47 | 6763.46 | 1.26 | 3.94 | 2.31 |
| Q231I | 4161.47 | 3993.24 | 1.04 | 1.93 | 1.36 |
| Q231A | 6899.50 | 6650.48 | 1.04 | 3.19 | 2.27 |
| Q231F | 4049.29 | 4120.56 | 0.98 | 1.87 | 1.40 |
| Q231P | 613.73 | 591.34 | 1.04 | 0.28 | 0.20 |
| Q231Y | 2460.04 | 2960.32 | 0.83 | 1.14 | 1.01 |
| Q231R | 366.16 | 1013.08 | 0.36 | 0.17 | 0.35 |
| Q231L | 3744.09 | 2834.26 | 1.32 | 1.73 | 0.97 |
| Q231D | 7507.92 | 7957.26 | 0.94 | 3.48 | 2.71 |
| Q231G | 5743.70 | 6012.88 | 0.96 | 2.66 | 2.05 |
| Q231V | 6114.28 | 6172.88 | 0.99 | 2.83 | 2.10 |
| Q231W | 4910.80 | 4767.26 | 1.03 | 2.27 | 1.62 |
| Q231S | 6593.10 | 7180.32 | 0.92 | 3.05 | 2.45 |
| Q231H | 4961.12 | 5622.05 | 0.88 | 2.30 | 1.92 |
| Q231C | 970.74 | 697.37 | 1.39 | 0.45 | 0.24 |
| Q231M | 3314.86 | 4166.20 | 0.80 | 1.53 | 1.42 |
| D232H | 6046.51 | 7174.55 | 0.84 | 2.80 | 2.45 |
| D232G | 5492.29 | 7079.04 | 0.78 | 2.54 | 2.41 |
| D232R | 5077.01 | 5692.86 | 0.89 | 2.35 | 1.94 |
| D232P | 7665.95 | 8291.54 | 0.92 | 3.55 | 2.83 |
| D232Y | 3001.62 | 3628.15 | 0.83 | 1.39 | 1.24 |
| D232N | 825.42 | 739.97 | 1.12 | 0.38 | 0.25 |
| D232S | 14389.74 | 5104.26 | 2.82 | 6.66 | 1.74 |
| D232F | 3599.26 | 3719.64 | 0.97 | 1.67 | 1.27 |
| D232V | 7938.31 | 9176.75 | 0.87 | 3.68 | 3.13 |
| D232K | 4844.31 | 5829.58 | 0.83 | 2.24 | 1.99 |
| D232W | 8404.13 | 9037.60 | 0.93 | 3.89 | 3.08 |
| D232Q | 7550.58 | 8008.46 | 0.94 | 3.50 | 2.73 |
| D232E | 9294.39 | 9251.91 | 1.00 | 4.30 | 3.15 |
| D232T | 9434.20 | 9583.53 | 0.98 | 4.37 | 3.27 |
| D232L | 7603.68 | 4213.70 | 1.80 | 3.52 | 1.44 |
| D233Q | 653.34 | 640.95 | 1.02 | 0.30 | 0.22 |
| D233P | 629.51 | 626.42 | 1.00 | 0.29 | 0.21 |
| D233S | 637.89 | 623.07 | 1.02 | 0.30 | 0.21 |
| D233T | 621.24 | 615.06 | 1.01 | 0.29 | 0.21 |
| D233A | 650.58 | 634.46 | 1.03 | 0.30 | 0.22 |
| D233W | 644.19 | 649.94 | 0.99 | 0.30 | 0.22 |
| D233G | 657.96 | 666.27 | 0.99 | 0.30 | 0.23 |
| D233R | 715.14 | 467.03 | 1.53 | 0.33 | 0.16 |
| D233E | 2881.17 | 1918.57 | 1.50 | 1.33 | 0.65 |
| D233N | 580.50 | 572.32 | 1.01 | 0.27 | 0.20 |
| D233V | 609.36 | 603.42 | 1.01 | 0.28 | 0.21 |
| D233M | 581.45 | 593.79 | 0.98 | 0.27 | 0.20 |
| D233L | 584.42 | 597.47 | 0.98 | 0.27 | 0.20 |
| D233K | 608.53 | 615.71 | 0.99 | 0.28 | 0.21 |
| D233I | 682.66 | 661.78 | 1.03 | 0.32 | 0.23 |
| I234A | 1458.10 | 1018.50 | 1.43 | 0.31 | 0.18 |
| I234T | 1451.51 | 1188.67 | 1.22 | 0.31 | 0.21 |
| I234V | 3474.82 | 5245.94 | 0.66 | 0.73 | 0.91 |
| I234W | 743.35 | 570.02 | 1.30 | 0.16 | 0.10 |
| I234E | 1301.06 | 840.09 | 1.55 | 0.27 | 0.15 |
| I234G | 498.38 | 467.78 | 1.07 | 0.10 | 0.08 |
| I234L | 2584.47 | 3312.61 | 0.78 | 0.54 | 0.57 |
| I234H | 684.95 | 503.63 | 1.36 | 0.14 | 0.09 |
| I234M | 3478.87 | 4732.62 | 0.74 | 0.73 | 0.82 |
| I234N | 633.30 | 513.69 | 1.23 | 0.13 | 0.09 |
| I234Y | 749.28 | 930.94 | 0.80 | 0.16 | 0.16 |
| I234P | 470.41 | 431.33 | 1.09 | 0.10 | 0.07 |
| I234D | 428.28 | 397.06 | 1.08 | 0.09 | 0.07 |
| I234Q | 1095.18 | 837.53 | 1.31 | 0.23 | 0.15 |
| I234C | 702.09 | 483.22 | 1.45 | 0.15 | 0.08 |
| D235H | 5217.44 | 6443.75 | 0.81 | 1.10 | 1.12 |
| D235G | 5966.03 | 6875.44 | 0.87 | 1.26 | 1.19 |
| D235A | 5874.20 | 9191.04 | 0.64 | 1.24 | 1.59 |
| D235P | 488.90 | 464.91 | 1.05 | 0.10 | 0.08 |
| D235L | 6353.97 | 6868.10 | 0.93 | 1.34 | 1.19 |
| D235V | 4167.59 | 6418.00 | 0.65 | 0.88 | 1.11 |
| D235E | 8377.14 | 8154.10 | 1.03 | 1.76 | 1.41 |
| D235R | 7249.34 | 7013.16 | 1.03 | 1.53 | 1.22 |
| D235Q | 6969.55 | 7752.26 | 0.90 | 1.47 | 1.34 |
| D235T | 6608.45 | 7282.18 | 0.91 | 1.39 | 1.26 |
| D235C | 3805.25 | 5237.38 | 0.73 | 0.80 | 0.91 |
| D235S | 3798.36 | 6310.00 | 0.60 | 0.80 | 1.09 |
| D235N | 6427.98 | 6780.43 | 0.95 | 1.35 | 1.18 |
| D235Y | 3539.06 | 3728.70 | 0.95 | 0.75 | 0.65 |
| D235I | 5390.78 | 5499.20 | 0.98 | 1.14 | 0.95 |
| G236M | 8157.95 | 7452.06 | 1.09 | 1.72 | 1.29 |
| G236R | 8890.85 | 8115.23 | 1.10 | 1.87 | 1.41 |
| G236D | 3820.99 | 4576.94 | 0.83 | 0.80 | 0.79 |
| G236S | 9887.69 | 8558.48 | 1.16 | 2.08 | 1.48 |
| G236T | 6244.13 | 7949.24 | 0.79 | 1.32 | 1.38 |
| G236C | 8441.80 | 7993.25 | 1.06 | 1.78 | 1.39 |
| G236K | 9473.18 | 8370.29 | 1.13 | 2.00 | 1.45 |
| G236E | 7240.97 | 7573.43 | 0.96 | 1.53 | 1.31 |
| G236P | 969.12 | 668.23 | 1.45 | 0.20 | 0.12 |
| G236I | 5356.96 | 5189.19 | 1.03 | 1.13 | 0.90 |
| G236Y | 4511.53 | 5725.61 | 0.79 | 0.95 | 0.99 |
| G236L | 8099.87 | 7699.72 | 1.05 | 1.71 | 1.34 |
| G236V | 4448.72 | 6422.58 | 0.69 | 0.94 | 1.11 |
| G236N | 8477.40 | 8088.17 | 1.05 | 1.79 | 1.40 |
| G236F | 5761.86 | 5560.83 | 1.04 | 1.21 | 0.96 |
| I237S | 587.31 | 536.19 | 1.10 | 0.12 | 0.09 |
| I237L | 2880.14 | 2240.61 | 1.29 | 0.61 | 0.39 |
| I237R | 572.70 | 548.66 | 1.04 | 0.12 | 0.10 |
| I237Q | 552.21 | 543.69 | 1.02 | 0.12 | 0.09 |
| I237K | 571.56 | 531.05 | 1.08 | 0.12 | 0.09 |
| I237D | 567.39 | 512.64 | 1.11 | 0.12 | 0.09 |
| I237A | 1512.93 | 2138.58 | 0.71 | 0.32 | 0.37 |
| I237T | 572.40 | 524.74 | 1.09 | 0.12 | 0.09 |
| I237E | 555.97 | 535.94 | 1.04 | 0.12 | 0.09 |
| I237C | 565.11 | 532.36 | 1.06 | 0.12 | 0.09 |
| I237G | 620.23 | 586.38 | 1.06 | 0.13 | 0.10 |
| I237P | 554.04 | 497.24 | 1.11 | 0.12 | 0.09 |
| I237Y | 688.92 | 602.65 | 1.14 | 0.15 | 0.10 |
| I237W | 4188.38 | 5663.94 | 0.74 | 0.62 | 0.75 |
| I237N | 5368.49 | 6271.59 | 0.86 | 0.80 | 0.83 |
| Q238G | 1382.45 | 2524.64 | 0.55 | 0.29 | 0.44 |
| Q238H | 3150.20 | 5045.01 | 0.62 | 0.66 | 0.88 |
| Q238S | 3298.60 | 4524.89 | 0.73 | 0.69 | 0.78 |
| Q238Y | 2078.90 | 2953.44 | 0.70 | 0.44 | 0.51 |
| Q238F | 1342.33 | 1916.87 | 0.70 | 0.28 | 0.33 |
| Q238E | 4075.95 | 5719.51 | 0.71 | 0.86 | 0.99 |
| Q238L | 3030.44 | 4771.52 | 0.64 | 0.64 | 0.83 |
| Q238W | 3649.81 | 4317.00 | 0.85 | 0.77 | 0.75 |
| Q238P | 568.68 | 548.50 | 1.04 | 0.12 | 0.10 |
| Q238R | 4199.78 | 5952.76 | 0.71 | 0.88 | 1.03 |
| Q238C | 3179.60 | 4072.46 | 0.78 | 0.67 | 0.71 |
| Q238N | 3119.13 | 3894.89 | 0.80 | 0.66 | 0.68 |
| Q238I | 3863.21 | 5191.15 | 0.74 | 0.81 | 0.90 |
| Q238T | 7425.48 | 7998.67 | 0.93 | 1.56 | 1.39 |
| Q238K | 4717.87 | 5952.01 | 0.79 | 0.99 | 1.03 |
| A239S | 7235.02 | 7630.43 | 0.95 | 1.52 | 1.32 |
| A239Q | 4817.25 | 8226.13 | 0.59 | 1.01 | 1.43 |
| A239T | 1232.13 | 2351.52 | 0.52 | 0.26 | 0.41 |
| A239P | 606.39 | 564.14 | 1.07 | 0.13 | 0.10 |
| A239V | 6075.94 | 6919.38 | 0.88 | 1.28 | 1.20 |
| A239L | 7174.28 | 7878.58 | 0.91 | 1.51 | 1.37 |
| A239Y | 5570.87 | 6668.58 | 0.84 | 1.17 | 1.16 |
| A239I | 6821.36 | 7628.60 | 0.89 | 1.44 | 1.32 |
| A239C | 4986.74 | 5916.10 | 0.84 | 1.05 | 1.03 |
| A239G | 6430.98 | 7617.20 | 0.84 | 1.35 | 1.32 |
| A239W | 2215.28 | 4554.04 | 0.49 | 0.47 | 0.79 |
| A239F | 719.92 | 750.33 | 0.96 | 0.15 | 0.13 |
| A239K | 8365.39 | 8161.53 | 1.02 | 1.76 | 1.42 |
| A239H | 6013.93 | 6892.53 | 0.87 | 1.27 | 1.20 |
| A239R | 8860.20 | 8322.26 | 1.06 | 1.87 | 1.44 |
| A239D | 9256.74 | 8197.40 | 1.13 | 1.02 | 1.08 |
| I240G | 550.59 | 455.20 | 1.21 | 0.09 | 0.06 |
| I240Q | 1050.40 | 921.68 | 1.14 | 0.16 | 0.12 |
| I240P | 2259.38 | 3251.71 | 0.69 | 0.35 | 0.42 |
| I240R | 2771.00 | 3465.26 | 0.80 | 0.43 | 0.44 |
| I240S | 2033.91 | 1204.66 | 1.69 | 0.32 | 0.15 |
| I240K | 5557.21 | 6183.54 | 0.90 | 0.87 | 0.79 |
| I240V | 4682.76 | 6307.59 | 0.74 | 0.73 | 0.81 |
| I240D | 480.83 | 456.39 | 1.05 | 0.08 | 0.06 |
| I240A | 2099.13 | 1776.41 | 1.18 | 0.33 | 0.23 |
| I240C | 970.78 | 650.04 | 1.49 | 0.15 | 0.08 |
| I240L | 8303.04 | 8506.66 | 0.98 | 1.30 | 1.09 |
| I240F | 1345.29 | 2090.14 | 0.64 | 0.21 | 0.27 |
| I240Y | 1910.61 | 1482.53 | 1.29 | 0.30 | 0.19 |
| I240M | 8056.10 | 7463.56 | 1.08 | 1.26 | 0.95 |
| I240T | 2147.14 | 1862.29 | 1.15 | 0.34 | 0.24 |
| Y241V | 568.18 | 567.15 | 1.00 | 0.09 | 0.07 |
| Y241A | 514.00 | 498.25 | 1.03 | 0.08 | 0.06 |
| Y241G | 484.83 | 493.78 | 0.98 | 0.08 | 0.06 |
| Y241H | 555.41 | 547.86 | 1.01 | 0.09 | 0.07 |
| Y241R | 479.35 | 491.61 | 0.98 | 0.08 | 0.06 |
| Y241P | 542.62 | 468.74 | 1.16 | 0.09 | 0.06 |
| Y241Q | 494.42 | 468.65 | 1.05 | 0.08 | 0.06 |
| Y241L | 486.82 | 484.95 | 1.00 | 0.08 | 0.06 |
| Y241T | 574.08 | 548.16 | 1.05 | 0.09 | 0.07 |
| Y241S | 512.62 | 498.70 | 1.03 | 0.08 | 0.06 |
| Y241W | 592.83 | 556.32 | 1.07 | 0.09 | 0.07 |
| Y241N | 438.44 | 443.51 | 0.99 | 0.07 | 0.06 |
| Y241M | 488.24 | 448.13 | 1.09 | 0.08 | 0.06 |
| Y241I | 469.78 | 446.31 | 1.05 | 0.07 | 0.06 |
| Y241D | 454.99 | 443.57 | 1.03 | 0.07 | 0.06 |
| G242A | 1948.43 | 1965.86 | 0.99 | 0.31 | 0.25 |
| G242F | 2367.13 | 2861.31 | 0.83 | 0.37 | 0.37 |
| G242L | 4261.36 | 6043.02 | 0.71 | 0.67 | 0.77 |
| G242N | 1712.51 | 1893.88 | 0.90 | 0.27 | 0.24 |
| G242P | 2683.19 | 3311.87 | 0.81 | 0.42 | 0.42 |
| G242W | 1200.76 | 1522.34 | 0.79 | 0.19 | 0.19 |
| G242T | 1845.15 | 1926.23 | 0.96 | 0.29 | 0.25 |
| G242R | 1425.79 | 1462.84 | 0.97 | 0.22 | 0.19 |
| G242V | 1864.42 | 2075.51 | 0.90 | 0.29 | 0.27 |
| G242S | 3463.26 | 4491.54 | 0.77 | 0.54 | 0.57 |
| G242I | 881.04 | 2441.87 | 0.36 | 0.14 | 0.31 |
| G242Y | 895.61 | 928.34 | 0.96 | 0.14 | 0.12 |
| G242H | 1038.60 | 1063.68 | 0.98 | 0.16 | 0.14 |
| G242E | 1039.40 | 1200.19 | 0.87 | 0.16 | 0.15 |
| G242K | 1259.85 | 1404.80 | 0.90 | 0.20 | 0.18 |
| R243P | 3936.04 | 7438.61 | 0.53 | 0.62 | 0.95 |
| R243K | 8397.44 | 8514.77 | 0.99 | 1.32 | 1.09 |
| R243T | 7451.28 | 7306.32 | 1.02 | 1.17 | 0.93 |
| R243L | 6953.44 | 7458.28 | 0.93 | 1.09 | 0.95 |
| R243A | 8253.02 | 8378.15 | 0.99 | 1.29 | 1.07 |
| R243H | 6757.06 | 7710.25 | 0.88 | 1.06 | 0.99 |
| R243Q | 7563.55 | 8367.33 | 0.90 | 1.19 | 1.07 |
| R243S | 7872.26 | 8367.98 | 0.94 | 1.23 | 1.07 |
| R243I | 4421.12 | 8337.68 | 0.53 | 0.69 | 1.07 |
| R243C | 6128.24 | 6907.63 | 0.89 | 0.96 | 0.88 |
| R243N | 7064.71 | 7808.36 | 0.90 | 1.11 | 1.00 |
| R243Y | 6415.10 | 7427.20 | 0.86 | 1.01 | 0.95 |
| R243G | 9279.36 | 8697.39 | 1.07 | 1.46 | 1.11 |
| R243D | 5769.78 | 6318.28 | 0.91 | 0.90 | 0.81 |
| R243V | 6349.88 | 7531.41 | 0.84 | 1.00 | 0.96 |
| S244P | 7394.47 | 7844.16 | 0.94 | 1.16 | 1.00 |
| S244L | 3480.19 | 7154.31 | 0.49 | 0.55 | 0.91 |
| S244W | 10346.96 | 9035.98 | 1.15 | 1.62 | 1.15 |
| S244M | 728.14 | 748.02 | 0.97 | 0.11 | 0.10 |
| S244V | 6842.04 | 7456.02 | 0.92 | 1.07 | 0.95 |
| S244Q | 9318.66 | 8746.49 | 1.07 | 1.46 | 1.12 |
| S244D | 6915.01 | 7609.44 | 0.91 | 1.08 | 0.97 |
| S244E | 8814.39 | 8156.16 | 1.08 | 1.38 | 1.04 |
| S244T | 7442.34 | 8205.48 | 0.91 | 1.17 | 1.05 |
| S244H | 5019.42 | 8528.78 | 0.59 | 0.79 | 1.09 |
| S244G | 888.96 | 801.21 | 1.11 | 0.14 | 0.10 |
| S244A | 9174.68 | 8474.43 | 1.08 | 1.44 | 1.08 |
| S244F | 962.91 | 1017.49 | 0.95 | 0.15 | 0.13 |
| S244Y | 6333.20 | 6595.86 | 0.96 | 0.99 | 0.84 |
| S244R | 10483.23 | 9488.64 | 1.10 | 0.93 | 0.99 |
| Q245P | 8046.62 | 8690.91 | 0.93 | 1.26 | 1.11 |
| Q245I | 7611.43 | 8270.47 | 0.92 | 1.19 | 1.06 |
| Q245F | 3940.03 | 8048.83 | 0.49 | 0.62 | 1.03 |
| Q245V | 7785.27 | 8186.90 | 0.95 | 1.22 | 1.05 |
| Q245M | 494.18 | 323.62 | 1.53 | 0.08 | 0.04 |
| Q245T | 8684.29 | 8676.53 | 1.00 | 1.36 | 1.11 |
| Q245E | 10044.47 | 8646.78 | 1.16 | 1.58 | 1.10 |
| Q245S | 8700.39 | 8695.56 | 1.00 | 1.36 | 1.11 |
| Q245R | 8323.06 | 8629.37 | 0.96 | 1.31 | 1.10 |
| Q245G | 8495.47 | 8561.87 | 0.99 | 1.33 | 1.09 |
| Q245H | 8236.63 | 8640.32 | 0.95 | 1.29 | 1.10 |
| Q245L | 6762.99 | 6774.37 | 1.00 | 1.06 | 0.87 |
| Q245K | 347.86 | 290.28 | 1.20 | 0.05 | 0.04 |
| Q245W | 7517.93 | 8157.63 | 0.92 | 1.18 | 1.04 |
| Q245C | 7377.19 | 7707.00 | 0.96 | 1.16 | 0.98 |
| N246W | 3998.57 | 5256.41 | 0.76 | 0.55 | 0.68 |
| N246R | 6324.43 | 7263.78 | 0.87 | 0.87 | 0.93 |
| N246A | 7162.60 | 7821.64 | 0.92 | 0.98 | 1.00 |
| N246F | 5961.78 | 6704.16 | 0.89 | 0.82 | 0.86 |
| N246G | 7132.52 | 7954.86 | 0.90 | 0.98 | 1.02 |
| N246P | 5753.82 | 6382.30 | 0.90 | 0.79 | 0.82 |
| N246V | 7113.82 | 7563.12 | 0.94 | 0.98 | 0.97 |
| N246Q | 8249.22 | 7962.93 | 1.04 | 1.13 | 1.02 |
| N246Y | 6460.04 | 7091.47 | 0.91 | 0.89 | 0.91 |
| N246C | 3668.07 | 4131.97 | 0.89 | 0.50 | 0.53 |
| N246I | 6761.84 | 6684.68 | 1.01 | 0.93 | 0.86 |
| N246L | 7470.87 | 7558.25 | 0.99 | 1.03 | 0.97 |
| N246S | 7681.59 | 7872.41 | 0.98 | 1.05 | 1.01 |
| N246T | 7476.51 | 7504.31 | 1.00 | 1.03 | 0.96 |
| N246K | 8008.77 | 7820.31 | 1.02 | 1.10 | 1.00 |
| N246D | 7062.38 | 7827.99 | 0.90 | 0.78 | 1.03 |
| P247A | 8242.00 | 7947.96 | 1.04 | 1.13 | 1.02 |
| P247D | 6640.01 | 7179.97 | 0.92 | 0.91 | 0.92 |
| P247E | 8181.45 | 7231.43 | 1.13 | 1.12 | 0.93 |
| P247F | 8964.42 | 7929.76 | 1.13 | 1.23 | 1.02 |
| P247G | 7256.65 | 7455.20 | 0.97 | 1.00 | 0.96 |
| P247H | 8093.84 | 7667.72 | 1.06 | 1.11 | 0.99 |
| P247I | 7375.24 | 7729.05 | 0.95 | 1.01 | 0.99 |
| P247K | 8454.74 | 7912.16 | 1.07 | 1.16 | 1.02 |
| P247L | 8316.29 | 8009.70 | 1.04 | 1.14 | 1.03 |
| P247N | 8142.72 | 8006.73 | 1.02 | 1.12 | 1.03 |
| P247Q | 8231.43 | 7739.72 | 1.06 | 1.13 | 0.99 |
| P247R | 7029.19 | 7443.10 | 0.94 | 0.96 | 0.96 |
| P247S | 8040.91 | 7895.89 | 1.02 | 1.10 | 1.01 |
| P247T | 7243.03 | 7527.94 | 0.96 | 0.99 | 0.97 |
| P247V | 7907.19 | 7717.20 | 1.02 | 1.08 | 0.99 |
| V248W | 6631.29 | 6916.87 | 0.96 | 0.91 | 0.89 |
| V248L | 8767.88 | 8252.54 | 1.06 | 1.20 | 1.06 |
| V248Q | 6709.44 | 6735.33 | 1.00 | 0.92 | 0.87 |
| V248M | 7437.73 | 7338.43 | 1.01 | 1.02 | 0.94 |
| V248Y | 6509.63 | 6927.69 | 0.94 | 0.89 | 0.89 |
| V248G | 6438.97 | 6744.48 | 0.95 | 0.88 | 0.87 |
| V248C | 3692.16 | 3816.99 | 0.97 | 0.51 | 0.49 |
| V248R | 7253.81 | 7153.37 | 1.01 | 1.00 | 0.92 |
| V248A | 8420.73 | 7739.03 | 1.09 | 1.16 | 0.99 |
| V248H | 8578.28 | 7867.88 | 1.09 | 1.18 | 1.01 |
| V248I | 8473.10 | 8209.35 | 1.03 | 1.16 | 1.05 |
| V248T | 8100.54 | 7751.03 | 1.05 | 1.11 | 1.00 |
| V248K | 7147.53 | 7653.87 | 0.93 | 0.98 | 0.98 |
| V248S | 6899.65 | 6729.67 | 1.03 | 0.95 | 0.86 |
| V248F | 6736.68 | 6651.01 | 1.01 | 0.92 | 0.85 |
| V248E | 9210.34 | 8235.44 | 1.12 | 1.01 | 1.08 |
| Q249T | 6909.47 | 7370.27 | 0.94 | 0.95 | 0.95 |
| Q249W | 10145.55 | 8691.89 | 1.17 | 1.39 | 1.12 |
| Q249R | 7102.73 | 7103.74 | 1.00 | 0.97 | 0.91 |
| Q249E | 3987.22 | 7583.88 | 0.53 | 0.55 | 0.97 |
| Q249A | 8992.77 | 8414.65 | 1.07 | 1.23 | 1.08 |
| Q249P | 8376.56 | 8108.11 | 1.03 | 1.15 | 1.04 |
| Q249C | 5978.93 | 5496.03 | 1.09 | 0.82 | 0.71 |
| Q249G | 7612.71 | 7662.25 | 0.99 | 1.04 | 0.98 |
| Q249N | 7180.54 | 7257.66 | 0.99 | 0.99 | 0.93 |
| Q249K | 7772.72 | 7296.54 | 1.07 | 1.07 | 0.94 |
| Q249I | 7262.56 | 7159.06 | 1.01 | 1.00 | 0.92 |
| Q249Y | 6047.16 | 6053.08 | 1.00 | 0.83 | 0.78 |
| Q249V | 8717.93 | 8059.04 | 1.08 | 1.20 | 1.04 |
| Q249L | 6532.65 | 6824.78 | 0.96 | 0.90 | 0.88 |
| Q249H | 8441.70 | 7557.69 | 1.12 | 1.16 | 0.97 |
| P250L | 9455.47 | 8580.12 | 1.10 | 1.30 | 1.10 |
| P250S | 7684.90 | 7513.77 | 1.02 | 1.05 | 0.97 |
| P250R | 7701.81 | 7566.23 | 1.02 | 1.06 | 0.97 |
| P250Y | 7886.68 | 7534.22 | 1.05 | 1.08 | 0.97 |
| P250M | 8416.52 | 8221.73 | 1.02 | 1.15 | 1.06 |
| P250F | 8150.35 | 7703.24 | 1.06 | 1.12 | 0.99 |
| P250A | 8963.20 | 8460.94 | 1.06 | 1.23 | 1.09 |
| P250K | 7830.18 | 7732.04 | 1.01 | 1.07 | 0.99 |
| P250G | 7623.88 | 7834.34 | 0.97 | 1.05 | 1.01 |
| P250N | 7600.44 | 7961.88 | 0.95 | 1.04 | 1.02 |
| P250T | 1147.37 | 1489.99 | 0.77 | 0.16 | 0.19 |
| P250W | 7431.76 | 7755.84 | 0.96 | 1.02 | 1.00 |
| P250D | 7767.77 | 7525.09 | 1.03 | 1.07 | 0.97 |
| P250V | 7355.32 | 7719.82 | 0.95 | 1.01 | 0.99 |
| P250Q | 7797.52 | 8203.80 | 0.95 | 1.07 | 1.05 |
| I251A | 4953.41 | 8984.79 | 0.55 | 0.48 | 0.96 |
| I251Q | 10910.92 | 9221.40 | 1.18 | 1.07 | 0.98 |
| I251G | 11041.83 | 9640.57 | 1.15 | 1.08 | 1.03 |
| I251L | 11028.53 | 9408.72 | 1.17 | 1.08 | 1.00 |
| I251K | 11050.61 | 9421.73 | 1.17 | 1.08 | 1.01 |
| I251R | 10950.25 | 9220.62 | 1.19 | 1.07 | 0.98 |
| I251E | 10262.05 | 9115.62 | 1.13 | 1.00 | 0.97 |
| I251D | 10582.82 | 9557.71 | 1.11 | 1.04 | 1.02 |
| I251T | 10884.22 | 9485.20 | 1.15 | 1.06 | 1.01 |
| I251C | 10348.04 | 9428.04 | 1.10 | 1.01 | 1.01 |
| I251Y | 10319.00 | 9450.22 | 1.09 | 1.01 | 1.01 |
| I251P | 10762.38 | 9410.57 | 1.14 | 1.05 | 1.00 |
| I251S | 8445.88 | 7160.96 | 1.18 | 1.07 | 0.96 |
| I251W | 7305.95 | 6974.26 | 1.05 | 0.92 | 0.93 |
| I251V | 8343.83 | 7350.61 | 1.14 | 0.91 | 0.98 |
| G252F | 7921.80 | 7529.24 | 1.05 | 1.09 | 0.97 |
| G252W | 6989.36 | 7313.18 | 0.96 | 0.96 | 0.94 |
| G252A | 8567.46 | 8300.90 | 1.03 | 1.18 | 1.07 |
| G252R | 7756.55 | 7447.08 | 1.04 | 1.06 | 0.96 |
| G252L | 8684.63 | 8094.21 | 1.07 | 1.19 | 1.04 |
| G252E | 7651.86 | 7211.52 | 1.06 | 1.05 | 0.93 |
| G252D | 7977.50 | 7049.47 | 1.13 | 1.09 | 0.91 |
| G252K | 9685.27 | 8502.04 | 1.14 | 1.33 | 1.09 |
| G252S | 7596.71 | 6986.94 | 1.09 | 1.04 | 0.90 |
| G252T | 7242.98 | 7147.95 | 1.01 | 0.99 | 0.92 |
| G252P | 8175.79 | 8226.12 | 0.99 | 1.12 | 1.06 |
| G252H | 8030.53 | 7802.24 | 1.03 | 1.10 | 1.00 |
| G252C | 5540.29 | 5421.44 | 1.02 | 0.76 | 0.70 |
| G252V | 7910.50 | 7997.71 | 0.99 | 1.09 | 1.03 |
| G252I | 7702.75 | 7964.05 | 0.97 | 1.06 | 1.02 |
| P253C | 7906.13 | 8213.73 | 0.96 | 0.99 | 1.04 |
| P253G | 9640.00 | 8446.66 | 1.14 | 1.20 | 1.07 |
| P253Q | 9482.36 | 8631.24 | 1.10 | 1.18 | 1.09 |
| P253I | 6906.18 | 7721.21 | 0.89 | 0.86 | 0.97 |
| P253L | 8851.11 | 8489.29 | 1.04 | 1.10 | 1.07 |
| P253R | 9020.78 | 8580.86 | 1.05 | 1.12 | 1.08 |
| P253A | 8697.23 | 8410.29 | 1.03 | 1.08 | 1.06 |
| P253E | 9074.45 | 8476.99 | 1.07 | 1.13 | 1.07 |
| P253Y | 7935.28 | 8171.53 | 0.97 | 0.99 | 1.03 |
| P253W | 6635.85 | 7293.26 | 0.91 | 0.83 | 0.92 |
| P253M | 6895.66 | 7648.23 | 0.90 | 0.86 | 0.96 |
| P253V | 7058.87 | 7756.04 | 0.91 | 0.88 | 0.98 |
| P253T | 6728.25 | 7541.00 | 0.89 | 0.84 | 0.95 |
| P253K | 6929.49 | 7400.65 | 0.94 | 0.86 | 0.93 |
| P253N | 7354.73 | 7533.05 | 0.98 | 0.92 | 0.95 |
| Q254R | 9454.92 | 8474.29 | 1.12 | 1.18 | 1.07 |
| Q254G | 3549.45 | 3806.63 | 0.93 | 0.44 | 0.48 |
| Q254W | 3389.45 | 3326.38 | 1.02 | 0.42 | 0.42 |
| Q254T | 7491.28 | 7853.86 | 0.95 | 0.93 | 0.99 |
| Q254A | 7226.25 | 7451.70 | 0.97 | 0.90 | 0.94 |
| Q254F | 6263.95 | 6007.53 | 1.04 | 0.78 | 0.76 |
| Q254D | 9098.08 | 8154.92 | 1.12 | 1.13 | 1.03 |
| Q254P | 6827.99 | 7340.40 | 0.93 | 0.85 | 0.93 |
| Q254L | 7602.15 | 7940.64 | 0.96 | 0.95 | 1.00 |
| Q254C | 9284.18 | 8479.77 | 1.09 | 1.16 | 1.07 |
| Q254Y | 8847.02 | 7831.28 | 1.13 | 1.10 | 0.99 |
| Q254I | 9340.36 | 8662.75 | 1.08 | 1.16 | 1.09 |
| Q254E | 9466.76 | 8516.08 | 1.11 | 1.18 | 1.07 |
| Q254V | 9803.92 | 8575.31 | 1.14 | 1.22 | 1.08 |
| Q254S | 7768.13 | 8801.19 | 0.88 | 1.15 | 1.17 |
| T255I | 9880.58 | 8415.65 | 1.17 | 1.23 | 1.06 |
| T255Q | 9537.20 | 8410.86 | 1.13 | 1.19 | 1.06 |
| T255P | 7468.08 | 7296.37 | 1.02 | 0.93 | 0.92 |
| T255R | 5740.42 | 4974.50 | 1.15 | 0.72 | 0.63 |
| T255C | 2626.79 | 2503.21 | 1.05 | 0.33 | 0.32 |
| T255N | 5128.08 | 4479.75 | 1.14 | 0.64 | 0.57 |
| T255S | 7334.60 | 6905.71 | 1.06 | 0.91 | 0.87 |
| T255V | 5463.42 | 5187.78 | 1.05 | 0.68 | 0.65 |
| T255E | 7691.31 | 7194.23 | 1.07 | 0.96 | 0.91 |
| T255G | 8166.77 | 7682.14 | 1.06 | 1.02 | 0.97 |
| T255K | 6636.15 | 5647.18 | 1.18 | 0.83 | 0.71 |
| T255A | 4436.98 | 4322.98 | 1.03 | 0.55 | 0.55 |
| T255F | 3562.89 | 3107.64 | 1.15 | 0.44 | 0.39 |
| T255L | 4904.06 | 4266.71 | 1.15 | 0.61 | 0.54 |
| T255H | 8243.01 | 7352.60 | 1.12 | 1.22 | 0.98 |
| P256S | 10876.81 | 9018.60 | 1.21 | 1.36 | 1.14 |
| P256V | 10408.68 | 8594.11 | 1.21 | 1.30 | 1.08 |
| P256F | 6020.49 | 5181.94 | 1.16 | 0.75 | 0.65 |
| P256Y | 10270.90 | 8699.77 | 1.18 | 1.28 | 1.10 |
| P256I | 9089.54 | 7980.23 | 1.14 | 1.13 | 1.01 |
| P256A | 9426.67 | 8868.67 | 1.06 | 1.18 | 1.12 |
| P256L | 8342.08 | 7217.69 | 1.16 | 1.04 | 0.91 |
| P256G | 4631.84 | 4679.24 | 0.99 | 0.58 | 0.59 |
| P256N | 4406.75 | 3946.90 | 1.12 | 0.55 | 0.50 |
| P256R | 4975.17 | 4155.27 | 1.20 | 0.62 | 0.52 |
| P256Q | 6177.77 | 5546.92 | 1.11 | 0.77 | 0.70 |
| P256E | 9266.75 | 8366.07 | 1.11 | 1.16 | 1.06 |
| P256K | 5919.72 | 5928.31 | 1.00 | 0.74 | 0.75 |
| P256M | 8787.02 | 8554.52 | 1.03 | 1.10 | 1.08 |
| P256C | 4674.45 | 4633.67 | 1.01 | 0.69 | 0.62 |
| K257C | 4327.83 | 4267.45 | 1.01 | 0.54 | 0.54 |
| K257M | 5985.85 | 5236.20 | 1.14 | 0.75 | 0.66 |
| K257V | 7316.42 | 7115.65 | 1.03 | 0.91 | 0.90 |
| K257A | 9355.23 | 8528.52 | 1.10 | 1.17 | 1.08 |
| K257E | 10237.19 | 9141.73 | 1.12 | 1.28 | 1.15 |
| K257S | 9952.97 | 8464.79 | 1.18 | 1.24 | 1.07 |
| K257L | 10053.73 | 8711.61 | 1.15 | 1.25 | 1.10 |
| K257I | 8609.80 | 7806.76 | 1.10 | 1.07 | 0.98 |
| K257G | 8280.79 | 7718.36 | 1.07 | 1.03 | 0.97 |
| K257N | 8528.22 | 7707.49 | 1.11 | 1.06 | 0.97 |
| K257F | 7720.51 | 6633.90 | 1.16 | 0.96 | 0.84 |
| K257W | 7039.69 | 7120.56 | 0.99 | 0.88 | 0.90 |
| K257R | 9688.77 | 9114.18 | 1.06 | 1.21 | 1.15 |
| K257P | 8039.60 | 7464.88 | 1.08 | 1.19 | 0.99 |
| K257T | 9346.88 | 8849.42 | 1.06 | 1.39 | 1.18 |
| A258Q | 7000.31 | 6977.33 | 1.00 | 0.87 | 0.88 |
| A258Y | 6636.02 | 5998.83 | 1.11 | 0.83 | 0.76 |
| A258W | 9438.05 | 8527.86 | 1.11 | 1.18 | 1.08 |
| A258G | 7204.05 | 7778.97 | 0.93 | 0.90 | 0.98 |
| A258L | 1222.62 | 1226.40 | 1.00 | 0.15 | 0.15 |
| A258F | 9548.91 | 8531.04 | 1.12 | 1.19 | 1.08 |
| A258M | 8161.79 | 8061.20 | 1.01 | 1.02 | 1.02 |
| A258N | 7808.83 | 6968.56 | 1.12 | 0.97 | 0.88 |
| A258V | 8395.80 | 8391.43 | 1.00 | 1.05 | 1.06 |
| A258T | 8674.71 | 7958.00 | 1.09 | 1.08 | 1.00 |
| A258I | 8452.43 | 7509.34 | 1.13 | 1.05 | 0.95 |
| A258D | 7741.51 | 6346.88 | 1.22 | 0.97 | 0.80 |
| A258R | 9008.56 | 7908.51 | 1.14 | 1.12 | 1.00 |
| A258E | 10198.40 | 8709.16 | 1.17 | 1.27 | 1.10 |
| A258P | 10414.06 | 9178.82 | 1.13 | 1.55 | 1.22 |

From the initial screen, twenty-six mutants were selected as primary candidates for further evaluation based on decreased catalytic activity at 37° C. relative to 25° C. and/or sufficient protein expression or activity (Table 11). In Table 11, the positions indicated are with respect to positions corresponding to amino acid residues of hMMP-1 set forth in SEQ ID NO:2.

TABLE 11
Selected Positions for Generating Combinatorial Library

| | | | |
|---|---|---|---|
| L95K | D105I | D105N | D105L |
| D105A | D105G | R150P | D156R |
| D156H | D156K | D156T | G159V |
| G159T | D179N | E180T | E180F |
| E182T | T185Q | N187I | A198L |
| S208K | I213G | G214E | V227E |
| I234E | I240S | | |

C. Combinatorial MMP-1 Library

A combinatorial hMMP-1 variant library of double mutants containing two mutations from among L95K, D105N, R150P, D156K, D156T, G159V, D179N, E180T, A198L, V27E and 1240S, with reference to positions set forth in SEQ ID NO:2, was generated as described in U.S. Published Application No. 2010/0284995. The library was generated to theoretically contain every possible combination of amino acid variants for each of the selected mutants. The constructed library (designated CPS library) contained a total of 1238 mutants, including the wild-type and 9 single amino acid hits, which was 81% of the maximal diversity. The generated library of combinatorial mutants was screened as described in subsection B above. Identified candidates based on decreased catalytic activity at 37° C. relative to 25° C. and/or sufficient protein expression or activity are set forth in Table 12. All of the mutants tested exhibited less activity than wild-type at the corresponding temperature, although many exhibited substantial activity.

TABLE 12
Activity of Selected Mutants from Combinatorial Library

| | | | | 25° C.: | 37° C.: |
|---|---|---|---|---|---|
| | | | Ratio | % Act. | % Act. |
| | Avg. RFU | Avg. RFU | (25° C./ | of wt | of wt |
| Variant | 25° C. | 37° C. | 37° C.) | 25° C. | 25° C. |
| D156K/G159V/ | 1261.31 | 786.28 | 1.60 | 4.73 | 2.95 |
| D179N | | | | | |
| R150P/V227E | 1801.03 | 859.01 | 2.10 | 6.44 | 3.07 |
| D156T/V227E | 2021.29 | 864.71 | 2.34 | 7.22 | 3.09 |
| G159V/A198L | 1684.53 | 863.78 | 1.95 | 6.06 | 3.11 |
| D105N/A198L | 1422.45 | 919.80 | 1.55 | 5.34 | 3.45 |
| L95K | 1389.81 | 969.67 | 1.43 | 5.00 | 3.49 |
| D179N/V227E | 1446.86 | 948.41 | 1.53 | 5.43 | 3.56 |
| A198L/V227E | 2740.04 | 1036.69 | 2.64 | 9.79 | 3.70 |
| E180T/V227E | 2549.76 | 1038.44 | 2.46 | 9.11 | 3.71 |
| D179N/A198L | 1411.89 | 968.14 | 1.46 | 5.45 | 3.74 |
| D156K/D179N | 1227.63 | 973.51 | 1.26 | 4.74 | 3.76 |
| D105N/R150P/ | 1668.82 | 1002.65 | 1.66 | 6.26 | 3.76 |
| D156K/G159V/ | | | | | |
| D179N/E180T | | | | | |
| D105N/R150P/ | 1846.75 | 1003.36 | 1.84 | 6.93 | 3.76 |
| E180T | | | | | |
| G159V/I240S | 2565.48 | 1031.27 | 2.49 | 9.45 | 3.80 |
| D156T/D179N/ | 1326.33 | 774.68 | 1.71 | 6.53 | 3.81 |
| I240S | | | | | |
| D156T/G159V | 1521.88 | 1048.30 | 1.45 | 5.71 | 3.93 |
| R150P/E180T | 1636.14 | 1112.37 | 1.47 | 5.85 | 3.98 |
| D156T/D179N | 3855.30 | 1049.65 | 3.67 | 14.72 | 4.01 |
| D179N/I240S | 1890.16 | 826.28 | 2.29 | 9.30 | 4.07 |
| L95K/D156T/ | 2075.52 | 1194.20 | 1.74 | 7.79 | 4.48 |
| D179N | | | | | |
| D156T | 5564.55 | 1304.31 | 4.27 | 26.15 | 6.13 |
| G159V | 6330.31 | 1716.35 | 3.49 | 24.17 | 6.94 |
| G159V/D179N | 4741.70 | 1896.45 | 2.50 | 17.79 | 7.12 |
| A198L | 4888.05 | 1555.23 | 3.14 | 22.97 | 7.31 |
| L95K/D105N/ | 3640.58 | 2177.79 | 1.67 | 13.66 | 8.17 |
| E180T | | | | | |
| R150P/D156T/ | 2554.33 | 1770.29 | 1.44 | 12.00 | 8.32 |
| A198L | | | | | |
| V227E | 21170.85 | 2439.36 | 9.01 | 76.14 | 8.45 |
| I240S | 5525.59 | 1486.79 | 3.72 | 33.21 | 8.94 |
| L95K/D105N/ | 2930.99 | 2217.79 | 1.32 | 14.58 | 11.03 |
| R150P/D156T/ | | | | | |
| G159V/A198L/ | | | | | |
| V227E/I240S | | | | | |
| L95K/R150P | 6360.67 | 3108.26 | 2.05 | 30.68 | 14.99 |
| D105N/E180T | 13018.08 | 4994.85 | 2.61 | 46.52 | 17.85 |
| R150P | 11979.01 | 4261.20 | 2.81 | 56.29 | 20.02 |
| D105N | 12356.79 | 4628.13 | 2.67 | 58.06 | 21.75 |
| E180T | 26456.92 | 11205.01 | 2.36 | 94.55 | 40.04 |
| Wild-type | 26316.84 | 22348.45 | 1.18 | 94.64 | 80.37 |

In addition, a library of double mutants was generated, whereby mutations S208K, I213G and G214E were combined with each of the mutations L95K, D105N, R150P, D156K, D156T, G159V, D179N, E180T, A198L, V27E and 1240S. Six (6) double mutants were identified based on decreased catalytic activity at 37° C. relative to 25° C. and/or sufficient protein expression or activity. The double mutants, and their ratio of activity (25° C./37° C.), were as follows: almost 14-fold for the S208K/G159V mutant; about 14-fold for the S208K/D179N mutant; about 13-fold for the S208K/C227E mutant; about 8-fold for the G214E/G159V mutant; almost 14-fold for the G214E/D179N mutant; and about 14-fold for the 1213G/D179N mutant. As expected, wild-type hMMP-1 exhibited a ratio of activity of about 1-fold.

### Example 2

### Expression, Purification and Activation of Wild-Type or Mutant MMP-1

A. Expression in E. coli

DNA encoding Wild-type hMMP-1 (clone BAP006_{—}10, having a sequence of nucleotides set forth as nucleotides in SEQ ID NO:3) or encoding a mutant MMP-1 was subcloned into vector pET26b (EMD Biosciences, Catalog No. 69862; SEQ ID NO:131) by ligation using standard molecular biology techniques. The pET26b carries an N-terminal pelB signal sequence (set forth in SEQ ID NO:130) for periplasmic localization. For protein expression, the vector was transformed into BL21 (DE3) E. coli cells (NEB) using manufacturers recommendations.

B. Large Scale Purification

A transformed colony was used to inoculate 50 mL LB medium containing Kanamycin. The culture was grown at 37° C. with shaking overnight. The next day, 15 mL of the overnight culture were used to innoculate 0.8 L of LB media with Kanamycin (50 μg/mL final concentration) in a 2-L flask. A total of 4 2-L flasks were grown for each clone. The cultures were grown at 37° C., shaking at 250 rpm, until the cultures reached an OD600 of 0.8. MMP-1 expression was then induced with 0.4 mM IPTG. The cultures were kept at 4° C. for 1 hour, followed by overnight growth at 25° C. The following day, the cells were harvested by centrifugation (20 minutes at 3000×g), and the pellets were resuspended in 5% culture volume of buffer I (200 mM Tris/HcL pH7.5, 20% sucrose, 1 mM EDTA). Lysozyme (0.5 mg/mL) was added to the suspension, which was then incubated at room temperature for 30 min, while shaking. An equal volume of ice cold ddH₂O was added to the lysate, followed by incubation on ice for 20 min. The lysate was then centrifuged at 6000×g for 30 min to pellet cell debris.

Q-sepharose beads (50% of supernatant volume) were washed with 10×CV (column volume) of Q-bind buffer. The lysate supernatant was added to the washed Q-sepharose beads, and incubated at 4° C. for 1 hr, while stirring. The supernatant flow-through (FT) was collected after passing through a 0.22 μm filter. The Q-sepharose beads were then washed with 2×CV of Q-bind buffer. The washes were collected, combined with the FT, and loaded onto a pre-equilibrated 5-mL Sepharose Fast Flow column (GE Healthcare). Bound protein was eluted twice from the column with Buffer A (25 mM Tris-HCl, pH 7.5, 75 mM NaCl, 10 mM CaCl₂) and Buffer B (25 mM Tris-HCl, pH 7.5, 1 M NaCl, 10 mM CaCl₂), over the following gradient: 0%, 1×CV; 0-10% Buffer B, 10×CV; 10-70% Buffer B, 15×CV. The fractions of eluate containing the MMP-1 peak were pooled and diluted 1:8 in 25 mM Tris-HCl, pH7.5, 50 mM NaCl, 5 mM CaCl₂ and 0.05% Brij-35.

C. Activation

Purified full length MMP-1 (wild-type or mutant) was activated by proteolytic cleavage using immobilized trypsin, whereby trypsin from bovine pancreas treated with L-1-tosylamido-2-phenylethyl chloromethyl ketone (TPCK) to inhibit contaminating chymotrypsin is immobilized to 4% beaded agarose (Catalog No. 20230; Thermo Scientific Pierce; Rockford, Ill.). Two hundred (200) μL of the immobilized TPCK trypsin slurry was washed three times with 1 mL of TCN buffer (50 mM Tris pH 7.5, 150 mM NaCl, 10 mM CaCl₂). The trypsin beads were then mixed with 1 mL of purified MMP-1 mutant (approximately 3 mg/mL) and incubated for two hours at room temperature with rotation. Activated MMP-1 or mutant was separated from the trypsin beads by using a 7 kDa or 40 kDa molecular weight cut-off (MWCO) Zeba desalt spin column (Thermo Scientific). The concentration of the activated protein was determined by OD280 and the extinction of coefficient of the proteins.

To confirm that the pro-domain of the enzyme was cleaved through the trypsin activation step, untreated enzyme and trypsin-treated enzyme were visualized on a 4-20% SDS-PAGE gel followed by Coomassie blue staining. The untreated protein had an apparent molecular weight of 57 kDa whereas the trypsin-treated enzyme had an apparent molecular weight of approximately 43 kDa. In addition to the activated protein, two other proteins near the 22 kDa marker were visualized in trypsin-treated samples, which, based on N-terminal sequencing, were determined to be the catalytic and hemopexin domains. These were probably generated by autocleavage in the linker region.

D. In Vitro MMP-1 Activity

Once activated, MMP-1 (wild-type or mutant) also was assayed against a fluorogenic peptide substrate to determine its activity. The assay to assess activity monitors the rate of fluorescence production as a result of cleavage of the commercially available fluorogenic substrate, peptide IX, designated as Mca-K-P-L-G-L-Dpa-A-R-NH₂ (SEQ ID NO:88; Mca=(7-methoxycoumarin-4-yl)acetyl; Dpa=N-3-(2,4,-dinitrophenyl)-L-2,3-diaminopropionyl; R&D Systems, Minneapolis, Minn., Cat#ES010). The peptide substrate contains a highly fluorescent 7-methoxycoumarin group that is quenched by resonance energy transfer to the 2,4-dinitrophenyl group. Activated hMMP-1 cleaves the amide bond between glycine and leucine resulting in an increase in released fluorescence. Briefly, 1.6 μL of TCNB (50 nM Tris, 10 mM CaCl₂, 150 mM NaCl, 0.05% Brij 35, pH 7.5) containing 620 μM peptide IX fluorescent substrate were added to each well to a final concentration of 10 μM, followed immediately by detection of the rate of fluorescence production (relative fluorescence units (RFU)/sec) in a SpectraMax M3 fluorescent plate reader (Molecular Devices) at 320 nm exitation/405 nm emission in kinetic mode.

The results show that activated MMP-1 exhibited extensive activity against the peptide substrate that was about 78 times more active than the non-trypsin activated full-length MMP-1 containing the pro-domain. The result was similar for tested mutants (e.g., the GVSK mutant, G159V/S208K). This result further confirms that the change in molecular weight of the protein resulted in an active protein.

### Example 3

### Effect of Ca²⁺ Concentration on Activity of Selected Mutants

From the mutants identified in Table 11 above, it was observed that many obtained mutations at or near residues involved in metal binding. Mutants were selected that contained a point mutation(s) located in amino acids that were either directly involved in or adjacent to amino acids that participate in the coordination of calcium or zinc ions in order to assess the effects of calcium concentration on activity. A representative mutant was selected that has a glycine to a valine mutation at amino acid 159, which is a residue directly involved in Ca²⁺ coordination, and a serine to lysine mutation at amino acid 208, which is a residue adjacent to an amino acid involved in the binding of the catalytic Zn²⁺ molecule (G159V/S208K; GVSK, set forth in SEQ ID NO:155). Other mutants, D156T/D179N (SEQ ID NO:156) and V227E (SEQ ID NO:157), and single mutants G159V (SEQ ID NO:158) and S208K (SEQ ID NO:159), also were tested for increased calcium dependent activity.

A. GVSK Mutant, G159V/S208K

1. MMP-1 Activity as a Function of Calcium Concentration

MMP-1 and MMP-1 mutant GVSK (G159V/S208K), purified and activated as described in Example 2, were tested in vitro for catalytic activity against the fluorescent peptide substrate IX as a function of Ca²⁺ concentration. Purified and activated MMP-1 or GVSK (G159V/S208K) was diluted with TCN buffer (50 mM Tris pH 7.5, 150 mM NaCl) containing either 1, 2, 5, or 10 mM Ca²⁺ to a concentration of 1 μg/mL. From this concentration, a series of seven, three fold serial dilutions were then made. One hundred (100) μL of each sample was then added to a 96 well Fluotrac 200 black plate (Greiner Bio-One) containing 5 μL of the fluorogenic substrate peptide IX substrate as described in Example 2C (at a final concentration of 200 μm). The enzyme and substrate were incubated at 37° C. for 2 hours. The rate of fluorescence was measured using a SpectraMax M3 fluorescent plate reader (Molecular Devices) in kinetic mode at an excitation wavelength of 320 nm and an emission wavelength of 405 nm immediately after the samples were added to the peptide IX substrate and 2 hours after the addition of sample. Activity was depicted as specific activity (RFU/min/ng). Each sample was measured in duplicate and the activity was calculated by averaging the readings for each sample within the linear range of the assay.

The results show that wild-type MMP-1 had substantially similar specific activities at the varying concentrations of Ca²⁺ ranging from about 8.0 to 10.0 RFU/min/ng. The highest specific activity of about 10.0 RFU/min/ng for wild-type MMP-1 was seen at Ca²⁺ concentrations of 1 and 2 mM. For the GVSK mutant G159V/S208K, the highest activity was observed in the presence of 10 mM Ca²⁺, although the observed activity was reduced by about 25% compared to wild-type MMP-1 in the presence of 10 mM Ca²⁺ with GVSK (G159V/S208K) having a specific activity of about 6.0 RFU/min/ng and wild-type MMP-1 having a specific activity of about 8.0 RFU/min/ng. The results show that the GVSK (G159V/S208K) mutant had decreased activity at decreasing concentrations of Ca²⁺. The activity decreased at lower calcium concentrations. At 2 mM Ca²⁺, the specific activity of the GVSK (G159V/S208K) mutant was decreased 3-fold compared to its activity at 10 mM Ca²⁺. As the concentration further decreased to 1 mM, the activity of the GVSK (G159V/S208K) mutant decreased approximately 10-fold compared to its activity at 10 mM Ca²⁺.

The experiment also was performed by incubating the enzyme and substrate at 25° C. instead of 37° C. The results were generally similar, except that the results showed that the GVSK (G159V/S208K) mutant lost less activity at 25° C. compared to at 37° C. For example, in the presence of 1 mM Ca²⁺, the GVSK (G159V/S208K) mutant lost only about half of its activity compared to its activity in the presence of 10 mM Ca²⁺ when tested at 25° C. Thus, the results show that temperature and Ca²⁺ concentration both had an effect on activity.

2. Time Course of MMP-1 Activity in Presence of Calcium

The calcium-dependent activity of activated GVSK (G159V/S208K) as compared to activated wild-type MMP-1 was further characterized by assaying the activity as a function of time and calcium concentration. Enzyme activity was examined using the same fluorometric assay as described above. Briefly, purified and activated MMP-1 or GVSK (G159V/S208K) was diluted with TCN buffer (50 mM Tris pH 7.5, 150 mM NaCl) containing either 1 or 10 mM Ca²⁺ to a concentration of 1 μg/mL. From this concentration, a series of seven, three fold serial dilutions were then made. 100 μL of each sample was then added to a 96 well Fluotrac 200 black plate (Greiner Bio-One) containing 5 μL of a 200 μm final concentration fluorogenic substrate. The enzyme and substrate were incubated at 37° C., and the plates were read just after addition and 15, 60, 90, and 180 minutes later using a SpectraMax M3 fluorescent plate reader (Molecular Devices). Activity was depicted as specific activity (RFU/min/ng). Relative fluorescence units (RFU) were determined at an excitation wavelength of 320 nm and an emission wavelength of 405 nm.

The results show that the activity of wild-type MMP-1 at 37° C. at 10 mM calcium concentration increased slightly at the early time points with a specific activity of almost 4 RFU/min/ng immediately after addition of sample to substrate, increasing to about 5 RFU/min/ng at 60 and 90 minutes after addition of sample to substrate. Then, at later time points up until 180 minutes after addition of sample to substrate the specific activity decreased and was similar to the starting activity. Similar results were generally observed at the 1 mM calcium concentration, although the observed specific activity was slightly less than at the higher concentration of calcium. For example, for wild-type MMP-1, the specific activity measured immediately after addition of sample to substrate incubated in the presence of 10 mM Ca²⁺ was about 4.0 RFU/min/ng, whereas it was about 3.0 RFU/min/ng in the presence of 1 mM Ca²⁺.

As above, the highest activity of the GVSK (G159V/S208K) mutant was observed at the 10 mM higher concentration of Ca²⁺, although it was slightly less than wild-type MMP-1. For example, for wild-type MMP-1, the specific activity measured immediately after addition of sample to substrate incubated in the presence of 10 mM Ca²⁺ was about 4.0 RFU/min/ng, whereas it was about 3.5 RFU/min/ng for the GVSK (G159V/S208K) mutant. At the higher concentration of Ca²⁺, the GVSK (G159V/S208K) mutant showed the same general temporal pattern of activity as wild-type MMP-1, although the increase in fluorescence at earlier times was less than for the wild-type MMP-1. This result shows that for the GVSK (G159V/S208K) mutant there was no loss of activity during the course of the experiment at 10 mM Ca²⁺. In contrast, GVSK (G159V/S208K) in low calcium of 1 mM had a lower starting specific activity of only about 2.0 RFU/min/ng, which decreased a further 2-fold after 15 minute incubation with substrate. The loss of activity continued over time and by 180 minutes there was almost no detectable activity measured for the GVSK (G159V/S208K) mutant in the presence of 1 mM calcium.

3. Kinetics of MMP-1 Activity in Presence of Calcium

The calcium-dependent activity of activated GVSK (G159V/S208K) as compared to activated wild-type MMP-1 was further characterized by determining the enzyme kinetics of the two enzymes as a function calcium concentration. Purified MMP-1 and GVSK, at a concentration of 5 nM were incubated with a series of two-fold dilutions (0.95-62 μM final concentration) of fluorogenic peptide substrate IX in either 1 or 10 mM CaCl₂ TCN buffer. One hundred μL reactions were incubated at 37° C. in a 96-well Fluotrac 200 black plate. Plates were read using the SpectraMax M3 fluorescent plate reader (Molecular Devices) using an excitation wavelength of 320 nm and an emission wavelength of 405 nm. The conversion factor (i.e., complete hydrolysis) was obtained by incubation of the fluorescent substrate with 0.25 μM MMP-1 for 24 hours at 25° C. Hydrolysis rates were obtained from fluorescence versus time plots using data points from the linear portion of the curve. The slope of these plots was divided by the fluorescence change corresponding to complete hydrolysis and then multiplied by the substrate concentration to obtain hydrolysis rates in μM s⁻¹. Kinetic parameters were obtained by Lineweaver-Burk, Eadie-Hofstee, and Hanes-Woolf analysis. Each experiment was performed three times and each sample assayed in duplicate. The results are set forth in Table 13 below.

TABLE 13
Enzyme Kinetics of Tested Mutants

| Sample | [Ca²⁺] mM | k_{cat} (s⁻¹) | K_{M} (μM) | k_{cat}/K_{M} (s⁻¹ M⁻¹) |
|---|---|---|---|---|
| MMP-1 | 10 | 4.54 ± 1.49 | 21.13 ± 7.00 | 216,000 ± 41,500 |
| MMP-1 | 1 | 5.34 ± 2.07 | 20.57 ± 5.35 | 254,000 ± 52,400 |
| GVSK | 10 | 2.97 ± 1.00 | 11.0 ± 2.56 | 265,800 ± 44,600 |
| GVSK | 1 | 2.26 ± 0.64 | 16.53 ± 3.47 | 135,300 ± 15,600 |

The results show that wild-type MMP-1 exhibits similar enzyme kinetics whether in the presence of high (10 mM) or low (1 mM) concentrations of Ca²⁺. In contrast, GVSK (G159V/S208K) kinetics are highly dependent on calcium concentration. For example, the specificity constant (k_{cat}/K_{M}), used to represent catalytic efficiency, for GVSK in the presence of 10 mM Ca²⁺ is similar to those of wild-type MMP-1 in the presence of either 1 mM or 10 mM Ca²⁺. However, in the presence of 1 mM Ca²⁺, GVSK has a specificity constant that is approximately half that of GVSK in the presence of 10 mM Ca²⁺. The reduced catalytic efficiency of GVSK in buffer containing 1 mM Ca²⁺ can be attributed to the reduced affinity of GVSK for the substrate, as indicated by the higher K_{M} value at the lower calcium concentration.

B. Comparison of Ca²⁺-Dependence of Tested Mutants

The activity of GVSK (G159V/S208K; SEQ ID NO:155) was compared to other MMP-1 mutants, D156T/D179N (SEQ ID NO:156) and V227E (SEQ ID NO:157), and wild-type MMP-1 (SEQ ID NO:2) following purification and activation as described in Example 2. Activity against the fluorogenic substrate peptide IX was determined as described in above in part A and compared as between and among wild-type MMP-1 and MMP-1 mutants GVSK (G159V/S208K), D156T/D179N and V227E.

The results are set forth in Table 14. The results show that like GVSK (G159V/S208K), the V227E mutant exhibited calcium-dependent activity in the presence of high calcium compared to low calcium. The activity of the V227E mutant decreased in the presence of 10 mM and 1 mM Ca²⁺ over time to about 60% and about 20%, respectively (activity measured immediately after addition of sample to substrate versus 2 hours after incubation of sample with substrate). The mutant D156T/D179N also exhibited calcium-dependence with higher specific activity measured at 10 mM Ca²⁺ than 1 mM Ca²⁺, although its activity did not change with time at either of the tested concentrations of calcium and in fact slightly increased. The mutants V227E and D156T/D179N, however, exhibited a low level of activity such that their specific activity was barely detectable at any of the time points tested. Thus, the results show that GVSK (G159V/S208K) had the highest specific activity of the mutants tested, as well as a Ca²⁺-dependent and time-dependent activity.

TABLE 14
Calcium-dependence of activity

| | Specific Activity (RFU/min/ng) | | | |
|---|---|---|---|---|
| | Time | | | |
| | immediately after | | 2 hour incubation | |
| | incubation with substrate | | with substrate | |
| [Ca²⁺] (mM) | 10 | 1 | 10 | 1 |
| wild-type | 6.48 | 5.67 | 6.76 | 4.41 |
| GVSK (G159V/S208K) | 7.04 | 2.62 | 7.83 | 0.68 |
| D156T/D179N | 0.22 | 0.06 | 0.29 | 0.10 |
| V227E | 1.60 | 0.77 | 1.04 | 0.12 |

C. G159V and S208K Single Mutants and Increased Calcium Dependency

In a further experiment, G159V (SEQ ID NO:158) and S208K (SEQ ID NO:159) single mutants were tested for increased calcium-dependent activity. The G159V and S208K muteins were expressed, purified and activated as described in Example 2. The catalytic activities of the muteins were then determined in the presence of 1 mM and 10 mM Ca²⁺, as described in above in part A, and compared. The S208K mutant demonstrated similar levels of activity at 1 mM and 10 mM Ca²⁺, which was comparable to the behavior of wild-type MMP-1 described in part A above. In contrast, the G159V mutant exhibited increased calcium dependency, exemplified by a decrease in activity of approximately 4-fold in 1 mM Ca²⁺ compared to its activity at 10 mM Ca²⁺. These results show that the calcium sensitivity of the GVSK mutant is likely due to the G159V mutation.

### Example 4

### Effect of Zinc Concentration on Activity of Selected Mutants

MMP-1 and MMP-1 mutant GVSK (G159V/S208K), purified and activated as described in Example 2, were tested in vitro for catalytic activity against a fluorescent peptide substrate as a function of Zn²⁺ concentration. Purified and activated MMP-1 or GVSK (G159V/S208K) was diluted with TCN buffer (50 mM Tris pH 7.5, 150 mM NaCl) containing either 1 or 10 μM Zn²⁺ and either 1 and 10 mM Ca²⁺. From this concentration, a series of seven, three-fold serial dilutions were then made. One hundred (100) μL of each sample was then added to a 96 well Fluotrac 200 black plate (Greiner Bio-One) containing 5 μL of the fluorogenic substrate peptide 1× substrate as described in Example 2C (at a final concentration of 200 μm). The enzyme and substrate were incubated at 37° C. for 2 hours. Fluorescence was measured using a SpectraMax M3 fluorescent plate reader (Molecular Devices) and relative fluorescence units (RFU) were determined at an excitation wavelength of 320 nm and an emission wavelength of 405 nm immediately after the samples were added to the peptide IX substrate and 2 hours after the addition of sample. Specific activity (RFU/min/ng) was determined. Each sample was measured in duplicate and the activity was calculated by averaging the readings for each sample within the linear range of the assay.

The results show no difference in the in vitro activity of wild-type MMP-1 or GVSK (G159V/S208K) as a function of the Zn²⁺ concentration in either the 1 or 10 mM Ca²⁺ TCN buffer.

### Example 5

### Assessment of Protein Degradation as a Function of Calcium Concentration

To determine if the loss of activity in GVSK (G159V/S208K) at low concentrations of calcium was due to autolysis, the degradation of the protein was assessed by visualizing protein fragments by SDS-PAGE.

1. GVSK (G159V/S208K) Mutant

MMP-1 and MMP-1 mutant GVSK (G159V/S208K), purified and activated as described in Example 2, were incubated at a concentration of 0.1 mg/mL in either 1 mM Ca²⁺ or 10 mM Ca²⁺ TCN buffer at 25° C. and 37° C. Samples were collected at 0, 30, 60 and 120 minutes, and reaction conditions were stopped by the addition of gel sample buffer. Samples were placed in 99° C. water for 5 minutes, and then loaded onto a 4-20% SDS polyacrylamide gel followed by Coomassie staining.

For wild-type MMP-1, clear bands were observed representing active MMP-1 forms after incubation of samples at Ca²⁺ concentrations of 10 mM or 1 mM, and either 25° C. or 37° C., for all collected time points. Thus, the results showed that MMP-1 showed no evidence of degradation, and hence was stable. For the GVSK (G159V/S208K) mutant, clear bands also were observed representing stable, non-degraded protein in samples incubated in 10 mM Ca²⁺, incubated at either 25° C. or 37° C., for all collected time points. Additionally, incubation of the GVSK (G159V/S208K) mutant at Ca²⁺ concentrations of 1 mM and at 25° C. also showed the presence of stable, non-degraded protein. In contrast, incubation of the GVSK (G159V/S208K) MMP-1 mutant in 1 mM Ca²⁺ at 37° C. showed that degradation of protein product had occurred as evidenced by a significant decrease in the band corresponding to the activated form after 30 minutes, which was almost gone after two hours. For the GVSK (G159V/S208K) mutant, Ca²⁺-dependent activity appears to correlate with protein instability.

2. Other Tested Mutants

MMP-1 mutants V227E and D156T/D179N were activated as described in Example 2, except that activation was allowed to proceed for 15 minutes or 60 minutes. Then, unactivated and activated samples were incubated at a concentration of 0.1 mg/mL in either 1 mM Ca²⁺ or 10 mM Ca²⁺ TCN buffer at 37° C. Samples were collected at 0 and 120 minutes, and reaction conditions were stopped by the addition of gel sample buffer. Samples were placed in 99° C. water for 5 minutes, and then loaded onto a 4-20% SDS polyacrylamide gel followed by Coomassie staining.

The results show that unactivated forms of both mutants exhibited the expected higher molecular weight compared to the activated products. There was no difference in detected protein for either of the unactivated forms at either 1 mM or 10 mM Ca²⁺ showing that Ca²⁺ concentration does not affect degradation of the unactivated forms. Activation of both mutants was observed, based on a decreased size of the protein products, after activation for 15 minutes and 60 minutes. For the D156T/D179N mutant, clear bands were observed representing active MMP-1 forms after incubation of samples at Ca²⁺ concentrations of 10 mM or 1 mM, and the amount of detected protein was not changed after incubation for 2 hours. Thus, the results showed that D156T/D179N mutant showed no evidence of degradation as a function of calcium concentration, and hence was stable. In contrast, the activated form of mutant V227E, like GVSK (G159V/S208K), was degraded upon incubation at 1 mM Ca²⁺, but not 10 mM Ca²⁺, as evidenced by a loss of any detectable protein product after incubation for 2 hours in the presence of 1 mM Ca²⁺. For the V227E mutant, Ca²⁺-dependent activity appears to correlate with protein instability.

### Example 6

### Assessment of Soluble Collagen I Digestion as a Function of Calcium Concentration

The enzymatic activities of MMP-1 and GVSK (G159V/S208K) were monitored by assessing digestion of soluble collagen I, a physiological substrate of MMP-1. MMP-1 and GVSK (G159V/S208K) at a concentration of 0.1 mg/mL were pre-incubated in TCN buffer for 2 hours with either 1 mM Ca²⁺ or 10 mM Ca²⁺, and at either 25° C. or 37° C. Collagen I isolated from calf skin and labeled with fluorescein isothiocyanate (Elastin Products Company, Inc.) was diluted 10-fold to a final concentration of 0.35 mg/mL with either 10 mM Ca²⁺ TCN buffer or 1 mM Ca²⁺ TCN buffer. After pre-incubation of the enzyme with calcium, 5 μL of enzyme was mixed with 45 μL of the diluted collagen I substrate solution in a total reaction volume of 50 μL at 25° C. for 2 hours. Reaction conditions were stopped by the addition of gel sample buffer. Samples were placed in 99° C. water for 5 minutes, and then loaded onto a 4-20% SDS polyacrylamide gel followed by Coomassie staining.

Three bands were visualized by Coomassie blue staining for untreated soluble collagen I fraction that was not treated with MMP-1 or GVSK (G159V/S208K) mutant: a high molecular weight band greater than 250 kDa, a doublet having a molecular weight slightly less than 250 kDa and a doublet of about 120 kDa. Treatment of collagen I with wild-type MMP-1 pre-incubated in either 10 mM Ca²⁺ or 1 mM Ca²⁺ and at 25° C. or 37° C. resulted in a new digestion pattern, whereby the three bands seen in the untreated collagen I sample were reduced in molecular weight in addition to the appearance of additional bands of about 50 and 36 kDa. A similar digestion pattern resulted when GVSK (G159V/S208K) was pre-incubated at either 25° C. or 37° C. with 10 mM Ca²⁺ or when GVSK was pre-incubated at 25° C. with 1 mM Ca²⁺. In contrast, when GVSK (0159V/S208K) was pre-incubated at 37° C. in the presence of 1 mM Ca²⁺, the digestion pattern was more similar to the untreated collagen-I molecular weight profile with the three molecular weight bands observed showing that the GVSK was not able to digest soluble collagen Ito any significant extent. The results also showed the minor presence of additional digestion products, but they were not as apparent as in the other enzyme treated samples. The results show that in the presence of low calcium of 1 mM and at 37° C., the ability of the GVSK (G159V/S208K) mutant to digest collagen was abolished, since it was not able to digest collagen I to any significant extent.

### Example 7

### In Vivo Collagen I Degradation by MMP-1 and GVSK (G159V/S208K) Mutant

To test the in vivo degradation of collagen I, MMP-1 and GVSK (G159V/S208K) mutant enzymes, activated and purified as described in Example 2, were injected into Zucker rat ventral skin. Enzyme activity of perfusates was analyzed by examining collagen I degradation by Western blot.

Briefly, 6-12 month old male Zucker rats (Harlan labs) were shaved on the ventral surface. Then, rats were perfused with 1 ml TCN buffer (either 1 or 10 mM CaCl₂) containing 1 μg/mL of rHuPH20 (U.S. Pat. No. 7,767,429; prepared from expression in CHO cells of nucleic acid encoding the amino acid sequence set forth in SEQ ID NO:99) and 2.5 μg/mL of epinephrine (Sigma) using a PHD 2000 Infuse/Withdraw Pump (Harvard Apparatus) at a rate of 0.12 mL/min at five different sites or less. After 10 minutes, 1 mL of wild-type MMP-1 or GVSK (G159V/S208K) mutant at a concentration of 50 μg/mL in TCN buffer (either 1 or 10 mM CaCl₂) was perfused at a rate of 0.12 mL/min. As a control, Zucker rat skin was perfused with TCN buffer (1 mM Ca²⁺) without enzyme (vehicle control). At various time points after the start of the second perfusion (2 min, 60 min or 90 min), 1 cm full thickness biopsies of treated sites were taken and fixed in Bousin solution (Sigma) for histology. Perfusate that collected in the area vacated by the biopsied tissue was transferred with a pipette to Eppendorf tubes and kept at −20° C. until further analysis.

Perfusate was analyzed by Western Blot. Perfusate samples (5-20 μL) containing 4 μg of protein were mixed with gel sample buffer and heated for five minutes at 99° C. All samples were electrophoresed on either 8% or 4-20% pre-cast SDS-polyacrylamide gels (Invitrogen). Gels were blotted onto a nitrocellulose membrane (Invitrogen). Blots were blocked for 30 minutes in blocking buffer containing 1% milk in PBS-T (0.05% Tween 20 (Sigma) in phosphate buffered saline (EMD Chemicals)). Blots were washed three times in PBS-T. The blots were then incubated with a 1:1000 dilution of rabbit polyclonal anti-collagen I antibody (Abcam) primary antibody for 1 hour at room temperature in blocking buffer. After the primary antibody was incubated, the membrane was washed with PBS-T. The blots were then incubated with a 1:2,000 dilution of anti-rabbit HRP (Calbiochem) secondary antibody in blocking buffer. The blots were washed with PBS-T. Blots were developed using TMB insoluble reagent (Tetramethylbenzidine Calbiochem).

The results showed that wild-type MMP-1 and the GVSK (G159V/S208K) mutant were able to degrade collagen I in skin in vivo. Western blot results analyzed using an anti-collagen antibody showed that both GVSK (G159V/S208K) and MMP-1 at either calcium concentration were able to degrade collagen I. In samples that were perfused with vehicle control, prominent bands greater than 100 kDa were observed. Western blot analysis of 60 minute perfusate samples from skin sites treated with both the wild-type and mutant enzyme revealed a prominent band at approximately 98 kDa that was not present in the site treated with vehicle alone as well as other minor degradation products. Histological analysis of GVSK (G159V/S208K) or MMP-1 treated skin confirmed that collagen degradation had occurred as the normal organization of collagenous fibrous septa in the hypodermis was disrupted.

### Example 8

### Assessment of MMP-1 Complex Formation Following In Vivo Perfusion

To assess the fate of the perfused MMP-1 protein in vivo, perfusate samples from Zucker rat skin treated with either activated MMP-1 (1 mM Ca²⁺), GVSK (1 mM Ca²⁺) or GVSK (10 mM Ca²⁺) also were analyzed by Western blot with an anti-MMP-1 antibody or by ELISA. Perfusion was performed as described in Example 7 and perfusate samples collected for analysis.

1. Western Blot

Perfusate was analyzed by Western Blot. As a control, 100 ng of either MMP-1 or GVSK (G159V/S208K) mutant that had not been perfused into the skin also were prepared to show the composition of the protein prior to perfusion. Negative control samples of perfusates not containing perfused enzyme also were analyzed by preparing perfusate samples of Zucker rat skin that was perfused with TCN buffer only (1 mM or 10 mM Ca²⁺). A final sample also was prepared by incubating 1 μg purified wild-type MMP-1 with 1 μl Zucker rat serum in a final volume of 30 μL (final serum concentration 3.33%) for 30 minutes at room temperature. Perfusate samples (5-20 μL) containing 4 μg of protein and other samples were mixed with gel sample buffer and heated for five minutes at 99° C. All samples were electrophoresed on either 8% or 4-20% pre-cast SDS-polyacrylamide gels (Invitrogen). Gels were blotted onto a nitrocellulose membrane (Invitrogen). Blots were blocked for 30 minutes in blocking buffer containing 1% milk in PBS-T (0.05% Tween 20 (Sigma) in phosphate buffered saline (EMD Chemicals)). Blots were washed three times in PBS-T. The blots were then incubated with a goat anti-human MMP-1 antibody (R&D systems) primary antibody for 1 hour at room temperature in blocking buffer. After the primary antibody was incubated, the membrane was washed with PBS-T. The blots were then incubated with a 1:2,000 dilution of anti-goat HRP (Calbiochem) secondary antibody in blocking buffer. The blots were washed with PBS-T. Blots were developed using TMB insoluble reagent (Tetramethylbenzidine Calbiochem).

2. ELISA

The amount of MMP-1 or GVSK (G159V/S208K) present in perfusate samples was quantified by ELISA. Immulon 4HBX 96-well plates were coated overnight with 100 pt of anti-human MMP-1 antibody (R&D) at 1 μg/mL in 100 mM sodium phosphate buffer pH 7.2 at 4° C. Plates were washed five times with 300 μL/well of PBS and blocked with 200 μL/well of PBS-T (0.05% Tween 20, Sigma) for 1 hour at room temperature. Purified human MMP-1 (R&D) standards were prepared by dilution in PBS-T to an initial concentration of 200 μg/mL. A series of six three-fold dilutions were prepared from this initial concentration. Perfusate samples were initially diluted 1:100 followed by six three-fold dilutions.

100 μL of each standard and diluted sample were added per well of the coated and blocked plates and incubated for two hours at room temperature. Plates were washed five times with 300 μL per well with PBS-T followed by a two hour incubation at room temperature with 100 μL per well of a biotin conjugated anti-human MMP-1 antibody (R&D) at 0.25 μg/mL in PBS-T. Plates were washed five times with 300 μL PBS-T per well followed by incubation with 100 μL per well of 1 μg/mL streptavidin-HRP (Jackson ImmunoResearch) for one hour at room temperature. A final wash was done followed by the addition of 100 per well of Sure Blue TMB microwell peroxidase substrate (KPL). After five minutes, 100 μL per well of TMB Stop Solution (KPL) was added to the reactions, and the plates were read using the SpectraMax M3 fluorescent plate reader (Molecular Devices) at 450 nm. Perfusate samples were assayed in duplicate and calculated by averaging the readings for each sample within the linear range of the assay. The amount of uncomplexed protein detected in the perfusate at each time point was determined as a percentage of the starting concentration.

3. Results

Western Blot of pre-injection controls of 100 ng of either activated MMP-1 or activated GVSK (G159V/S208K) showed a prominent monomer band near 50 kDa. The negative control perfusate samples that were not perfused with enzyme did not show any prominent equivalent bands. Perfusate samples from skin perfused with MMP-1 (in 1 mM Ca²⁺) and GVSK (in 10 mM Ca²⁺) samples were analyzed at 2 minutes and 90 minutes after perfusion. As early as 2 minutes after perfusion, there were multiple higher molecular weight bands in addition to the activated monomer protein band near 50 kDa that were present in both samples. The presence of multiple high molecular weight bands that were reactive with the MMP-1 antibody also was observed in the sample containing purified MMP-1 mixed with Zucker rat serum. The higher molecular weight bands indicate that the enzyme is interacting with a serum-derived protein in vivo. Also in both perfusate samples, the percentage of higher molecular weight bands increased concomitant with a decrease in monomer band with time in the 90 minute perfusate samples. For the samples perfused with wild-type MMP-1, the monomer band was only slightly decreased, whereas in the perfusate sample from skin perfused with GVSK (in 10 mM Ca²⁺), there was no monomer remaining by 90 minutes after perfusion.

The amount of MMP-1 or GVSK (G159V/S208K) in the perfusates was quantitated by solid phase capture ELISA using an antibody that recognizes predominantly the monomeric or uncomplexed form of the protein. The percent of uncomplexed protein detected in perfusate samples collected 2 minutes after perfusion with MMP-1 (in 1 mM Ca²⁺) or GVSK (in 10 mM Ca²⁺) was about 90% of the starting concentration, whereas in samples perfused with GVSK (in 1 mM Ca²⁺) it was only about 45% of the starting concentration. For all proteins, the amount of uncomplexed protein as a percentage of the starting concentration decreased over time, although the decrease was most striking for GVSK (in 1 mM Ca²⁺) samples where less than one tenth of uncomplexed form was detectable by 30 minutes post-perfusion. In contrast, for the GVSK (in 10 mM Ca²⁺) samples, the concentration of the protein in perfusate decreased to 50% of the starting concentration 30 minutes after perfusion, and further decreased to 30% and to about 13% in perfusate samples collected 60 minutes and 90 minutes, respectively. Wild-type MMP (in 1 mM Ca²⁺) was the most stable, where the concentration of the protein in the perfusate was approximately half of the starting concentration in perfusate samples collected 30 minutes after start of perfusion and was approximately one third in samples 90 minutes after perfusion. Thus, the results show that at the lower calcium concentration, the GVSK (G159V/S208K) mutant was more susceptible to complex formation.

### Example 9

### Assessment of MMP-1 Complex Formation with α-2 Macroglobulin

To determine whether the higher molecular weight bands observed in perfusate samples were due to MMP-1 or GVSK (G159V/S208K) bound to a-2 macroglobulin, an immunoprecipitation was performed. For the immunoprecipitation, 12 μg of total protein from the rat perfusates (approximately 15-30 μL) were mixed with 5 μL of an anti-rat a-2 macroglobulin rabbit antibody (Alpha Diagnostic International) and brought to a final volume of 200 μL with PBS-T. After an overnight incubation at 4° C., 40 μL of protein A/G beads (Pall Scientific) were added and incubated with rotation for 3 hours at room temperature. Each sample was washed four times with 1 ml of PBS-T and 30 μL of gel sample buffer was added. The samples were heated for five minutes at 99° C., electrophoresed on a 4-20% SDS-polyacrylamide gel, and then transferred onto a nitrocellulose membrane. The blots were probed with the anti-human MMP-1 antibody as described in Example 8.

Western blot with anti-MMP-1 antibody of perfusate samples from animals perfused with buffer alone (i.e. not treated with either MMP-1 or GVSK), when immunoprecipitated with an anti-a-2 macroglobulin, revealed a non-specific band at 60 kDa, but no higher molecular weight bands. In contrast, the same higher molecular weight bands observed in either MMP-1 or GVSK (G159V/S208K) treated skin perfusates were present in perfusate samples obtained 2 minutes and 90 minutes after perfusion, indicating that MMP-1 is complexing with a-2-macroglobulin in the skin in vivo.

### Example 10

### Effect of Serum on MMP-1 Activity

To assess the effect that formation of complexes with a-2-macroglobulin, or other serum components, had on MMP-1 activity, proteins were incubated with serum and activity assessed. Activated MMP-1 and GVSK, at 1 μg/mL, were incubated in 10% Zucker rat serum containing either 1 mM or 10 mM Ca²⁺ in a final volume of 120 μL for 5, 15, 30, 60, and 120 minutes at 25° C. After incubation, the enzymatic activity was determined, using a fluorogenic peptide substrate, as described in Example 2C.

The activity of MMP-1 in 1 mM Ca²⁺ buffer was unaffected by incubation with serum over the course of the study. In contrast, incubation with serum resulted in a gradual decrease in activity of GVSK in 10 mM Ca²⁺ over the course of the study, with approximately 74% of the original activity remaining by the 2 hour time point. Decreasing the calcium concentration of the GVSK sample to 1 mM Ca²⁺ resulted in a rapid reduction of activity of GVSK in the presence of serum. After 15 minutes of serum incubation, one-third of the activity of GVSK in 1 mM Ca²⁺ was lost, and less than 50% of activity remained after 30 minutes. By the 2 hour end time point, only approximately 17% of the activity of GVSK in 1 mM Ca²⁺ remained, indicating that a decrease in calcium concentration rendered GVSK more susceptible to serum-mediated enzymatic inactivation.

### Example 11

### GVSK (G159V/S208K) Digestion of Keloidal Collagen

Eight micron cryosections were prepared from a human keloid biopsy. The sections were placed on slides and incubated in 150 mL of either 10 mM TCN (50 mM Tris, pH 7.5, 10 mM CaCl₂, 150 mM NaCl) buffer alone or 1.6 mg/mL modified MMP-1 G159V/S208K in 10 mM TCN buffer for two hours at 37° C. Following the incubation, the sections were fixed for 5 minutes in 10% neutral buffered formalin (VWR) at room temperature. The sections were then stained with hematoxylin and eosin (H&E), using standard procedures, and analyzed by light microscopy. Micrographs were taken using a Nikon Eclipse TE2000-U microscope equipped with Spot software, version 4.6 (Spot Imaging Solutions).

The sections treated with G159V/S208K, exhibited a dramatic decrease in the intensity of H&E staining compared to buffer treatment alone, indicating that a majority of the collagen had been degraded by the G159V/S208K enzyme. In particular, the smaller collagen bundles in the hyperdermis appeared to be completely digested and the majority of the larger bundles of collagen, found deep within the keloid, also were digested as compared to buffer treatment alone. These results show that, in the presence of 10 mM Ca²⁺, the G159V/S208K mutant enzyme is capable of digesting human fibrotic collagen.

Since modifications will be apparent to those of skill in this art, it is intended that this invention be limited only by the scope of the appended claims.

1. A method of treating a fibrotic disease or condition, wherein:
degrading a component of the extracellular matrix effects treatment of the disease or condition; and
the method comprises:
a) administering to a locus of a subject a therapeutically effective amount of a modified matrix metalloprotease-1 (MMP-1) or a catalytically active fragment thereof to degrade an extracellular matrix substrate of the MMP-1, wherein:
the modified MMP-1 comprises an amino acid replacement in an unmodified MMP-1 polypeptide or catalytically active fragment thereof; and
the modification confers to the modified MMP-1 or catalytically active fragment thereof reduced activity in the presence of physiological levels of extracellular calcium compared to its activity in the presence of a calcium concentration that is greater than the physiological level, whereby the activity of the modified MMP-1 decreases upon exposure to physiological conditions; and
b) administering at or near the same locus a composition containing calcium in a concentration that is greater than physiological levels of extracellular calcium, whereby the modified MMP-1 is conditionally active after administration so that the component of the extracellular matrix is degraded for a limited time to thereby treat the disease or condition.

2. The method of claim 1, wherein the unmodified MMP-1 or catalytically active fragment comprises the sequence of amino acids set forth in SEQ ID NO:5 or is a catalytically active fragment thereof, or a sequence of amino acids that exhibits at least 85% sequence identity to SEQ ID NO:5 or a catalytically active fragment thereof.

3. The method of claim 1, wherein the modified MMP-1 and calcium are administered separately or together in the same composition.

4. The method of claim 3, wherein:
the modified MMP-1 and calcium are administered together; and
the administered composition comprises the modified MMP-1 and calcium in a concentration that is greater than physiological levels of extracellular calcium.

5. The method of claim 3, wherein:
the modified MMP-1 and calcium are administered separately; and
the calcium is administered prior to, intermittently with, subsequently to, or simultaneously with administration of the modified MMP-1 or catalytically active fragment.

6. The method of claim 1, wherein the modified MMP-1 or catalytically active fragment thereof exhibits reduced activity at physiological levels of extracellular calcium compared to the activity of the corresponding unmodified MMP-1 not containing the modification(s).

7. The method of claim 1, wherein the modified MMP-1 or catalytically active fragment comprises an amino acid replacement at or near an amino acid residue that is a metal-binding site.

8. The method of claim 7, wherein the metal-binding site is a zinc- or calcium-binding site.

9. The method of claim 8, wherein the modified MMP-1 or catalytically active fragment comprises a modification at an amino acid position corresponding to a position selected from among 102, 103, 104, 105, 106, 107, 108, 136, 137, 138, 139, 140, 141, 142, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 196, 197, 198, 199, 201, 202, 203, 204, 205, 206, 207, 208, 209, 210, 211, 212, 263, 264, 265, 266, 267, 268, 269, 307, 308, 309, 310, 311, 312, 313, 356, 357, 358, 359, 360, 361, 362, 405, 406, 407, 408, 409, 410 and 411, with reference to amino acid positions set forth in SEQ ID NO:2, wherein corresponding amino acid positions are identified by alignment of the MMP-1 polypeptide with the polypeptide set forth in SEQ ID NO:2.

10. The method of claim 9, wherein the modified MMP-1 or catalytically active fragment comprises an amino acid replacement selected from among replacement with: R at a position corresponding to position 102; K at a position corresponding to position 102; V at a position corresponding to position 102; M at a position corresponding to position 102; P at a position corresponding to position 102; N at a position corresponding to position 102; G at a position corresponding to position 102; L at a position corresponding to position 102; D at a position corresponding to position 102; S at a position corresponding to position 102; F at a position corresponding to position 102; A at a position corresponding to position 102; E at a position corresponding to position 102; Q at a position corresponding to position 102; C at a position corresponding to position 102; N at position corresponding to position 103; E at a position corresponding to position 104; T at a position corresponding to position 104; R at a position corresponding to position 104; D at a position corresponding to position 104; Q at a position corresponding to position 104; V at a position corresponding to position 104; Y at a position corresponding to position 104; H at a position corresponding to position 104; L at a position corresponding to position 104; A at a position corresponding to position 104; M at a position corresponding to position 104; A at a position corresponding to position 105; C at a position corresponding to position 105; F at a position corresponding to position 105; G at a position corresponding to position 105; I at a position corresponding to position 105; L at a position corresponding to position 105; M at a position corresponding to position 105; N at a position corresponding to position 105; P at a position corresponding to position 105; R at a position corresponding to position 105; S at a position corresponding to position 105; T at a position corresponding to position 105; V at a position corresponding to position 105; W at a position corresponding to position 105; E at a position corresponding to position 105; M at a position corresponding to position 106; A at a position corresponding to position 106; Y at a position corresponding to position 106; V at a position corresponding to position 106; I at a position corresponding to position 106; L at a position corresponding to position 107; T at a position corresponding to position 107; S at a position corresponding to position 107; R at a position corresponding to position 107; M at a position corresponding to position 107; V at a position corresponding to position 107; D at a position corresponding to position 107; A at a position corresponding to position 107; K at a position corresponding to position 107; G at a position corresponding to position 107; P at a position corresponding to position 108; G at a position corresponding to position 108; E at a position corresponding to position 108; A at a position corresponding to position 108; Y at a position corresponding to position 108; K at a position corresponding to position 108; C at a position corresponding to position 108; S at a position corresponding to position 108; S at a position corresponding to position 108; F at a position corresponding to position 108; I at a position corresponding to position 108; L at a position corresponding to position 108; N at a position corresponding to position 108; D at a position corresponding to position 136; M at a position corresponding to position 136; N at a position corresponding to position 136; A at a position corresponding to position 136; L at a position corresponding to position 136; P at a position corresponding to position 136; T at a position corresponding to position 136; R at a position corresponding to position 136; S at a position corresponding to position 136; H at a position corresponding to position 136; E at a position corresponding to position 136; A at a position corresponding to position 137; R at a position corresponding to position 137; G at a position corresponding to position 137; K at a position corresponding to position 137; H at a position corresponding to position 137; P at a position corresponding to position 137; S at a position corresponding to position 137; L at a position corresponding to position 137; W at a position corresponding to position 137; F at a position corresponding to position 137; T at a position corresponding to position 137; Y at a position corresponding to position 137; E at a position corresponding to position 137; G at a position corresponding to position 138; R at a position corresponding to position 139; V at a position corresponding to position 139; M at a position corresponding to position 139; C at a position corresponding to position 139; P at a position corresponding to position 139; P at a position corresponding to position 139; S at a position corresponding to position 139; L at a position corresponding to position 139; I at a position corresponding to position 139; H at a position corresponding to position 139; A at a position corresponding to position 139; G at a position corresponding to position 139; F at a position corresponding to position 139; N at a position corresponding to position 139; W at a position corresponding to position 139; Y at a position corresponding to position 139; E at a position corresponding to position 139; E at a position corresponding to position 141; I at a position corresponding to position 141; R at a position corresponding to position 141; S at a position corresponding to position 141; L at a position corresponding to position 141; A at a position corresponding to position 141; D at a position corresponding to position 141; W at a position corresponding to position 141; H at a position corresponding to position 141; N at a position corresponding to position 141; L at a position corresponding to position 142; M at a position corresponding to position 142; V at a position corresponding to position 142; T at a position corresponding to position 146; N at a position corresponding to position 146; Q at a position corresponding to position 146; K at a position corresponding to position 146; S at a position corresponding to position 146; D at a position corresponding to position 146; A at a position corresponding to position 146; Y at a position corresponding to position 146; V at a position corresponding to position 146; R at a position corresponding to position 147; F at a position corresponding to position 147; H at a position corresponding to position 147; W at a position corresponding to position 147; T at a position corresponding to position 147; C at a position corresponding to position 147; S at a position corresponding to position 147; V at a position corresponding to position 147; Q at a position corresponding to position 147; M at a position corresponding to position 147; R at a position corresponding to position 148; R at a position corresponding to position 148; I at a position corresponding to position 148; T at a position corresponding to position 148; G at a position corresponding to position 148; G at a position corresponding to position 148; V at a position corresponding to position 148; A at a position corresponding to position 148; A at a position corresponding to position 148; W at a position corresponding to position 148; P at a position corresponding to position 148; S at a position corresponding to position 148; N at a position corresponding to position 148; S at a position corresponding to position 150; E at a position corresponding to position 150; G at a position corresponding to position 150; M at a position corresponding to position 150; M at a position corresponding to position 150; T at a position corresponding to position 150; W at a position corresponding to position 150; A at a position corresponding to position 150; N at a position corresponding to position 150; K at a position corresponding to position 150; L at a position corresponding to position 150; L at a position corresponding to position 150; V at a position corresponding to position 150; D at a position corresponding to position 150; H at a position corresponding to position 150; G at a position corresponding to position 152; C at a position corresponding to position 152; F at a position corresponding to position 152; L at a position corresponding to position 152; L at a position corresponding to position 152; L at a position corresponding to position 152; P at a position corresponding to position 152; R at a position corresponding to position 152; H at a position corresponding to position 152; T at a position corresponding to position 152; Y at a position corresponding to position 152; K at a position corresponding to position 152; D at a position corresponding to position 152; W at a position corresponding to position 152; I at a position corresponding to position 152; A at a position corresponding to position 152; S at a position corresponding to position 152; R at a position corresponding to position 152; G at a position corresponding to position 153; H at a position corresponding to position 153; V at a position corresponding to position 153; T at a position corresponding to position 153; P at a position corresponding to position 153; F at a position corresponding to position 153; D at a position corresponding to position 153; Q at a position corresponding to position 153; Y at a position corresponding to position 153; L at a position corresponding to position 154; C at a position corresponding to position 154; S at a position corresponding to position 154; I at a position corresponding to position 154; M at a position corresponding to position 155; H at a position corresponding to position 156; L at a position corresponding to position 156; E at a position corresponding to position 156; A at a position corresponding to position 156; W at a position corresponding to position 156; C at a position corresponding to position 156; P at a position corresponding to position 156; P at a position corresponding to position 156; V at a position corresponding to position 156; V at a position corresponding to position 156; K at a position corresponding to position 156; S at a position corresponding to position 156; G at a position corresponding to position 156; T at a position corresponding to position 156; Y at a position corresponding to position 156; R at a position corresponding to position 156; M at a position corresponding to position 156; K at a position corresponding to position 157; D at a position corresponding to position 157; F at a position corresponding to position 157; R at a position corresponding to position 157; H at a position corresponding to position 157; L at a position corresponding to position 157; N at a position corresponding to position 157; N at a position corresponding to position 157; Y at a position corresponding to position 157; S at a position corresponding to position 157; T at a position corresponding to position 157; A at a position corresponding to position 157; A at a position corresponding to position 157; Q at a position corresponding to position 157; P at a position corresponding to position 157; P at a position corresponding to position 157; V at a position corresponding to position 157; V at a position corresponding to position 157; M at a position corresponding to position 157; S at a position corresponding to position 158; Y at a position corresponding to position 158; R at a position corresponding to position 158; L at a position corresponding to position 158; V at a position corresponding to position 158; V at a position corresponding to position 158; C at a position corresponding to position 158; A at a position corresponding to position 158; W at a position corresponding to position 158; I at a position corresponding to position 158; F at a position corresponding to position 158; Q at a position corresponding to position 158; T at a position corresponding to position 158; G at a position corresponding to position 158; K at a position corresponding to position 158; N at a position corresponding to position 158; D at a position corresponding to position 158; R at a position corresponding to position 159; S at a position corresponding to position 159; Q at a position corresponding to position 159; P at a position corresponding to position 159; V at a position corresponding to position 159; K at a position corresponding to position 159; A at a position corresponding to position 159; Y at a position corresponding to position 159; E at a position corresponding to position 159; T at a position corresponding to position 159; M at a position corresponding to position 159; I at a position corresponding to position 159; W at a position corresponding to position 159; W at a position corresponding to position 159; L at a position corresponding to position 159; C at a position corresponding to position 159; A at a position corresponding to position 160; H at a position corresponding to position 160; N at a position corresponding to position 160; W at a position corresponding to position 160; R at a position corresponding to position 160; M at a position corresponding to position 160; Q at a position corresponding to position 160; V at a position corresponding to position 160; S at a position corresponding to position 160; E at a position corresponding to position 160; L at a position corresponding to position 160; T at a position corresponding to position 160; S at a position corresponding to position 161; C at a position corresponding to position 161; L at a position corresponding to position 161; R at a position corresponding to position 161; R at a position corresponding to position 161; G at a position corresponding to position G; W at a position corresponding to position 161; Y at a position corresponding to position 161; E at a position corresponding to position 161; P at a position corresponding to position 161; T at a position corresponding to position 161; H at a position corresponding to position 161; I at a position corresponding to position 161; V at a position corresponding to position 161; F at a position corresponding to position 161; Q at a position corresponding to position 161; S at a position corresponding to position 164; W at a position corresponding to position 166; D at a position corresponding to position 167; R at a position corresponding to position 167; A at a position corresponding to position 167; S at a position corresponding to position 167; S at a position corresponding to position 167; F at a position corresponding to position 167; Y at a position corresponding to position 167; P at a position corresponding to position 167; T at a position corresponding to position 167; V at a position corresponding to position 167; L at a position corresponding to position 167; M at a position corresponding to position 167; N at a position corresponding to position 167; G at a position corresponding to position 167; K at a position corresponding to position 167; E at a position corresponding to position 167; R at a position corresponding to position 168; L at a position corresponding to position 170; R at a position corresponding to position 170; R at a position corresponding to position 170; I at a position corresponding to position 170; T at a position corresponding to position 170; Q at a position corresponding to position 170; G at a position corresponding to position 170; S at a position corresponding to position 170; H at a position corresponding to position 170; M at a position corresponding to position 170; K at a position corresponding to position 170; S at a position corresponding to position 171; M at a position corresponding to position 171; N at a position corresponding to position 171; P at a position corresponding to position 171; R at a position corresponding to position 171; Y at a position corresponding to position 171; A at a position corresponding to position 171; Q at a position corresponding to position 171; H at a position corresponding to position 171; L at a position corresponding to position 171; W at a position corresponding to position 171; C at a position corresponding to position 171; K at a position corresponding to position 171; E at a position corresponding to position 171; D at a position corresponding to position 171; Y at a position corresponding to position 172; T at a position corresponding to position 172; P at a position corresponding to position 172; A at a position corresponding to position 172; L at a position corresponding to position 172; Q at a position corresponding to position 172; E at a position corresponding to position 172; M at a position corresponding to position 172; D at a position corresponding to position 172; V at a position corresponding to position 172; R at a position corresponding to position 172; W at a position corresponding to position 172; N at a position corresponding to position 172; C at a position corresponding to position 173; L at a position corresponding to position 173; K at a position corresponding to position 173; W at a position corresponding to position 173; W at a position corresponding to position 173; S at a position corresponding to position 173; A at a position corresponding to position 173; R at a position corresponding to position 173; N at a position corresponding to position 173; T at a position corresponding to position 173; D at a position corresponding to position 173; V at a position corresponding to position 173; F at a position corresponding to position 173; M at a position corresponding to position 173; Y at a position corresponding to position 173; P at a position corresponding to position 173; I at a position corresponding to position 175; T at a position corresponding to position 175; N at a position corresponding to position 175; V at a position corresponding to position 175; S at a position corresponding to position 175; R at a position corresponding to position 175; G at a position corresponding to position 175; A at a position corresponding to position 175; F at a position corresponding to position 175; C at a position corresponding to position 175; Q at a position corresponding to position 175; Y at a position corresponding to position 175; L at a position corresponding to position 175; H at a position corresponding to position 175; P at a position corresponding to position 175; E at a position corresponding to position 175; F at a position corresponding to position 176; Q at a position corresponding to position 176; V at a position corresponding to position 176; T at a position corresponding to position 176; C at a position corresponding to position 176; L at a position corresponding to position 176; P at a position corresponding to position 179; L at a position corresponding to position 179; E at a position corresponding to position 179; G at a position corresponding to position 179; G at a position corresponding to position 179; S at a position corresponding to position 179; A at a position corresponding to position 179; K at a position corresponding to position 179; T at a position corresponding to position 179; I at a position corresponding to position 179; R at a position corresponding to position 179; N at a position corresponding to position 179; W at a position corresponding to position 179; Q at a position corresponding to position 179; V at a position corresponding to position 179; C at a position corresponding to position 179; M at a position corresponding to position 180; P at a position corresponding to position 180; K at a position corresponding to position 180; Y at a position corresponding to position 180; Q at a position corresponding to position 180; R at a position corresponding to position 180; A at a position corresponding to position 180; T at a position corresponding to position 180; I at a position corresponding to position 180; F at a position corresponding to position 180; C at a position corresponding to position 180; G at a position corresponding to position 180; S at a position corresponding to position 180; N at a position corresponding to position 180; D at a position corresponding to position 180; S at a position corresponding to position 181; Q at a position corresponding to position 181; A at a position corresponding to position 181; T at a position corresponding to position 181; E at a position corresponding to position 181; C at a position corresponding to position 182; P at a position corresponding to position 182; P at a position corresponding to position 182; S at a position corresponding to position 182; T at a position corresponding to position 182; R at a position corresponding to position 182; D at a position corresponding to position 182; A at a position corresponding to position 182; F at a position corresponding to position 182; L at a position corresponding to position 182; I at a position corresponding to position 182; Y at a position corresponding to position 182; Q at a position corresponding to position 182; W at a position corresponding to position 182; M at a position corresponding to position 182; G at a position corresponding to position 182; K at a position corresponding to position 183; W at a position corresponding to position 183; W at a position corresponding to position 183; E at a position corresponding to position 183; A at a position corresponding to position 183; T at a position corresponding to position 183; N at a position corresponding to position 183; H at a position corresponding to position 183; V at a position corresponding to position 183; C at a position corresponding to position 183; M at a position corresponding to position 183; G at a position corresponding to position 183; S at a position corresponding to position 183; S at a position corresponding to position 185; C at a position corresponding to position 197; V at a position corresponding to position 201; M at a position corresponding to position 201; E at a position corresponding to position 203; A at a position corresponding to position 204; M at a position corresponding to position 205; I at a position corresponding to position 205; A at a position corresponding to position 207; M at a position corresponding to position 207; D at a position corresponding to position 208; V at a position corresponding to position 208; P at a position corresponding to position 208; G at a position corresponding to position 208; A at a position corresponding to position 208; K at a position corresponding to position 208; N at a position corresponding to position 208; F at a position corresponding to position 208; Q at a position corresponding to position 208; W at a position corresponding to position 208; T at a position corresponding to position 208; E at a position corresponding to position 208; C at a position corresponding to position 208; R at a position corresponding to position 208; L at a position corresponding to position 208; T at a position corresponding to position 210; P at a position corresponding to position 211; Rat a position corresponding to position 211; K at a position corresponding to position 211; G at a position corresponding to position 211; M at a position corresponding to position 211; M at a position corresponding to position 211; N at a position corresponding to position 211; N at a position corresponding to position 211; V at a position corresponding to position 211; Q at a position corresponding to position 211; S at a position corresponding to position 211; A at a position corresponding to position 211; E at a position corresponding to position 212; T at a position corresponding to position 212; N at a position corresponding to position 212; S at a position corresponding to position 212; P at a position corresponding to position 212; Q at a position corresponding to position 212; F at a position corresponding to position 212; H at a position corresponding to position 212; and Y at a position corresponding to position 212, with reference to amino acid positions set forth in SEQ ID NO:2, wherein corresponding amino acid positions are identified by alignment of the MMP-1 polypeptide with the polypeptide set forth in SEQ ID NO:2.

11. The method of claim 8, wherein the modified MMP-1 or catalytically active fragment comprises an amino acid replacement at a calcium binding site and the amino acid replacement is at an amino acid position corresponding to a position selected from among 105, 139, 156, 157, 159, 161, 171, 173, 175, 179, 180, 182, 266, 310, 359 and 408, with reference to amino acid positions set forth in SEQ ID NO:2, wherein corresponding amino acid positions are identified by alignment of the MMP-1 polypeptide with the polypeptide set forth in SEQ ID NO:2.

12. The method of claim 11, wherein:
the unmodified MMP-1 comprises an acidic amino acid at the modified amino acid position that is an aspartic acid (D) or glutamic acid (E); and
the amino acid replacement is replacement by an amino acid residue that is a non-acidic amino acid residue.

13. The method of claim 12, wherein the amino acid replacement is selected from among:
replacement by a neutral amino acid residue selected from among cysteine (C), asparagine (N), glutamine (Q), threonine (T), tyrosine (Y), serine (S) and glycine (G);
replacement by a hydrophobic amino acid residue selected from among phenylalanine (F), methionine (M), tryptophan (W), isoleucine (I), valine (V), leucine (L), alanine (A) and proline (P); and
replacement by a basic amino acid residue selected from among histidine (H), lysine (K) and arginine (R).

14. The method of claim 13, wherein the modified MMP-1 or catalytically active fragment comprises an amino acid replacement selected from among replacement with: A at a position corresponding to position 105; I at a position corresponding to position 105; N at a position corresponding to position 105; L at a position corresponding to position 105; G at a position corresponding to position 105; R at a position corresponding to position 156; H at a position corresponding to position 156; K at a position corresponding to position 156; T at a position corresponding to position 156; N at a position corresponding to position 179; T at a position corresponding to position 180; F at a position corresponding to position 180; and T at a position corresponding to position 182, with reference to amino acid positions set forth in SEQ ID NO:2.

15. The method of claim 14, wherein the modified MMP-1 or catalytically active fragment comprises an amino acid replacement that is replacement with T at a position corresponding to position 156, with reference to amino acid positions set forth in SEQ ID NO:2.

16. The method of claim 14, wherein the modified MMP-1 or catalytically active fragment comprises an amino acid replacement that is replacement with N at a position corresponding to position 179, with reference to amino acid positions set forth in SEQ ID NO:2.

17. The method of claim 14, wherein the modified MMP-1 or catalytically active fragment comprises replacement with T at a position corresponding to position 156 and replacement with N at a position corresponding to position 179, with reference to amino acid positions set forth in SEQ ID NO:2.

18. The method of claim 11, wherein:
the modified MMP-1 or catalytically active fragment comprises an amino acid replacement at an amino acid position corresponding to position 159, with reference to amino acid positions set forth in SEQ ID NO:2;
the amino acid replacement is replacement by a hydrophobic amino acid residue; and
corresponding amino acid positions are identified by alignment of the MMP-1 polypeptide with the polypeptide set forth in SEQ ID NO:2.

19. The method of claim 18, wherein the amino acid replacement is by a hydrophobic amino acid residue selected from among phenylalanine (F), methionine (M), tryptophan (W), isoleucine (I), valine (V), leucine (L), alanine (A) and proline (P).

20. The method of claim 19, wherein the modified MMP-1 or catalytically active fragment comprises an amino acid replacement with V at a position corresponding to position 159.

21. The method of claim 20, wherein the modified MMP-1 or catalytically active fragment comprises an amino acid replacement of the amino acid at a position corresponding to position 159 with V and replacement of the amino acid at a position corresponding to position 208 with K.

22. The method of claim 1, wherein the modified MMP-1 or catalytically active fragment thereof comprises an amino acid replacement of an amino acid at a position corresponding to position 227 with glutamic acid (E), with reference to amino acid positions set forth in SEQ ID NO:2; and
corresponding amino acid positions are identified by alignment of the MMP-1 polypeptide with the polypeptide set forth in SEQ ID NO:2.

23. The method of claim 22, wherein the amino acid replacement is V227E, with reference to amino acid positions set forth in SEQ ID NO:2.

24. The method of claim 1, wherein the modified MMP-1 or catalytically active fragment comprises the sequence of amino acids set forth in any of SEQ ID NOS:162-167, or a sequence of amino acids that exhibits at least 85% sequence identity to any of SEQ ID NOS:162-167 and contains the amino acid replacement(s).

25. The method of claim 1, wherein the composition comprises calcium at a concentration that is from or from about 1.5 mM to 50 mM.

26. The method of claim 1, wherein the composition comprises calcium at a concentration that is at least or about at least or is greater than 10 mM.

27. The method of claim 1, wherein the modified MMP-1 or catalytically active fragment exhibits at least 2-fold decreased matrix metalloprotease activity in the presence of physiological levels of extracellular calcium compared to its activity in the presence of a calcium concentration that is greater than the physiological level.

28. The method of claim 1, wherein the modified MMP or catalytically active fragment exhibits at least 85% sequence identity to SEQ ID NO:5 or a catalytically active fragment thereof.

29. The method of claim 1, wherein the modified MMP or catalytically active fragment comprises up to 1, 2, 3, 4, 5, 6, 7, 8, 9, 10, 11, 12, 13, 14, 15, 16, 17, 18, 19 or 20 amino acid replacements compared to the unmodified MMP-1.

30. The method of claim 1, wherein the modified MMP-1 is a zymogen and the method further comprises processing the zymogen to a mature enzyme prior to administration.

31. The method of claim 1, comprising administering a calcium-chelating agent to reduce the activity of the modified MMP-1 or catalytically active fragment.

32. The method of claim 31, wherein the calcium-chelating agent is administered at a concentration of 1 μM to 100 mM.

33. The method of claim 31, wherein the calcium-chelating agent is administered prior to, intermittently with, subsequently to, or simultaneously with administration of the composition comprising the modified MMP-1 or catalytically active fragment.

34. The method of claim 1, wherein the composition(s) is(are) administered to the extracellular matrix (ECM) of the subject.

35. The method of claim 1, wherein administration is selected from among subcutaneous, intramuscular, intralesional and intradermal routes of administration.

36. The method of claim 1, wherein the modified MMP-1 or catalytically active fragment is administered in an amount that is from or from about 10 μg to 100 mg.

37. The method of claim 1, wherein:
the component of the extracellular matrix is a collagen;
the fibrotic disease or condition is a collagen-mediated disease or condition; and
the modified MMP-1 or catalytically active fragment exhibits activity to cleave a collagen.

38. The method of claim 37, wherein the component of the extracellular matrix is a collagen and the modified MMP-1 degrades one or both of collagen type I and collagen type III.

39. The method of claim 37, wherein:
the collagen-mediated disease or condition is associated with irregular formation of collagen fibers; and
the modified MMP severs the fibers, thereby treating the disease or condition.

40. The method of claim 37, wherein the collagen-mediated disease or condition is selected from among cellulite, Dupuytren's disease, Peyronie's disease, Ledderhose fibrosis, stiff joints, existing scars, scleroderma, lymphedema and collagenous colitis.

41. The method of claim 40, wherein the collagen-mediated disease or condition is stiff joints that is frozen shoulder.

42. The method of claim 40, wherein the collagen-mediated disease or condition is existing scars that is selected from among surgical adhesions, keloids, hypertrophic scars and depressed scars.

43. The method of claim 1, wherein the fibrotic disease or condition is herniated protruding discs.
